(19) United States
(12) Patent Application Publication
(10) Pub. No.: US 2026/0183437 A1
(43) Pub. Date: Jul. 2, 2026

(54) VHH ANTIBODY CONJUGATES WITH HETEROARYL CHELATORS

(71) Applicant: ABDERA THERAPEUTICS INC., South San Francisco, CA (US)

(72) Inventors: Michael J. ABRAMS, Custer, WA (US); Adam Daniel JUDGE, Bainbridge Island, WA (US); Alexander Laurence MANDEL, Vancouver, BC (CA); Douglas Bruce MACKAY, Ottawa, ON (CA)

(21) Appl. No.: 19/104,953

(22) PCT Filed: Aug. 21, 2023
(86) PCT No.: PCT/US2023/072591

§ 371 Date: Feb. 19, 2025

Related U.S. Application Data
(60) Provisional application No. 63/373,190, filed on Aug. 22, 2022.

(51) Int. Cl. A61K 51/10; A61K 51/04; A61P 35/00

(52) U.S. Cl. CPC A61K 51/1096; A61K 51/0478; A61K 51/1051; A61K 51/1054; A61P 35/00; A61K 2121/00; A61K 2123/00

(57) ABSTRACT

Described herein are immunoconjugates comprising an: a) antigen binding region; b) an immunoglobulin heavy chain constant region; and c) a radioisotope chelating agent; wherein the molecular weight of said immunoconjugate is between 60 and 110 kDa. The immunoconjugates can be used to deliver alpha and beta emitters for the treatment of tumors or cancer.

### CROSS REFERENCE

This application claims the benefit of U.S. Application No. 63/373,190, filed Aug. 22, 2022, which is hereby incorporated by reference in its entirety.

### BACKGROUND

The exquisite specificity of antibodies, such as IgGs, to their antigens makes antibodies a premier targeting platform for therapeutics; however, the typical serum half-life of at least three weeks for an IgG is disadvantageous for the delivery of radioisotopes including alpha-emitting isotopes such as actinium-225 (²²⁵Ac) and beta-emitting isotopes such as lutetium-177 (¹⁷⁷Lu) and yttrium-90 (⁹⁰Y), in particular due to prolonged exposure and chronic off-target toxicities. The advent of engineered smaller antibody formats (e.g. monomeric scFv's, heavy-chain only antibodies, or single-domain antibody fragments) provides the exquisite specificity of a full-size antibody (e.g. an IgG (˜150 kDa)) in a smaller format (e.g. 15 to 30 kDa) and with a much shorter serum half-life (e.g. 30 minutes to 2 hours) (Bates A, Power, C, Antibodies (Basel) 8: 28 (2019)). Unfortunately, these short half-lives do not allow sufficient time for efficacious target binding due to poor retention and tumor uptake, and furthermore plasma clearance of these small antibody formats by the renal system can lead to isotope accumulation in renal tissues and problematic off-target toxicities.

²²⁵Ac is among the most cytotoxic of the α-emitting radioisotopes, and a single decay event can effectively destroy a cancer cell by causing double-strand DNA breaks and subsequent cell death. The potency of α-emitting radioisotopes makes them attractive as cell killing agents, capable of overcoming the acquired resistance observed in response to other therapies. Unfortunately, however, numerous challenges remain with respect to systemic administration and the achievement of desired dosimetry in target versus non-target tissues as a result of decay events in different locations in vivo. Key to the application of α-emitting radionuclides as targeted therapeutics is the ability to modulate the distribution of daughter nuclides in vivo so as to limit toxicity. This in turn relates to the timing of creation of parent nuclide, the time of therapeutic administration, the decay path and half-lives of daughter nuclides, circulation time, and the biodistribution and pharmacokinetics of delivery vehicles. Unfortunately, the emission of an α particle also typically produces a recoil energy large enough to decouple the daughter nuclide from a chelator, with the potential to separate daughter nuclide from its targeting vehicle, resulting in the subsequent redistribution of ‘free’ daughter nuclides that can induce multiple toxicities. See e.g. Robertson A et al., Curr Radiopharm 11:156 (2018). Accordingly, renal toxicity caused by ²²⁵Ac recoil daughter nuclides (e.g. 213-Bi) has thus far been a major constraint on the therapeutic use of ²²⁵Ac (see e.g. Jaggi J et al., Cancer Res. 65:4888 (2005)).

A further and confounding issue with respect to the use of antibodies and antibody fragments with α-emitting radioisotopes in therapeutics is that intervening radioactive decay can damage antibody components and targeting sequences in particular, even prior to treatment. Before an α-emitter labelled antibody fragment can be administered to a patient, radiolysis of the antibody fragment may occur thereby reducing the amount of targeting (see e.g., Larsen R, Bruland O, J Labelled Cmpd Radiopharm. 36: 1009-18 (1995)), and at the higher specific activities needed for therapeutic dosing immunoreactivity can fall rapidly along with radiochemical quality. Salako et al., J Nucl Med. 39(4):667-670 (1998). For example, the high ionization density released by an α-emitter compromised the immunoreactivity of isotope-labeled Fab fragments via radiolysis at doses of 1,000 gray (Gy) or higher. Similarly, significant radiolysis of α-emitting isotope-labeled antibodies was observed at doses over 1,200 Gy (Zalutsky M et al., J Nucl Med. 42(10):1508-15 (2001)). As such, the identification of an appropriate targeted delivery vehicle for α-emitting radioisotopes is not straightforward.

Moreover, there are additional issues for targeted radioscope delivering platforms, including for alpha-emitting and beta-emitting radioisotopes, requiring simultaneous optimization when designing such platforms, such as, e.g., immunogenicity, specificity, tissue penetration, stability, ease of manufacturing, and acceptable therapeutic window.

### SUMMARY

The present invention relates to immunoconjugates or radioimmunoconjugate, compositions comprising the same, and methods of using such immunoconjugates and compositions. The immunoconjugates and compositions of the present invention have numerous uses, e.g., for delivery of a radioisotope to kill a target cell (e.g. a cancer cell expressing a target antigen bound by the radioimmunoconjugate); for detection and characterization of malignant cells within a subject (e.g. target antigen expression); and for diagnosis and treatment of a variety of diseases and conditions, such as, e.g., cancers, tumors, and other growth abnormalities involving antigen-expressing cells.

The present invention addresses a number of challenges inherent in the targeted delivery of alpha particle emitters in vivo through the selection and particular combination of specific delivery platform components. The alpha particle emitting radioisotope-delivery platforms of the present invention provide shorter half-lives compared to traditional IgGs, but longer half-lives than smaller monomeric antibody fragment formats. Such half-lives allow for a reduction in toxicity due to the alpha emitter, while preserving the antibody fragment long enough in the body to exert therapeutic activity. For example, the alpha particle emitting radioisotope-delivery platforms of the current disclosure exhibit enhanced tumor targeting and reduced accumulation in radiosensitive tissues such as the bone-marrow and kidney. Further and surprisingly, the alpha particle emitting radioisotope-delivery platforms of the present invention exhibit excellent tumor binding and labeling properties for tumors with different antigen densities, which can be a limitation for some use of some immunoconjugates.

Described herein in one aspect is an immunoconjugate comprising an: a) antigen binding region; b) an immunoglobulin heavy chain constant region; and c) a chelating agent; wherein the molecular weight of the immunoconjugate is between 60 and 110 kDa. In certain embodiments, the antigen binding region comprises an scFv polypeptide or a VHH polypeptide. In certain embodiments, the antigen binding region comprises an scFv polypeptide. In certain embodiments, the antigen binding region comprises a VHH polypeptide. In certain embodiments, the antigen binding region is humanized.

In some embodiments, described herein is a compound of Formula (I), or a pharmaceutically acceptable salt thereof:

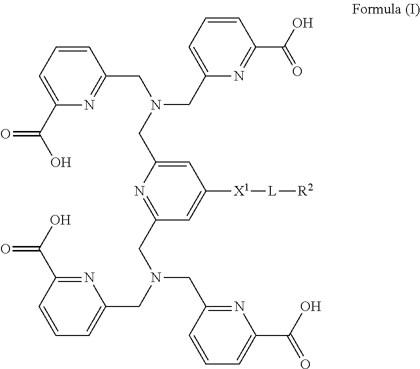

wherein:
X¹ is —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—, -(unsubstituted or substituted C₁-C₆alkylene)-X²—, —O-(unsubstituted or substituted C₁-C₆alkylene)-X²—, or —(C₄-C₂₀polyethylene glycol)-X²—;
X² is absent, —C(═O)—, —NR^{a}C(═O)—, —C(═O)NR^{a}—, or —C(═O)X⁴—;
   each R^{a} is independently selected from hydrogen, and C₁-C₄alkyl;
X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(═O)R⁵, and wherein any free carboxylic acid of any amino acid is optionally replaced with —C(═O)NH—R⁵;
R² is a moiety that is capable of reacting with an amine (—NH₂) or thiol (—SH) of a tumor targeting moiety R³;
L is a linker that is -L¹-L²-L³-L⁴-L⁵-;
L¹ is absent, unsubstituted or substituted C₁-C₁₀alkylene, unsubstituted or substituted C₁-C₁₀heteroalkylene, unsubstituted or substituted C₂-C₂₀alkenylene, unsubstituted or substituted C₂-C₂₀alkynylene, C₄-C₂₀polyethylene glycol, —(X³CH₂CH₂)ₜ—, unsubstituted or substituted cycloalkylene, unsubstituted or substituted heterocycloalkylene, unsubstituted or substituted arylene, unsubstituted or substituted heteroarylene;
   each X³ is independently selected from 0 and NR⁴;
   each t is independently 1, 2, 3, 4, 5, 6, 7, 8, 9, 10, 11, or 12;
L² is absent, unsubstituted or substituted C₁-C₁₀alkylene, unsubstituted or substituted C₁-C₁₀heteroalkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₘ—(CH₂)_{P}—, —C(═O)NR⁴—(CH₂CH₂X³)ₘ—(CH₂)ₚ—, —NR⁴C(═O)—(CH₂CH₂X³)ₘ—(CH₂)ₚ—, or —(CH₂CH₂X³)ₘ—(CH₂)ₚ—;
   each R⁴ is independently selected from hydrogen, and C₁-C₆alkyl;
   each m is independently 1, 2, 3, 4, 5, 6, 7, 8, 9, 10, 11, or 12;
   each p is independently 0, 1, or 2;
L³ is absent, or one or more independently selected groups selected from: natural or unnatural amino acids, and an optional amino(unsubstituted or substituted benzyl)carbamate; wherein any free amine (—NH₂) of an amino acid is optionally independently substituted with R⁵ or —C(═O)R⁵, wherein any free carboxylic acid of any amino acid is optionally replaced with —C(═O)NH—R⁵, and wherein substituted benzyl is substituted with 1 or 2 groups selected from halogen, —OH, —OR⁵, —CO₂H, —NHR⁵, —C(═O)NHR⁵, —NHC(═O)R⁵ and substituted C₁-C₆alkyl, wherein the substituted C₁-C₆alkyl is substituted with, —OH, —CO₂H, —NHR⁵, —C(═O)NHR⁵, and —NHC(═O)R⁵;
L⁴ is absent, unsubstituted or substituted C₁-C₁₀alkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, or —(X³CH₂CH₂)ₙ—;
   each n is independently 1, 2, 3, 4, 5, 6, 7, 8, 9, 10, 11, or 12;
   each q is independently 0, 1, or 2;
L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, —C(═O)—(X³CH₂CH₂)ₙ—, or —(X³CH₂CH₂)ₙ—;
   each n is independently 1, 2, 3, 4, 5, 6, 7, 8, 9, 10, 11, or 12;
   each q is independently 0, 1, or 2;
   each R^{a} is independently selected from hydrogen, and C₁-C₄alkyl;
   each R^{b} is independently selected from hydrogen, and C₁-C₄alkyl;
wherein heteroalkylene is an alkylene where one carbon atom is replaced with —O—, —S—, —S(═O)—, —S(═O)₂—, —S(═O)(═NH)—, —S(═O)(═NR⁶)—, —NR⁶—, —P(═O)OR^{b}—, —NR^{a}C(═O)—, —C(═O)NR^{a}—, —OC(═O)NR^{a}—, —NR^{a}C(═O)NR^{a}—, —NR^{a}C(═N—CN)NR^{a}—, —NR^{a}C(═N—R⁶)NR^{a}—, or —NR^{a}C(═O)O—;
wherein when any one of -L¹-, -L²-, -L³-, -L⁴-, and -L⁵- is substituted then -L¹-, -L²-, -L³-, -L⁴-, and -L⁵- is substituted with 1, 2, 3, or 4 groups selected from halogen, —OH, —OR⁵, —CO₂H, —NHR⁵, —C(═O)NHR⁵, —NHC(═O)R⁵ and unsubstituted or substituted C₁-C₆alkyl, wherein the substituted C₁-C₆alkyl is substituted with, —OH, —CO₂H, —NHR⁵, —C(═O)NHR⁵, or —NHC(═O)R⁵;
each R⁵ is independently selected from C₁-C₁₀alkyl, C₄-C₃₀polyethylene glycol, and unsubstituted or substituted arylene, unsubstituted or substituted heteroarylene;
each R⁶ is independently selected from C₄-C₃₀polyethylene glycol, unsubstituted or substituted arylene, unsubstituted or substituted heteroarylene, unsubstituted or substituted C₁-C₆alkyl, wherein the substituted C₁-C₆alkyl is substituted with halogen, —OH, —CO₂H, —NHR⁵, —C(═O)NHR⁵, or —NHC(═O)R⁵;
provided that when L³ is absent then at least one R⁵ is present or —NH-L¹- is one or more independently selected natural or unnatural amino acids; or
provided that —X¹-L-R² is not

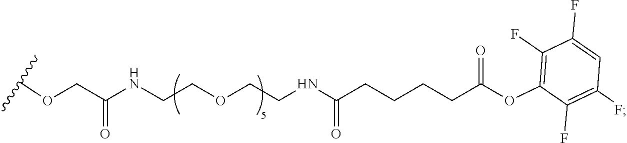

or a radionuclide complex thereof.

In some embodiments, R² is a moiety that is capable of reacting with an amine (—NH₂) of the tumor targeting moiety R³ and comprises a tetrafluorophenyl ester, pentafluorophenyl ester, dinitrophenyl ester, succinimide ester, sulfosuccinimide ester, or isothiocyanate.

In some embodiments, R² is a moiety that is capable of reacting with an amine (—NH₂) of the tumor targeting moiety R³ and comprises:

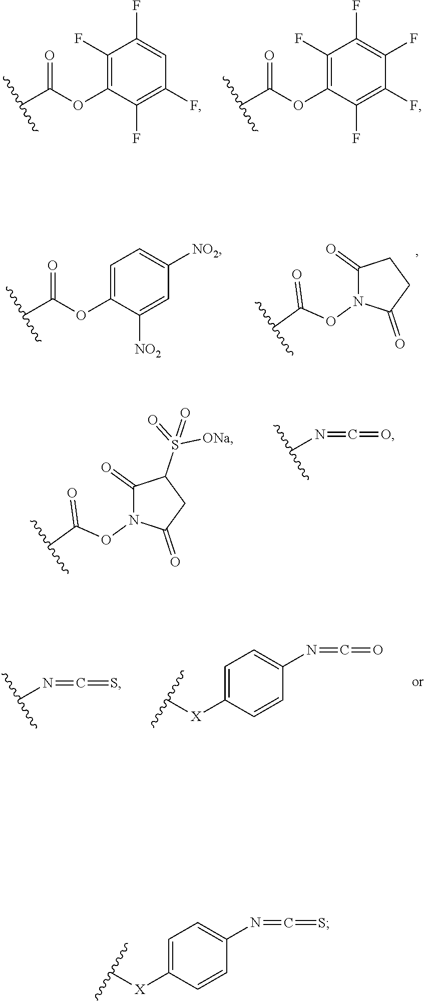

X is absent, —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—; each R^{a} is independently selected from hydrogen, and C₁-C₄alkyl.

In some embodiments, described herein is an immunoconjugate that has the structure of Formula (II), or a pharmaceutically acceptable salt thereof:

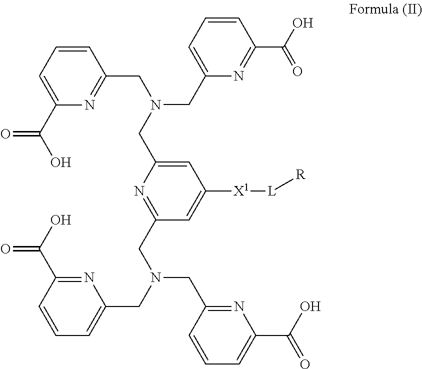

wherein:
   R is —C(═O)NHR³,

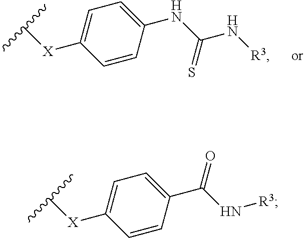

X is absent, —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—;
each R^{a} is independently selected from hydrogen, and C₁-C₄alkyl;
—NH—R³ is a tumor targeting moiety;
X¹ is —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—, -(unsubstituted or substituted C₁-C₆alkylene)-X²—, —O-(unsubstituted or substituted C₁-C₆alkylene)-X²—, or —(C₄-C₂₀polyethylene glycol)-X²—;
X² is absent, —C(═O)—, —NR^{a}C(═O)—, —C(═O)NR^{a}—, or —C(═O)X⁴—;
X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(═O)R⁵, and wherein any free carboxylic acid of any amino acid is optionally replaced with —C(═O)NH—R⁵;
L is a linker that is -L¹-L²-L³-L⁴-L⁵-;
L¹ is absent, unsubstituted or substituted C₁-C₁₀alkylene, unsubstituted or substituted C₁-C₁₀heteroalkylene, unsubstituted or substituted C₂-C₂₀alkenylene, unsubstituted or substituted C₂-C₂₀alkynylene, C₄-C₂₀polyethylene glycol, —(X³CH₂CH₂)ₜ—, unsubstituted or substituted cycloalkylene, unsubstituted or substituted heterocycloalkylene, unsubstituted or substituted arylene, unsubstituted or substituted heteroarylene;
   each X³ is independently selected from O and NR⁴;
   each t is independently 1, 2, 3, 4, 5, 6, 7, 8, 9, 10, 11, or 12;
L² is absent, unsubstituted or substituted C₁-C₁₀alkylene, unsubstituted or substituted C₁-C₁₀heteroalkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₘ—(CH₂)_{P}—, —C(═O)NR⁴—(CH₂CH₂X³)ₘ—(CH₂)ₚ—, —NR⁴C(═O)—(CH₂CH₂X³)ₘ—(CH₂)ₚ—, or —(CH₂CH₂X³)ₘ—(CH₂)ₚ—;
   each R⁴ is independently selected from hydrogen, and C₁-C₆alkyl;
   each m is independently 1, 2, 3, 4, 5, 6, 7, 8, 9, 10, 11, or 12;
   each p is independently 0, 1, or 2;
L³ is absent, or one or more independently selected groups selected from: natural or unnatural amino acids, and an optional amino(unsubstituted or substituted benzyl)carbamate; wherein any free amine (—NH₂) of an amino acid is optionally independently substituted with R⁵ or —C(═O)R⁵, wherein any free carboxylic acid of any amino acid is optionally replaced with —C(═O)NH—R⁵, and wherein substituted benzyl is substituted with 1 or 2 groups selected from halogen, —OH, —OR⁵, —CO₂H, —NHR⁵, —C(═O)NHR⁵, —NHC(═O)R⁵ and substituted C₁-C₆alkyl, wherein the substituted C₁-C₆alkyl is substituted with, —OH, —CO₂H, —NHR⁵, —C(═O)NHR⁵, and —NHC(═O)R⁵;
L⁴ is absent, unsubstituted or substituted C₁-C₁₀alkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, or —(X³CH₂CH₂)ₙ—;
   each n is independently 1, 2, 3, 4, 5, 6, 7, 8, 9, 10, 11, or 12;
   each q is independently 0, 1, or 2;
L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, —C(═O)—(X³CH₂CH₂)ₙ—, or —(X³CH₂CH₂)ₙ—;
   each n is independently 1, 2, 3, 4, 5, 6, 7, 8, 9, 10, 11, or 12;
   each q is independently 0, 1, or 2;
   each R^{a} is independently selected from hydrogen, and C₁-C₄alkyl;
   each R^{b} is independently selected from hydrogen, and C₁-C₄alkyl;
wherein heteroalkylene is an alkylene where one carbon atom is replaced with —O—, —S—, —S(═O)—, —S(═O)₂—, —S(═O)(═NH)—, —S(═O)(═NR⁶)—, —NR⁶—, —P(═O)OR^{b}—, —NR^{a}C(═O)—, —C(═O)NR^{a}—, —OC(═O)NR^{a}—, —NR^{a}C(═O)NR^{a}—, —NR^{a}C(═N—CN)NR^{a}—, —NR^{a}C(═N—R⁶)NR^{a}—, or —NR^{a}C(═O)O—;
wherein when any one of -L¹-, -L²-, -L³-, -L⁴-, and -L⁵- is substituted then -L¹-, -L²-, -L³-, -L⁴-, and -L⁵- is substituted with 1, 2, 3, or 4 groups selected from halogen, —OH, —OR⁵, —CO₂H, —NHR⁵, —C(═O)NHR⁵, —NHC(═O)R⁵ and unsubstituted or substituted C₁-C₆alkyl, wherein the substituted C₁-C₆alkyl is substituted with, —OH, —CO₂H, —NHR⁵, —C(═O)NHR⁵, or —NHC(═O)R⁵;
each R⁵ is independently selected from C₁-C₁₀alkyl, C₄-C₃₀polyethylene glycol, and unsubstituted or substituted arylene, unsubstituted or substituted heteroarylene;
each R⁶ is independently selected from C₄-C₃₀polyethylene glycol, unsubstituted or substituted arylene, unsubstituted or substituted heteroarylene, unsubstituted or substituted C₁-C₆alkyl, wherein the substituted C₁-C₆alkyl is substituted with halogen, —OH, —CO₂H, —NHR⁵, —C(═O)NHR⁵, or —NHC(═O)R⁵;
provided that when L³ is absent then at least one R⁵ is present or —NH-L¹- is one or more independently selected natural or unnatural amino acids;
or provided that —X¹-L-R is not

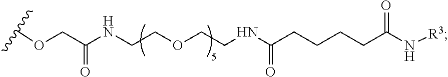

or a radionuclide complex thereof.

In some embodiments, R² is a moiety that is capable of reacting with a thiol (—SH) of a tumor targeting moiety R³ and comprises a maleimide group, a haloacetamide group, a haloacetyl group, a haloacetate group, a pyrdinylthio group, a vinylcarbonyl group, an aziridinyl group, a disulfide group, an acetylene group, a hydroxysuccinimide group or a thiol group.

In some embodiments, R² is a moiety that is capable of reacting with a thiol (—SH) of a tumor targeting moiety R³ and comprises:

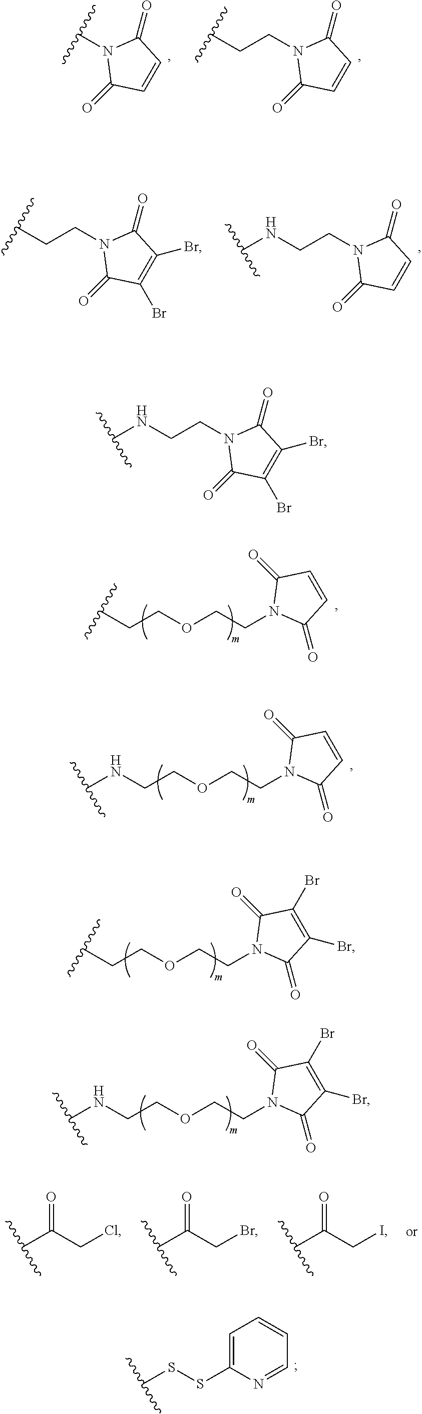

m is 0, 1, 2, 3, 4, or 5.

In some embodiments, described herein is an immunoconjugate that has the structure of Formula (III), or a pharmaceutically acceptable salt thereof:

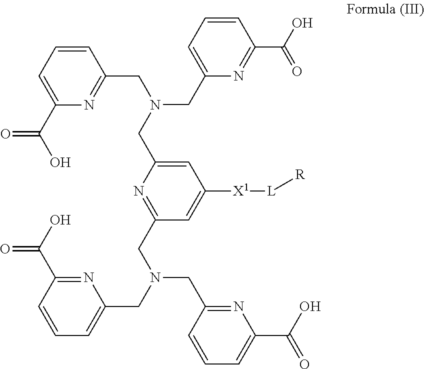

wherein:
R is

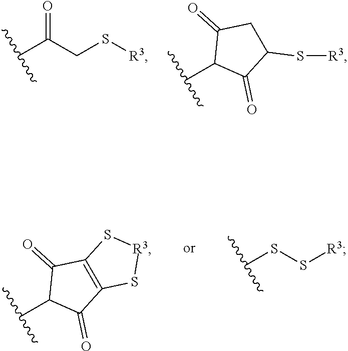

—S—R³ is a tumor targeting moiety.
X¹ is —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—, -(unsubstituted or substituted C₁-C₆alkylene)-X²—, —O-(unsubstituted or substituted C₁-C₆alkylene)-X²—, or —(C₄-C₂₀polyethylene glycol)-X²—;
X² is absent, —C(═O)—, —NR^{a}C(═O)—, —C(═O)NR^{a}—, or —C(═O)X⁴—;
   each R^{a} is independently selected from hydrogen, and C₁-C₄alkyl;
X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(═O)R⁵, and wherein any free carboxylic acid of any amino acid is optionally replaced with —C(═O)NH—R⁵;
L is a linker that is -L¹-L²-L³-L⁴-L⁵-;
L¹ is absent, unsubstituted or substituted C₁-C₁₀alkylene, unsubstituted or substituted C₁-C₁₀heteroalkylene, unsubstituted or substituted C₂-C₂₀alkenylene, unsubstituted or substituted C₂-C₂₀alkynylene, C₄-C₂₀polyethylene glycol, —(X³CH₂CH₂)ₜ—, unsubstituted or substituted cycloalkylene, unsubstituted or substituted heterocycloalkylene, unsubstituted or substituted arylene, unsubstituted or substituted heteroarylene;
   each X³ is independently selected from 0 and NR⁴;
   each t is independently 1, 2, 3, 4, 5, 6, 7, 8, 9, 10, 11, or 12;
L² is absent, unsubstituted or substituted C₁-C₁₀alkylene, unsubstituted or substituted C₁-C₁₀heteroalkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₘ—(CH₂)_{P}—, —C(═O)NR⁴—(CH₂CH₂X³)ₘ—(CH₂)ₚ—, —NR⁴C(═O)—(CH₂CH₂X³)ₘ—(CH₂)ₚ—, or —(CH₂CH₂X³)ₘ—(CH₂)ₚ—;
   each R⁴ is independently selected from hydrogen, and C₁-C₆alkyl;
   each m is independently 1, 2, 3, 4, 5, 6, 7, 8, 9, 10, 11, or 12;
   each p is independently 0, 1, or 2;
L³ is absent, or one or more independently selected groups selected from: natural or unnatural amino acids, and an optional amino(unsubstituted or substituted benzyl)carbamate; wherein any free amine (—NH₂) of an amino acid is optionally independently substituted with R⁵ or —C(═O)R⁵, wherein any free carboxylic acid of any amino acid is optionally replaced with —C(═O)NH—R⁵, and wherein substituted benzyl is substituted with 1 or 2 groups selected from halogen, —OH, —OR⁵, —CO₂H, —NHR⁵, —C(═O)NHR⁵, —NHC(═O)R⁵ and substituted C₁-C₆alkyl, wherein the substituted C₁-C₆alkyl is substituted with, —OH, —CO₂H, —NHR⁵, —C(═O)NHR⁵, and —NHC(═O)R⁵;
L⁴ is absent, unsubstituted or substituted C₁-C₁₀alkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, or —(X³CH₂CH₂)ₙ—;
   each n is independently 1, 2, 3, 4, 5, 6, 7, 8, 9, 10, 11, or 12;
   each q is independently 0, 1, or 2;
L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, —C(═O)—(X³CH₂CH₂)ₙ—, or —(X³CH₂CH₂)ₙ—;
   each n is independently 1, 2, 3, 4, 5, 6, 7, 8, 9, 10, 11, or 12;
   each q is independently 0, 1, or 2;
   each R^{a} is independently selected from hydrogen, and C₁-C₄alkyl;
   each R^{b} is independently selected from hydrogen, and C₁-C₄alkyl;
wherein heteroalkylene is an alkylene where one carbon atom is replaced with —O—, —S—, —S(═O)—, —S(═O)₂—, —S(═O)(═NH)—, —S(═O)(═NR⁶)—, —NR⁶—, —P(═O)OR^{b}—, —NR^{a}C(═O)—, —C(═O)NR^{a}—, —OC(═O)NR^{a}—, —NR^{a}C(═O)NR^{a}—, —NR^{a}C(═N—CN)NR^{a}—, —NR^{a}C(═N—R⁶)NR^{a}—, or —NR^{a}C(═O)O—;
wherein when any one of -L¹-, -L²-, -L³-, -L⁴-, and -L⁵- is substituted then -L¹-, -L²-, -L³-, -L⁴-, and -L⁵- is substituted with 1, 2, 3, or 4 groups selected from halogen, —OH, —OR⁵, —CO₂H, —NHR⁵, —C(═O)NHR⁵, —NHC(═O)R⁵ and unsubstituted or substituted C₁-C₆alkyl, wherein the substituted C₁-C₆alkyl is substituted with, —OH, —CO₂H, —NHR⁵, —C(═O)NHR⁵, and —NHC(═O)R⁵;
each R⁵ is independently selected from C₁-C₁₀alkyl, C₄-C₃₀polyethylene glycol, and unsubstituted or substituted arylene, unsubstituted or substituted heteroarylene;
each R⁶ is independently selected from C₄-C₃₀polyethylene glycol, unsubstituted or substituted arylene, unsubstituted or substituted heteroarylene, unsubstituted or substituted C₁-C₆alkyl, wherein the substituted C₁-C₆alkyl is substituted with halogen, —OH, —CO₂H, —NHR⁵, —C(═O)NHR⁵, or —NHC(═O)R⁵;
or a radionuclide complex thereof.

In some embodiments, described herein the immunoconjugate of Formula (II) has the structure of Formula (IIa), or Formula (IIb), or a pharmaceutically acceptable salt thereof:

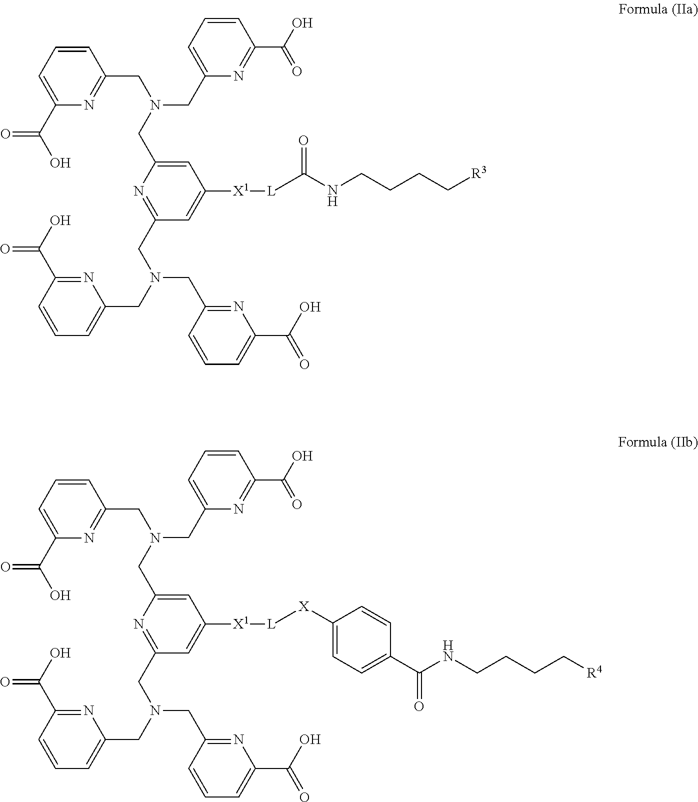

wherein: —NHCH₂CH₂CH₂CH₂— is the side chain of a lysine residue of the tumor targeting moiety R³; or a radionuclide complex thereof.

In some embodiments, described herein the immunoconjugate of Formula (II) has the structure of Formula (IIc), or a pharmaceutically acceptable salt thereof:

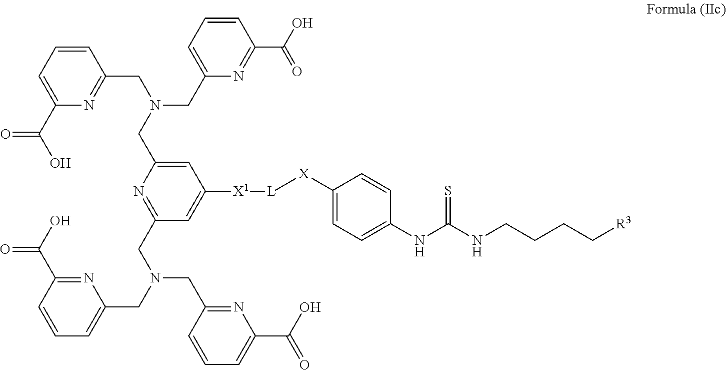

wherein: —NHCH₂CH₂CH₂CH₂— is the side chain of a lysine residue of the tumor targeting moiety R³; or a radionuclide complex thereof.

In some embodiments, described herein the immunoconjugate of Formula (III) has the structure of Formula (IIIa), or a pharmaceutically acceptable salt thereof:

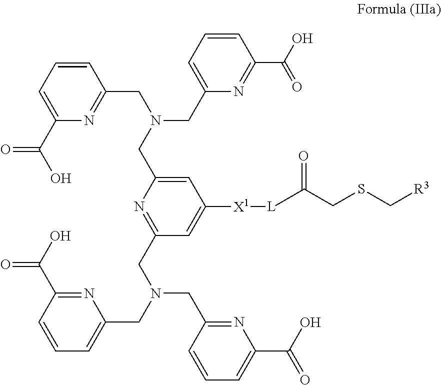

wherein: —SCH₂— is the thiol (—SH) of the side chain of a cysteine residue of the tumor targeting moiety R³; or a radionuclide complex thereof.

In some embodiments, described herein the an immunoconjugate of Formula (III) has the structure of Formula (IIIb), or Formula (IIc), or a pharmaceutically acceptable salt thereof:

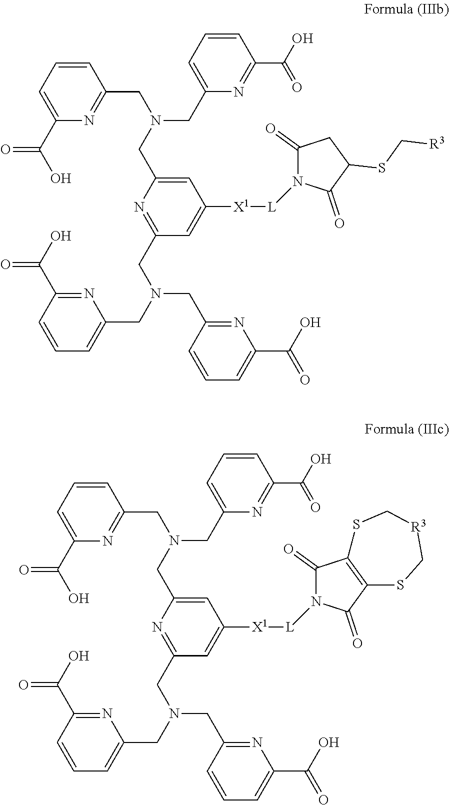

wherein: —SCH₂— is the thiol (—SH) of the side chain of a cysteine residue of the tumor targeting moiety R³; or a radionuclide complex thereof.

In some embodiments, described herein the an immunoconjugate of Formula (III) has the structure of Formula (IIId), or a pharmaceutically acceptable salt thereof:

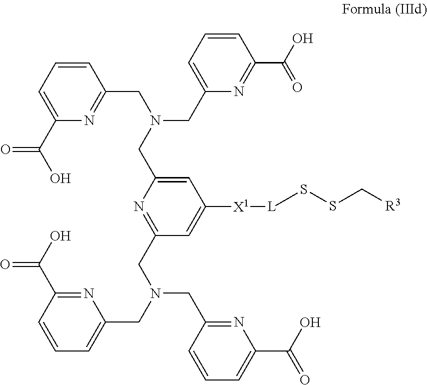

wherein: —SCH₂— is the thiol (—SH) of the side chain of a cysteine residue of the tumor targeting moiety R³; or a radionuclide complex thereof.

In some embodiments, the tumor targeting moiety R³ is a polypeptide comprising an antigen binding region and an immunoglobulin heavy chain constant region, wherein the molecular weight of the polypeptide is between 60 and 110 kDa.

In some embodiments, the antigen binding region comprises an scFv polypeptide or a VHH polypeptide. In some embodiments, the immunoglobulin heavy chain constant region comprises a CH2 domain of an immunoglobulin, CH3 domain of an immunoglobulin, or a CH2 and a CH3 domain of an immunoglobulin. In some embodiments, the immunoglobulin heavy chain constant region is an IgA, IgG1, IgG2, IgG3, or IgG4 isotype. In some embodiments, the antigen binding region is humanized, the immunoglobulin heavy chain constant region is a human immunoglobulin heavy chain constant region, or both. In some embodiments, the immunoglobulin heavy chain constant region comprises an alteration to one or more amino acid residues that reduces an effector function of the immunoglobulin heavy chain constant region or alters binding of the immunoconjugate to the neonatal Fc receptor (FcRn); or the immunoglobulin heavy chain constant region comprises an alteration to one or more amino acid residues that reduces an effector function of the immunoglobulin heavy chain constant region and alters binding of the immunoconjugate to the neonatal Fc receptor (FcRn). In some embodiments, the immunoglobulin heavy chain constant region comprises an alteration to one or more amino acid residues that reduces an effector function of the immunoglobulin heavy chain constant region; or the immunoglobulin heavy chain constant region comprises an alteration to one or more amino acid residues that alters binding of the immunoconjugate to the neonatal Fc receptor (FcRn); or both. In some embodiments, the alteration to one or more amino acid residues that reduces the effector function of the immunoglobulin heavy chain constant region is an alteration that reduces complement dependent cytotoxicity (CDC), antibody-dependent cell-cytotoxicity (ADCC), antibody-dependent cell-phagocytosis ADCP, or a combination thereof.

In certain embodiments, the antigen binding region specifically binds to HER2 or to DLL3. In certain embodiments, the antigen binding region specifically binds to HER2. In certain embodiments, the antigen binding region of the immunoconjugate comprises: a) a heavy chain CDR1 comprising the amino acid sequence set forth in SEQ ID NO: 21; b) a heavy chain CDR2 comprising the amino acid sequence set forth in SEQ ID NO: 22; and c) a heavy chain CDR3 comprising the amino acid sequence set forth in SEQ ID NO: 23 and that binds to HER2. In certain embodiments, the antigen binding region of the immunoconjugate comprises a sequence that is at least 85%, 90%, 95%, 97%, 98%, 99%, or 100% identical to the sequence set forth in SEQ ID NO: 20 and that binds to HER2. In certain embodiments, the antigen binding region specifically binds to DLL3. In certain embodiments, the antigen binding region of the immunoconjugate comprises: a) a heavy chain CDR1 comprising the amino acid sequence set forth in SEQ ID NO: 31; b) a heavy chain CDR2 comprising the amino acid sequence set forth in SEQ ID NO: 32; and c) a heavy chain CDR3 comprising the amino acid sequence set forth in SEQ ID NO: 33 and that binds to DLL3. In certain embodiments, the antigen binding region of the immunoconjugate comprises a sequence that is at least 85%, 90%, 95%, 97%, 98%, 99%, or 100% identical to the sequence set forth in SEQ ID NO: 30 and that binds to DLL3. In certain embodiments, the immunoglobulin heavy chain constant region comprises a CH2 domain of an immunoglobulin, CH3 domain of an immunoglobulin, or a CH2 and a CH3 domain of an immunoglobulin. In certain embodiments, the immunoglobulin heavy chain constant region comprises a CH2 and a CH3 domain of an immunoglobulin. In certain embodiments, the immunoglobulin heavy chain constant region is a human immunoglobulin heavy chain constant region. In certain embodiments, the immunoglobulin heavy chain constant region is an IgA, IgG1, IgG2, IgG3, or IgG4 isotype. In certain embodiments, the immunoglobulin heavy chain constant region is an IgG1 isotype. In certain embodiments, the immunoglobulin heavy chain constant region is an IgG4 isotype. In certain embodiments, the immunoglobulin heavy chain constant region comprises an alteration to one or more amino acid residues that reduces an effector function of the immunoglobulin heavy chain constant region or alters binding of the immunoconjugate to the neonatal Fc receptor (FcRn). In certain embodiments, the immunoglobulin heavy chain constant region comprises an alteration to one or more amino acid residues that reduces an effector function of the immunoglobulin heavy chain constant region and alters binding of the immunoconjugate to the neonatal Fc receptor (FcRn). In certain embodiments, the immunoglobulin heavy chain constant region comprises an alteration to one or more amino acid residues that reduces an effector function of the immunoglobulin heavy chain constant region. In certain embodiments, the immunoglobulin heavy chain constant region comprises an alteration to one or more amino acid residues that alters binding of the immunoconjugate to the neonatal Fc receptor (FcRn). In certain embodiments, the alteration to one or more amino acid residues that reduces the effector function of the immunoglobulin heavy chain constant region is an alteration that reduces complement dependent cytotoxicity (CDC), antibody-dependent cell-cytotoxicity (ADCC), antibody-dependent cell-phagocytosis ADCP, or a combination thereof. In certain embodiments, the alteration to one or more amino acid residues that reduces the effector function of the immunoglobulin heavy chain constant region is selected from the list consisting of: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271 T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(ppp), per EU numbering. In certain embodiments, the alteration to one or more amino acid residues that reduces the effector function of the immunoglobulin heavy chain constant region comprises L234A, L235E, G237A, A330S, and P331S per EU numbering. In certain embodiments, the amino acid alteration to one or more amino acid residues that alters binding of the immunoconjugate to the neonatal Fc receptor (FcRn) reduces the serum half-life of the immunoconjugate. In certain embodiments, the alteration to one or more amino acid residues that alters binding of the immunoconjugate to the neonatal Fc receptor (FcRn) is to an amino acid residue selected from the list consisting of: 251, 252, 253, 254, 255, 288, 309, 310, 312, 385, 386, 388, 400, 415, 433, 435, 436, 439, 447, and combinations thereof per EU numbering. In certain embodiments, the alteration to one or more amino acid residues that alters binding of the immunoconjugate to the neonatal Fc receptor (FcRn) is to an amino acid residue selected from the list consisting of: 253, 254, 310, 435, 436 and combinations thereof per EU numbering. In certain embodiments, the alteration to one or more amino acid residues that alters binding of the immunoconjugate to the neonatal Fc receptor (FcRn) is to an amino acid residue selected from the list consisting of: I253A, I253D, I253P, S254A, H310A, H310D, H310E, H310Q, H435A, H435Q, Y436A, and combinations thereof per EU numbering. In certain embodiments, the alteration to one or more amino acid residues that alters binding of the immunoconjugate to the neonatal Fc receptor (FcRn) is to an amino acid residue selected from the list consisting of: I253A, S254A, H310A, H435Q, Y436A and combinations thereof per EU numbering. In certain embodiments, the alteration to one or more amino acid residues that alters binding of the immunoconjugate to the neonatal Fc receptor (FcRn) is to an amino acid residue selected from the list consisting of: I253A, H310A, H435Q, and combinations thereof per EU numbering. In certain embodiments, the immunoconjugate has a serum half-life of less than 15 days. In certain embodiments, the immunoconjugate has a serum half-life of less than 10 days. In certain embodiments, the immunoconjugate has a serum half-life of less than 120 hours. In certain embodiments, the immunoconjugate has a serum half-life of less than 72 hours. In certain embodiments, the antigen binding region is coupled to the immunoglobulin heavy chain constant region by a linker amino acid sequence or a human IgG hinge region. In certain embodiments, the antigen binding region is coupled to the immunoglobulin heavy chain constant region by a human IgG hinge region.

In certain embodiments, the chelating agent is coupled to the antigen binding region and/or the immunoglobulin heavy chain constant region at a ratio of 1:1 to 8:1. In certain embodiments, the chelating agent is coupled to the antigen binding region and/or the immunoglobulin heavy chain constant region at a ratio of 1:1 to 6:1. In certain embodiments, the chelating agent is coupled to the antigen binding region and/or the immunoglobulin heavy chain constant region at a ratio of 2:1 to 6:1. In certain embodiments, the immunoconjugate further comprises a radioisotope. In certain embodiments, the radioisotope is an alpha emitter. In certain embodiments, the radioisotope is an alpha emitter selected from the list consisting of actinium-225 (²²⁵Ac), radium-223 (²²³Ra), radium-224 (²²⁴Ra), thorium-227 (²²⁷Th), lead-212 (²¹²Pb), bismuth-212 (²¹²Bi), and bismuth-213 (²¹³Bi). In certain embodiments, the radioisotope is ²²⁵Ac. In certain embodiments, the radioisotope is a beta emitter. In certain embodiments, the radioisotope is a beta emitter selected from ¹⁷⁷Lu, ⁹⁰Y, copper-67 (⁶⁷Cu), and samarium-153 (¹⁵³Sm). In certain embodiments, the molecular weight of the immunoconjugate is between 60 and 100 kDa. In certain embodiments, the molecular weight of the immunoconjugate is between 60 and 90 kDa. In certain embodiments, the molecular weight of the immunoconjugate is between 65 and 90 kDa. In certain embodiments, the molecular weight of the immunoconjugate is between 70 and 90 kDa. In certain embodiments, the immunoconjugate forms a dimer with another immunoconjugate. In certain embodiments, the immunoconjugate further comprises a pharmaceutically acceptable excipient or carrier. In certain embodiments, the immunoconjugate is formulated for intravenous administration.

Also described herein is a method of making the immunoconjugate comprising loading the immunoconjugate with a radioisotope. In certain embodiments, the radioisotope is an alpha emitter. In certain embodiments, the radioisotope is an alpha emitter selected from the list consisting of ²²⁵Ac, ²²³Ra, ²²⁴Ra, ²²⁷Th, ²¹²Pb, ²¹²-Bi, and ²¹³Bi. In certain embodiments, the radioisotope is ²²⁵Ac. In certain embodiments, the radioisotope is a beta emitter. In certain embodiments, the radioisotope is a beta emitter selected from ¹⁷⁷Lu, ⁹⁰Y, ⁶⁷Cu, and ¹⁵³Sm. In certain embodiments, the radioisotope is ¹⁷⁷Lu.

Also described herein is a method of treating a cancer or a tumor in an individual comprising administering to the individual the immunoconjugate, thereby treating the cancer or the tumor. In certain embodiments, the individual is a human individual. In certain embodiments, the cancer or tumor is a solid cancer or tumor. In certain embodiments, the cancer or the tumor comprises lung cancer, breast cancer, ovarian cancer, or a neuroendocrine cancer. In certain embodiments the method further comprises administering from 0.5 μCi to 30.0 μCi per kilogram to the individual. In certain embodiments, the cancer or tumor expresses an antigen specifically bound by the immunoconjugate.

Also described herein is the immunoconjugate for use in a method of treating a cancer or a tumor in an individual. In certain embodiments, the individual is a human individual. In certain embodiments, the cancer or tumor is a solid cancer or tumor. In certain embodiments, the cancer or the tumor comprises lung cancer, breast cancer, ovarian cancer, or a neuroendocrine cancer. In certain embodiments, from 0.5 μCi to 30.0 μCi per kilogram is administered to the individual. In certain embodiments, the cancer or tumor expresses an antigen specifically bound by the immunoconjugate.

Also described herein is a method of killing a cancer cell in an individual comprising administering to the individual the immunoconjugate, thereby killing the cancer cell. In certain embodiments, the individual is a human individual. In certain embodiments, the cancer cell comprises a lung cancer cell, a breast cancer cell, an ovarian cancer cell, or a neuroendocrine cancer cell. In certain embodiments, the method comprises administering from 0.1 μCi to 30.0 μCi per kilogram to the individual. In certain embodiments, the method comprises administering from 10 mCi to 75 mCi per meter squared of body area to the individual. In certain embodiments, the cancer cell expresses an antigen specifically bound by the immunoconjugate.

Also described herein is use of the immunoconjugate in a method of killing a cancer cell in an individual. In certain embodiments, the individual is a human individual. In certain embodiments, the cancer cell comprises a lung cancer cell, a breast cancer cell, an ovarian cancer cell, or a neuroendocrine cancer cell. In certain embodiments, the method comprises administering from 0.5 μCi to 30.0 μCi per kilogram to the individual. In certain embodiments, the cancer cell expresses an antigen specifically bound by the immunoconjugate.

Also described herein is a method of delivering a radioisotope to a cancer cell or a tumor cell in an individual comprising administering to the individual the immunoconjugate, thereby delivering the radioisotope to the cancer cell or the tumor cell. In certain embodiments, the individual is a human individual. In certain embodiments, the cancer cell or the tumor cell comprises a lung cancer cell, a breast cancer cell, an ovarian cancer cell, or a neuroendocrine cancer cell. In certain embodiments, the method comprises administering from 0.5 μCi to 30.0 μCi per kilogram to the individual. In certain embodiments, the cancer cell or the tumor cell expresses an antigen specifically bound by the immunoconjugate.

Also described herein is the immunoconjugate for use in delivering a radioisotope to a cancer cell or a tumor cell in an individual. In certain embodiments, the individual is a human individual. In certain embodiments, the cancer cell or the tumor cell comprises a lung cancer cell, a breast cancer cell, an ovarian cancer, or a neuroendocrine cancer cell. In certain embodiments, the cancer cell or the tumor cell expresses an antigen specifically bound by the immunoconjugate.

Also described herein is a method of imaging a tumor in an individual comprising administering to the individual the immunoconjugate. In certain embodiments, the individual is a human individual. In certain embodiments, the cancer or the tumor comprises lung cancer, breast cancer, ovarian cancer, or a neuroendocrine cancer. In certain embodiments, the tumor expresses an antigen specifically bound by the immunoconjugate.

Also described herein is the immunoconjugate for use in a method of imaging a tumor in an individual. In certain embodiments, the individual is a human individual. In certain embodiments, the cancer or the tumor comprises lung cancer, breast cancer, ovarian cancer, or a neuroendocrine cancer. In certain embodiments, the tumor expresses an antigen specifically bound by the immunoconjugate.

Also described herein is a nucleic acid encoding the immunoconjugate. In certain embodiments, an expression vector comprises the nucleic acid. In certain embodiments, A cell comprises the nucleic acid or the expression vector. In certain embodiments, the cell is a eukaryotic cell. In certain embodiments, the eukaryotic cell is a CHO cell.

In some embodiments, the subject radioisotope delivery platforms have a molecular size large enough (e.g., 60 kDa to 110 kDa) to substantially reduce off-target toxicities, especially renal damage (e.g., from an alpha emitting isotope cargo) and a small enough size for increased tissue penetration as compared to traditional IgGs, with maintained target specificity, and increased probability of first decay event in target tissue. Such sizes provide for preferential elimination by the liver as opposed to the kidney, sparing the kidney from radiotoxicity.

In some embodiments, the subject radioisotope delivery platforms are useful for in vivo targeted delivery of alpha emitters safely and effectively by, in part, reducing certain adverse effects caused by platforms having half-lives over 5 days and/or molecular weights under 60 kDa.

In some embodiments, the subject radioisotope delivery platforms are useful for in vivo targeted delivery of alpha emitters safely and effectively, in part, by exhibiting decreased loss of targeting capacity due to radiolysis as compared to other possible delivery platforms.

In some embodiments, the subject radioisotope delivery platforms are useful for in vivo targeted delivery of alpha emitters safely and effectively, in part, by exhibiting increased stability in manufacturing under the temperatures required for certain radiolabeling processes (e.g., high temperature chelation with certain chelators) as compared to other possible delivery platforms using antibody fragments.

In one embodiment, the invention provides immunoconjugates for delivering α-emitting radioisotopes in vivo. In one embodiment, the immunoconjugates are also capable of delivering other atoms in vivo. In one embodiment, the immunoconjugates are capable of delivering imaging metals (e.g., indium-111 (¹¹¹In), zirconium-89 (⁸⁹Zr), copper-64 (⁶⁴Cu), gallium-68 (⁶⁸Ga), or cesium-134 (¹³⁴Ce)) in vivo.

In one embodiment, the immunoconjugate comprises an antibody construct and a chelating agent, and has a molecular weight between 60 and 110 kDa, preferably between 60 and 100 kDa, preferably between 60 and 90 kDa, preferably between 65 and 90 kDa, preferably between 70 and 90 kDa. The chelating agent is capable of chelating an α-emitting radioisotope such that the antibody construct is linked to the α-emitting radioisotope.

At least one of the variant constant regions in the immunoconjugate has at least one FcRn binding mutation. In a preferred embodiment, each of the two variant constant regions of the immunoconjugate has at least one FcRn binding mutation, which FcRn binding mutations are the same or different.

In one embodiment, the chelating agent comprises DOTA or a DOTA derivative. In one embodiment, the chelating agent comprises DOTAGA. In one embodiment, the chelating agent comprises macropa or a macropa derivative. In one embodiment, the chelating agent comprises Py4Pa or a Py4Pa derivative. In one embodiment, the chelating agent comprises siderocalin or a siderocalin derivative.

In one embodiment, the chelating agent comprises a radioisotope chelating component and a functional group that allows for covalent linkage to the antigen binding arm. In one embodiment, the functional group is directly linked to the radioisotope chelating component. In one embodiment the chelating agent further comprises a linker between the functional group and the radioisotope chelating component.

In one embodiment, the radioisotope chelating component comprises DOTA or a DOTA derivative. In one embodiment, the radioisotope chelating component comprises DOTAGA. In one embodiment, the radioisotope chelating component comprises macropa or a macropa derivative. In one embodiment, the radioisotope chelating component comprises Py4Pa or a Py4Pa derivative.

In one embodiment, the invention provides a pharmaceutical composition, comprising a radioimmunoconjugate of the invention and a pharmaceutically acceptable carrier.

In one embodiment, the invention provides a method of delivering an α-emitting radioisotope to a cancer cell in vivo in a patient, comprising administering a radioimmunoconjugate or pharmaceutical composition of the invention to the patient. In one embodiment, the patient is a human patient.

In one embodiment, the invention provides a method of inhibiting the growth of a cancer cell, comprising contacting the cancer cell with a radioimmunoconjugate of the invention. In one embodiment, the cancer cell is in vivo in a patient. In one embodiment, the method involves administering a pharmaceutical composition of the invention to the patient. In one embodiment, the patient is a human patient.

In one embodiment, the invention provides a method of killing a cancer cell, comprising contacting the cancer cell with a radioimmunoconjugate of the invention. In one embodiment, the cancer cell is in vivo in a patient. In one embodiment, the method involves administering a pharmaceutical composition of the invention to the patient. In one embodiment, the patient is a human patient.

In one embodiment, the invention provides a method of treating cancer in a patient in need thereof, comprising administering to the patient a radioimmunoconjugate or pharmaceutical composition of the invention. In one embodiment, the patient is a human patient.

In one embodiment, the invention provides a targeted imaging complex, comprising an immunoconjugate of the invention and further comprising an imaging metal. In one aspect, the invention provides a targeted imaging complex, comprising an antibody construct of an immunoconjugate of the invention and further comprising an imaging metal. In one embodiment, the imaging metal is a radioisotope. In one embodiment, the imaging metal is selected from the group comprising: ¹¹¹In, ⁸⁹Zr, ⁶⁴Cu, ⁶⁸Ga, and ¹³⁴Ce. In one embodiment, the imaging metal is selected from the group consisting of ¹¹¹In, ⁸⁹Zr, ⁶⁴Cu, ⁶⁸Ga, and ¹³⁴Ce. In one embodiment, the imaging metal is ¹¹¹In. In one embodiment, the imaging metal is covalently bound to the immunoconjugate or antibody construct. In one embodiment, the imaging metal is associated with the chelating agent of an immunoconjugate. In one embodiment, the invention provides a method of determining the location of a cancer cell in vivo in a patient, comprising administering to the patient a targeted imaging complex of the invention. In one embodiment, the patient is a human patient.

In one embodiment, the invention provides a kit for preparing a radiopharmaceutical of the invention, comprising an immunoconjugate of the invention. In one embodiment, the invention provides a kit comprising a radioimmunoconjugate of the invention. In one embodiment, the invention provides a kit for preparing a pharmaceutical composition of the invention, comprising an immunoconjugate of the invention. In one embodiment, the invention provides a kit for preparing a pharmaceutical composition of the invention, comprising a radioimmunoconjugate of the invention. In one embodiment, the invention provides a kit comprising a pharmaceutical composition of the invention.

In some embodiments, the immunoconjugate or radioimmunoconjugate of the invention comprises a dimerization domain or motif. In some further embodiments, the dimerization domain or motif is in the hinge region and/or the variant constant region.

In some embodiments, the immunoconjugate or radioimmunoconjugate or pharmaceutical composition of the invention has a half-life in human serum of less than 96 hours. In some further embodiments, a half-life in human serum of less than 72 hours. In some further embodiments, the half-life is less than 48, 36, 24, and/or 12 hours. In some embodiments, the half-life is between 4 and 8 hours, between 6 and 12 hours, between 8 and 16 hours, between 12 and 24 hours, or between 24 and 48.

In one aspect, the invention provides a radioimmunoconjugate, comprising an immunoconjugate of the invention and further comprising a beta particle emitter, such as, e.g., ¹⁷⁷Lu, ⁹⁰Y, ⁶⁷Cu, or ¹⁵³Sm. In one aspect, the invention provides a pharmaceutical composition comprising such radioimmunoconjugate.

In one aspect, the invention provides a radioimmunoconjugate, comprising an immunoconjugate of the invention and further comprising an alpha particle emitter and a beta and/or gamma particle emitter. In one aspect, the invention provides a pharmaceutical composition comprising such radioimmunoconjugate.

In some embodiments, a kit of the invention includes a reagent or pharmaceutical device in addition to the immunoconjugate, radioimmunoconjugate or pharmaceutical composition of the invention.

In some embodiments, the kit of the present invention is an immunoassay kit for specifically detecting an antigen in a biological sample, comprising: (a) immunoconjugate, radioimmunoconjugate or targeted imaging complex as described herein and/or a composition thereof; and (b) instructions for detecting the immunoconjugate, radioimmunoconjugate or targeted imaging complex.

In another aspect, the invention provides an isolated nucleic acid encoding an antigen binding arm or a component thereof as provided herein. In one aspect, the invention provides an isolated nucleic acid encoding an antigen binding region of an immunoconjugate herein. In one aspect, the invention provides an isolated nucleic acid encoding a VHH polypeptide of an immunoconjugate herein. In one aspect, the invention provides an isolated nucleic acid encoding a hinge region of an immunoconjugate herein. In one aspect, the invention provides an isolated nucleic acid encoding a variant constant region of an immunoconjugate herein. In one aspect, the invention provides an isolated nucleic acid encoding a VHH polypeptide of an immunoconjugate herein and a hinge region of an immunoconjugate herein. In one aspect, the invention provides an isolated nucleic acid encoding a VHH polypeptide of an immunoconjugate herein, a hinge region of an immunoconjugate herein, and a variant constant region of an immunoconjugate herein.

In another aspect, the invention provides a vector comprising a nucleic acid as provided herein. In some embodiments, the vector is an expression vector.

In another aspect, the invention provides methods of using an immunoconjugate, radioimmunoconjugate, targeted imaging complex or pharmaceutical composition of the present invention. In some embodiments, the invention provides a method of treating a disease, disorder, or condition, the method comprising administering to patient in need thereof a pharmaceutically effective amount of a radioimmunoconjugate or pharmaceutical composition herein.

In some embodiments, a method of the invention comprises the step of administering to a subject, in need thereof, any of the radioimmunoconjugates or pharmaceutical compositions described herein. For some further embodiments, the method is for inhibiting the growth and/or the killing of a cancer cell or tumor.

In some embodiments, the use of an immunoconjugate or radioimmunoconjugate described herein is provided for the manufacture of a medicament for treating a disease, disorder, or condition in a subject, such as, e.g., cancer.

In another aspect, the invention provides a process for making a radioimmunoconjugate or pharmaceutical composition of the present invention, the method comprising radiolabeling the immunoconjugate with an appropriate isotope, such as, e.g., an alpha or beta particle emitter.

These and other features, aspects and advantages of the present invention will become better understood with regard to the following description and appended claims. The aforementioned elements of the invention may be individually combined or removed freely in order to make other embodiments of the invention, without any statement to object to such combination or removal hereinafter.

### BRIEF DESCRIPTION OF THE DRAWINGS

FIGS. 1A and 1B show binding of anti-HER2 and anti-DLL3 VHH-Fc constructs.

FIGS. 2A, 2B, and 2C show binding of anti-HER2 and anti-DLL3 VHH-Fc constructs to cells expressing HER2 and/or DLL3.

FIGS. 3A and 3B show internalization of anti-HER2 and anti-DLL3 VHH-Fc constructs in cells expressing HER2 and DLL3.

FIG. 4 shows self-interaction data for anti-HER2 and anti-DLL3 VHH-Fc constructs.

FIG. 5 shows a diagram for chemical synthesis of linker molecules.

FIG. 6 shows a diagram for chemical synthesis of linker molecules.

FIGS. 7A, 7B, and 7C shows the immunoreactive fraction of different VHH-Fc constructs.

FIG. 8 shows a comparison of imaging with ¹¹¹In labeled VHH-Fc compared to biodistribution of ²²⁵Ac labeled VHH-Fc.

FIGS. 9A, 9B, 9C, and 9D show biodistribution over time for labeled anti-HER2 VHH-Fc constructs.

FIGS. 10A, 10B and 10C show tumor:non-tumor tissue ratios for labeled anti-HER2 VHH-Fc constructs.

FIG. 11 shows biodistribution for labeled anti-HER2 VHH-Fc constructs.

FIG. 12 shows whole body clearance of VHH-Fc (H101) and VHH-Fc variants (H105, H107, and H108) labeled with ¹¹¹In.

FIG. 13 shows biodistribution over time for labeled anti-DLL3 VHH-Fc constructs.

FIG. 14 shows biodistribution for labeled anti-DLL3 VHH-Fc constructs.

FIGS. 15A and 15B show biodistribution for ²²⁵Ac labeled anti-HER2 (15A) and anti-DLL3 (15B) VHH-Fc constructs.

FIGS. 16A, 16B, and 16C show the results of a toxicity study carried out with ²²⁵Ac labeled anti-HER2 VHH-Fc constructs.

FIG. 17 shows the immunoreactive fraction of different anti-DDL3 VHH-Fc constructs loaded with ¹⁷⁷Lu.

### DETAILED DESCRIPTION

The present invention is described more fully hereinafter using illustrative, non-limiting embodiments. This invention may, however, be embodied in many different forms and should not be construed as to be limited to the embodiments set forth below. Rather, these embodiments are provided so that this disclosure is thorough and conveys the scope of the invention to those skilled in the art. In order that the present invention may be more readily understood, certain terms are defined below. Additional definitions may be found within the detailed description of the invention.

In particular, in embodiments, the present invention addresses a number of challenges inherent in the targeted delivery of radioisotopes in vivo through the selection and particular assembly of specific immunoconjugate and radioimmunoconjugate components. The radioisotope-delivering platforms of the present invention provide shorter half-lives compared to traditional IgGs, but longer half-lives than smaller monomeric antibody fragment formats. In some embodiments, the subject radioisotope delivering platforms have a molecular size large enough (e.g., 60 kDa to 110 kDa) to substantially reduce off-target toxicities, especially renal damage (e.g., from an alpha- or beta-emitting isotope cargo) and a small enough size for increased tissue penetration as compared to traditional IgGs, with maintained target specificity, and increased probability of first decay event in target tissue. In some embodiments, the subject radioisotope delivering platforms are useful for in vivo targeted delivery of radioisotopes (such as alpha- or beta-emitters) safely and effectively by, in part, reducing certain adverse effects caused by platforms having half-lives over 5 days and/or molecular weights under 60 kDa. In some embodiments, the subject radioisotope delivering platforms are useful for in vivo targeted delivery of radioisotopes (such as alpha- or beta-emitters) safely and effectively, in part, by exhibiting decreased loss of targeting capacity due to radiolysis as compared to other possible delivery platforms. In some embodiments, the subject radioisotope delivering platforms are useful for in vivo targeted delivery of radioisotopes (such as alpha- or beta-emitters) safely and effectively, in part, by exhibiting increased stability in manufacturing under the temperatures required for certain radiolabeling processes (e.g., high temperature chelation with certain chelators) as compared to other possible delivery platforms using antibody fragments.

### Immunoconjugates

In one aspect, the invention provides immunoconjugates that specifically bind to a target antigen with high affinity. In some embodiments, the present invention provides an immunoconjugate that specifically binds to a cell-surface antigen of a cancer cell. In some embodiments, the immunoconjugate comprises three, four, five, six, or more CDRs or HVRs (Kabat). In some embodiments, the immunoconjugate binds a specific antigen and/or epitope with an affinity characterized by a K_{D} of ≤1 μM, <100 nM, <10 nM, <1 nM, <0.1 nM, <0.01 nM, or <0.001 nM (e.g. 10⁻⁸ M or less, e.g. from 10⁻⁸ M to 10⁻¹³ M, e.g., from 10⁻⁹ M to 10⁻¹³ M).

The immunoconjugates described herein may serve as a platform for radio isotope delivery. Radioisotope delivering platforms are provided herein that have a relatively short half-life (e.g., less than one or two weeks but greater than two to eight hours).

In one embodiment, an immunoconjugate of the current disclosure comprises an: a) antigen binding region; b) an immunoglobulin heavy chain constant region; and c) a chelating moiety or a radionuclide complex thereof. In one embodiment an immunoconjugate of the current disclosure comprises an: a) antigen binding region; b) an immunoglobulin heavy chain constant region; and c) a chelating moiety or a radionuclide complex thereof; wherein the molecular weight of said immunoconjugate is between 60 and 110 kDa.

In one embodiment, an immunoconjugate of the current disclosure comprises an: a) VHH antigen binding region; b) an immunoglobulin heavy chain constant region; and c) a chelating moiety or a radionuclide complex thereof. In one embodiment an immunoconjugate of the current disclosure comprises an: a) VHH antigen binding region; b) an immunoglobulin heavy chain constant region; and c) a chelating moiety or a radionuclide complex thereof; wherein the molecular weight of said immunoconjugate is between 60 and 110 kDa.

In one embodiment, an immunoconjugate of the current disclosure comprises an: a) VHH antigen binding region; b) an immunoglobulin Fc region, together referred to as aVHH-Fc; and c) a chelating moiety or a radionuclide complex thereof. In one embodiment an immunoconjugate of the current disclosure comprises an: a) VHH antigen binding region; b) an immunoglobulin Fe region; and c) a chelating moiety or a radionuclide complex thereof; wherein the molecular weight of said immunoconjugate is between 60 and 110 kDa.

In one embodiment an immunoconjugate of the current disclosure comprises an: a) VHH antigen binding region; b) a variant immunoglobulin Fc region; and c) a chelating moiety or a radionuclide complex thereof. In one embodiment an immunoconjugate of the current disclosure comprises an: a) VHH antigen binding region; b) a variant immunoglobulin Fc region; and c) a chelating moiety or a radionuclide complex thereof; wherein the molecular weight of said immunoconjugate is between 60 and 110 kDa. In certain embodiments, the variant immunoglobulin Fc region comprises one or more amino acid alterations to reduce the serum or plasma half-life of the immunoconjugate.

In some embodiments, the radioisotope delivering platforms have sizes larger than about 60 kDa, in order to avoid certain toxicities from an alpha emitting isotope cargo, such as, e.g., off-target renal toxicities. In some embodiments, the radioisotope delivering platforms have sizes less than about 110 kDa in order to improve tumor penetration. In some embodiments, the radioisotope delivering platform has size between 60 and 110 kDa due to its dimeric structure of two individual antigen binding arms each having a VHH polypeptide fused to a hinge region and a wild-type or variant constant region. In some embodiments, the variant constant region has specific amino acid substitution(s) relatively to a wildtype Fc region in order to reduce half-life and/or eliminate Fc effector function(s).

In one embodiment, the antibody construct of the immunoconjugate consists of two antigen binding arms that are covalently linked to each other (for example via a disulfide linkage between associated heavy chain constant regions or immunoglobulin hinge regions). Each of the antigen binding arms independently consists of an antigen binding region, a hinge region, and a variant constant region. Within each antigen binding arm, the antigen binding region of the arm is covalently linked to the hinge region of the arm and the hinge region of the arm is covalently linked to the variant constant region of the arm, such that the hinge region is interposed between and thereby links the antigen binding region and the variant constant region within the antigen binding arm.

In a preferred embodiment, at least one of the two antigen binding regions in the immunoconjugate consists of one or two heavy chain only variable (VHH) polypeptides. In a preferred embodiment at least one of the two antigen binding regions consists of one VHH polypeptide. In a preferred embodiment, each of the two antigen binding regions of the immunoconjugate consists of one VHH polypeptide, which VHH polypeptides are the same or different.

In one embodiment, the antigen binding regions of the immunoconjugate bind to the same antigen. In one embodiment, the antigen binding regions of the immunoconjugate bind to different antigens. In one embodiment, the antigen binding regions of the immunoconjugate are the same. In one embodiment, the antigen binding regions of the immunoconjugate are different. In one embodiment, the antigen binding region of each antigen binding arm consists of one or two VHH polypeptides.

In one embodiment, the antigen binding region of one antigen binding arm consists of two VHH polypeptides and the antigen binding region of the other antigen binding arm does not comprise a VHH polypeptide. In one embodiment, the two antigen binding arms bind the same antigen. In one embodiment, the two antigen binding arms bind different antigens. In one embodiment, the two VHH polypeptides are the same. In one embodiment, the two VHH polypeptides are different. In one embodiment, the immunoconjugate is bispecific.

In one embodiment, the antigen binding region of one antigen binding arm consists of one VHH polypeptide and the antigen binding region of the other antigen binding arm consists of two VHH polypeptides. In one embodiment, the two antigen binding arms bind the same antigen. In one embodiment, the two antigen binding arms bind different antigens. In one embodiment, the three VHH polypeptides are the same. In one embodiment, two of the three VHH polypeptides are the same and are different from the third VHH polypeptide. In one embodiment, the three VHH polypeptides are different. In one embodiment, the immunoconjugate is bispecific.

In one embodiment, the antigen binding region of each antigen binding arm of the immunoconjugate consists of one VHH polypeptide. In one embodiment, the VHH polypeptides bind to the same antigen. In one embodiment, the VHH polypeptides bind to different antigens. In one embodiment, the VHH polypeptides are the same. In one embodiment, the VHH polypeptides are different. In one embodiment, the immunoconjugate is bispecific.

### Antigen Binding Regions

The antigen binding region confers specificity to the immunoconjugate and may suitably comprise a small antigen binding polypeptide. Such small antigen binding polypeptides confer advantages such as reducing the overall size of the immunoconjugate molecule allowing for tumor penetration and labeling. The small antigen binding polypeptide may lack certain regions dispensable for binding such as a light chain constant region, a heavy chain constant region, a CH1 region or a hinge region. In certain embodiments, the antigen binding region may lack a light chain variable region. In certain embodiments, the small antigen binding region may possess a molecular weight of between 10 kDa and 40 kDa.

In some embodiments, the small antigen binding region possesses a molecular weight of about 10 kDa to about 40 kDa. In some embodiments, the small antigen binding region possesses a molecular weight of about 10 kDa to about 15 kDa, about 10 kDa to about 20 kDa, about 10 kDa to about 25 kDa, about 10 kDa to about 30 kDa, about 10 kDa to about 35 kDa, about 10 kDa to about 40 kDa, about 15 kDa to about 20 kDa, about 15 kDa to about 25 kDa, about 15 kDa to about 30 kDa, about 15 kDa to about 35 kDa, about 15 kDa to about 40 kDa, about 20 kDa to about 25 kDa, about 20 kDa to about 30 kDa, about 20 kDa to about 35 kDa, about 20 kDa to about 40 kDa, about 25 kDa to about 30 kDa, about 25 kDa to about 35 kDa, about 25 kDa to about 40 kDa, about 30 kDa to about 35 kDa, about 30 kDa to about 40 kDa, or about 35 kDa to about 40 kDa. In some embodiments, the small antigen binding region possesses a molecular weight of about 10 kDa, about 15 kDa, about 20 kDa, about 25 kDa, about 30 kDa, about 35 kDa, or about 40 kDa. In some embodiments, the small antigen binding region possesses a molecular weight of at least about 10 kDa, about 15 kDa, about 20 kDa, about 25 kDa, about 30 kDa, or about 35 kDa. In some embodiments, the small antigen binding region possesses a molecular weight of at most about 15 kDa, about 20 kDa, about 25 kDa, about 30 kDa, about 35 kDa, or about 40 kDa.

The antigen binding region may comprise a VHH polypeptide, an scFv polypeptide, or a VNAR polypeptide. In certain embodiments, the antigen binding region comprises a VHH polypeptide. In certain embodiments, the antigen binding region comprises a ScFv polypeptide. In certain embodiments, the antigen binding region comprises a VNAR polypeptide. In certain embodiments, the antigen binding region is humanized.

The antigen region can comprise a specificity to an antigen selected by the skilled artisan to achieve a desired function, such as targeting a particular cancer, tumor, or cell type amenable to treatment with the described immunoconjugates or radioimmunoconjugates. As described herein antigen binding regions can be fragments or formats of antibodies known in the art. Intact antibodies can be engineered to conform to various small antigen binding region formats described herein (e.g., scFv). The antigen binding region may specifically bind to tumor antigen (e.g., an antigen specifically expressed or enriched in cancerous cells). IN certain embodiments, the tumor antigen comprises Her2, Trop2, CEA, NaPi2b, uPAR, CDCP1, MUC-1, MUC-16, CEACAM-5, MR-1, Fn14, MAGE-3, NY-ESO-1, EGFR, PDGFR, IGF1R, CSF-1R, PSMA, PSCA, STEAP-1, FAP, TEM8, 5T4, VEGFR, NRP1, CD19, CD20, CD22, CD25, CD30, CD33, CD37, CD38, CD39, CD44, CD47, CD52, CD70, CD71, CD74, CD79b, CD132, CD133, CD138, CD166, CD205, CD276, ROR1, ROR2, Glypican 3, Trail Receptor 2 (DR5), PD-L1, Mesothein, Bombesin, EpCAM, DARPP, CSPG4, Galectin-3, Integrin αvβ1, Integrin αvβ3, Integrin αvβ5, Integrin αvβ6, Integrin α5β1, Integrin alpha-3, Integrin alpha-5, Integrin beta-6, Nectin-4, Wnt activated inhibitory factor 1, DLL3, Transferrin Receptor, Folate Receptor alpha, Tissue Factor, BCMA, c-Met, LIV-1, AXL, AFP, ENPP3, CLDN6/9, DPEP3, RNF43, LRRC15, PTK7, P-cadherin, FLT3, EphA2, MTI-MMP, CXCR6, GD2, or Smoothened antigen (Smo). In certain embodiments, the tumor antigen comprises human epidermal growth factor receptor 2 (HER2), Delta-like ligand 3 (DLL3), folate receptor alpha (FOLR1), or Wnt activated inhibitory factor 1 (WAIF1). In certain embodiments, the tumor antigen comprises HER2. In certain embodiments, the tumor antigen comprises DLL3. In certain embodiments, the tumor antigen comprises FOLR1. In certain embodiments, the tumor antigen comprises WAIF1. In certain embodiments, the tumor antigen comprises TROP2. In certain embodiments, the tumor antigen comprises EGFR. In certain embodiments, the tumor antigen comprises PSA. In certain embodiments, the tumor antigen comprises MUC-1. In certain embodiments, the tumor antigen comprises CEA. In certain embodiments, the tumor antigen comprises NY-ESO-1.

In certain embodiments, the antigen binding region of the immunoconjugate comprises a sequence that is at least 85%, 90%, 95%, 97%, 98%, 99%, or 100% identical to the sequence set forth in SEQ ID NO: 20 and that binds to HER2.

In certain embodiments the antigen binding region of the immunoconjugate comprises: a) a CDR1 comprising the amino acid sequence set forth in SEQ ID NO: 21; b) a CDR2 comprising the amino acid sequence set forth in SEQ ID NO: 22; and c) a CDR3 comprising the amino acid sequence set forth in SEQ ID NO: 23.

In certain embodiments, the antigen binding region of the immunoconjugate comprises a sequence that is at least 85%, 90%, 95%, 97%, 98%, 99%, or 100% identical to the sequence set forth in SEQ ID NO: 30 and that binds to DLL3.

In certain embodiments the antigen binding region of the immunoconjugate comprises: a) a CDR1 comprising the amino acid sequence set forth in SEQ ID NO: 31; b) a CDR2 comprising the amino acid sequence set forth in SEQ ID NO: 32; and c) a CDR3 comprising the amino acid sequence set forth in SEQ ID NO: 33.

In some embodiments, the immunoconjugate of the present invention comprises a synthetically engineered antibody derivate, such as, e.g. a protein or polypeptide comprising an autonomous V_{H} domain (such as, e.g., from camelids, murine, or human sources), single-domain antibody domain (sdAb), heavy-chain antibody domains derived from a camelid (V_{H}H fragment or V_{H} domain fragment), heavy-chain antibody domains derived from a camelid V_{H}H fragments or V_{H} domain fragments, heavy-chain antibody domain derived from a cartilaginous fish, immunoglobulin new antigen receptor (IgNAR), VNAR fragment, single-chain variable (scFv) fragment, nanobody, “camelized” or “camelised” scaffold comprising a V_{H} domain, Fd fragment consisting of the heavy chain and C_{H}1 domains, single chain Fv-C_{H}3 minibody, Fc antigen binding domain (Fcabs), scFv-Fc fusion, multimerizing scFv fragment (diabodies, triabodies, tetrabodies), disulfide-stabilized antibody variable (Fv) fragment (dsFv), disulfide-stabilized antigen-binding (Fab) fragment consisting of the V_{L}, V_{H}, C_{L} and C_{H}1 domains, scFv comprising a disulfide-stabilized heavy and light chain (sc-dsFvs), bivalent nanobodies, bivalent minibodies, bivalent F(ab′)₂ fragments (Fab dimers), bispecific tandem V_{H}H fragments, bispecific tandem scFv fragments, bispecific nanobodies, bispecific minibodies, and any genetically manipulated counterparts of the foregoing that retain paratope and target antigen binding function.

In some embodiments, the immunoconjugate is monovalent. In other embodiments, the immunoconjugate is multivalent, such as, e.g., bivalent. In some further embodiments, the immunoconjugate is bivalent and dimeric. In some further embodiments, the bivalent immunoconjugate is homodimeric.

In one aspect, the present invention provides antibody constructs (alone or in the context of immunoconjugates, radioimmunoconjugates, or targeted imaging complexes, each of the invention), comprising a VHH fragment comprising a heavy chain variable region comprising three heavy chain CDRs derived from a camelid, which bind to an antigen with specificity and high affinity.

In some embodiments, the antibody construct, immunoconjugate, radioimmunoconjugate, or targeted imaging complex specifically binds to at least one extracellular part of an antigen expressed on a cellular surface. In some embodiments, the immunoconjugate specifically binds to at least one extracellular part of antigen expressed by a target cell, such as, e.g., a tumor cell.

In some embodiments, the disclosure provides immunoconjugate that specifically binds to an antigen. In some embodiments, the immunoconjugate comprises an antibody construct comprising a heavy chain variable region (HVR-H) comprising three CDRs: hCDR1, hCDR2, and hCDR3, such as, e.g., derived from a camelid antibody or IgNAR. In some embodiments, the immunoconjugate comprises: (a) a light chain variable region (HVR-L) comprising three CDRs: lCDR1, lCDR2, and lCDR3, and (b) a heavy chain variable region (HVR-H) comprising three CDRs: hCDR1, hCDR2, and hCDR3. In some embodiments, the antibody construct is chimeric or humanized.

In some embodiments, the immunoconjugate of the present invention comprises an antibody construct comprising an antigen binding domain which is an antibody fragment, including but not limited to, e.g., a Fv, Fab, Fab′, scFv, HcAb fragment, VHH fragment, sdAb fragment, diabody, or F(ab′)2 fragment. In some further embodiments, the immunoconjugate of the present invention comprises a multimer of two or more antibody fragments, such as, e.g., a homodimer or heterodimer comprising two antibody fragments each capable of binding to an antigen with specificity and high affinity and each comprising a heavy chain variable region (HVR-H) comprising three CDRs: hCDR1, hCDR2, and hCDR3.

### Heavy Chain Constant Regions

The antigen binding regions of the immunoconjugates described herein may comprise an Fc or heavy chain constant region. The antigen binding molecules can be coupled to the Fc or heavy chain constant region directly, by a suitable linker, or by an IgG hinge region. The inclusion of the heavy chain constant region or Fc region confers such advantages as allowing for optimization and tuning of serum half-life, the addition of additional sites to conjugate a chelating or cytotoxic agent, and allow for purification of the immunoconjugates using standard processes and methods. The addition of a heavy chain constant region also increases the size which may shift the catabolisis and elimination of the immunoconjugate to the liver from the kidney. This can confer safety advantages especially for radioimmunoconjugates as the kidney is more sensitive to radiation than the liver. Alterations, that affect the effector function or the serum half-life of can be made to residues present in the heavy chain constant region responsible for binding the neonatal Fc receptor (FcRn). Binding to the FcRn, in general contributes to the increased half-life of molecules that comprise an immunoglobulin Fc, thus reducing binding to FcRn can reduce the half-life of molecules comprising an Fc. Reduction in FcRn binding can confer advantages such as a reduction in the half-life of immunoconjugates, and, thus, subsequent toxicity attributed to cytotoxic agents or radioisotopes. In certain embodiments, the immunoglobulin constant region comprises or consists of an Fc region. In certain embodiments, the immunoglobulin heavy chain constant region comprises a CH2 domain of an immunoglobulin, CH3 domain of an immunoglobulin, or a CH2 and a CH3 domain of an immunoglobulin. In certain embodiments, the immunoglobulin heavy chain constant region comprises a CH2 and a CH3 domain of an immunoglobulin. For treatment or imaging of human individuals the immunoglobulin heavy chain constant region may be human, preventing or reducing an endogenous immune response against the immunoconjugate. In certain embodiments, the immunoglobulin heavy chain constant region is a human immunoglobulin heavy chain constant region. In certain embodiments, the immunoglobulin heavy chain constant region is an IgA, IgG1, IgG2, IgG3, or IgG4 isotype. In certain embodiments, the immunoglobulin heavy chain constant region is an IgG1 isotype. In certain embodiments, the immunoglobulin heavy chain constant region is an IgG4 isotype.

The immunoglobulin heavy chain constant region can be a variant constant region that comprises one or more alterations to amino acid residue(s) that confers additional utility and advantageous properties to the immunoconjugates described herein. In certain embodiments, the immunoglobulin heavy chain constant region comprises an alteration to one or more amino acid residues that reduces an effector function of the immunoglobulin heavy chain constant region or alters binding of the immunoconjugate to the neonatal Fc receptor (FcRn). In certain embodiments, the immunoglobulin heavy chain constant region comprises an alteration to one or more amino acid residues that reduces an effector function of the immunoglobulin heavy chain constant region or reduces binding of the immunoconjugate to the neonatal Fc receptor (FcRn). In certain embodiments, the immunoglobulin heavy chain constant region comprises an alteration to one or more amino acid residues that reduces an effector function of the immunoglobulin heavy chain constant region and reduces binding of the immunoconjugate to the neonatal Fc receptor (FcRn). In certain embodiments, the immunoglobulin heavy chain constant region comprises an alteration to one or more amino acid residues that reduces an effector function of the immunoglobulin heavy chain constant region. In certain embodiments, the immunoglobulin heavy chain constant region comprises an alteration to one or more amino acid residues that reduces binding of the immunoconjugate to the neonatal Fc receptor (FcRn).

The alterations to heavy chain constant regions of the immunoconjugate can reduce effector function associated with a heavy chain constant region, such as, the ability to fix complement, promote phagocytosis, or recruit other immune effector cells (e.g., NK cells) to the heavy chain constant region. In certain embodiments, the alteration to one or more amino acid residues that reduces the effector function of the immunoglobulin heavy chain constant region is an alteration that reduces complement dependent cytotoxicity (CDC), antibody-dependent cell-cytotoxicity (ADCC), antibody-dependent cell-phagocytosis ADCP, or a combination thereof. In certain embodiments, the alteration to one or more amino acid residues that reduces the effector function of the immunoglobulin heavy chain constant region is selected from the list consisting of: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311 S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(ooo), per EU numbering. In certain embodiments, the alteration to one or more amino acid residues that reduces the effector function of the immunoglobulin heavy chain constant region comprises L234A, L235E, G237A, A330S, and P331S per EU numbering.

The alterations to heavy chain constant regions of the immunoconjugate can reduce the serum half-life of the immunoconjugate. In certain embodiments, the amino acid alteration that alters or reduces binding of the immunoconjugate to the neonatal Fc receptor (FcRn) reduces the serum half-life of the immunoconjugate. In certain embodiments, the alteration that alters or reduces binding of the immunoconjugate to the neonatal Fc receptor (FcRn) is to an amino acid residue selected from the list consisting of: 251, 252, 253, 254, 255, 288, 309, 310, 312, 385, 386, 388, 400, 415, 433, 435, 436, 439, 447, and combinations thereof per EU numbering. In certain embodiments, the alteration that alters or reduces binding of the immunoconjugate to the neonatal Fc receptor (FcRn) is to an amino acid residue selected from the list consisting of: 253, 254, 310, 435, 436 and combinations thereof per EU numbering. In certain embodiments, the alteration that alters or reduces binding of the immunoconjugate to the neonatal Fc receptor (FcRn) is to an amino acid residue selected from the list consisting of: I253A, I253D, I253P, S254A, H310A, H310D, H310E, H310Q, H435A, H435Q, Y436A, and combinations thereof per EU numbering. In certain embodiments, the alteration that alters or reduces binding of the immunoconjugate to the neonatal Fc receptor (FcRn) is to an amino acid residue selected from the list consisting of: I253A, S254A, H310A, H435Q, Y436A and combinations thereof per EU numbering. In certain embodiments, the alteration that alters or reduces binding of the immunoconjugate to the neonatal Fc receptor (FcRn) is to an amino acid residue selected from the list consisting of: I253A, H310A, H435Q, and combinations thereof per EU numbering. In certain embodiments, the alteration that alters or reduces binding of the immunoconjugate to the neonatal Fc receptor (FcRn) is to an amino acid residue selected from the list consisting of: H310A, H435Q, and combinations thereof per EU numbering.

In certain embodiments, a heavy chain constant regions of the immunoconjugate comprises a sequence at least 90%, 95%, 97%, 98%, or 99% identical to the sequence set forth in SEQ ID NO: 1. In certain embodiments, a heavy chain constant regions of the immunoconjugate comprises a sequence identical to SEQ ID NO: 1. In certain embodiments, a heavy chain constant regions of the immunoconjugate comprises a sequence at least 90%, 95%, 97%, 98%, or 99% identical to the sequence set forth in SEQ ID NO: 1, wherein the heavy chain constant region comprises an I253A substitution per EU numbering.

In certain embodiments, a heavy chain constant regions of the immunoconjugate comprises a sequence at least 90%, 95%, 97%, 98%, or 99% identical to the sequence set forth in SEQ ID NO: 2. In certain embodiments, a heavy chain constant regions of the immunoconjugate comprises a sequence identical to SEQ ID NO: 2. In certain embodiments, a heavy chain constant regions of the immunoconjugate comprises a sequence at least 90%, 95%, 97%, 98%, or 99% identical to the sequence set forth in SEQ ID NO: 2, wherein the heavy chain constant region comprises an S254A substitution per EU numbering.

In certain embodiments, a heavy chain constant regions of the immunoconjugate comprises a sequence at least 90%, 95%, 97%, 98%, or 99% identical to the sequence set forth in SEQ ID NO: 3. In certain embodiments, a heavy chain constant regions of the immunoconjugate comprises a sequence identical to SEQ ID NO: 3. In certain embodiments, a heavy chain constant regions of the immunoconjugate comprises a sequence at least 90%, 95%, 97%, 98%, or 99% identical to the sequence set forth in SEQ ID NO: 3, wherein the heavy chain constant region comprises an H310A substitution per EU numbering.

In certain embodiments, a heavy chain constant regions of the immunoconjugate comprises a sequence at least 90%, 95%, 97%, 98%, or 99% identical to the sequence set forth in SEQ ID NO: 4. In certain embodiments, a heavy chain constant regions of the immunoconjugate comprises a sequence identical to SEQ ID NO: 4. In certain embodiments, a heavy chain constant regions of the immunoconjugate comprises a sequence at least 90%, 95%, 97%, 98%, or 99% identical to the sequence set forth in SEQ ID NO: 4, wherein the heavy chain constant region comprises an H435Q substitution per EU numbering.

In certain embodiments, a heavy chain constant regions of the immunoconjugate comprises a sequence at least 90%, 95%, 97%, 98%, or 99% identical to the sequence set forth in SEQ ID NO: 5. In certain embodiments, a heavy chain constant regions of the immunoconjugate comprises a sequence identical to SEQ ID NO: 5. In certain embodiments, a heavy chain constant regions of the immunoconjugate comprises a sequence at least 90%, 95%, 97%, 98%, or 99% identical to the sequence set forth in SEQ ID NO: 5, wherein the heavy chain constant region comprises an Y436A substitution per EU numbering.

In certain embodiments, a heavy chain constant regions of the immunoconjugate comprises a sequence at least 90%, 95%, 97%, 98%, or 99% identical to the sequence set forth in SEQ ID NO: 6. In certain embodiments, a heavy chain constant regions of the immunoconjugate comprises a sequence identical to SEQ ID NO: 6. In certain embodiments, a heavy chain constant regions of the immunoconjugate comprises a sequence at least 90%, 95%, 97%, 98%, or 99% identical to the sequence set forth in SEQ ID NO: 6, wherein the heavy chain constant region comprises an H310A/H435Q substitution per EU numbering.

In certain embodiments, a heavy chain constant regions of the immunoconjugate comprises a sequence at least 90%, 95%, 97%, 98%, or 99% identical to the sequence set forth in SEQ ID NO: 7. In certain embodiments, a heavy chain constant regions of the immunoconjugate comprises a sequence identical to SEQ ID NO: 7. In certain embodiments, a heavy chain constant regions of the immunoconjugate comprises a sequence at least 90%, 95%, 97%, 98%, or 99% identical to the sequence set forth in SEQ ID NO: 7, wherein the heavy chain constant region comprises a L234A, L235E, G237A, A330S, and P331S substitution per EU numbering.

In certain embodiments, a heavy chain constant regions of the immunoconjugate comprises a sequence at least 90%, 95%, 97%, 98%, or 99% identical to the sequence set forth in SEQ ID NO: 8. In certain embodiments, a heavy chain constant regions of the immunoconjugate comprises a sequence identical to SEQ ID NO: 8, wherein the heavy chain constant region comprises a L234A, L235E, G237A, H310A, A330S, and P331 S substitution per EU numbering.

In certain embodiments, a heavy chain constant regions of the immunoconjugate comprises a sequence at least 90%, 95%, 97%, 98%, or 99% identical to the sequence set forth in SEQ ID NO: 9. In certain embodiments, a heavy chain constant regions of the immunoconjugate comprises a sequence identical to SEQ ID NO: 9. In certain embodiments, a heavy chain constant regions of the immunoconjugate comprises a sequence identical to SEQ ID NO: 9, wherein the heavy chain constant region comprises a L234A, L235E, G237A, H435Q, A330S, and P331S substitution per EU numbering.

In certain embodiments, a heavy chain constant regions of the immunoconjugate comprises a sequence at least 90%, 95%, 97%, 98%, or 99% identical to the sequence set forth in SEQ ID NO: 10. In certain embodiments, a heavy chain constant regions of the immunoconjugate comprises a sequence identical to SEQ ID NO: 10 per EU numbering.

In one embodiment, each of the two variant constant regions has at least one FcRn binding mutation. In one embodiment, each of the two variant constant regions has the same FcRn binding mutation. In one embodiment, each of the two variant constant regions has a different FcRn binding mutation.

In one embodiment, at least one of the variant constant regions in the immunoconjugate has at least one FcRn binding mutation. In a preferred embodiment, each of the two variant constant regions of the immunoconjugate has at least one FcRn binding mutation, which FcRn binding mutations are the same or different.

Alterations that effect FcRn binding can reduce the serum half-life of the immunoconjugate, thus allowing the skilled artisan to choose a half-life that is suitable for a particular imaging or therapeutic goal. In certain embodiments, the immunoconjugate has a serum half-life of about 12 hours to about 120 hours. In certain embodiments, the immunoconjugate has a serum half-life of about 12 hours to about 24 hours, about 12 hours to about 36 hours, about 12 hours to about 48 hours, about 12 hours to about 60 hours, about 12 hours to about 72 hours, about 12 hours to about 84 hours, about 12 hours to about 96 hours, about 12 hours to about 108 hours, about 12 hours to about 120 hours, about 24 hours to about 36 hours, about 24 hours to about 48 hours, about 24 hours to about 60 hours, about 24 hours to about 72 hours, about 24 hours to about 84 hours, about 24 hours to about 96 hours, about 24 hours to about 108 hours, about 24 hours to about 120 hours, about 36 hours to about 48 hours, about 36 hours to about 60 hours, about 36 hours to about 72 hours, about 36 hours to about 84 hours, about 36 hours to about 96 hours, about 36 hours to about 108 hours, about 36 hours to about 120 hours, about 48 hours to about 60 hours, about 48 hours to about 72 hours, about 48 hours to about 84 hours, about 48 hours to about 96 hours, about 48 hours to about 108 hours, about 48 hours to about 120 hours, about 60 hours to about 72 hours, about 60 hours to about 84 hours, about 60 hours to about 96 hours, about 60 hours to about 108 hours, about 60 hours to about 120 hours, about 72 hours to about 84 hours, about 72 hours to about 96 hours, about 72 hours to about 108 hours, about 72 hours to about 120 hours, about 84 hours to about 96 hours, about 84 hours to about 108 hours, about 84 hours to about 120 hours, about 96 hours to about 108 hours, about 96 hours to about 120 hours, or about 108 hours to about 120 hours. In certain embodiments, the immunoconjugate has a serum half-life of about 12 hours, about 24 hours, about 36 hours, about 48 hours, about 60 hours, about 72 hours, about 84 hours, about 96 hours, about 108 hours, or about 120 hours. In certain embodiments, the immunoconjugate has a serum half-life of at least about 12 hours, about 24 hours, about 36 hours, about 48 hours, about 60 hours, about 72 hours, about 84 hours, about 96 hours, or about 108 hours. In certain embodiments, the immunoconjugate has a serum half-life of at most about 24 hours, about 36 hours, about 48 hours, about 60 hours, about 72 hours, about 84 hours, about 96 hours, about 108 hours, or about 120 hours.

In certain embodiments, the immunoconjugate has a serum half-life of about 1 day to about 10 days. In certain embodiments, the immunoconjugate has a serum half-life of about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 3 days to about 4 days, about 3 days to about 5 days, about 3 days to about 6 days, about 3 days to about 7 days, about 3 days to about 8 days, about 3 days to about 9 days, about 3 days to about 10 days, about 4 days to about 5 days, about 4 days to about 6 days, about 4 days to about 7 days, about 4 days to about 8 days, about 4 days to about 9 days, about 4 days to about 10 days, about 5 days to about 6 days, about 5 days to about 7 days, about 5 days to about 8 days, about 5 days to about 9 days, about 5 days to about 10 days, about 6 days to about 7 days, about 6 days to about 8 days, about 6 days to about 9 days, about 6 days to about 10 days, about 7 days to about 8 days, about 7 days to about 9 days, about 7 days to about 10 days, about 8 days to about 9 days, about 8 days to about 10 days, or about 9 days to about 10 days. In certain embodiments, the immunoconjugate has a serum half-life of about 1 day, about 2 days, about 3 days, about 4 days, about 5 days, about 6 days, about 7 days, about 8 days, about 9 days, or about 10 days. In certain embodiments, the immunoconjugate has a serum half-life of at least about 1 day, about 2 days, about 3 days, about 4 days, about 5 days, about 6 days, about 7 days, about 8 days, or about 9 days. In certain embodiments, the immunoconjugate has a serum half-life of at most about 2 days, about 3 days, about 4 days, about 5 days, about 6 days, about 7 days, about 8 days, about 9 days, or about 10 days.

In certain embodiments, the heavy chain constant region has a molecular weight of about 10 kDa to about 25 kDa. In certain embodiments, the heavy chain constant region has a molecular weight of about 10 kDa to about 15 kDa, about 10 kDa to about 20 kDa, about 10 kDa to about 25 kDa, about 15 kDa to about 20 kDa, about 15 kDa to about 25 kDa, or about 20 kDa to about 25 kDa. In certain embodiments, the heavy chain constant region has a molecular weight of about 10 kDa, about 15 kDa, about 20 kDa, or about 25 kDa. In certain embodiments, the heavy chain constant region has a molecular weight of at least about 10 kDa, about 15 kDa, or about 20 kDa. In certain embodiments, the heavy chain constant region has a molecular weight of at most about 15 kDa, about 20 kDa, or about 25 kDa.

In some embodiments, the immunoconjugate of the present invention comprises a linker or hinge region, which is a polypeptide linking an antigen binding region to a heavy chain constant region or a variant constant region in the instant invention. Naturally occurring and synthetic hinge regions linking immunoglobulin components are well known in the art and available for use in the present invention. For example, see U.S. Pat. No. 8,067,548 and references therein.

In one embodiment, the hinge regions of the immunoconjugate are the same. In one embodiment, the hinge regions of the immunoconjugate are different.

The antigen binding regions and the heavy chain constant regions (with or without an altered amino acid sequence) can be connected by a suitable hinge or linker sequence. In certain embodiments, the antigen binding region is coupled to the immunoglobulin heavy chain constant region by a linker amino acid sequence or a human IgG hinge region. Appropriate IgG hinge regions comprise and include IgG1 or IgG4 hinge regions. In certain embodiments, the hinge region is an IgG1 hinge region. In certain embodiments, the hinge region is an IgG1 hinge regions with a with a C220S substitution per EU numbering. Suitable hinge regions include those described in Wu et al., “Multimerization of a chimeric anti-CD20 single-chain Fv-Fc fusion protein is mediated through variable domain exchange,” Protein Engineering, Design and Selection, Volume 14, Issue 12, December 2001, Pages 1025-1033; Shu et al, “Secretion of a single-gene-encoded immunoglobulin from myeloma cells.” Proceedings of the National Academy of Sciences September 1993, 90 (17) 7995-7999; Davis et al., “Abatacept binds to the Fc receptor CD64 but does not mediate complement-dependent cytotoxicity or antibody-dependent cellular cytotoxicity.” J Rheumatol. 2007 November; 34(11):2204-10. Appropriate hinges may also include a non-IgG based polypeptide linker. The linker amino acid sequence may predominantly include the following amino acid residues: Gly, Ser, Ala, or Thr. The linker peptide should have a length that is adequate to link two molecules in such a way that they assume the correct conformation relative to one another, and so that they retain the desired activity. In one embodiment, the linker is from about 1 to 50 amino acids in length or about 1 to 30 amino acids in length. In one embodiment, linkers of 1 to 20 amino acids in length may be used. Useful linkers include glycine-serine polymers, including for example (GS)n, (GSGGS)n, (GGGGS)n, and (GGGS)n, where n is an integer of at least one, glycine-alanine polymers, alanine-serine polymers, and other flexible linkers. Exemplary, linkers for linking antibody fragments or single chain variable fragments can include AAEPKSS, AAEPKSSDKTHTCPPCP, GGGG, or GGGGDKTHTCPPCP. Alternatively, a variety of non-proteinaceous polymers, including but not limited to polyethylene glycol (PEG), polypropylene glycol, polyoxyalkylenes, or copolymers of polyethylene glycol and polypropylene glycol, may find use as linkers, that is may find use as linkers.

The total size of the immunoconjugate may be such that it promotes tissue penetration, stability, and/or clearance. In certain embodiments, the immunoconjugate has a molecular weight of about 60 kDa to about 120 kDa. In certain embodiments, the immunoconjugate has a molecular weight of about 60 kDa to about 65 kDa, about 60 kDa to about 70 kDa, about 60 kDa to about 75 kDa, about 60 kDa to about 80 kDa, about 60 kDa to about 90 kDa, about 60 kDa to about 100 kDa, about 60 kDa to about 110 kDa, about 60 kDa to about 120 kDa, about 65 kDa to about 70 kDa, about 65 kDa to about 75 kDa, about 65 kDa to about 80 kDa, about 65 kDa to about 90 kDa, about 65 kDa to about 100 kDa, about 65 kDa to about 110 kDa, about 65 kDa to about 120 kDa, about 70 kDa to about 75 kDa, about 70 kDa to about 80 kDa, about 70 kDa to about 90 kDa, about 70 kDa to about 100 kDa, about 70 kDa to about 110 kDa, about 70 kDa to about 120 kDa, about 75 kDa to about 80 kDa, about 75 kDa to about 90 kDa, about 75 kDa to about 100 kDa, about 75 kDa to about 110 kDa, about 75 kDa to about 120 kDa, about 80 kDa to about 90 kDa, about 80 kDa to about 100 kDa, about 80 kDa to about 110 kDa, about 80 kDa to about 120 kDa, about 90 kDa to about 100 kDa, about 90 kDa to about 110 kDa, about 90 kDa to about 120 kDa, about 100 kDa to about 110 kDa, about 100 kDa to about 120 kDa, or about 110 kDa to about 120 kDa. In certain embodiments, the immunoconjugate has a molecular weight of about 60 kDa, about 65 kDa, about 70 kDa, about 75 kDa, about 80 kDa, about 90 kDa, about 100 kDa, about 110 kDa, or about 120 kDa. In certain embodiments, the immunoconjugate has a molecular weight of at least about 60 kDa, about 65 kDa, about 70 kDa, about 75 kDa, about 80 kDa, about 90 kDa, about 100 kDa, or about 110 kDa. In certain embodiments, the immunoconjugate has a molecular weight of at most about 65 kDa, about 70 kDa, about 75 kDa, about 80 kDa, about 90 kDa, about 100 kDa, about 110 kDa, or about 120 kDa.

In some embodiments, the immunoconjugate has a molecular weight greater than 60, 70, 75, 80, 82, 83, 85, 86, 87, 88 or 89 kDa. In some embodiments, the immunoconjugate has a molecular weight less than 110, 100, 95, 93, 91, 90, 89, 88, 87, 86, 85, 84, 83, 82, 81, or 80 kDa. In some embodiments, the immunoconjugate has a molecular weight greater than 60, 65, 70, 71, 72, 73, 74, 75, 76, 77, 78, or 79 kDa and less than 110, 100, 95, 93, 91, or 90 kDa.

The sizes of the immunoconjugates and/or the heavy chain constant region variants described herein allow for an increased safety profile or therapeutic index of the immunoconjugates included herein. Such a safety profile may be reflected in the reduction of accumulation of radiation in radio sensitive major tissues such as kidney and bone marrow and/or an increase in radiation accumulation in target tissues (i.e., a tumor or cancerous tissue) or more radio tolerant organs such as the liver.

In certain embodiments, the immunoconjugates of this disclosure result in a total radiation exposure per treatment as measured in Gray (Gy). In certain embodiments, the kidney is exposed to 20 Gy or less per treatment. In certain embodiments, the kidney is exposed to 19 Gy or less per treatment. In certain embodiments, the kidney is exposed to 18 Gy or less per treatment. In certain embodiments, the kidney is exposed to 17 Gy or less per treatment. In certain embodiments, the kidney is exposed to 16 Gy or less per treatment. In certain embodiments, the kidney is exposed to 15 Gy or less per treatment. In certain embodiments, the kidney is exposed to 14 Gy or less per treatment. In certain embodiments, the kidney is exposed to 13 Gy or less per treatment. In certain embodiments, the kidney is exposed to 12 Gy or less per treatment. In certain embodiments, the kidney is exposed to 11 Gy or less per treatment. In certain embodiments, the kidney is exposed to 10 Gy or less per treatment. In certain embodiments, the kidney is exposed to 9 Gy or less per treatment. In certain embodiments, the kidney is exposed to 8 Gy or less per treatment. In certain embodiments, the kidney is exposed to 5 Gy or less per treatment.

In certain embodiments, the immunoconjugates of this disclosure result in a total radiation exposure per treatment as measured in Gray (Gy). In certain embodiments, the bone marrow is exposed to 4 Gy or less per treatment. In certain embodiments, the bone marrow is exposed to 3 Gy or less per treatment. In certain embodiments, the bone marrow is exposed to 2 Gy or less per treatment. In certain embodiments, the bone marrow is exposed to 1.5 Gy or less per treatment. In certain embodiments, the bone marrow is exposed to 1.0 Gy or less per treatment. In certain embodiments, the bone marrow is exposed to 0.5 Gy or less per treatment.

In certain embodiments, the immunoconjugates of this disclosure result in an increased amount of radiation in the tumor compared to the kidney when measured as a percent injected dose per gram. In certain embodiments, the ratio of tumor percent injected dose per gram to kidney percent injected dose per gram is greater than 2:1, 3:1, 4:1, 5:1, 6:1, 7:1, 8:1, 9:1, or 10:1.

In certain embodiments, the immunoconjugates of this disclosure result in an increased amount of radiation in the tumor compared to the blood when measured as percent injected dose per gram. In certain embodiments, the ratio of tumor percent injected dose per gram to blood percent injected dose per gram is greater than 2:1, 3:1, 4:1, 5:1, 6:1, 7:1, 8:1, 9:1, or 10:1.

In certain embodiments, the immunoconjugates of this disclosure result in an increased amount of radiation in the tumor compared to the bone marrow when measured as percent injected dose per gram. In certain embodiments, the ratio of tumor percent injected dose per gram to bone marrow percent injected dose per gram is greater than 2:1, 3:1, 4:1, 5:1, 6:1, 7:1, 8:1, 9:1, or 10:1.

In certain embodiments, the immunoconjugates of this disclosure result in an increased amount of radiation in the liver compared to the kidney when measured as an injected dose per gram. In certain embodiments, the ratio of tumor percent injected dose per gram to bone marrow percent injected dose per gram is greater than 3:1, 4:1, 5:1, 6:1, 7:1, 8:1, 9:1, or 10:1.

In some embodiments, the invention contemplates a variant of an immunoconjugate of the invention that comprises a Fc region wherein the variant possesses some but not all effector functions, which make it a desirable candidate for applications in which the half-life of the immunoconjugate in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the immunoconjugate lacks FcγγR binding (hence likely lacking ADCC activity), but retains FcRn binding ability. The primary cells for mediating ADCC, NK cells, express FcγγRIII only, whereas monocytes express FcγγRI, FcγγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see e.g. Hellstrom, I. et al. Proc Natl Acad Sci USA 83:7059-7063 (1986)) and Hellstrom, I et al., Proc Natl Acad Sci USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc Natl Acad Sci USA 95:652-656 (1998). Clq binding assays may also be carried out to confirm that the immunoconjugate is unable to bind Clq and hence lacks CDC activity (see e.g., Clq and C3c binding ELISA in WO 2006/029879 and WO 2005/100402). To assess complement activation, a CDC assay may be performed (see e.g., Gazzano-Santoro et al., J. Immunol. Methods 202:163 (1996); Cragg, M. S. et al., Blood 101:1045-1052 (2003); Cragg, M. S. and M. J. Glennie, Blood 103:2738-2743 (2004)). FcRn binding and in vivo clearance/half-life determinations can also be performed using methods known in the art (see e.g., Petkova, S. B. et al., Int'l. Immunol. 18(12): 1759-1769 (2006)).

Immunoconjugates with reduced effector function include those with substitution of one or more of Fc region residues 238, 265, 269, 270, 297, 327, and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

The immunoconjugate may have altered effector function by comprising the following alterations L234A, L235E, G237A, A330S, and P331S per EU numbering, which reduce Fc receptor binding. See e.g., U.S. Pat. No. 8,613,926 or Andersson C, Wenander et al., “Rapid-onset clinical and mechanistic effects of anti-C5aR treatment in the mouse collagen-induced arthritis model.” Clin Exp Immunol. 2014 July; 177(1):219-33.

Certain immunoconjugate variants with improved or diminished binding to FcRs are described (see e.g., U.S. Pat. No. 6,737,056; WO 2004/056312; Shields et al., J. Biol. Chem. 9(2): 6591-6604 (2001)).

In some embodiments, alterations are made in the Fc region that result in altered (i.e., either improved or diminished) Clq binding and/or Complement Dependent Cytotoxicity (CDC), e.g., as described in U.S. Pat. No. 6,194,551; WO 1999/051642; Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976); Kim et al., J. Immunol. 24:249 (1994)), are described in US2005/0014934. Those antibodies comprise an Fe region with one or more substitutions therein which improve binding of the Fc region to FcRn. Such Fe variants include those with substitutions at one or more of Fc region residues: 434 or 435, e.g., substitution of Fc region residue N434A or R435A (U.S. Pat. No. 7,371,826). See also Duncan and Winter, Nature 322:738-40 (1988); U.S. Pat. Nos. 5,648,260; 5,624,821; and WO 1994/029351 concerning other examples of Fe region variants.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

As will be recognized by the person of ordinary skill in the art, certain teachings herein apply to antibody constructs, targeted imaging complexes, immunoconjugates and radioimmunoconjugates of the invention, notwithstanding that reference is made in the text to one only or two such compositions (e.g., immunoconjugate) as a non-limiting example. All such applications and embraced by the present invention.

### Chelating Agents

As described herein, in some embodiments a chelating agent is coupled to the tumor targeting moiety (e.g., the polypeptides comprising an antigen binding region and an immunoglobulin heavy chain constant region des cribbed herein). The chelating moiety allows for the tumor targeting moieties to be loaded with an appropriate radioisotope, such as a beta emitter or an alpha emitter. The chelator can be coupled to the antigen binding region, the heavy chain constant region, the immunoglobulin Fc region, or any combination thereof. Such coupling can suitably be by a covalent attachment to one or more amino acids of the immunoconjugate, the antigen binding region, the heavy chain constant region, the immunoglobulin Fc region, or any combination thereof.

In one embodiment, a chelating agent of the immunoconjugate is covalently linked to an antigen binding region, the heavy chain constant region, the immunoglobulin Fc region, or any combination thereof. In one embodiment, a chelating agent is covalently linked to the antigen binding region, the heavy chain constant region, the immunoglobulin Fc region, or any combination thereof directly (e.g., without the use of a spacer, stretcher or linker). In one embodiment the chelating agent is covalently linked to the antigen binding arm through a linker that is covalently linked to the chelating agent and covalently linked to the antigen binding arm. In one embodiment, the linker is hydrophilic (e.g., a PEG chain). In one embodiment, the linker is hydrophobic (e.g., an alkyl or alkene chain). Chelators may be linked or coupled to the immunoconjugates as described in Sadiki, A. et al. “Site-specific conjugation of native antibody.” Antibody Therapeutics 2020, 3, 271-284.

In some embodiments, the immunoconjugate is formed through the attachment of the chelator-linker in a site-specific manner, directed into a specific amino acid or glycan residue. In some embodiments, the site-specific conjugation involves directed functionalization of a specific lysine residue in the framework region with the chelator-linker. In other embodiments, this residue may be functionalized with a different reactive functional group which then reacts in a second step with chelator-linker to furnish the immunoconjugate. In some embodiments, this reactive functional group is thiopropionate.

In some embodiments, a non-native cysteine residue is engineered into the framework of the antibody as a site for thiol directed conjugation to furnish the immunoconjugate. In some embodiments, other non-native amino acids or an amino acid sequence is engineered into the framework to serve as the attachment site for the chelator-linker or for a secondary reactive group upon which the chelator-linker will be conjugated to furnish the immunoconjugate.

In some embodiments, a non-natural amino acid containing a cross-linking group is engineered into the framework for attachment of the chelator-linker. In some embodiments, this non-natural amino-acid contains an azide.

In some embodiments, the chelator-linker is attached to a glutamine residue through the action of a transglutaminase enzyme. In other embodiments, a secondary reactive group is attached by transglutaminase upon which the chelator-linker is added to furnish the immunoconjugate.

In some embodiments, the chelator-linker is attached by modifying one or more N-glycans with a reactive functional group through the action of a glycosidase, then conjugation of the chelator-linker to that site. In some embodiments, the glycan is modified through the action of β-galactosidase. In some embodiments, the glycan is modified with a glycoside that contains an azide for attachment of a properly functionalized chelator-linker.

In one embodiment, the immunoconjugate comprises more than one chelating agent, which are the same or different.

In one embodiment, an immunoconjugate having more than one chelating agent has more than one chelating agent attached to the same antigen binding arm.

In one embodiment, an immunoconjugate having more than one chelating agent and less than eleven chelating agents has more than two chelating agents, more than three chelating agents, more than four chelating agents, more than five chelating agents, more than six chelating agents, more than seven chelating agents, more than eight chelating agents, or more than nine chelating agents. In one embodiment, the chelating agents are the same. In one embodiment, each antigen binding arm is linked directly or indirectly to more than one chelating agent.

In one embodiment, the chelating agent comprises a radioisotope chelating component and a functional group that allows for covalent attachment to the antigen binding arm. In one embodiment, the functional group is directly attached to the radioisotope chelating component. In one embodiment the chelating agent further comprises a linker between the functional group and the radioisotope chelating component.

In one embodiment, the radioisotope chelating component comprises DOTA or a DOTA derivative. In one embodiment, the radioisotope chelating component comprises DOTAGA. In one embodiment, the radioisotope chelating component comprises macropa or a macropa derivative. In one embodiment, the radioisotope chelating component comprises Py4Pa or a Py4Pa derivative.

In a preferred embodiment, the chelating agent of an immunoconjugate is not attached to the antigen binding region in the antigen binding arm of the immunoconjugate.

In one embodiment, the chelating agent of the immunoconjugate is non-covalently associated with an antigen binding arm. In a preferred embodiment, the chelator is not associated with the antigen binding region in the antigen binding arm of the immunoconjugate.

In one embodiment, the chelating agent comprises DOTA or a DOTA derivative. In one embodiment, the chelating agent comprises DOTAGA. In one embodiment, the chelating agent comprises macropa or a macropa derivative. In one embodiment, the chelating agent comprises Py4Pa or a Py4Pa derivative. In one embodiment, the chelating agent comprises siderocalin or a siderocalin derivative.

In certain embodiments, the chelating agent is a radioisotope chelating agent. In certain embodiments, the radioisotope chelating agent is selected from the list consisting of: tetraazacyclododecane-1,4,7,10-tetraacetic acid (DOTA), α-(2-carboxyethyl)-1,4,7,10-tetraazacyclododecane-1,4,7,10-tetraacetic acid (DOTAGA), or (Py4Pa). In certain embodiments, the radioisotope chelating agent is DOTA. In certain embodiments, the radioisotope chelating agent is DOTAGA. In certain embodiments, the radioisotope chelating agent is Py4Pa. In certain embodiments, the radioisotope wherein the radioisotope chelating agent is directly coupled to the antigen binding region and/or the immunoglobulin heavy chain constant region. In certain embodiments, the radioisotope chelating agent is coupled to the antigen binding region or the immunoglobulin heavy chain constant region by a linker. In certain embodiments, the linker is selected from: 6-maleimidocaproyl (MC), maleimidopropanoyl (MP), valine-citrulline (val-cit), alanine-phenylalanine (ala-phe), p-aminobenzyloxycarbonyl (PAB), and those resulting from conjugation with linker reagents: N-Succinimidyl 4-(2-pyridylthio) pentanoate forming linker moiety 4-mercaptopentanoic acid (SPP), Succinimidyl 4-(N-maleimidomethyl)cyclohexane-1-carboxylate (SMCC), N-Succinimidyl 4-(2-pyridyldithio)butanoate (SPDB), N-Succinimidyl (4-iodo-acetyl) aminobenzoate (SIAB), polyethylene glycol (PEG), a polyethylene glycol polymers (PEGn), and S-2-(4-Isothiocyanatobenzyl) (SCN). In certain embodiments, the linker is selected from: polyethylene glycol (PEG), a polyethylene glycol polymers (PEG), and S-2-(4-isothiocyanatobenzyl) (SCN). In certain embodiments, the linker is PEG5. In certain embodiments, the linker is SCN. In certain embodiments, the radioisotope chelating agent is a linker-chelator selected from the list consisting of: TFP-Ad-PEG5-DOTAGA, p-SCN-Bn-DOTA, p-SCN-Ph-Et-Py4Pa, and TFP-Ad-PEG5-Ac-Py4Pa.

In some embodiments, the chelator is conjugated at a predefined ratio of polypeptide (i.e., antigen binding region and/or the immunoglobulin heavy chain constant) to chelator. In certain embodiments, the radioisotope chelating agent is coupled to the antigen binding region and/or the immunoglobulin heavy chain constant region at a ratio of 1:1 to 8:1. In certain embodiments, the radioisotope chelating agent is coupled to the antigen binding region and/or the immunoglobulin heavy chain constant region at a ratio of 1:1 to 6:1. In certain embodiments, the radioisotope chelating agent is coupled to the antigen binding region and/or the immunoglobulin heavy chain constant region at a ratio of 2:1 to 6:1.

For example, a bifunctional chelator is used to conjugate a radioisotope to a radioisotope delivery platform of the invention to create an immunoconjugate of the invention. (See e.g., Scheinberg D, McDevitt M, Curr Radiopharm 4: 306-20 (2011)). Examples of bifunctional chelators known in the art include DOTA, DTPA, DO3A-NHS, DOTAGA-NHS, DOTAGA-anhydride DOTAGA-TFP, p-SCN-Bn-DOTA, p-SCN-Bn-DTPA, p-SCN-Bn-CHX′A″-DTPA, p-SCN-Bn-TCMC, macropa-NCS, crown, p-SCN-Ph-Et-Py4Pa, 3,2-HOPO, and TCMC.

Examples of bifunctional chelators are 1,4,7,10-tetraazacyclododecane-1,4,7,10-tetraacetic acid (DOTA), diethylene triamine pentaacetic acid (DTPA), and related analogs of the aforementioned. Such chelators are suitable for coordinating metal ions like α and β-emitting radionuclides.

In some embodiments the chelating agent of an immunoconjugate or radioimmunoconjugate of the invention is selected from the group comprising bifunctional chelator, DOTA, D03A-NHS, DOTAGA-NHS, DOTAGA-anhydride DOTAGA-TFP, p-SCN-Bn-DOTA, p-SCN-Bn-DTPA, p-SCN-Bn-CHX-A″-DTPA, p-SCN-Bn-TCMC, macropa-NCS (Thiele N A, et al. Angew. Chem. Int. Ed. 56:1 (2017)), crown (Yang H, et al. Chem. Eur. J. 26:11435 (2020)), P—SCN-Ph-Et-Py4Pa (Li L, et al. Bioconjugate Chem. ASAP (2020)), 3,2-HOPO (Wickstroem K, et al. Int. J. Rad. Onc. Biol. Phys. 105:410 (2019)) (For a review of these and other bifunctional chelators See e.g., Price E W and Orvig C Chem. Soc. Rev., 2014, 43:260 (2014) and Brechbiel M W Q. J. Nucl. Med. Mol. Imaging 52:166 (2008)).

In some embodiments the chelating agent of an immunoconjugate or radioimmunoconjugate of the invention is selected from the group consisting of bifunctional chelator, DOTA, D03A-NHS, DOTAGA-NHS, DOTAGA-anhydride DOTAGA-TFP, p-SCN-Bn-DOTA, p-SCN-Bn-DTPA, p-SCN-Bn-CHX-A″-DTPA, p-SCN-Bn-TCMC, macropa-NCS (Thiele N A, et al. Angew. Chem. Int. Ed. 56:1 (2017)), crown (Yang H, et al. Chem. Eur. J. 26:11435 (2020)), P—SCN-Ph-Et-Py4Pa (Li L, et al. Bioconjugate Chem. ASAP (2020)), 3,2-HOPO (Wickstroem K, et al. Int. J. Rad. Onc. Biol. Phys. 105:410 (2019)) (For a review of these and other bifunctional chelators see e.g., Price E W and Orvig C Chem. Soc. Rev., 2014, 43:260 (2014) and Brechbiel M W Q. J. Nucl. Med. Mol. Imaging 52:166 (2008)).

For ²²⁵Ac immunoconjugates, there are a variety of acyclic and cyclic ligands known in the art as suitable chelators (see e.g., Davis I, et al., Nucl Med Biol 26: 581 (1999); Chappell L, et al., Bioconjug Chem 11: 510 (2000); Chappell, L, et al., Nucl Med Biol 30: 581 (2003); McDevitt M, et al., Appl Radiat Isot 57: 841 (2002); Gouin S, et al., Org Biomol Chem 3: 453 (2005); Thiele N, et al., Angew Chem Int Ed Engl 56: 14712 (2017)).

In certain embodiments, the chelator is a chelator suitable for alpha emitter chelation. Some chelators suitable for alpha emitters are described in Yang et al, “Harnessing α-Emitting Radionuclides for Therapy: Radiolabeling Method Review.” J Nucl Med. 2022 January; 63(1):5-13.

It is understood that the immunoconjugates described herein comprise one or more chelating moieties. In some embodiments, the immunoconjugates described herein comprise one or more than one chelating moieties, wherein each chelating moiety is the same.

### Radionuclides

In some embodiments, the radionuclide in the immunoconjugates described herein is an Auger electron-emitting radionuclide. In some embodiments, the radionuclide is an α-emitting radionuclide. In some embodiments, the radionuclide is a β-emitting radionuclide. In some embodiments, the radionuclide is a γ-emitting radionuclide. In some embodiments, the type of radionuclide used in a non-peptide targeted therapeutic compound can be tailored to the specific type of cancer, the type of targeting moiety (e.g., non-peptide ligand), etc. Radionuclides that undergo α-decay emit α-particles (helium ions with a +2 charge) from their nuclei. As a result of α-decay the daughter nuclide has 2 protons less and 2 neutrons less than the parent nuclide. This means that in α-decay, the proton number is reduced by 2 while the nucleon number is reduced by 4. Radionuclides that undergo β-decay emit β-particles (electrons) from their nuclei. During β-decay, one of the neutrons changes into a proton and an electron. The proton remains in the nucleus while the electron is emitted as a β-particle. This means that in β-decay, the nucleus loses a neutron but gains a proton. In γ-decay, a nucleus in an excited state (higher energy state) emits a γ-ray photon to change to a lower energy state. There is no change in the proton number and nucleon number during the γ-decay. The emission of γ-rays often accompanies the emission of α-particles and β-particles.

Auger electrons (AEs) are very low energy electrons that are emitted by radionuclides that decay by electron capture (EC) (e.g., ¹¹¹In, ⁶⁷Ca, technetium-99m (^{99m}Tc) platinum-195m (^{195m}Pt), iodine-125 (¹²⁵I), and iodine-123 (¹²³I). This energy is deposited over nanometer-micrometer distances, resulting in high linear energy transfer that is potent for causing lethal damage in cancer cells. Thus, AE-emitting radiotherapeutic agents have great potential for treatment of cancer.

β-Particles are electrons emitted from the nucleus. They typically have a longer range in tissue (of the order of 1-5 mm) and are the most frequently used.

α-Particles are helium nuclei (two protons and two neutrons) that are emitted from the nucleus of a radioactive atom. Depending on their emission energy, they can travel 50-100 μm in tissue. They are positively charged and are orders of magnitude larger than electrons. The amount of energy deposited per path length travelled (designated ‘linear energy transfer’) of α-particles is approximately 400 times greater than that of electrons. This leads to substantially more damage along their path than that caused by electrons. An α-particle track leads to a preponderance of complex and largely irreparable DNA double-strand breaks. The absorbed dose required to achieve cytotoxicity relates to the number of α-particles traversing the cell nucleus. With use of this as a measure, cytotoxicity may be achieved with a range of 1 to 20 α-particle traversals of the cell nucleus. The resulting high potency, combined with the short range of α-particles (which reduces normal organ toxicity), has led to substantial interest in developing α-particle-emitting agents. The α-particle emitters typically used include ²¹²Bi, ²¹²Pb, ²¹³Bi, ²²⁵Ac, ²²³Ra, and ²²⁹Th.

In some embodiments, the radionuclide is a diagnostic or therapeutic radionuclide.

### Representative Radionuclides

| Isotope | t_{1/2} (h) | Decay mode |
|---|---|---|
| ⁶⁶Ga | 9.5 | β+ (56%), EC (44%) |
| ⁶⁷Ga | 78.2 | EC (100%) |
| ⁶⁸Ga | 1.1 | β+ (90%), EC (10%) |
| ¹¹¹In | 67.2 | EC (100%) |
| ^{114m}In | 49.5 d | EC (100%) |
| ¹¹⁴In (daughter) | 73 s | β− (100%) |
| ¹⁷⁷Lu | 159.4 | β− (100%) |
| ⁸⁹Zr | 78.5 | β+ (23%), EC (77%) |
| ²¹²Bi | 1.1 | α (36%), β− (64%) |
| ²¹³Bi | 0.76 | α (2.2%), β− (97.8%) |
| ²¹²Pb (daughter is ²¹²Bi) | 10.6 | β− (100%) |
| ²²⁵Ac | 240 | α (100%) |

In some embodiments, the radionuclide is an Auger electron-emitting radionuclide. In some embodiments, the radionuclide is an Auger electron-emitting radionuclide that is ¹¹¹In, ⁶⁷Ga, ⁶⁸Ga, ^{99m}Tc, or ^{195m}Pt. In some embodiments, the radionuclide is an Auger electron-emitting radionuclide that is ¹¹¹In, ⁶⁷Ga, ⁶⁸Ga, or ^{99m}Tc.

In some embodiments, the radionuclide is an α-emitting radionuclide. In some embodiments, the radionuclide is an α-emitting radionuclide that is ²²⁵Ac, ²¹³Bi, ²²³Ra, or ²¹²Pb. In some embodiments, the radionuclide is an α-emitting radionuclide that is ²²⁵Ac. In some embodiments, the radionuclide is an β-emitting radionuclide. In some embodiments, the radionuclide is a β-emitting radionuclide that is ⁹⁰Y, ¹⁷⁷Lu, ¹⁸⁶Re, ¹⁸⁸Re, ⁶⁴Cu, ⁶⁷Cu, ¹⁵³Sm, ⁸⁹Sr, ¹⁹⁸Au, ¹⁶⁹Er, ¹⁶⁵Dy, ^{99m}Tc, ⁸⁹Zr, or ⁵²Mn. In some embodiments, the radionuclide is a β-emitting radionuclide that is ⁹⁰Y, ¹⁷⁷Lu, ^{99m}Tc, or ⁸⁹Zr.

In some embodiments, the radionuclide is a γ-emitting radionuclide. In some embodiments, the radionuclide is a γ-emitting radionuclide that is cobalt-60 (⁶⁰Co), palladium-103 (¹⁰³Pd), cesium-137 (¹³⁷Cs), ytterbium-169 (¹⁶⁹Yb), iridium-192 (¹⁹²Ir), ²¹²Bi, ²¹³Bi, or ²²⁶Ra.

### Linkers

In some embodiments, a linker is used that connects the tumor targeting moiety and the radionuclide chelator moiety. In some embodiments, L is a hydrophobic linker. In some other embodiments, L is a hydrophilic linker. In some embodiments, the linker is flexible. In some embodiments, the linker is rigid. In some embodiments, the linker is linear. In other embodiments, the linker is branched. In some embodiments, a branched linker allows for conjugation to more than one chelator moieties. In some embodiments, a branched linker allows for conjugation to cell-penetrating peptides (CPPs) to enhance cell penetration. In some embodiments, the linker comprises a linear structure. In some embodiments, the linker comprises a non-linear structure. In some embodiments, the linker comprises a branched structure. In some embodiments, the linker comprises a cyclic structure. In some embodiments, the linker comprises one or more linear structures, one or more non-linear structures, one or more branched structures, one or more cyclic structures, one or more flexible moieties, one or more rigid moieties, or combinations thereof.

Linker length may be viewed in terms of the number of linear atoms between the radionuclide chelator moiety and the tumor targeting moiety, with cyclic moieties to be counted by taking the shortest route around the ring. In some embodiments, the linker has a linear stretch of between 1 to 100 atoms, between 1-50 atoms, between 1-40 atoms, between 1-30 atoms, in other embodiments 1-20 atoms, in still other embodiments 1-15 atoms, in still other embodiments 1-10 atoms, and in still other embodiments 1-5 atoms. In some embodiments, the length of the linker is a range with a lower limit selected from the group consisting of 1, 2, 3, 4, 5, 6, 7, 8, 9, 10, and an upper limit selected from the group consisting of 5, 6, 7, 8, 9, 10, 11, 12, 13, 14, 15, 16, 17, 18, 19, 20, 25, and 30. In some embodiments, the length of the linker is a range with a lower limit selected from the group consisting of 10, 11, 12, 13, 14, 15, 16, 17, 18, 19, 20, and an upper limit selected from the group consisting of 30, 31, 32, 33, 34, 35, 36, 37, 38, 39, 40, 41, 42, 43, 44, 45, 46, 47, 48, 49, and 50.

Linker considerations include the effect on physical or pharmacokinetic properties of the resulting radioimmunoconjugate, such as solubility, lipophilicity, hydrophilicity, hydrophobicity, stability (more or less stable as well as planned degradation), rigidity, flexibility, immunogenicity, modulation of antibody binding, and the like.

An optimal linker should link the carrier mAb to the radiochelator without impairing the functionality of either, provide a stable linkage in circulation, and degrade under specific conditions. Stability in circulation has proven to constitute a particularly important factor for success using this strategy.

In malignancies, the accessibility of tumor cells enables targeting by specific antibodies. Antigens that are expressed on the tumor cells but not on normal healthy cells allow selective targeting of the tumor cells while sparing the non-tumor cells. mAb tagged with short-lived radionuclides emitting short-ranged, high linear energy transfer particles represent an attractive means of treating tumors. These short-ranged particles may be capable of single-cell kill while sparing bystanders. High energy alpha-emitters used in radioimmunotherapy include bismuth-212 and -213, astatine-211, and actinium-225. This latter isotope is an in vivo isotope generator, in that it has a long (10 day) half-life and decays via alpha-emission through three short-lived atoms, each of which yields an alpha particle.

In some instances, a limitation to the general use of tumor targeted ²²⁵-Ac radioimmunotherapy is the excessive radiation of normal tissues by the radioimmunoconjugate. With the exception of antibodies targeting leukemias, most (>99%) of the injected antibody dose remains circulating in the blood or is not associated with the target tumor. The prolonged circulation time of untargeted antibodies, typically days to weeks, results in radiation exposure of normal organs that catabolize or retain proteins and peptides, primarily liver and kidneys.

In the case of in vivo decay of alpha-emitters, an additional major potential obstacle to their safe use is the sequential release of three unchelated alpha-emitting daughter atoms following initial decay of the initial radionuclide. Uptake into organs such as the kidney or the liver could induce toxicity. One attractive approach to overcome the long circulation time of untargeted radioimmunoconjugates is the development of linkers that allow conditional release of the chelated radioactive payload. The introduction of a cleavage site in the linkage between the radiochelate and the mAb is meant to permit the release of a low molecular weight radiochelate from its carrier mAb and its subsequent rapid clearance once the radioconjugate has accumulated in metabolizing organs. The chelated radiometal then rapidly clears the body through the kidneys, thereby reducing toxicity. This strategy has been attempted using disulfide, ester, tartramide, and peptide bonds (Kukis D L, et al., Cleavable linkers to enhance selectivity of antibody-targeted therapy of cancer. Cancer Biother Radiopharm. 2001; 16:457-467; Quadri S M, Vriesendorp H M. Effects of linker chemistry on the pharmacokinetics of radioimmunoconjugates. Q J Nucl Med. 1998; 42:250-261). An optimal linker should link the carrier mAb to the radiochelator without impairing the functionality of either, provide a stable linkage in circulation, and degrade under specific conditions. Stability in circulation has proven to constitute a particularly important factor for success using this strategy.

Peptide linkers, which are usually more stable than esters and disulfides in serum, have been most successful in radioimmunoconjugates and in the design of antibody-drug conjugates (ADC). The most extensively studied peptide linkers are sensitive to cathepsins. More precisely, ADCs incorporating the dipeptide valine-citrulline have been shown to enter the target cells and migrate to the lysosomes, where they release their drug specifically under the action of cysteine proteases. The most advanced strategy for the release of a radionuclide from a radioimmunoconjugate is based on cathepsin-sensitive peptide linkers (DeNardo G L, et al., Preclinical evaluation of cathepsin-degradable peptide linkers for radioimmunoconjugates. Clin Cancer Res. 2003; 9:3865S-3872S; DeNardo G L, et al. Comparison of 1,4,7,10-tetraazacyclododecane-N,N′,N″,N′″-tetraacetic acid (DOTA)-peptide-ChL6, a novel immunoconjugate with catabolizable linker, to 2-iminothiolane-2-[p-(bromoacetamido)benzyl]-DOTA-ChL6 in breast cancer xenografts. Clin Cancer Res. 1998; 4:2483-2490; DeNardo S J, et al. Enhanced therapeutic index of radioimmunotherapy (RIT) in prostate cancer patients: comparison of radiation dosimetry for 1,4,7,10-tetraazacyclododecane-N,N′,N″,N′″-tetraacetic acid (DOTA)-peptide versus 21T-DOTA monoclonal antibody linkage for RIT. Clin Cancer Res. 2003; 9:3938S-3944S). In some instances, the use of cathepsin B labile radioconjugates (e.g., glycylglycylglycyl-L-p-isothiocyanatophenylalanine-) demonstrated a decrease in the liver dose and a slightly increased tumor dose was observed (DeNardo G L, et al., Clin Cancer Res. 2003; 9:3865S-3872S; DeNardo G L, et al. Clin Cancer Res. 1998; 4:2483-2490).

In some embodiments, a linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids.

In some embodiments, a linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences, proline-rich sequences, and regions rich in double and/or triple bonds.

In some embodiments, the linker comprises a peptidyl linker. In some embodiments, the peptidyl linker may be between 3-20 amino acids long, such as repeats of a single amino acid residue (e.g. polyglycine) or combinations of amino acid residues to give a peptide linker which imparts favorable pharmacokinetics.

In some embodiments, the linker comprises a peptide linkage. The peptide linkage comprises L-amino acids and/or D-amino acids. In some embodiments, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a linker is cleavable. In some embodiments, a linker is designed for cleavage in the presence of particular conditions or in a particular environment, such conditions or environments near such targeted cells, tissues, or regions. Cleavable linkers rely on the inherent properties of a cell's cytoplasmic compartments for selective release of the cytotoxic drug. Such linkers mainly include chemically cleavable linkers that respond to low pH (acid-labile linkers) or reducing environment (disulfide linkers), and enzymatically cleavable linkers that are susceptible to the action of certain lysosomal enzymes (peptide linkers or β-glucuronide linkers).

In some embodiments, a linker is cleavable under physiological conditions. In some embodiments, a linker is cleavable under intracellular conditions. In some embodiments, the linker is chemically cleavable. In some embodiments, the linker is enzymatically cleavable (e.g., protease-sensitive, peptidase-sensitive) linker. In some embodiments, the linker is pH-sensitive, i.e., sensitive to hydrolysis at certain pH values. For example, the pH-sensitive linker can be hydrolyzable under acidic conditions. For example, a linker can be an acid-labile linker that is hydrolyzable in the lysosome (e.g., a hydrazone, semicarbazone, thiosemicarbazone, cis-aconitic amide, orthoester, acetal, ketal, or the like). Such linkers can be relatively stable under neutral pH conditions, such as those in the blood, but are unstable below pH 7.0, such as pH 6.5 to 4.5, the approximate pH of the lysosome and/or endosome.

In some embodiments, the linker is cleaved by an intracellular peptidase or protease enzyme, including, but not limited to, a lysosomal or endosomal protease. In some embodiments, the linker is cleaved by a glycosidase, e.g., glucuronidase. β-glucuronide linkers can be readily cleaved by the abundant lysosomal enzyme β-glucuronidase, facilitating facile and selective release of the active drug. In other embodiments, the linker is not cleavable.

In some embodiments, a linker component comprises an amino acid unit. In one such embodiment, the amino acid unit allows for cleavage of the linker by a protease, thereby facilitating release of the drug from the immunoconjugate upon exposure to intracellular proteases, such as lysosomal enzymes. Exemplary amino acid units include, but are not limited to, a dipeptide, a tripeptide, a tetrapeptide, and a pentapeptide. Exemplary dipeptides include: valine-citrulline (vc or val-cit), alanine-phenylalanine (af or ala-phe); phenylalanine-lysine (fk or phe-lys); or N-methyl-valine-citrulline (Me-val-cit). These dipeptide linkers show good stability in serum, yet can be recognized and rapidly hydrolyzed by certain lysosomal proteases, such as cathepsin B, following internalization. Exemplary tripeptides include: glycine-valine-citrulline (gly-val-cit) and glycine-glycine-glycine (gly-gly-gly).

An amino acid unit may comprise amino acid residues that occur naturally, as well as minor amino acids and non-naturally occurring amino acid analogs, such as citrulline. Amino acid units can be designed and optimized in their selectivity for enzymatic cleavage by a particular enzyme, for example, a tumor-associated protease, cathepsin B, C and D, or a plasmin protease.

In some embodiments, the linker is cleaved by a protease, a matrix metalloproteinase, a serine protease, or a combination thereof. In some embodiments, the linker is cleaved by a reducing agent. In some embodiments, the linker is cleaved by an oxidizing agent or oxidative stress.

In some embodiments, the linker is cleaved by an MMP. The hydrolytic activity of matrix metalloproteinases (MMPs) has been implicated in the invasive migration of metastatic tumor cells. In some embodiments, a linker includes the amino-acid sequences PLG-C(Me)-AG, PLGLAG which are cleaved by the metalloproteinase enzymes MMP-2, MMP-9, or MMP-7 (MMPs involved in cancer and inflammation).

In some embodiments, the linker is cleaved by proteolytic enzymes or reducing environment, as may be found near cancerous cells. Such an environment, or such enzymes, are typically not found near normal cells.

In some embodiments, the linker is cleaved by serine proteases including but not limited to thrombin and cathepsins. In some embodiments, the linker is cleaved by cathepsin K, cathepsin S, cathepsin D, cathepsin E, cathepsin W, cathepsin F, cathepsin A, cathepsin C, cathepsin H, cathepsin Z, or any combinations thereof. In some embodiments, the linker is cleaved by cathepsin K and/or cathepsin S.

In some embodiments, the linker is cleaved in a necrotic environment. Necrosis often leads to the release of enzymes or other cell contents that may be used to trigger cleavage of a linker. In some embodiments, cleavage of the linker occurs by necrotic enzymes (e.g., by calpains).

In some embodiments, the linker comprises one or more of di-sulfide bonds.

Alternatively, the linker may be a non-peptidyl linker. Typical examples of these types of linker would be those based on straight or branched chain hydrocarbons or polyethylene glycols of varying lengths. These may incorporate other groups to effect solubility, rigidity, isoelectric point, such as aromatic or non-aromatic rings, halogens, ketones, aldehydes, esters, sulfonyls, phosphate groups, and so on.

In some embodiments, a linker component is “self-immolative” or a “non-self-immolative.” A “non-self-immolative” spacer unit is one in which part or all of the spacer unit remains bound to the conjugate moiety upon enzymatic (e.g., proteolytic) cleavage. Examples of non-self-immolative spacer units include, but are not limited to, a glycine spacer unit and a glycine-glycine spacer unit. Other combinations of peptidic spacers susceptible to sequence-specific enzymatic cleavage are also contemplated. For example, enzymatic cleavage of a glycine-glycine spacer unit by a tumor-cell associated protease would result in release of a glycine-glycine-drug moiety from the remainder of the immunoconjugate. In one such embodiment, the glycine-glycine-drug moiety is then subjected to a separate hydrolysis step in the tumor cell, thus cleaving the glycine-glycine spacer unit from the chelator moiety.

A “self-immolative” spacer unit allows for release of the drug moiety without a separate hydrolysis step. In certain embodiments, a spacer unit of a linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent (see, e.g., Hamann et al. (2005) Expert Opin. Ther. Patents (2005) 15: 1087-103. In one embodiment, the spacer unit is p-aminobenzyloxycarbonyl (PAB). In certain embodiments, the phenylene portion of a p-aminobenzyl unit is substituted with Qm, wherein Q is halogen, nitro, cyano, —OH, —OR⁵, —CO₂H, —NHR⁵, —C(═O)NHR⁵, —NHC(═O)R⁵ and substituted C₁-C₆alkyl, wherein the substituted C₁-C₆alkyl is substituted with, —OH, —CO₂H, —NHR⁵, —C(═O)NHR⁵, and —NHC(═O)R⁵; and m is an integer ranging from 0-4 (e.g., see Yam B. Poudel, et al., ACS Medicinal Chemistry Letters 2020 11 (11), 2190-2194). Examples of self-immolative spacer units further include, but are not limited to, aromatic compounds that are electronically similar to p-aminobenzyl alcohol (see, e.g., US 2005/0256030 A1), such as 2-aminoimidazol-5-methanol derivatives (Hay et al. (1999) Bioorg. Med. Chem. Lett. 9: 2237) and ortho- or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223); appropriately substituted bicyclo[2.2.1] and bicyclo[2.2.2] ring systems (Storm, et al., J Amer. Chem. Soc., 1972, 94: 5815); and 2-aminophenylpropionic acid amides (Amsberry, et al., J. Org. Chem., 1990, 55: 5867). Elimination of amine-containing drugs that are substituted at the a-position of glycine (Kingsbury, et al., J. Med. Chem., 1984, 27: 1447) are also examples of self-immolative spacers useful in ADCs.

In some embodiments, the immunoconjugate comprises a linker, such as, e.g., a dendritic type linker for covalent attachment of more than one drug moiety through a branching, multifunctional linker moiety to an antibody (Sun et al (2002) Bioorganic & Medicinal Chemistry Letters 12: 2213-5; Sun et al (2003) Bioorganic & Medicinal Chemistry 11: 1761-8). Dendritic linkers can increase the molar ratio of drug to antibody, i.e., loading, which is related to the potency of the ADC. Thus, where a cysteine-engineered antibody bears only one reactive cysteine thiol group, a multitude of drug moieties may be attached through a dendritic linker.

In some embodiments, the linker that connects the tumor targeting moiety to the chelating moiety (R¹) or a radionuclide complex thereof comprises —X¹-L-L^{c}-; L is as described herein; L^{c} is —C(═O)—, -phenyl-C(═O)—, —NHC(═S)—, —X-phenyl-NHC(═S)—, —S—, —C(═O)CH₂—,

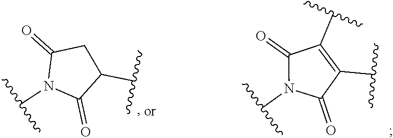 [SMILES: *C1CC(=O)N(*)C1=O ; *C1=C(*)C(=O)N(*)C1=O]

X is absent, —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}, —NR^{a}C(═S)NR^{a}—, —NR^{a}C(═O)O—; each R^{a} is independently selected from hydrogen, and C₁-C₄alkyl.

In some embodiments, L^{c} is —C(═O)—. In some embodiments, L^{c} is -phenyl-C(═O)—. In some embodiments, L^{c} is —NHC(═S)—. In some embodiments, L^{c} is —X-phenyl-NHC(═S)—. In some embodiments, X is absent or —NHC(═S)NH—. In some embodiments, L^{c} is —NHC(═S)NH—. In some embodiments, L^{c} is —S—, —C(═O)CH₂—,

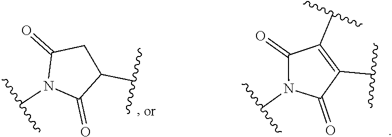 [SMILES: *C1CC(=O)N(*)C1=O ; *C1=C(*)C(=O)N(*)C1=O]

In some embodiments, L^{c} is —C(═O)CH₂—. In some embodiments, L^{c} is

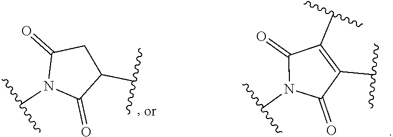 [SMILES: *C1CC(=O)N(*)C1=O ; *C1=C(*)C(=O)N(*)C1=O]

In some embodiments, X¹ is —O—, —S—, —S(═O)—, —S(═O)₂—, —NR^{a}—, —C(═O)—, —NR^{a}C(═O)—, —C(═O)NR^{a}—, —(C₁-C₆alkylene)-X²—, —O—(C₁-C₆alkylene)-X²—, or —(C₄-C₂₀polyethylene glycol)-X²—; X² is absent, —C(═O)—, —C(═O)NR^{a}—, or —C(═O)X⁴—; X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(═O)R⁵, and wherein any free carboxylic acid of any amino acid is optionally replaced with —C(═O)NH—R⁵.

In some embodiments, X¹ is absent, —O—, —S—, —S(═O)—, —S(═O)₂, —NR^{a}—, —C(═O)—, —NR^{a}C(═O)—, or —C(═O)NR^{a}—. In some embodiments, X¹ is absent, —O—.

In some embodiments, X¹ is —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —OCH₂—X²—, —OCH₂CH₂—X²—, —OCH₂CH₂CH₂—X²—, —OCH₂CH₂CH₂CH₂—X²—, —OCH₂CH₂CH₂CH₂CH₂—X², —OCH₂CH₂CH₂CH₂CH₂CH₂—X²—;

In some embodiments, X¹ is —OCH₂—, —OCH₂—X²—, —OCH₂CH₂— or —OCH₂CH₂—X²—; X² is —C(═O)X⁴—; X⁴ is —NH—, —N(CH₃)—, —N(CH₂CH₃)—, —NHS(═O)₂—, —N(CH₃)S(═O)₂—, —N(CH₂CH₃)S(═O)₂—, lysine, glutamine, glutamate, glycine, leucine, lysine, methionine, phenylalanine, serine, tyrosine, valine, citrulline, methionine-valine-lysine, glycine-phenylalanine-glycine-glycine, tyrosine-arginine-valine, arginine-valine, or combination thereof, wherein any free amine (—NH₂) of an amino acid is optionally independently substituted with R⁵ or —C(═O)R⁵, and wherein any free carboxylic acid of any amino acid is optionally replaced with —C(═O)NH—R⁵.

In some embodiments, X¹ is —OCH₂—, —OCH₂—X²—, —OCH₂CH₂— or —OCH₂CH₂—X²—; X² is —C(═O)X⁴—; X⁴ is —NH—, —N(CH₃)—, —N(CH₂CH₃)—, —NHS(═O)₂—, —N(CH₃)S(═O)₂—, or —N(CH₂CH₃)S(═O)₂—.

In some embodiments, X¹ is —OCH₂—, —OCH₂—X²—, —OCH₂CH₂— or —OCH₂CH₂—X²—; X² is —C(═O)X⁴—; X⁴ is lysine, glutamine, glutamate, glycine, leucine, lysine, methionine, phenylalanine, serine, tyrosine, valine, citrulline, methionine-valine-lysine, glycine-phenylalanine-glycine-glycine, tyrosine-arginine-valine, arginine-valine, or combination thereof, wherein any free amine (—NH₂) of an amino acid is optionally independently substituted with R⁵ or —C(═O)R⁵, and wherein any free carboxylic acid of any amino acid is optionally replaced with —C(═O)NH—R⁵.

In some embodiments, L is -L¹-, -L²-, -L³-, -L⁴-, -L⁵-, -L¹-L²-L³-L⁴-L⁵-, or combinations thereof;
L¹ is absent, unsubstituted or substituted C₁-C₁₀alkylene, unsubstituted or substituted C₁-C₁₀heteroalkylene, unsubstituted or substituted C₂-C₂₀alkenylene, unsubstituted or substituted C₂-C₂₀alkynylene, C₄-C₂₀polyethylene glycol, —(X³CH₂CH₂)ₜ—, unsubstituted or substituted cycloalkylene, unsubstituted or substituted heterocycloalkylene, unsubstituted or substituted arylene, unsubstituted or substituted heteroarylene;
   each X³ is independently selected from 0 and NR⁴;
   each t is independently 1, 2, 3, 4, 5, 6, 7, 8, 9, 10, 11, or 12;
L² is absent, unsubstituted or substituted C₁-C₁₀alkylene, unsubstituted or substituted C₁-C₁₀heteroalkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₘ—(CH₂)_{P}—, —C(═O)NR⁴—(CH₂CH₂X³)ₘ—(CH₂)ₚ—, —NR⁴C(═O)—(CH₂CH₂X³)ₘ—(CH₂)ₚ—, or —(CH₂CH₂X³)ₘ—(CH₂)ₚ—;
   each R⁴ is independently selected from hydrogen, and C₁-C₆alkyl;
   each m is independently 1, 2, 3, 4, 5, 6, 7, 8, 9, 10, 11, or 12;
   each p is independently 0, 1, or 2;
L³ is absent, or one or more independently selected groups selected from: natural or unnatural amino acids, and an optional amino(unsubstituted or substituted benzyl)carbamate; wherein any free amine (—NH₂) of an amino acid is optionally independently substituted with R⁵ or —C(═O)R⁵, wherein any free carboxylic acid of any amino acid is optionally replaced with —C(═O)NH—R⁵, and wherein substituted benzyl is substituted with 1 or 2 groups selected from halogen, —OH, —OR⁵, —CO₂H, —NHR⁵, —C(═O)NHR⁵, —NHC(═O)R⁵ and substituted C₁-C₆alkyl, wherein the substituted C₁-C₆alkyl is substituted with, —OH, —CO₂H, —NHR⁵, —C(═O)NHR⁵, and —NHC(═O)R⁵;
L⁴ is absent, unsubstituted or substituted C₁-C₁₀alkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, or —(X³CH₂CH₂)ₙ—;
   each n is independently 1, 2, 3, 4, 5, 6, 7, 8, 9, 10, 11, or 12;
   each q is independently 0, 1, or 2;
L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, —C(═O)—(X³CH₂CH₂)ₙ—, or —(X³CH₂CH₂)ₙ—;
   each n is independently 1, 2, 3, 4, 5, 6, 7, 8, 9, 10, 11, or 12;
   each q is independently 0, 1, or 2;
   each R^{a} is independently selected from hydrogen, and C₁-C₄alkyl;
   each R^{b} is independently selected from hydrogen, and C₁-C₄alkyl;
wherein heteroalkylene is an alkylene where one carbon atom is replaced with —O—, —S—, —S(═O)—, —S(═O)₂—, —S(═O)(═NH)—, —S(═O)(═NR⁶)—, —NR⁶—, —P(═O)OR^{b}—, —NR^{a}C(═O)—, —C(═O)NR^{a}—, —OC(═O)NR^{a}—, —NR^{a}C(═O)NR^{a}—, —NR^{a}C(═N—CN)NR^{a}—, —NR^{a}C(═N—R⁶)NR^{a}—, or —NR^{a}C(═O)O—;
wherein when any one of -L¹-, -L²-, -L³-, -L⁴-, and -L⁵- is substituted then -L¹-, -L²-, -L³-, -L⁴-, and -L⁵- is substituted with 1, 2, 3, or 4 groups selected from halogen, —OH, —OR⁵, —CO₂H, —NHR⁵, —C(═O)NHR⁵, —NHC(═O)R⁵ and unsubstituted or substituted C₁-C₆alkyl, wherein the substituted C₁-C₆alkyl is substituted with, —OH, —CO₂H, —NHR⁵, —C(═O)NHR⁵, or —NHC(═O)R⁵;
each R⁵ is independently selected from C₁-C₁₀alkyl, C₄-C₃₀polyethylene glycol, and unsubstituted or substituted arylene, unsubstituted or substituted heteroarylene;
each R⁶ is independently selected from C₄-C₃₀polyethylene glycol, unsubstituted or substituted arylene, unsubstituted or substituted heteroarylene, unsubstituted or substituted C₁-C₆alkyl, wherein the substituted C₁-C₆alkyl is substituted with halogen, —OH, —CO₂H, —NHR⁵, —C(═O)NHR⁵, or —NHC(═O)R⁵.

In some embodiments, L is -L¹-. In some embodiments, L is -L²-. In some embodiments, L is -L3-. In some embodiments, L is -L⁴-. In some embodiments, L is -L⁵-. In some embodiments, L is -L¹-L²-. In some embodiments, L is -L¹-L³-. In some embodiments, L is -L¹-L⁴-. In some embodiments, L is -L¹-L⁵-. In some embodiments, L is -L²-L³-. In some embodiments, L is -L²-L⁴-. In some embodiments, L is -L²-L⁵-. In some embodiments, L is -L³-L⁴-. In some embodiments, L is -L³-L⁵-. In some embodiments, L is -L⁴-L⁵-. In some embodiments, L is -L¹-L²-L³-. In some embodiments, L is -L¹-L²-L⁴-. In some embodiments, L is -L¹-L²-L⁵-. In some embodiments, L is -L²-L⁴-L⁵-. In some embodiments, L is -L¹-L²-L³-L⁴-. In some embodiments, L is -L²-L³-L⁴-L⁵-. In some embodiments, L is -L¹-L²-L⁴-L⁵-. In some embodiments, L is -L¹-L²-L³-L⁴-L⁵-.

In some embodiments, heteroalkylene is an alkylene where one carbon atom is replaced with —O—, —S—, —S(═O)—, —S(═O)₂—, —S(═O)(═NH)—, —S(═O)(═NR⁶)—, —NR⁶—, —P(═O)OH—, —NHC(═O)—, —C(═O)NH—, —OC(═O)NH—, —NHC(═N—CN)NH—, or —NHC(═N—R⁶)NH—. In some embodiments, heteroalkylene is an alkylene where one carbon atom is replaced with —S(═O)(═NH)—, —S(═O)(═NR⁶)—, —P(═O)OH—, —NHC(═N—CN)NH—, or —NHC(═N—R⁶)NH—.

In some embodiments, each R⁶ is independently selected from C₄-C₂₀polyethylene glycol, unsubstituted or substituted C₁-C₆alkyl, wherein the substituted C₁-C₆alkyl is substituted with halogen, —OH, —CO₂H, —NHR⁵, —C(═O)NHR⁵, or —NHC(═O)R⁵. In some embodiments, each R⁶ is independently selected from C₄-C₂₀polyethylene glycol, unsubstituted or substituted C₁-C₆alkyl, wherein the substituted C₁-C₆alkyl is substituted with —OH, —CO₂H, —NHR⁵, —C(═O)NHR⁵, or —NHC(═O)R⁵. In some embodiments, each R⁶ is independently selected from C₄-C₂₀polyethylene glycol, unsubstituted or substituted C₁-C₆alkyl, wherein the substituted C₁-C₆alkyl is substituted with —CO₂H, —NHR⁵, —C(═O)NHR⁵, or —NHC(═O)R⁵. In some embodiments, each R⁶ is independently selected from C₄-C₂₀polyethylene glycol, unsubstituted or substituted C₁-C₆alkyl, wherein the substituted C₁-C₆alkyl is substituted with —C(═O)NHR⁵, or —NHC(═O)R⁵. In some embodiments, each R⁶ is independently selected from C₄-C₂₀polyethylene glycol, unsubstituted or substituted C₁-C₆alkyl, wherein the substituted C₁-C₆alkyl is substituted with —C(═O)NHR⁵.

In some embodiments, L¹ is unsubstituted or substituted C₁-C₂₀alkylene, unsubstituted or substituted C₁-C₂₀heteroalkylene, C₄-C₂₀polyethylene glycol, unsubstituted or substituted C₃-C₈cycloalkylene, unsubstituted or substituted monocyclic C₃-C₈heterocycloalkylene, unsubstituted or substituted phenylene, unsubstituted or substituted monocyclic heteroarylene.

In some embodiments, L¹ is unsubstituted or substituted C₁-C₆alkylene, unsubstituted or substituted C₁-C₁₀heteroalkylene, C₄-C₂₀polyethylene glycol, unsubstituted or substituted cyclohexylene, or unsubstituted or substituted phenylene.

In some embodiments, L² is absent, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂O)ₘ—(CH₂)_{P}—, —C(═O)NR⁴—(CH₂CH₂O)ₙ—(CH₂)_{P}—, —NR⁴C(═O)—(CH₂CH₂O)ₙ—(CH₂)_{P}—, or —(CH₂CH₂O)ₙ—(CH₂)_{P}—; each m is independently 1, 2, 3, 4, 5, or 6; each p is independently 1 or 2.

In some embodiments, L³ is absent, or one or more independently selected groups selected from: alanine, arginine, asparagine, aspartate, cysteine, glutamine, glutamate, glycine, leucine, lysine, methionine, phenylalanine, proline, serine, tyrosine, valine, and amino(unsubstituted or substituted benzyl)carbamate; wherein any free amine of an amino acid is optionally substituted with R⁵ or —C(═O)(R⁵) and any free carboxylic acid of an amino acid is optionally replaced with —C(═O)NH(R⁵).

In some embodiments, L³ is lysine, glutamine, glutamate, glycine, leucine, lysine, methionine, phenylalanine, serine, tyrosine, valine, citrulline, methionine-valine-lysine, glycine-phenylalanine-glycine-glycine, tyrosine-arginine-valine, arginine-valine, valine-citrulline-(para-aminobenzyl carbamate), or combination thereof; wherein any free amine (—NH₂) of an amino acid is optionally substituted with —C(═O)(unsubstituted or substituted C₁-C₂₀alkylene) or —C(═O)—C₄-C₂₀polyethylene glycol; wherein any free carboxylic acid (—CO₂H) of any amino acid is optionally replaced with —C(═O)NH-(2,4,6-trimethyl-3-bromophenyl), —C(═O)NH-(unsubstituted or substituted C₁-C₂₀alkylene), or —C(═O)NH—(C₄-C₂₀polyethylene glycol).

In some embodiments, L³ is:

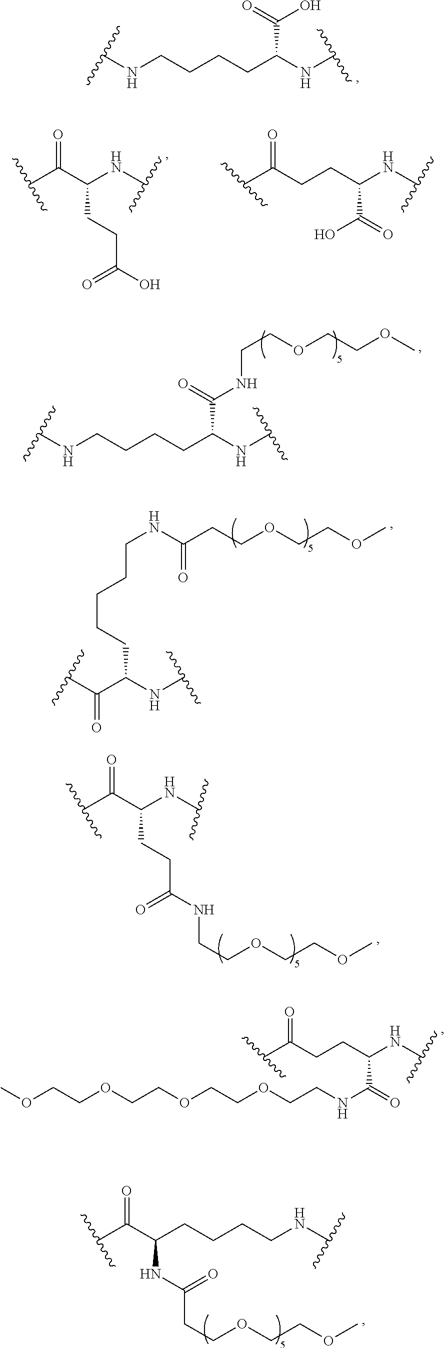

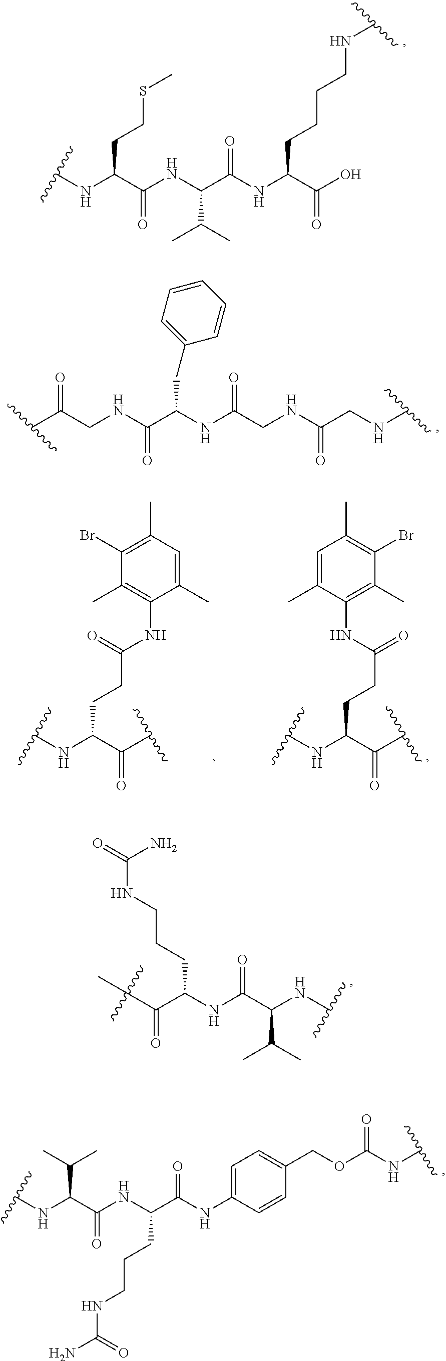

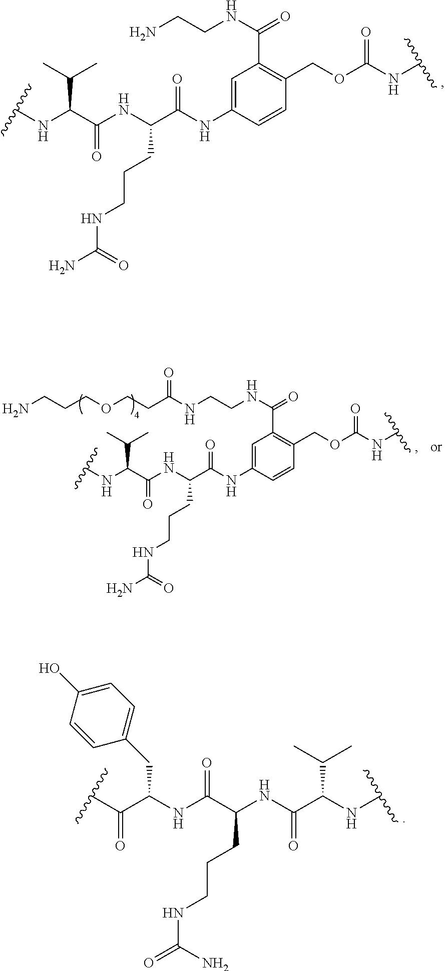

In some embodiments, L⁴ is absent, —C(═O)-(unsubstituted or substituted C₁-C₆alkylene)-, —C(═O)NR⁴-(unsubstituted or substituted C₁-C₆alkylene)-, —NR⁴C(═O)-(unsubstituted or substituted C₁-C₆alkylene)-, —C(═O)—(CH₂CH₂O)ₙ—(CH₂)_{q}—, —C(═O)NR⁴—(CH₂CH₂O)ₙ—(CH₂)_{q}—, —NR⁴C(═O)—(CH₂CH₂O)ₙ—(CH₂)_{q}—, or —(CH₂CH₂O)ₙ—(CH₂)_{q}—; each n is independently 1, 2, 3, 4, 5, or 6; each q is independently 1 or 2.

In some embodiments, L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂O)ₙ—(CH₂)_{q}, —C(═O)NR⁴—(CH₂CH₂O)ₙ—(CH₂)_{q}—, —NR⁴C(═O)—(CH₂CH₂O)ₙ—(CH₂)_{q}—, —(CH₂CH₂O)ₙ—(CH₂)_{q}—, —C(═O)—(OCH₂CH₂)ₙ—, or —(OCH₂CH₂)ₙ—; each p is independently 0, 1, or 2.

In some embodiments, L⁵ is absent, —NR⁴C(═O)—(CH₂)ₙ—, —C(═O)—(CH₂CH₂O)ₙ—(CH₂)_{q}—, —C(═O)NR⁴—(CH₂CH₂O)ₙ—(CH₂)_{q}—, or —NR⁴C(═O)—(CH₂CH₂O)ₙ—(CH₂)_{q}—; each q is independently 1, or 2.

In some embodiments, —X¹-L- is —O—CH₂C(═O)NH-L- and —CH₂C(═O)NH-L- is:

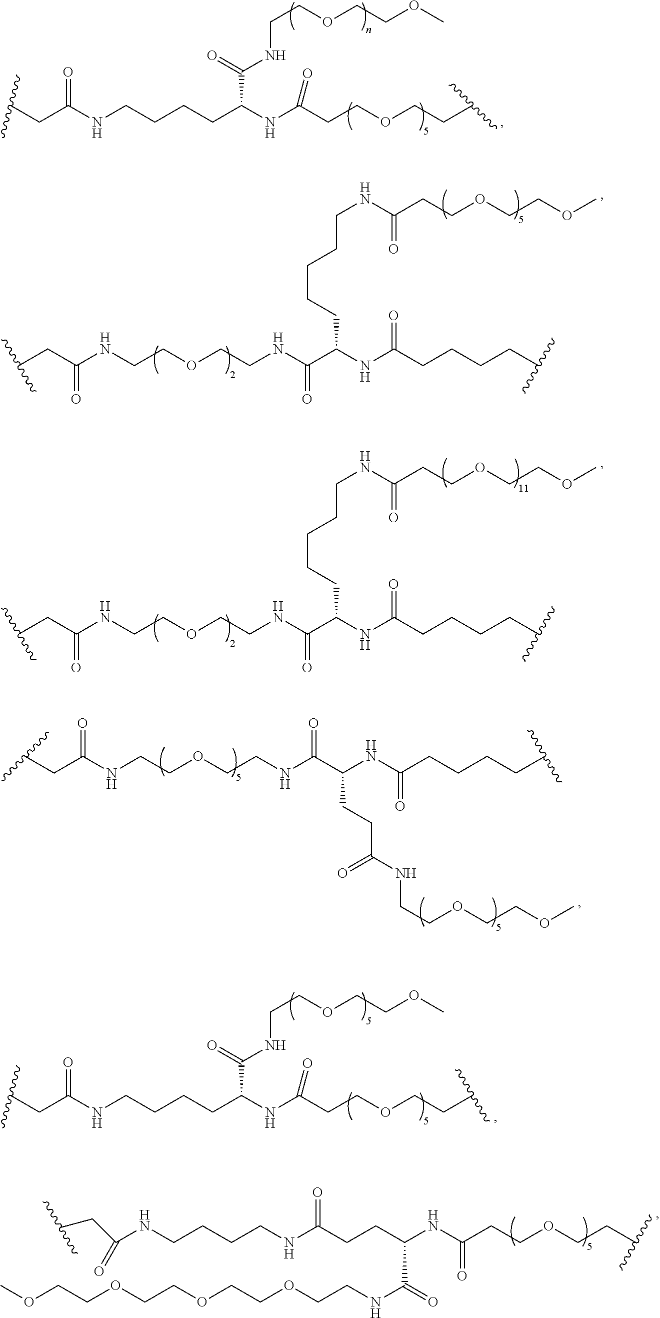

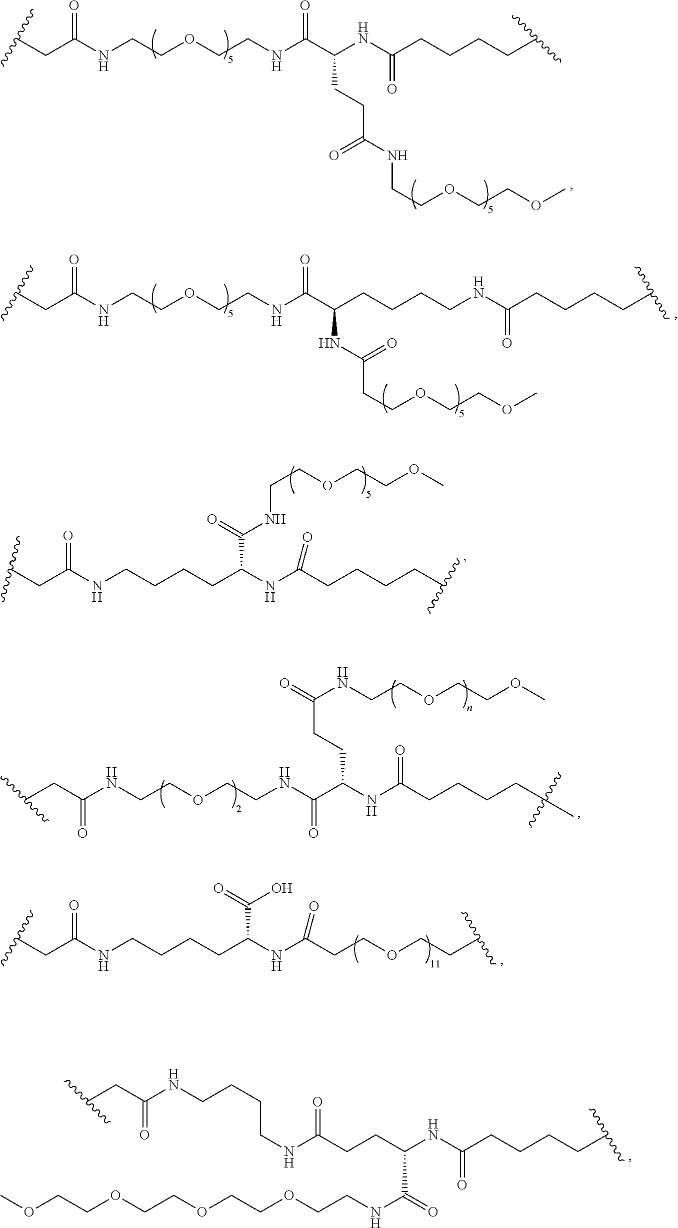

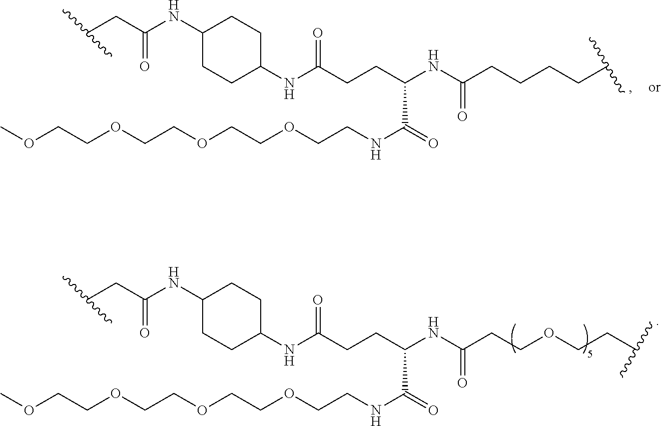

In some embodiments, —X¹-L- is —O—CH₂C(═O)NH-L- and —CH₂C(═O)NH-L- is:

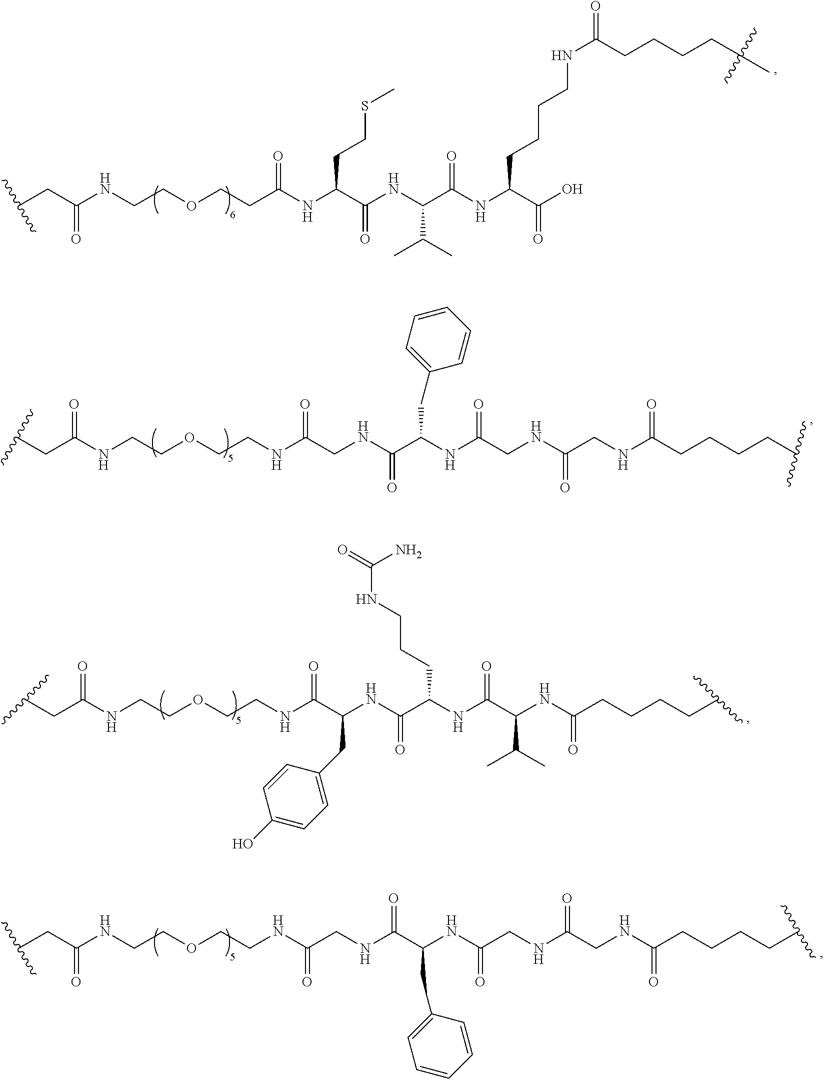

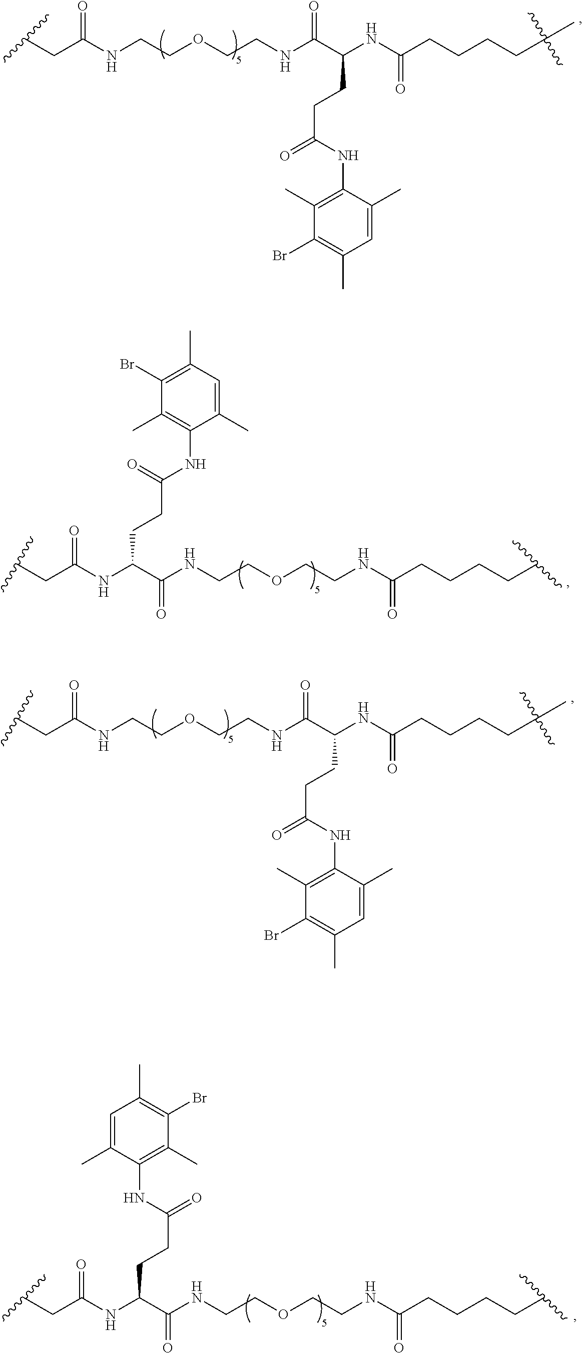

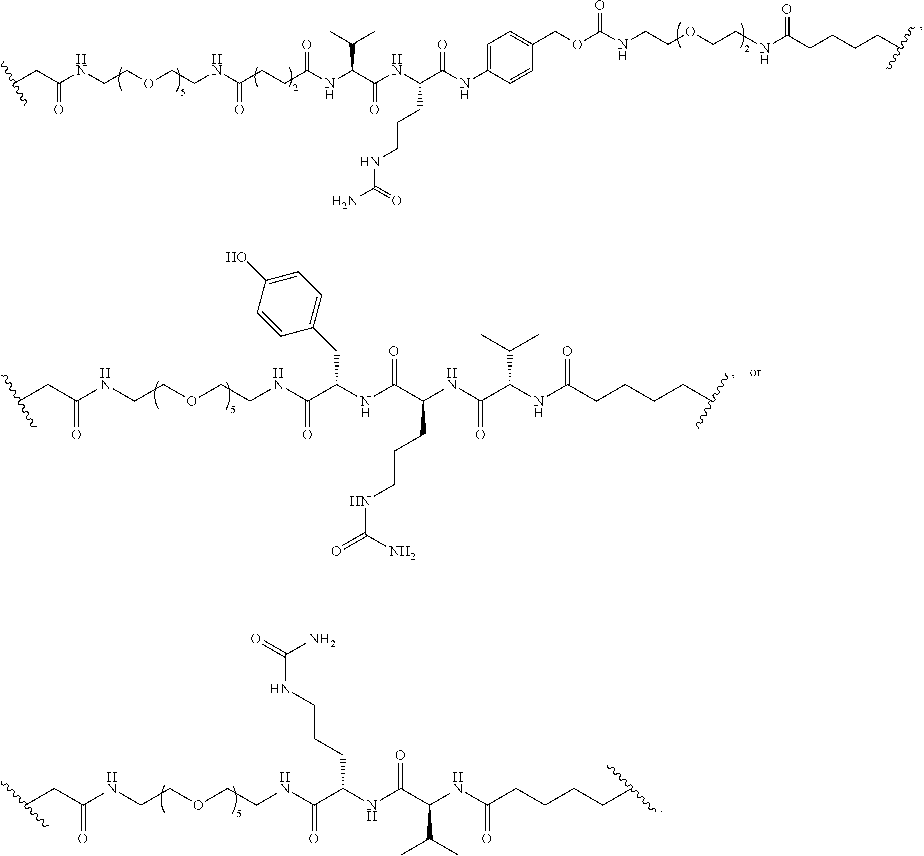

In some embodiments, X¹ is —O-L- and —O-L- is:

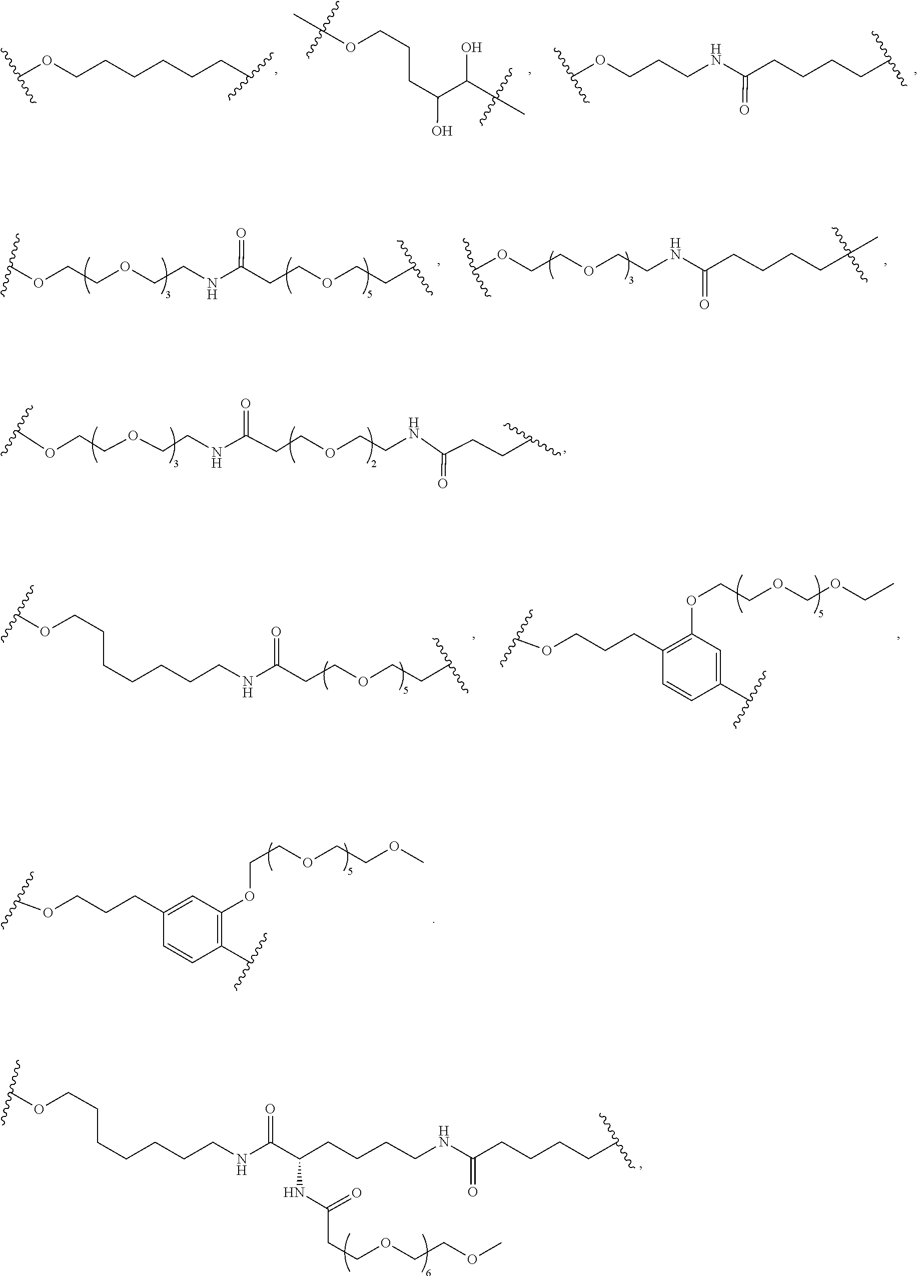

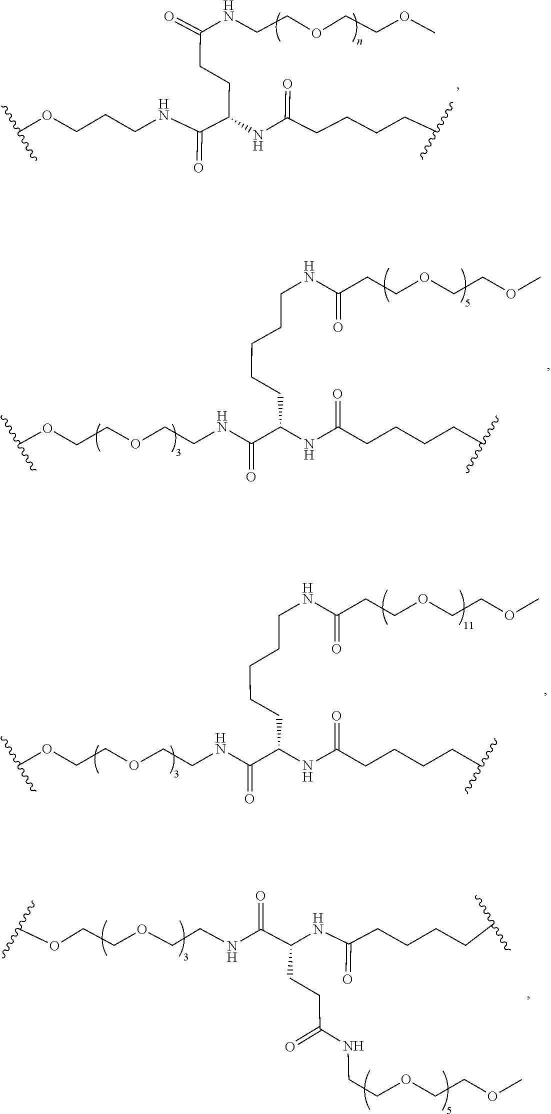

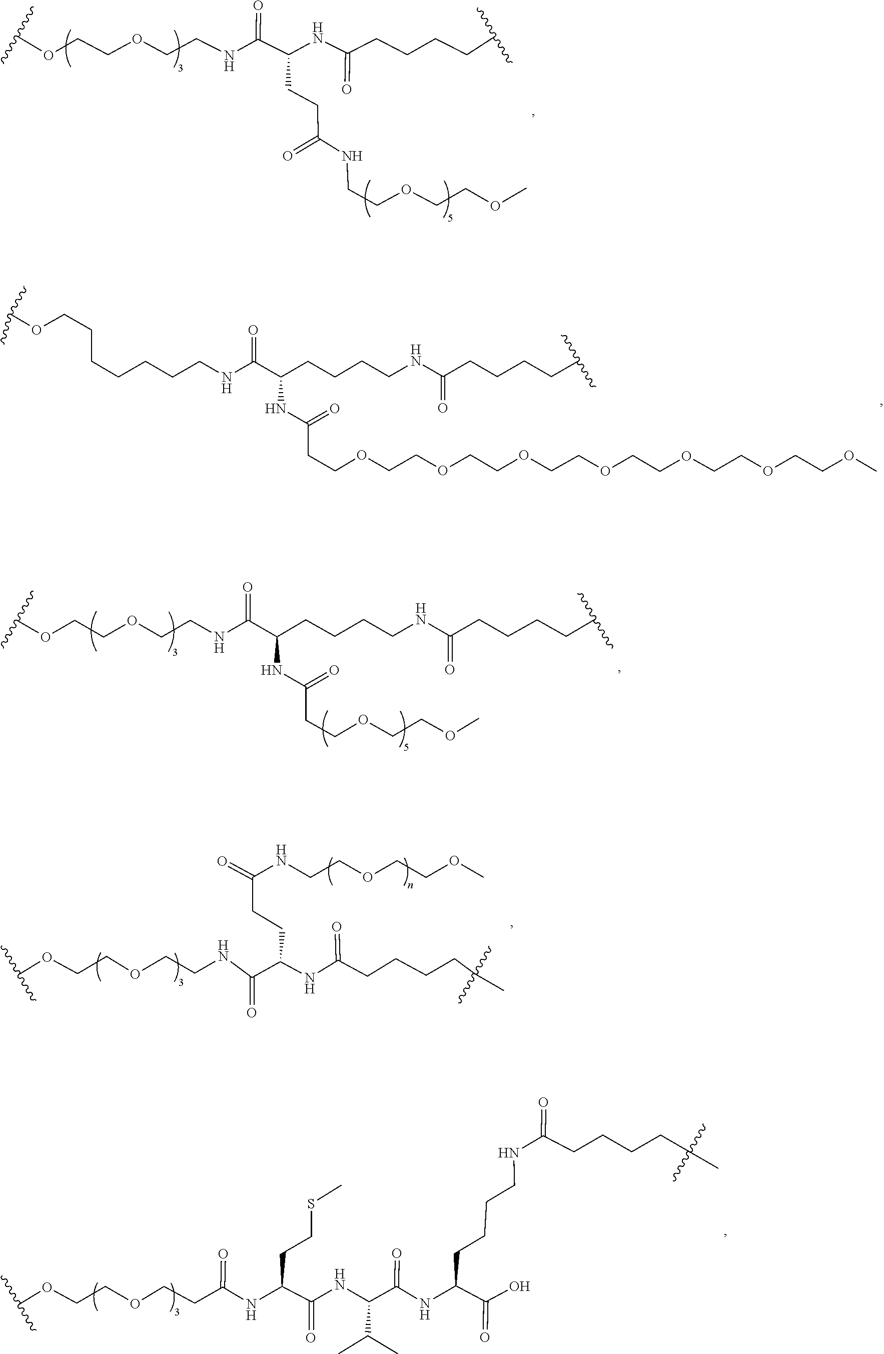

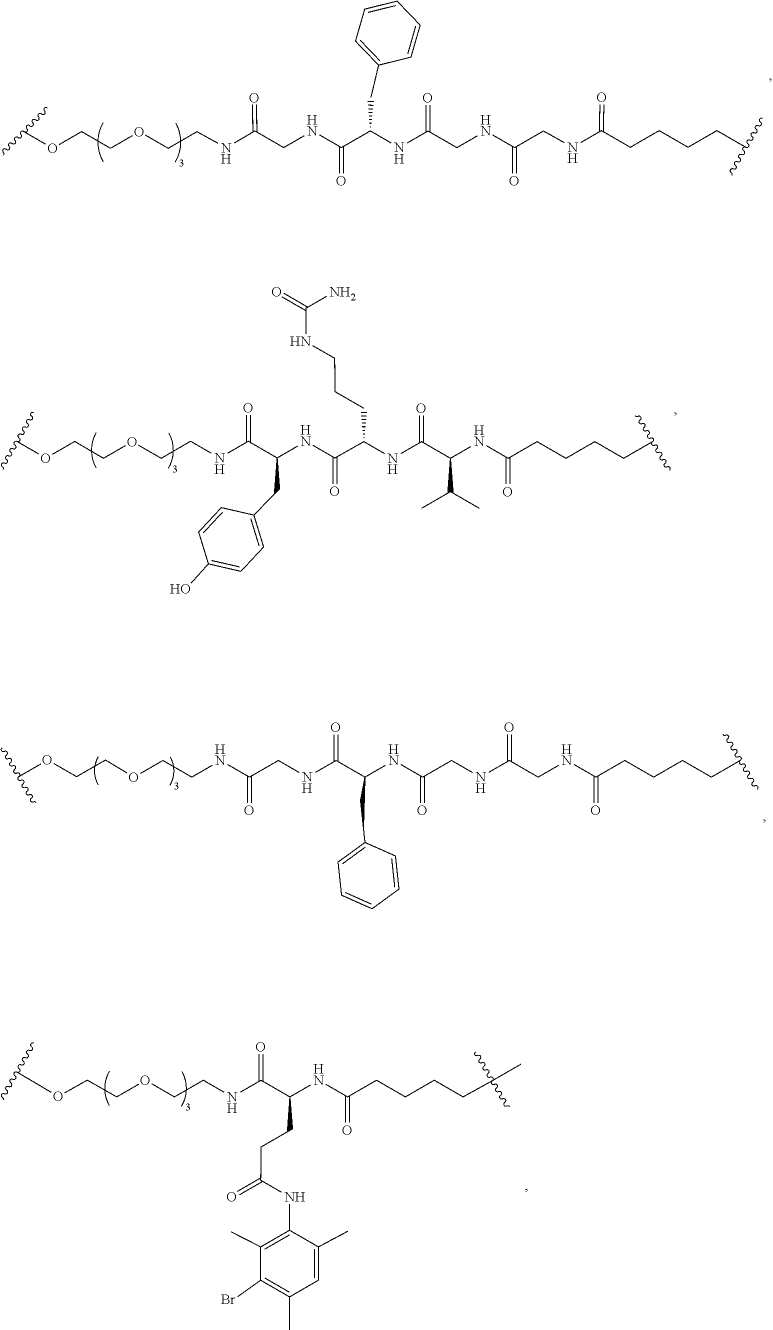

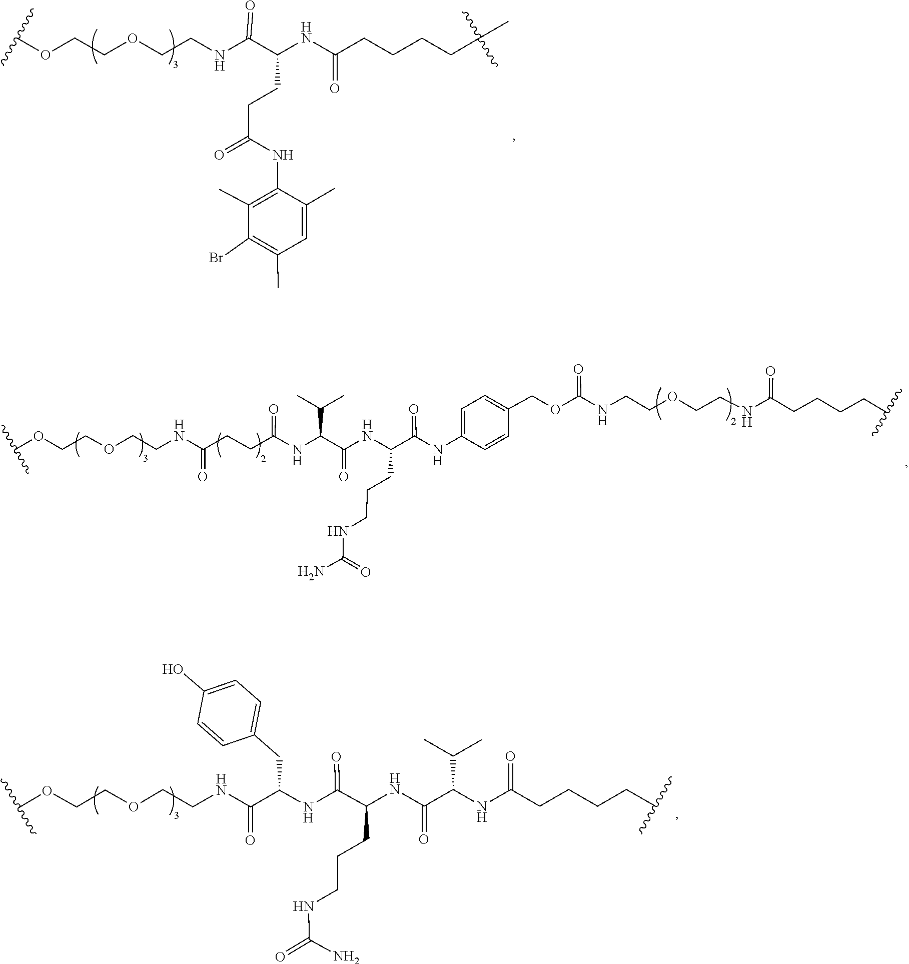

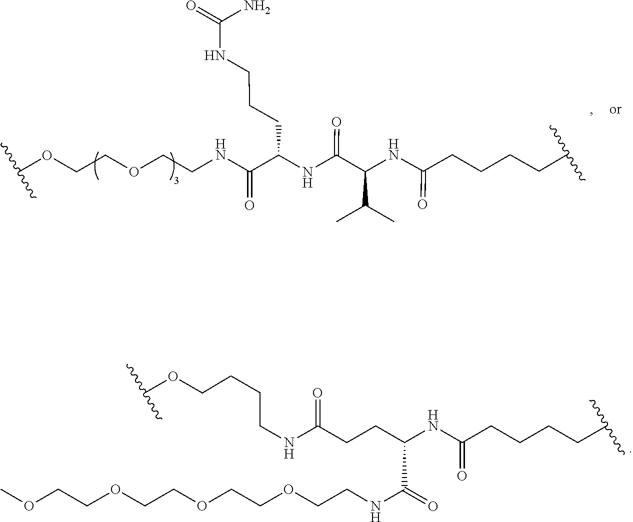

### Conjugating Moieties (R²)

In addition, conjugating moieties (R²) are used to facilitate the attachment to the tumor targeting moieties. In some embodiments, R² is a moiety that is capable of reacting with an amine (—NH₂) or thiol (—SH) of a tumor targeting moiety R³. In some embodiments, R² is a moiety that is capable of reacting with an amine (—NH₂) of the side chain of a lysine residue of the tumor targeting moiety R³.

In some embodiments, R² is a moiety that is capable of reacting with an amine (—NH₂) of the tumor targeting moiety R³ and comprises a tetrafluorophenyl ester, pentafluorophenyl ester, dinitrophenyl ester, succinimide ester, sulfosuccinimide ester, or isothiocyanate.

In some embodiments, R² is a moiety that is capable of reacting with an amine (—NH₂) of the tumor targeting moiety R³ and comprises:

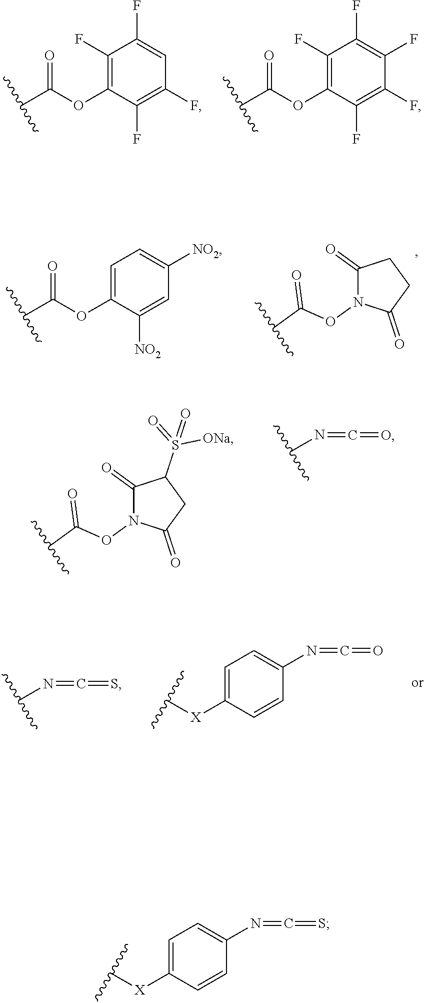

X is absent, —O—, —S—, —S(═O)—, —S(═O)₂, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—; each R^{a} is independently selected from hydrogen, and C₁-C₄alkyl.

In some embodiments, R² is a moiety that is capable of reacting with an amine (—NH₂) of the tumor targeting moiety R³ and comprises:

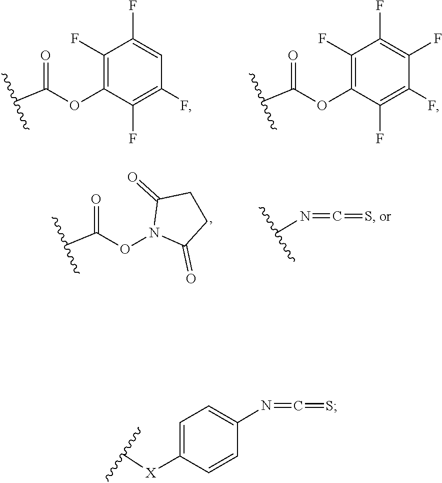

absent or —NR^{a}C(═S)NR^{a}—; each R^{a} is independently selected from hydrogen, and C₁-C₄alkyl. In some embodiments, X is absent or —NHC(═S)NH—.

In some embodiments, R² is a moiety that is capable of reacting with an amine (—NH₂) of the tumor targeting moiety R³ and comprises:

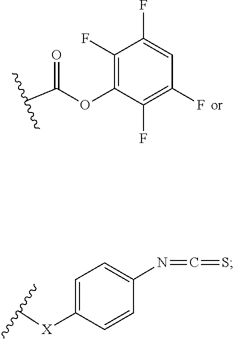

X is absent or —NHC(═S)NH—.

In some embodiments, R² is a moiety that is capable of reacting with an amine (—NH₂) of the tumor targeting moiety R³ and comprises:

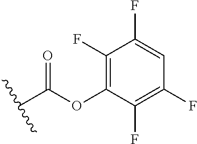 [SMILES: *C(=O)Oc1c(F)c(F)cc(F)c1F]

In some embodiments, R² is a moiety that is capable of reacting with an amine (—NH₂) of the tumor targeting moiety R³ and comprises:

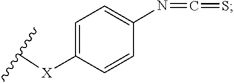

X is absent or —NHC(═S)NH—.

Alternatively, or in addition, an isothiocyanate linker may be used, such as p-SCN-Bn-DOTA, involving a lysine residue within an immunoconjugate of the invention.

In some embodiments, R² is a moiety that is capable of reacting with a thiol (—SH) of the side chain of a cysteine residue of the tumor targeting moiety R³ and comprises: a maleimide group, a haloacetamide group, a haloacetyl group, a haloacetate group, a pyrdinylthio group, a vinylcarbonyl group, an aziridinyl group, a disulfide group, an acetylene group, a hydroxysuccinimide group or a thiol group.

In some embodiments, R² is a moiety that is capable of reacting with a thiol (—SH) of a tumor targeting moiety R³ and comprises

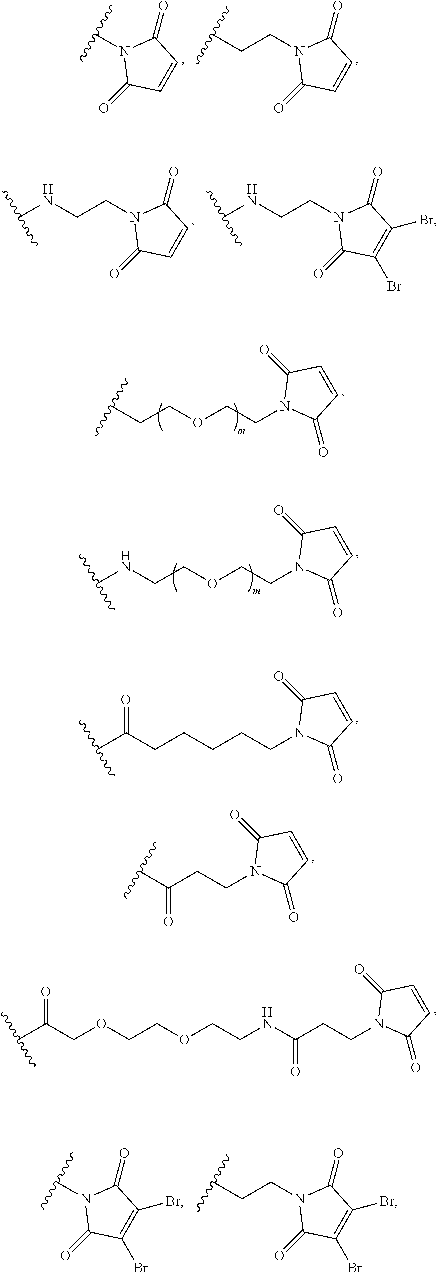

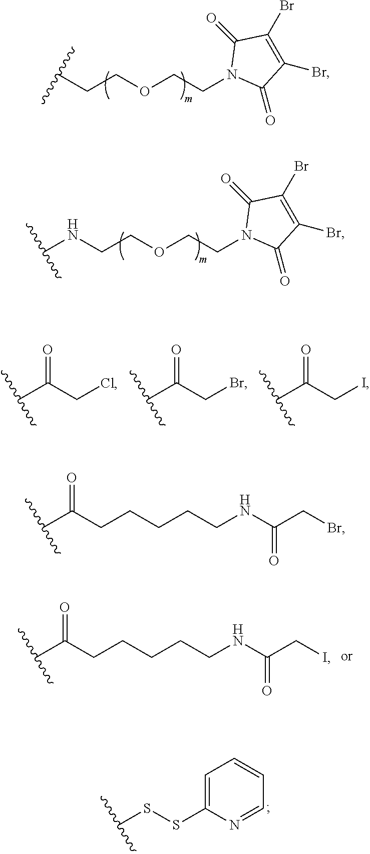

m is 0, 1, 2, 3, 4, or 5.

In some embodiments, R² is a moiety that is capable of reacting with a thiol (—SH) of a tumor targeting moiety R³ and comprises

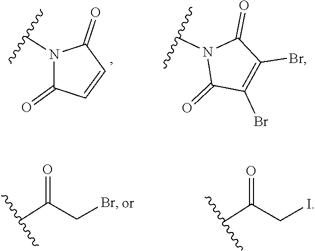 [SMILES: *N1C(=O)C=CC1=O ; *N1C(=O)C(Br)=C(Br)C1=O ; *C(=O)CBr ; *C(=O)CI]

In some embodiments, a linker may be conjugated to an antibody through a cysteine bridging functionality such as ThioBridge® or DBM (dibromomaleimide). These linkers can act to restabilize intrachain disulfides after reduction and conjugation (Bird M, et al., Antibody-Drug Conjugates pp. 113-129 (2019) and Behrens C R, et al. Mol. Pharmaceutics 12:3986 (2015)). Exemplary rebridging stretcher elements are shown below (wherein the wavy line indicates sites of covalent attachment to an immunoconjugate):

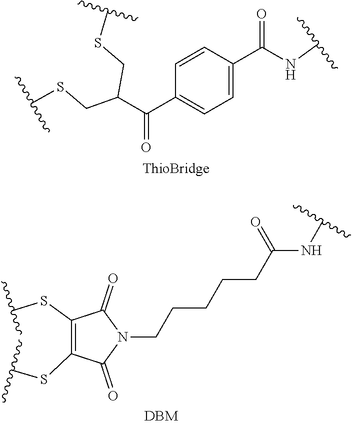 [SMILES: *NC(=O)c1ccc(C(=O)C(CS*)CS*)cc1 ; *NC(=O)CCCCCN1C(=O)C(S*)=C(S*)C1=O]

For additional details regarding linkers and their use in the compounds described herein, see: Wu, A. M.; Senter, P. D. Nat. Biotechnol 2005, 23(9): 1137-1146; Beck, A.; et al. Discov. Med. 2010, 10(53): 329-339; Nolting, B.; et al. Methods. Mol. Biol. 2013, 1045: 71-100; Jain, N.; et al. Pharm. Res. 2015, 32: 3526-3540; McCombs, J. R.; Owen, S. C. AAPS J. 2015, 17(2): 339-351; Jun Lu, et al., Int J Mol Sci. 2016 April; 17(4): 561; each of which is incorporated by reference for such linker disclosures.

### Exemplary Immunoconjugates

In some embodiments, described herein is a compound of Formula (I), or a pharmaceutically acceptable salt thereof:

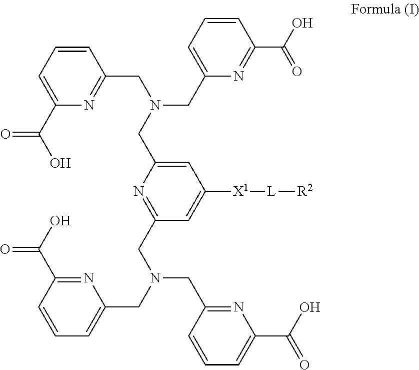

wherein:
X¹ is —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—, -(unsubstituted or substituted C₁-C₆alkylene)-X²—, —O-(unsubstituted or substituted C₁-C₆alkylene)-X²—, or —(C₄-C₂₀polyethylene glycol)-X²—;
X² is absent, —C(═O)—, —NR^{a}C(═O)—, —C(═O)NR^{a}—, or —C(═O)X⁴—;
   each R^{a} is independently selected from hydrogen, and C₁-C₄alkyl;
X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(═O)R⁵, and wherein any free carboxylic acid of any amino acid is optionally replaced with —C(═O)NH—R⁵;
R² is a moiety that is capable of reacting with an amine (—NH₂) or thiol (—SH) of a tumor targeting moiety R³;
L is a linker that is -L¹-L²-L³-L⁴-L⁵-;
L¹ is absent, unsubstituted or substituted C₁-C₁₀alkylene, unsubstituted or substituted C₁-C₁₀heteroalkylene, unsubstituted or substituted C₂-C₂₀alkenylene, unsubstituted or substituted C₂-C₂₀alkynylene, C₄-C₂₀polyethylene glycol, —(X³CH₂CH₂)ₜ—, unsubstituted or substituted cycloalkylene, unsubstituted or substituted heterocycloalkylene, unsubstituted or substituted arylene, unsubstituted or substituted heteroarylene;
   each X³ is independently selected from O and NR⁴;
   each t is independently 1, 2, 3, 4, 5, 6, 7, 8, 9, 10, 11, or 12;
L² is absent, unsubstituted or substituted C₁-C₁₀alkylene, unsubstituted or substituted C₁-C₁₀heteroalkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₘ—(CH₂)_{P}—, —C(═O)NR⁴—(CH₂CH₂X³)ₘ—(CH₂)ₚ—, —NR⁴C(═O)—(CH₂CH₂X³)ₘ—(CH₂)ₚ—, or —(CH₂CH₂X³)ₘ—(CH₂)ₚ—;
   each R⁴ is independently selected from hydrogen, and C₁-C₆alkyl;
   each m is independently 1, 2, 3, 4, 5, 6, 7, 8, 9, 10, 11, or 12;
   each p is independently 0, 1, or 2;
L³ is absent, or one or more independently selected groups selected from: natural or unnatural amino acids, and an optional amino(unsubstituted or substituted benzyl)carbamate; wherein any free amine (—NH₂) of an amino acid is optionally independently substituted with R⁵ or —C(═O)R⁵, wherein any free carboxylic acid of any amino acid is optionally replaced with —C(═O)NH—R⁵, and wherein substituted benzyl is substituted with 1 or 2 groups selected from halogen, —OH, —OR⁵, —CO₂H, —NHR⁵, —C(═O)NHR⁵, —NHC(═O)R⁵ and substituted C₁-C₆alkyl, wherein the substituted C₁-C₆alkyl is substituted with, —OH, —CO₂H, —NHR⁵, —C(═O)NHR⁵, and —NHC(═O)R⁵;
L⁴ is absent, unsubstituted or substituted C₁-C₁₀alkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, or —(X³CH₂CH₂)ₙ—;
   each n is independently 1, 2, 3, 4, 5, 6, 7, 8, 9, 10, 11, or 12;
   each q is independently 0, 1, or 2;
L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, —C(═O)—(X³CH₂CH₂)ₙ—, or —(X³CH₂CH₂)ₙ—;
   each n is independently 1, 2, 3, 4, 5, 6, 7, 8, 9, 10, 11, or 12;
   each q is independently 0, 1, or 2;
   each R^{a} is independently selected from hydrogen, and C₁-C₄alkyl;
   each R^{b} is independently selected from hydrogen, and C₁-C₄alkyl;
wherein heteroalkylene is an alkylene where one carbon atom is replaced with —O—, —S—, —S(═O)—, —S(═O)₂—, —S(═O)(═NH)—, —S(═O)(═NR⁶)—, —NR⁶—, —P(═O)OR^{b}—, —NR^{a}C(═O)—, —C(═O)NR^{a}—, —OC(═O)NR^{a}—, —NR^{a}C(═O)NR^{a}—, —NR^{a}C(═N—CN)NR^{a}—, —NR^{a}C(═N—R⁶)NR^{a}—, or —NR^{a}C(═O)O—;
wherein when any one of -L¹-, -L²-, -L³-, -L⁴-, and -L⁵- is substituted then -L¹-, -L²-, -L³-, -L⁴-, and -L⁵- is substituted with 1, 2, 3, or 4 groups selected from halogen, —OH, —OR⁵, —CO₂H, —NHR⁵, —C(═O)NHR⁵, —NHC(═O)R⁵ and unsubstituted or substituted C₁-C₆alkyl, wherein the substituted C₁-C₆alkyl is substituted with, —OH, —CO₂H, —NHR⁵, —C(═O)NHR⁵, or —NHC(═O)R⁵;
each R⁵ is independently selected from C₁-C₁₀alkyl, C₄-C₃₀polyethylene glycol, and unsubstituted or substituted arylene, unsubstituted or substituted heteroarylene;
each R⁶ is independently selected from C₄-C₃₀polyethylene glycol, unsubstituted or substituted arylene, unsubstituted or substituted heteroarylene, unsubstituted or substituted C₁-C₆alkyl, wherein the substituted C₁-C₆alkyl is substituted with halogen, —OH, —CO₂H, —NHR⁵, —C(═O)NHR⁵, or —NHC(═O)R⁵;
provided that when L³ is absent then at least one R⁵ is present or —NH-L¹- is one or more independently selected natural or unnatural amino acids; or
provided that —X¹-L-R² is not

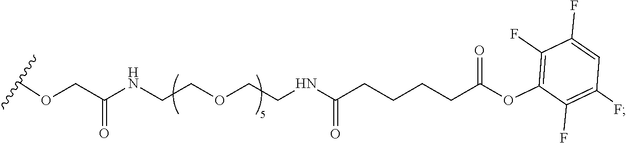

or a radionuclide complex thereof.

In some embodiments, R² is a moiety that is capable of reacting with an amine (—NH₂) of the tumor targeting moiety R³ and comprises a tetrafluorophenyl ester, pentafluorophenyl ester, dinitrophenyl ester, succinimide ester, sulfosuccinimide ester, or isothiocyanate.

In some embodiments, R² is a moiety that is capable of reacting with an amine (—NH₂) of the tumor targeting moiety R³ and comprises:

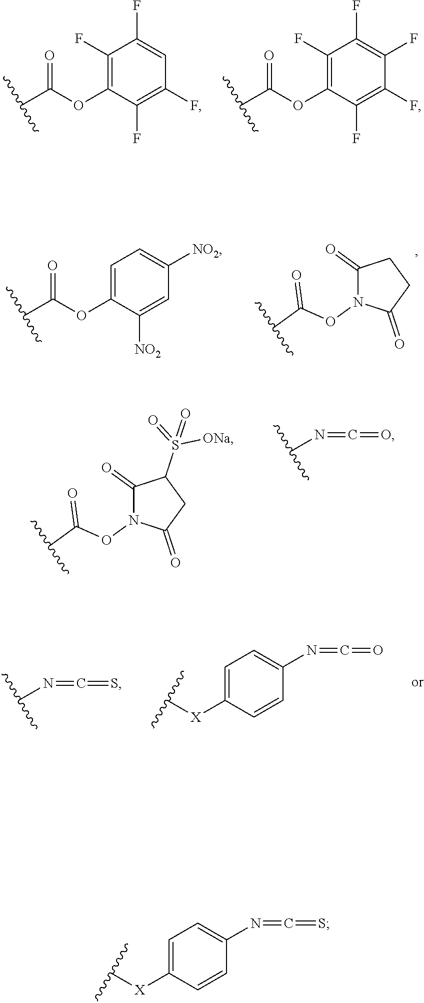

X is absent, —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—;
each R^{a} is independently selected from hydrogen, and C₁-C₄alkyl.

In some embodiments, described herein is an immunoconjugate of Formula (II), or a pharmaceutically acceptable salt thereof:

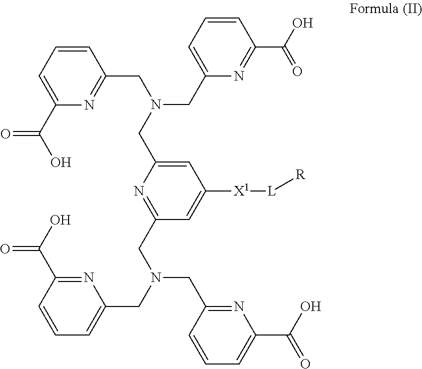

wherein:
   R is —C(═O)NHR³,

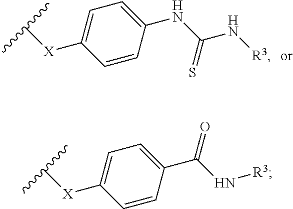

X is absent, —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—;
each R^{a} is independently selected from hydrogen, and C₁-C₄alkyl;
—NH—R³ is a tumor targeting moiety;
X¹ is —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—, -(unsubstituted or substituted C₁-C₆alkylene)-X²—, —O-(unsubstituted or substituted C₁-C₆alkylene)-X²—, or —(C₄-C₂₀polyethylene glycol)-X²—;
X² is absent, —C(═O)—, —NR^{a}C(═O)—, —C(═O)NR^{a}—, or —C(═O)X⁴—;
X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(═O)R⁵, and wherein any free carboxylic acid of any amino acid is optionally replaced with —C(═O)NH—R⁵;
L is a linker that is -L¹-L²-L³-L⁴-L⁵-;
L¹ is absent, unsubstituted or substituted C₁-C₁₀alkylene, unsubstituted or substituted C₁-C₁₀heteroalkylene, unsubstituted or substituted C₂-C₂₀alkenylene, unsubstituted or substituted C₂-C₂₀alkynylene, C₄-C₂₀polyethylene glycol, —(X³CH₂CH₂)ₜ—, unsubstituted or substituted cycloalkylene, unsubstituted or substituted heterocycloalkylene, unsubstituted or substituted arylene, unsubstituted or substituted heteroarylene;
   each X³ is independently selected from 0 and NR⁴;
   each t is independently 1, 2, 3, 4, 5, 6, 7, 8, 9, 10, 11, or 12;
L² is absent, unsubstituted or substituted C₁-C₁₀alkylene, unsubstituted or substituted C₁-C₁₀heteroalkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₘ—(CH₂)_{P}—, —C(═O)NR⁴—(CH₂CH₂X³)ₘ—(CH₂)ₚ—, —NR⁴C(═O)—(CH₂CH₂X³)ₘ—(CH₂)ₚ—, or —(CH₂CH₂X³)ₘ—(CH₂)ₚ—;
   each R⁴ is independently selected from hydrogen, and C₁-C₆alkyl;
   each m is independently 1, 2, 3, 4, 5, 6, 7, 8, 9, 10, 11, or 12;
   each p is independently 0, 1, or 2;
L³ is absent, or one or more independently selected groups selected from: natural or unnatural amino acids, and an optional amino(unsubstituted or substituted benzyl)carbamate; wherein any free amine (—NH₂) of an amino acid is optionally independently substituted with R⁵ or —C(═O)R⁵, wherein any free carboxylic acid of any amino acid is optionally replaced with —C(═O)NH—R⁵, and wherein substituted benzyl is substituted with 1 or 2 groups selected from halogen, —OH, —OR⁵, —CO₂H, —NHR⁵, —C(═O)NHR⁵, —NHC(═O)R⁵ and substituted C₁-C₆alkyl, wherein the substituted C₁-C₆alkyl is substituted with, —OH, —CO₂H, —NHR⁵, —C(═O)NHR⁵, and —NHC(═O)R⁵;
L⁴ is absent, unsubstituted or substituted C₁-C₁₀alkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, or —(X³CH₂CH₂)ₙ—;
   each n is independently 1, 2, 3, 4, 5, 6, 7, 8, 9, 10, 11, or 12;
   each q is independently 0, 1, or 2;
L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, —C(═O)—(X³CH₂CH₂)ₙ—, or —(X³CH₂CH₂)ₙ—;
   each n is independently 1, 2, 3, 4, 5, 6, 7, 8, 9, 10, 11, or 12;
   each q is independently 0, 1, or 2;
   each R^{a} is independently selected from hydrogen, and C₁-C₄alkyl;
   each R^{b} is independently selected from hydrogen, and C₁-C₄alkyl;
wherein heteroalkylene is an alkylene where one carbon atom is replaced with —O—, —S—, —S(═O)—, —S(═O)₂—, —S(═O)(═NH)—, —S(═O)(═NR⁶)—, —NR⁶—, —P(═O)OR^{b}—, —NR^{a}C(═O)—, —C(═O)NR^{a}—, —OC(═O)NR^{a}—, —NR^{a}C(═O)NR^{a}—, —NR^{a}C(═N—CN)NR^{a}—, —NR^{a}C(═N—R⁶)NR^{a}—, or —NR^{a}C(═O)O—;
wherein when any one of -L¹-, -L²-, -L³-, -L⁴-, and -L⁵- is substituted then -L¹-, -L²-, -L³-, -L⁴-, and -L⁵- is substituted with 1, 2, 3, or 4 groups selected from halogen, —OH, —OR⁵, —CO₂H, —NHR⁵, —C(═O)NHR⁵, —NHC(═O)R⁵ and unsubstituted or substituted C₁-C₆alkyl, wherein the substituted C₁-C₆alkyl is substituted with, —OH, —CO₂H, —NHR⁵, —C(═O)NHR⁵, or —NHC(═O)R⁵;
each R⁵ is independently selected from C₁-C₁₀alkyl, C₄-C₃₀polyethylene glycol, and unsubstituted or substituted arylene, unsubstituted or substituted heteroarylene;
each R⁶ is independently selected from C₄-C₃₀polyethylene glycol, unsubstituted or substituted arylene, unsubstituted or substituted heteroarylene, unsubstituted or substituted C₁-C₆alkyl, wherein the substituted C₁-C₆alkyl is substituted with halogen, —OH, —CO₂H, —NHR⁵, —C(═O)NHR⁵, or —NHC(═O)R⁵;
provided that when L³ is absent then at least one R⁵ is present or —NH-L¹- is one or more independently selected natural or unnatural amino acids;
or provided that —X¹-L-R is not

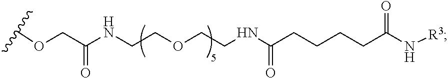

or a radionuclide complex thereof.

In some embodiments, R² is a moiety that is capable of reacting with a thiol (—SH) of a tumor targeting moiety R³ and comprises a maleimide group, a haloacetamide group, a haloacetyl group, a haloacetate group, a pyrdinylthio group, a vinylcarbonyl group, an aziridinyl group, a disulfide group, an acetylene group, a hydroxysuccinimide group or a thiol group.

In some embodiments, R² is a moiety that is capable of reacting with a thiol (—SH) of a tumor targeting moiety R³ and comprises:

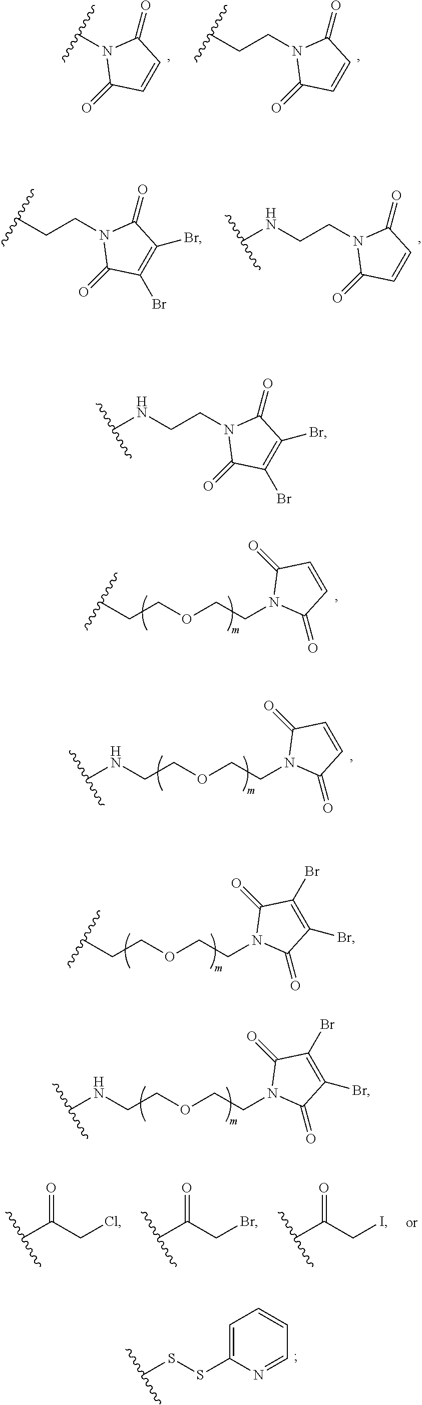

m is 0, 1, 2, 3, 4, or 5.

In some embodiments, described herein is an immunoconjugate of Formula (III), or a pharmaceutically acceptable salt thereof:

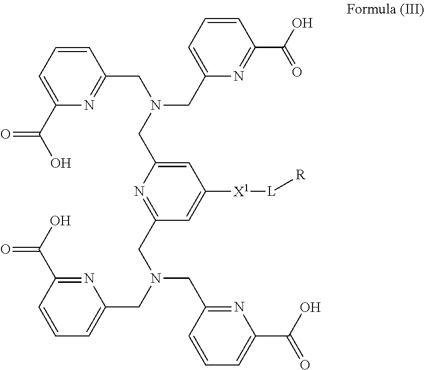

wherein:
R is

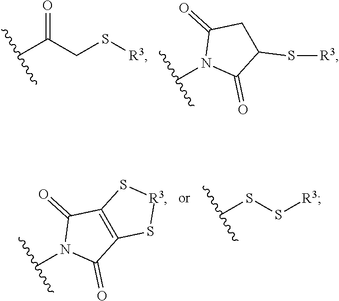

—S—R³ is a tumor targeting moiety.
X¹ is —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—, -(unsubstituted or substituted C₁-C₆alkylene)-X²—, —O-(unsubstituted or substituted C₁-C₆alkylene)-X²—, or —(C₄-C₂₀polyethylene glycol)-X²—;
X² is absent, —C(═O)—, —NR^{a}C(═O)—, —C(═O)NR^{a}—, or —C(═O)X⁴—;
   each R^{a} is independently selected from hydrogen, and C₁-C₄alkyl;
X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(═O)R⁵, and wherein any free carboxylic acid of any amino acid is optionally replaced with —C(═O)NH—R⁵;
L is a linker that is -L¹-L²-L³-L⁴-L⁵-;
L¹ is absent, unsubstituted or substituted C₁-C₁₀alkylene, unsubstituted or substituted C₁-C₁₀heteroalkylene, unsubstituted or substituted C₂-C₂₀alkenylene, unsubstituted or substituted C₂-C₂₀alkynylene, C₄-C₂₀polyethylene glycol, —(X³CH₂CH₂)ₜ—, unsubstituted or substituted cycloalkylene, unsubstituted or substituted heterocycloalkylene, unsubstituted or substituted arylene, unsubstituted or substituted heteroarylene;
   each X³ is independently selected from 0 and NR⁴;
   each t is independently 1, 2, 3, 4, 5, 6, 7, 8, 9, 10, 11, or 12;
L² is absent, unsubstituted or substituted C₁-C₁₀alkylene, unsubstituted or substituted C₁-C₁₀heteroalkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₘ—(CH₂)_{P}—, —C(═O)NR⁴—(CH₂CH₂X³)ₘ—(CH₂)ₚ—, —NR⁴C(═O)—(CH₂CH₂X³)ₘ—(CH₂)ₚ—, or —(CH₂CH₂X³)ₘ—(CH₂)ₚ—;
   each R⁴ is independently selected from hydrogen, and C₁-C₆alkyl;
   each m is independently 1, 2, 3, 4, 5, 6, 7, 8, 9, 10, 11, or 12;
   each p is independently 0, 1, or 2;
L³ is absent, or one or more independently selected groups selected from: natural or unnatural amino acids, and an optional amino(unsubstituted or substituted benzyl)carbamate; wherein any free amine (—NH₂) of an amino acid is optionally independently substituted with R⁵ or —C(═O)R⁵, wherein any free carboxylic acid of any amino acid is optionally replaced with —C(═O)NH—R⁵, and wherein substituted benzyl is substituted with 1 or 2 groups selected from halogen, —OH, —OR⁵, —CO₂H, —NHR⁵, —C(═O)NHR⁵, —NHC(═O)R⁵ and substituted C₁-C₆alkyl, wherein the substituted C₁-C₆alkyl is substituted with, —OH, —CO₂H, —NHR⁵, —C(═O)NHR⁵, and —NHC(═O)R⁵;
L⁴ is absent, unsubstituted or substituted C₁-C₁₀alkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, or —(X³CH₂CH₂)ₙ—;
   each n is independently 1, 2, 3, 4, 5, 6, 7, 8, 9, 10, 11, or 12;
   each q is independently 0, 1, or 2;
L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH₂)_{q}—, —(CH₂CH₂X³)ₙ—(CH₂)_{q}—, —C(═O)—(X³CH₂CH₂)ₙ—, or —(X³CH₂CH₂)ₙ—;
   each n is independently 1, 2, 3, 4, 5, 6, 7, 8, 9, 10, 11, or 12;
   each q is independently 0, 1, or 2;
   each R^{a} is independently selected from hydrogen, and C₁-C₄alkyl;
   each R^{b} is independently selected from hydrogen, and C₁-C₄alkyl;
wherein heteroalkylene is an alkylene where one carbon atom is replaced with —O—, —S—, —S(═O)—, —S(═O)₂—, —S(═O)(═NH)—, —S(═O)(═NR⁶)—, —NR⁶—, —P(═O)OR^{b}, —NR^{a}C(═O)—, —C(═O)NR^{a}—, —OC(═O)NR^{a}—, —NR^{a}C(═O)NR^{a}—, —NR^{a}C(═N—CN)NR^{a}—, —NR^{a}C(═N—R⁶)NR^{a}—, or —NR^{a}C(═O)O—;
wherein when any one of -L¹-, -L²-, -L³-, -L⁴-, and -L⁵- is substituted then -L¹-, -L²-, -L³-, -L⁴-, and -L⁵- is substituted with 1, 2, 3, or 4 groups selected from halogen, —OH, —OR⁵, —CO₂H, —NHR⁵, —C(═O)NHR⁵, —NHC(═O)R⁵ and unsubstituted or substituted C₁-C₆alkyl, wherein the substituted C₁-C₆alkyl is substituted with, —OH, —CO₂H, —NHR⁵, —C(═O)NHR⁵, or —NHC(═O)R⁵;
each R⁵ is independently selected from C₁-C₁₀alkyl, C₄-C₃₀polyethylene glycol, and unsubstituted or substituted arylene, unsubstituted or substituted heteroarylene;
each R⁶ is independently selected from C₄-C₃₀polyethylene glycol, unsubstituted or substituted arylene, unsubstituted or substituted heteroarylene, unsubstituted or substituted C₁-C₆alkyl, wherein the substituted C₁-C₆alkyl is substituted with halogen, —OH, —CO₂H, —NHR⁵, —C(═O)NHR⁵, or —NHC(═O)R⁵;
or a radionuclide complex thereof

In some embodiments, R² is a moiety that is capable of reacting with a thiol (—SH) of the side chain of a cysteine residue of the tumor targeting moiety R³.

In some embodiments, R² is a moiety that is capable of reacting with an amine (—NH₂) of the side chain of a lysine residue of the tumor targeting moiety R³.

In some embodiments, the compound Formula (II) has the structure of Formula (IIa), or Formula (IIb), or a pharmaceutically acceptable salt thereof:

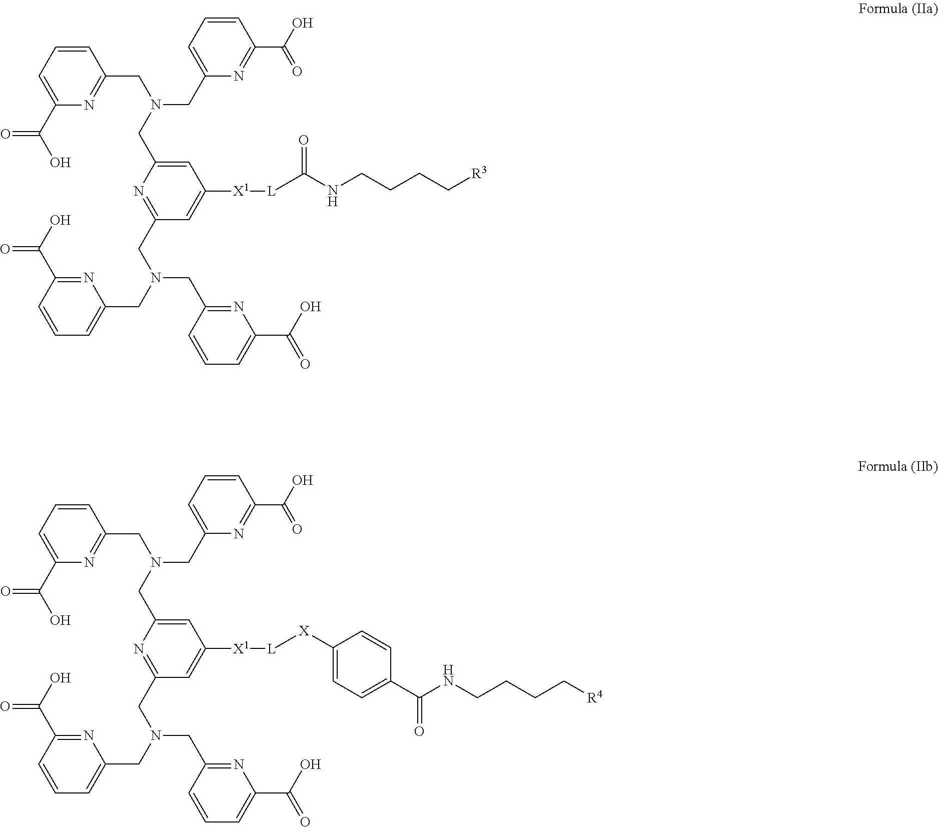

wherein: —NHCH₂CH₂CH₂CH₂— is the side chain of a lysine residue of the tumor targeting moiety R³; or a radionuclide complex thereof.

In some embodiments, the compound Formula (II) has the structure of Formula (IIc), or a pharmaceutically acceptable salt thereof:

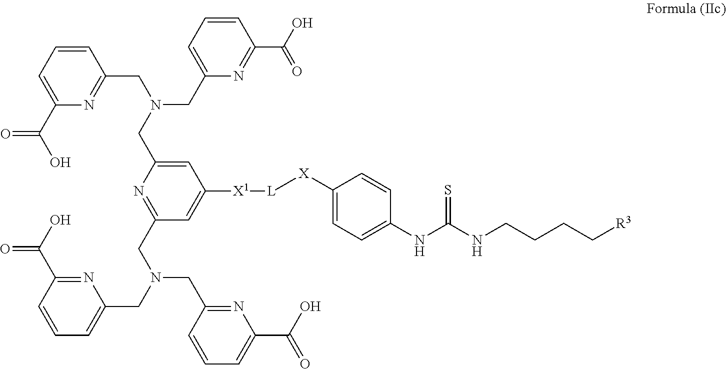

wherein: —NHCH₂CH₂CH₂CH₂— is the side chain of a lysine residue of the tumor targeting moiety R³; or a radionuclide complex thereof.

In some embodiments, the compound Formula (III) has the structure of Formula (IIIa), or a pharmaceutically acceptable salt thereof:

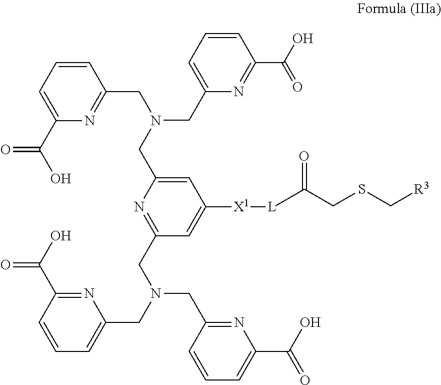

wherein: —SCH₂— is the thiol (—SH) of the side chain of a cysteine residue of the tumor targeting moiety R³; or a radionuclide complex thereof.

In some embodiments, the compound Formula (III) has the structure of Formula (IIIb), or Formula (IIIc), or a pharmaceutically acceptable salt thereof:

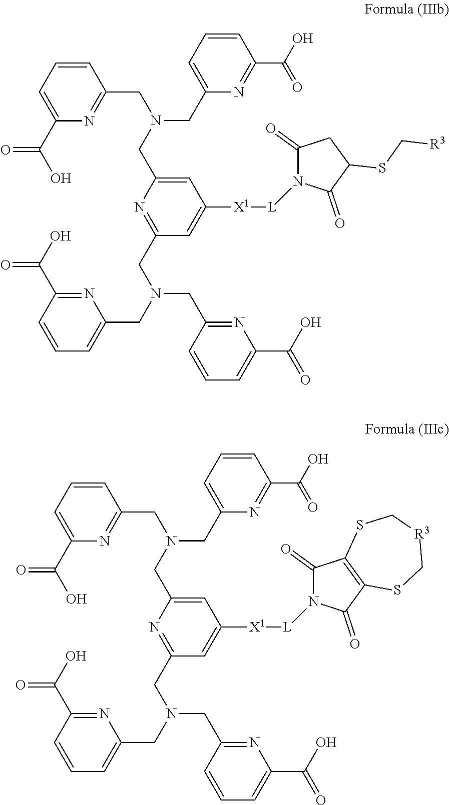

wherein: —SCH₂— is the thiol (—SH) of the side chain of a cysteine residue of the tumor targeting moiety R³; or a radionuclide complex thereof.

In some embodiments, the compound Formula (III) has the structure of Formula (IIId), or a pharmaceutically acceptable salt thereof:

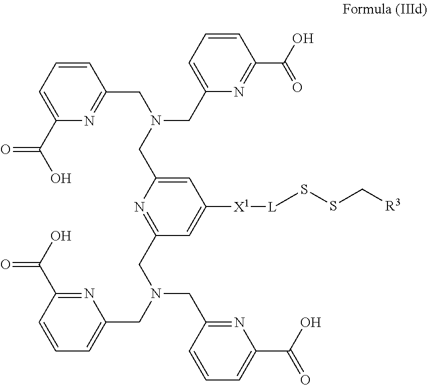

wherein: —SCH₂— is the thiol (—SH) of the side chain of a cysteine residue of the tumor targeting moiety R³; or a radionuclide complex thereof.

In some embodiments, X¹ is —O—, —S—, —S(═O)—, —S(═O)₂—, —NR^{a}—, —C(═O)—, —NR^{a}C(═O)—, —C(═O)NR^{a}—, —(C₁-C₆alkylene)-X²—, —O—(C₁-C₆alkylene)-X²—, or —(C₄-C₂₀polyethylene glycol)-X²—; X² is absent, —C(═O)—, —C(═O)NR^{a}—, or —C(═O)X⁴—; X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(═O)R⁵, and wherein any free carboxylic acid of any amino acid is optionally replaced with —C(═O)NH—R⁵.

In some embodiments, X¹ is absent, —O—, —S—, —S(═O)—, —S(═O)₂—, —NR^{a}—, —C(═O)—, —NR^{a}C(═O)—, or —C(═O)NR^{a}—. In some embodiments, X¹ is absent, —O—.

In some embodiments, X¹ is —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —OCH₂—X²—, —OCH₂CH₂—X²—, —OCH₂CH₂CH₂—X²—, —OCH₂CH₂CH₂CH₂—X²—, —OCH₂CH₂CH₂CH₂CH₂—X²—, —OCH₂CH₂CH₂CH₂CH₂CH₂—X²—.

In some embodiments, X¹ is —OCH₂—, —OCH₂—X²—, —OCH₂CH₂— or —OCH₂CH₂—X²—; X² is —C(═O)X⁴—; X⁴ is —NH—, —N(CH₃)—, —N(CH₂CH₃)—, —NHS(═O)₂—, —N(CH₃)S(═O)₂—, —N(CH₂CH₃)S(═O)₂—, lysine, glutamine, glutamate, glycine, leucine, lysine, methionine, phenylalanine, serine, tyrosine, valine, citrulline, methionine-valine-lysine, glycine-phenylalanine-glycine-glycine, tyrosine-arginine-valine, arginine-valine, or combination thereof; wherein any free amine (—NH₂) of an amino acid is optionally independently substituted with R⁵ or —C(═O)R⁵, and wherein any free carboxylic acid of any amino acid is optionally replaced with —C(═O)NH—R⁵.

In some embodiments, X¹ is —OCH₂—, —OCH₂—X²—, —OCH₂CH₂— or —OCH₂CH₂—X²—; X² is —C(═O)X⁴—; X⁴ is —NH—, —N(CH₃)—, —N(CH₂CH₃)—, —NHS(═O)₂—, —N(CH₃)S(═O)₂—, or —N(CH₂CH₃)S(═O)₂—.

In some embodiments, X¹ is —OCH₂—, —OCH₂—X²—, —OCH₂CH₂— or —OCH₂CH₂—X²—; X² is —C(═O)X⁴—; X⁴ is lysine, glutamine, glutamate, glycine, leucine, lysine, methionine, phenylalanine, serine, tyrosine, valine, citrulline, methionine-valine-lysine, glycine-phenylalanine-glycine-glycine, tyrosine-arginine-valine, arginine-valine, or combination thereof, wherein any free amine (—NH₂) of an amino acid is optionally independently substituted with R⁵ or —C(═O)R⁵, and wherein any free carboxylic acid of any amino acid is optionally replaced with —C(═O)NH—R⁵.

In some embodiments, heteroalkylene is an alkylene where one carbon atom is replaced with —O—, —S—, —S(═O)—, —S(═O)₂—, —S(═O)(═NH)—, —S(═O)(═NR⁵)—, —NR⁵—, —P(═O)OH—, —NHC(═O)—, —C(═O)NH—, —OC(═O)NH—, —NHC(═N—CN)NH—, or —NHC(═N—R⁵)NH—.

In some embodiments, heteroalkylene is an alkylene where one carbon atom is replaced with —S(═O)(═NH)—, —S(═O)(═NR⁵)—, —P(═O)OH—, —NHC(═N—CN)NH—, or —NHC(═N—R⁵)NH—.

In some embodiments, L¹ is unsubstituted or substituted C₁-C₂₀alkylene, unsubstituted or substituted C₁-C₂₀heteroalkylene, C₄-C₂₀polyethylene glycol, unsubstituted or substituted C₃-C₈cycloalkylene, unsubstituted or substituted monocyclic C₃-C₈heterocycloalkylene, unsubstituted or substituted phenylene, unsubstituted or substituted monocyclic heteroarylene.

In some embodiments, L¹ is unsubstituted or substituted C₁-C₆alkylene, unsubstituted or substituted C₁-C₁₀heteroalkylene, C₄-C₂₀polyethylene glycol, unsubstituted or substituted cyclohexylene, or unsubstituted or substituted phenylene.

In some embodiments, L² is absent, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂O)ₘ—(CH₂)_{P}—, —C(═O)NR⁴—(CH₂CH₂O)ₙ—(CH₂)_{P}—, —NR⁴C(═O)—(CH₂CH₂O)ₙ—(CH₂)_{P}—, or —(CH₂CH₂O)ₙ—(CH₂)_{P}—; each m is independently 1, 2, 3, 4, 5, or 6; each p is independently 1 or 2.

In some embodiments, L³ is absent, or one or more independently selected groups selected from: alanine, arginine, asparagine, aspartate, cysteine, glutamine, glutamate, glycine, leucine, lysine, methionine, phenylalanine, proline, serine, tyrosine, valine, and amino(unsubstituted or substituted benzyl)carbamate; wherein any free amine of an amino acid is optionally substituted with R⁵ or —C(═O)(R⁵) and any free carboxylic acid of an amino acid is optionally replaced with —C(═O)NH(R⁵).

In some embodiments, L³ is lysine, glutamine, glutamate, glycine, leucine, lysine, methionine, phenylalanine, serine, tyrosine, valine, citrulline, methionine-valine-lysine, glycine-phenylalanine-glycine-glycine, tyrosine-arginine-valine, arginine-valine, valine-citrulline-(para-aminobenzyl carbamate), or combination thereof; wherein any free amine (—NH₂) of an amino acid is optionally substituted with —C(═O)(unsubstituted or substituted C₁-C₂₀alkylene) or —C(═O)—C₄-C₂₀polyethylene glycol; wherein any free carboxylic acid (—CO₂H) of any amino acid is optionally replaced with —C(═O)NH-(2,4,6-trimethyl-3-bromophenyl), —C(═O)NH-(unsubstituted or substituted C₁-C₂₀alkylene), or —C(═O)NH—(C₄-C₂₀polyethylene glycol).

In some embodiments, L⁴ is absent, —C(═O)-(unsubstituted or substituted C₁-C₆alkylene)-, —C(═O)NR⁴-(unsubstituted or substituted C₁-C₆alkylene)-, —NR⁴C(═O)-(unsubstituted or substituted C₁-C₆alkylene)-, —C(═O)—(CH₂CH₂O)ₙ—(CH₂)_{q}—, —C(═O)NR⁴—(CH₂CH₂O)ₙ—(CH₂)_{q}—, —NR⁴C(═O)—(CH₂CH₂O)ₙ—(CH₂)_{q}—, or —(CH₂CH₂O)ₙ—(CH₂)_{q}—; each n is independently 1, 2, 3, 4, 5, or 6; each q is independently 1 or 2.

In some embodiments, L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂O)ₙ—(CH₂)_{q}, —C(═O)NR⁴—(CH₂CH₂O)ₙ—(CH₂)_{q}—, —NR⁴C(═O)—(CH₂CH₂O)ₙ—(CH₂)_{q}—, —(CH₂CH₂O)ₙ—(CH₂)_{q}—, —C(═O)—(OCH₂CH₂)ₙ—, or —(OCH₂CH₂)ₙ—; each p is independently 0, 1, or 2.

In some embodiments, L⁵ is absent, —NR⁴C(═O)—(CH₂)ₙ—, —C(═O)—(CH₂CH₂O)ₙ—(CH₂)_{q}—, —C(═O)NR⁴—(CH₂CH₂O)ₙ—(CH₂)_{q}—, or —NR⁴C(═O)—(CH₂CH₂O)ₙ—(CH₂)_{q}—; each q is independently 1, or 2.

In some embodiments, L¹ is unsubstituted or substituted C₁-C₆alkylene, unsubstituted or substituted C₁-C₁₀heteroalkylene, C₄-C₂₀polyethylene glycol, unsubstituted or substituted cyclohexylene, or unsubstituted or substituted phenylene; L² is absent, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂O)ₘ—(CH₂)_{P}—, —C(═O)NR⁴—(CH₂CH₂O)ₙ—(CH₂)_{P}—, —NR⁴C(═O)—(CH₂CH₂O)ₙ—(CH₂)_{P}—, or —(CH₂CH₂O)ₙ—(CH₂)_{P}—; each m is independently 1, 2, 3, 4, 5, or 6; each p is independently 1 or 2; L⁴ is absent, —C(═O)-(unsubstituted or substituted C₁-C₆alkylene)-, —C(═O)NR⁴-(unsubstituted or substituted C₁-C₆alkylene)-, —NR⁴C(═O)-(unsubstituted or substituted C₁-C₆alkylene)-, —C(═O)—(CH₂CH₂O)ₙ—(CH₂)_{q}—, —C(═O)NR⁴—(CH₂CH₂O)ₙ—(CH₂)_{q}—, —NR⁴C(═O)—(CH₂CH₂O)ₙ—(CH₂)_{q}—, or —(CH₂CH₂O)ₙ—(CH₂)_{q}—; L⁵ is —NR⁴C(═O)—(CH₂)ₙ—, —C(═O)—(CH₂CH₂O)ₙ—(CH₂)_{q}—, —C(═O)NR⁴—(CH₂CH₂O)ₙ—(CH₂)_{q}—, or —NR⁴C(═O)—(CH₂CH₂O)ₙ—(CH₂)_{q}—; each n is independently 1, 2, 3, 4, 5, or 6; each q is independently 1 or 2.

In some embodiments, L¹ is unsubstituted or substituted C₁-C₆alkylene, unsubstituted or substituted C₁-C₁₀heteroalkylene, C₄-C₂₀polyethylene glycol, unsubstituted or substituted cyclohexylene, or unsubstituted or substituted phenylene; L² is absent, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂O)ₘ—(CH₂)_{P}—, —C(═O)NR⁴—(CH₂CH₂O)_{P}—(CH₂)_{P}—, —NR⁴C(═O)—(CH₂CH₂O)ₙ—(CH₂)_{P}—, or —(CH₂CH₂O)ₙ—(CH₂)_{P}—; each m is independently 1, 2, 3, 4, 5, or 6; each p is independently 1 or 2; L⁴ is absent; L⁵ is absent, —NR⁴C(═O)—(CH₂)ₙ—, —C(═O)—(CH₂CH₂O)ₙ—(CH₂)_{q}—, —C(═O)NR⁴—(CH₂CH₂O)ₙ—(CH₂)_{q}—, or —NR⁴C(═O)—(CH₂CH₂O)ₙ—(CH₂)_{q}—; each n is independently 1, 2, 3, 4, 5, or 6; each q is independently 1 or 2.

In some embodiments, L¹ is unsubstituted or substituted C₁-C₆alkylene, unsubstituted or substituted C₁-C₁₀heteroalkylene, C₄-C₂₀polyethylene glycol, unsubstituted or substituted cyclohexylene, or unsubstituted or substituted phenylene; L² is absent, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂O)ₘ—(CH₂)_{P}—, —C(═O)NR⁴—(CH₂CH₂O)ₙ—(CH₂)_{P}—, —NR⁴C(═O)—(CH₂CH₂O)ₙ—(CH₂)_{P}—, or —(CH₂CH₂O)ₙ—(CH₂)_{P}—; each m is independently 1, 2, 3, 4, 5, or 6; each p is independently 1 or 2; L³ is one or more independently selected groups selected from: alanine, arginine, asparagine, aspartate, cysteine, glutamine, glutamate, glycine, leucine, lysine, methionine, phenylalanine, proline, serine, tyrosine, valine, and amino(unsubstituted or substituted benzyl)carbamate; wherein any free amine of an amino acid is optionally substituted with R⁵ or —C(═O)(R⁵) and any free carboxylic acid of an amino acid is optionally replaced with —C(═O)NH(R⁵); each R⁵ is independently an unsubstituted or substituted C₁-C₁₀alkylene, C₄-C₂₀polyethylene glycol, or an unsubstituted or substituted phenyl, wherein the substituted phenyl is substituted with 1, 2, 3, 4, or 5 groups independently selected from F, Cl, Br, I, —CH₃, and CF₃; L⁴ is absent; L⁵ is —NR⁴C(═O)—(CH₂)ₙ—, —C(═O)—(CH₂CH₂O)ₙ—(CH₂)_{q}—, —C(═O)NR⁴—(CH₂CH₂O)ₙ—(CH₂)_{q}—, or —NR⁴C(═O)—(CH₂CH₂O)ₙ—(CH₂)_{q}—; each n is independently 1, 2, 3, 4, 5, or 6; each q is independently 1 or 2.

In some embodiments, each m is independently 3, 4, 5, or 6. In some embodiments, each m is independently 4, 5, or 6. In some embodiments, each p is independently 1. In some embodiments, each p is independently 2.

In some embodiments, each n is independently 3, 4, 5, or 6. In some embodiments, each n is independently 4, 5, or 6. In some embodiments, each q is independently 1. In some embodiments, each q is independently 2.

In some embodiments, each m is independently 1, 2, 3, 4, 5, or 6; each p is independently 2; each n is independently 1, 2, 3, 4, 5, or 6; each q is independently 1 or 2. In some embodiments, each m is independently 1, 2, 3, 4, 5, or 6; each p is independently 2; each n is independently 1, 2, 3, 4, 5, or 6; each q is independently 2.

In some embodiments, each X³ is independently O. In some embodiments, each X³ is independently NR⁴. In some embodiments, each R⁴ is hydrogen. In some embodiments, each R⁴ is C₁-C₆alkyl.

In some embodiments, each R⁵ is independently selected from C₁-C₁₀alkyl, C₄-C₃₀polyethylene glycol, and unsubstituted or substituted arylene, unsubstituted or substituted heteroarylene. In some embodiments, each R⁵ is independently selected from C₁-C₁₀alkyl or C₄-C₃₀polyethylene glycol. In some embodiments, each R⁵ is independently selected from unsubstituted or substituted arylene or unsubstituted or substituted heteroarylene.

In some embodiments, each R^{a} is independently selected from hydrogen, and C₁-C₄alkyl. In some embodiments, each R^{a} is independently hydrogen. In some embodiments, each R^{a} is independently C₁-C₄alkyl.

In some embodiments, each R^{b} is independently selected from hydrogen, and C₁-C₄alkyl. In some embodiments, each R^{b} is independently hydrogen. In some embodiments, each R^{b} is independently C₁-C₄alkyl.

Exemplary compounds for use in preparing immunoconjugates described herein include the compounds depicted in Table A.

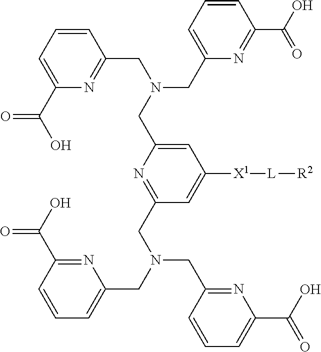

TABLE A

| Cmp. No. | 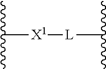 | R² |
|---|---|---|
| 3-1 | 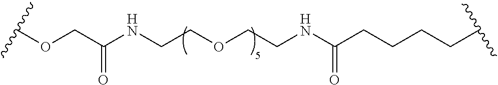 | 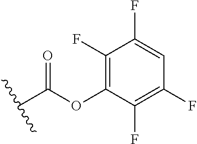 [SMILES: *C(=O)Oc1c(F)c(F)cc(F)c1F] |
| 3-2 | 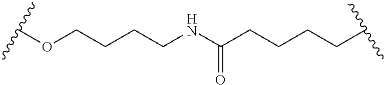 [SMILES: *CCCCC(=O)NCCCCO*] | 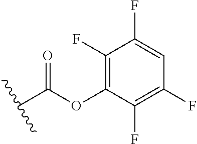 [SMILES: *C(=O)Oc1c(F)c(F)cc(F)c1F] |
| 3-3 | 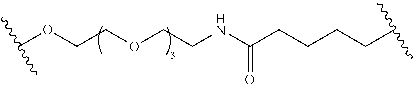 | 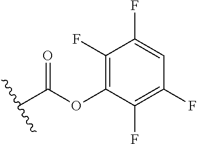 [SMILES: *C(=O)Oc1c(F)c(F)cc(F)c1F] |
| 3-4 | 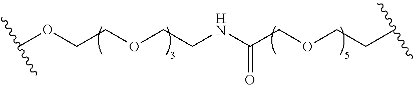 | 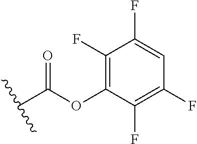 [SMILES: *C(=O)Oc1c(F)c(F)cc(F)c1F] |
| 3-5 | 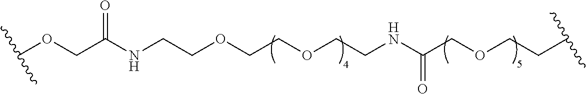 | 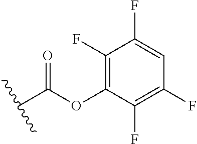 [SMILES: *C(=O)Oc1c(F)c(F)cc(F)c1F] |
| 3-6 | 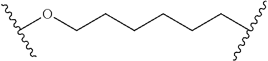 [SMILES: *CCCCCCO*] | 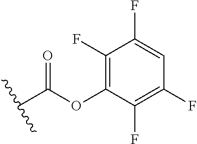 [SMILES: *C(=O)Oc1c(F)c(F)cc(F)c1F] |
| 3-7 | 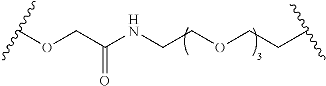 | 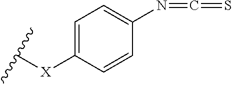 |
| 3-8 | 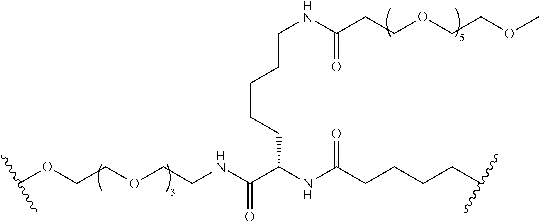 | 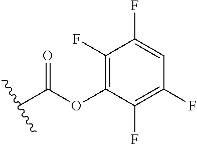 [SMILES: *C(=O)Oc1c(F)c(F)cc(F)c1F] |
| 3-9 | 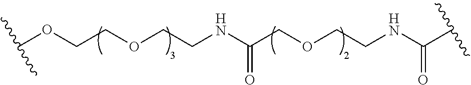 | 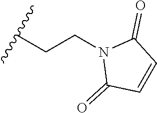 [SMILES: *CCN1C(=O)C=CC1=O] |

### Radioimmunoconjugates

In one embodiment, the invention provides immunoconjugates. In one embodiment, the immunoconjugates are capable of delivering α-emitters in vivo when so labeled, linked or loaded with an α-emitter. In one embodiment, the immunoconjugates are also capable of delivering other radioisotopes (β-emitters, and/or γ-emitters), and/or other atoms in vivo, when so labeled, linked or loaded. In one embodiment, the immunoconjugates are capable of delivering imaging metals (e.g., ¹¹¹In, ⁸⁹Zr, ⁶⁴Cu, ⁶⁸Ga, or ¹³⁴Ce) in vivo when so labeled, linked or loaded.

The immunoconjugates of the current disclosure may be loaded with a radioisotope for a therapeutic or diagnostic effect. In certain embodiments, the chelator may further comprise a radioisotope. In certain embodiments, the radioisotope is an alpha emitter. In certain embodiments, the radioisotope is an alpha emitter selected from the list consisting of ²²⁵Ac, ²²³Ra, ²²⁴Ra, ²²⁷Th, ²¹²Pb, ²¹²Bi, and ²¹³Bi. In certain embodiments, the radioisotope is ²²⁵Ac. In certain embodiments, the radioisotope is an beta emitter. In certain embodiments, the radioisotope is a beta emitter selected from ¹⁷⁷Lu, ⁹⁰Y, ⁶⁷Cu, and ¹⁵³Sm.

Also described herein is a method of making a radioimmunoconjugate comprising loading or complexing an immunoconjugate of the current disclosure to a radioisotope. In certain embodiments, the radioisotope is an alpha emitter. In certain embodiments, the radioisotope is an alpha emitter selected from the list consisting of ²²⁵Ac, ²²³Ra, ²²⁴Ra, ²²⁷Th, ²¹²Pb, ²¹²Bi, and ²¹³Bi. In certain embodiments, the radioisotope is 225-Ac. In certain embodiments, the radioisotope is an beta emitter. In certain embodiments, the radioisotope is a beta emitter selected from ¹⁷⁷Lu, ⁹⁰Y, ⁶⁷Cu, and ¹⁵³Sm.

In one aspect, the invention provides a radioimmunoconjugate, comprising an immunoconjugate of the invention and an α-emitting radioisotope. In one embodiment, the α-emitting radioisotope of the radioimmunoconjugate is selected from the group comprising: ²²⁵Ac, ²²³Ra, ²²⁴Ra, ²²⁷Th, ²¹²Pb, ²¹²Bi, and ²¹³Bi. In one embodiment, the α-emitting radioisotope of the radioimmunoconjugate is selected from the group consisting of: ²²⁵Ac, ²²³Ra, ²²⁴Ra, ²²⁷Th, ²¹²Pb, ²¹²Bi, and ²¹³Bi. In one embodiment, the α-emitting radioisotope of the radioimmunoconjugate is ²²⁵Ac. In one embodiment, the α-emitting radioisotope of the radioimmunoconjugate is ²²³-Ra. In one embodiment, the α-emitting radioisotope of the radioimmunoconjugate is ²²⁴Ra. In one embodiment, the α-emitting radioisotope of the radioimmunoconjugate is ²²⁷Th. In one embodiment, the α-emitting radioisotope of the radioimmunoconjugate is ²²Pb. In one embodiment, the α-emitting radioisotope of the radioimmunoconjugate is ²¹²Bi. In one embodiment, the α-emitting radioisotope of the radioimmunoconjugate is ²¹³Bi.

In some embodiments, the immunoconjugate of the present invention is combined with a radioisotope to provide a radioimmunoconjugate of the invention. In some embodiments, the radioisotope is ²²⁵Ac, ⁸⁶Y, ⁹⁰Y, ¹⁷⁷Lu, ¹⁸⁶Re, ¹⁸⁸Re, ⁸⁹Sr, ¹⁵³Sm, ²¹³Bi, ²¹³Po, ²¹²Bi, ²²³Ra, ²²⁴Ra, ²²⁷Th, ¹⁴⁹Tb, ⁶⁸Ga, ⁶⁴Cu, ⁶⁷Cu, ⁸⁹Zr, ¹³⁷Cs, ²¹²Pb, or ¹⁰³Pd. In some embodiments, the radioisotope is an alpha emitter, such as, e.g., ²²⁵Ac, ²²³Ra, ²²⁴Ra, ²²⁷Th, ²²Pb, ²¹²Bi, and ²¹³Bi. In some embodiments, the radioisotope is a beta particle emitter, such as, e.g., ¹⁷⁷Lu, ⁹⁰Y, ⁶⁷Cu, and ¹⁵³Sm. In some embodiments, the radioisotope is both an alpha particle emitter and a beta and/or gamma particle emitter. In some embodiments, the radioisotope is both a beta particle emitter and a gamma particle and/or photon emitter. In some embodiments, the radioimmunoconjugate is labeled, linked or loaded with, and accordingly comprises, both an α-emitter and a β-emitter. In some embodiments, the radioisotope is selected for use in radio-imaging, such as, e.g., from among ⁶⁸Ga, ⁶⁴Cu, ⁸⁹Zr, ¹¹¹In, and ¹³⁴Ce.

The immunoconjugates and radioimmunoconjugates of the invention may comprise other cargos or payloads besides a radioisotope, including various cytotoxic agents, such as, e.g., a small molecule chemotherapeutic agent, cytotoxic antibiotic, alkylating agent, antimetabolite, topoisomerase inhibitor, and/or tubulin inhibitor. For example, an immunoconjugate of the invention may be used to deliver a non-radioisotope cytotoxin to a target cell. Non-limiting examples of cytotoxic agents include aziridines, cisplatins, tetrazines, procarbazine, hexamethylmelamine, vinca alkaloids, taxanes, camptothecins, etoposide, doxorubicin, mitoxantrone, teniposide, novobiocin, aclarubicin, anthracyclines, actinomycin, bleomycin, plicamycin, mitomycin, daunorubicin, epirubicin, idarubicin, dolastatins, maytansines, docetaxel, adriamycin, calicheamicin, auristatins, pyrrolobenzodiazepine, carboplatin, 5-fluorouracil (5-FU), capecitabine, mitomycin C, paclitaxel, 1,3-Bis(2-chloroethyl)-1-nitrosourea (BCNU), rifampicin, cisplatin, methotrexate, and gemcitabine.

In some embodiments, a radioimmunoconjugate of the invention comprises a radioisotope selected from the group comprising ²²⁵Ac, ⁸⁶Y, ⁹⁰Y, ¹⁷⁷Lu, ¹⁸⁶Re, ¹⁸⁸Re, ⁸⁹Sr, ¹⁵³Sm, ²¹³Bi, ²¹³Po, ²¹¹At, ²¹²Bi ²²³Ra, ²²⁴Ra, ²²⁷Th, ¹⁴⁹Tb, ⁶⁸Ga, ⁶⁴Cu, ⁶⁷Cu, ⁸⁹Zr, ¹³⁷Cs, ²¹²Pb, and ¹⁰³Pd.

In some embodiments, a radioimmunoconjugate of the invention comprises a radioisotope selected from the group consisting of ²²⁵Ac, ⁸⁶Y ⁹⁰Y, ¹⁷⁷Lu, ¹⁸⁶Re, ¹⁸⁸Re, ⁸⁹Sr, ¹⁵³Sm, ²¹³Bi, ²¹³Po, ²¹¹At, ²¹²Bi, ²²³Ra, ²²⁴Ra, ²²⁷Th, ¹⁴⁹Tb, ⁶⁸Ga, ⁶⁴Cu, ⁶⁷Cu, ⁸⁹Zr, ¹³⁷Cs, ²¹²Pb, and ¹⁰³Pd.

In some embodiments, the radioisotope is an alpha-particle-emitting radioisotope comprises ²²Ac, ²²³Ra, ²²⁴Ra, ²²⁷Th, ²¹²Pb, ²¹²Bi, or ²¹³Bi.

In some embodiments, the radioisotope is an alpha-particle-emitting radioisotope selected from the group consisting of ²²⁵Ac, ²²³Ra, ²²⁴Ra, ²²⁷Th, ²¹²Pb, ²¹²Bi, and ²¹³Bi.

Further embodiments of the immunoconjugates, antigen binding regions, and heavy chain variable regions are described below:

In some embodiments, the immunoconjugate comprises a dimerization domain or motif. In some further embodiments, the dimerization domain or motif is in a variant constant region, linker or hinge region.

The skilled worker can engineer multimeric immunoconjugates of the present invention using approaches and methods known in the art. For example, engineered cysteine residues can form covalent bonds thereby stabilizing multimeric structures that spontaneously assemble (see e.g., Glockshuber R et al., Biochemistry 29: 1362-7 (1990)). For example, the introduction of cysteine residues at specific locations may be used to create disulfide stabilized structures like Cys-diabodies, scFv′ multimers, VHH multimers, VNAR multimers, and IgNAR multimers such as, e.g., by adding the following amino acid residues: GGGGC and SGGGGC (Tai M et al., Biochemistry 29: 8024-30 (1990); Caron P et al., J Exp Med 176: 1191-5 (1992); Shopes B, J Immunol 148: 2918-22 (1992); Adams G et al., Cancer Res 53: 4026-34 (1993); McCartney J et al., Protein Eng 18: 301-14 (1994); Perisic O et al., Structure 2: 1217-26 (1994); George A et al., Proc Natl Acad Sci USA 92: 8358-62 (1995); Tai M et al., Cancer Res (Suppl) 55: 5983-9 (1995); Olafsen T et al., Protein Eng Des Sel 17: 21-7 (2004)).

Alternatively, two or more polypeptide chains may be linked together using polypeptide domains which self-associate or multimerize with each other (see e.g., U.S. Pat. No. 6,329,507). For example, the addition of carboxy-terminal multimerization domains has been used to construct multivalent proteins comprising immunoglobulin domains, such as, e.g., scFvs, autonomous V_{H} domains, V_{H}Hs, V_{NAR}s, and IgNARs. Examples of self-associating domains known to the skilled worker include immunoglobulin constant domains (such as knobs-into-holes, electrostatic steering, and IgG/IgA strand exchange), immunoglobulin Fab chains (e.g., (Fab-scFv)₂ and (Fab′ scFv)₂), immunoglobulin Fc domains (e.g., (scDiabody-Fc)₂, (scFv-Fc)₂ and scFv-Fc-scFv), immunoglobulin CHX domains, immunoglobulin CH1-3 regions, immunoglobulin CH3 domains (e.g., (scDiabody-CH3)₂, LD minibody, and Flex-minibody), immunoglobulin CH4 domains, CHCL domains, amphiphilic helix bundles (e.g., scFv-HLX), helix-turn-helix domains (e.g., scFv-dHlx), coiled-coil structures including leucine zippers and cartilage oligometric matrix proteins (e.g., scZIP), cAMP-dependent protein kinase (PKA) dimerization and docking domains (DDDs) combined with an A kinase anchor protein (AKAP) anchoring domain (AD) (also referred to as “dock-and-lock” or “DNL”), streptavidin, verotoxin B multimerization domains, tetramerization regions from p53, and barnase-barstar interaction domains (Pack P, Plückthun A, Biochemistry 31: 1579-84 (1992); Holliger P et al., Proc Natl Acad Sci USA 90: 6444-8 (1993); Kipriyanov S et al., Hum Antibodies Hybridomas 6: 93-101 (1995); de Kruif J, Logtenberg T, J Biol Chem 271: 7630-4 (1996); Hu S et al., Cancer Res 56: 3055-61 (1996); Kipriyanov S et al., Protein Eng 9: 203-11 (1996); Rheinnecker M et al., J Immunol 157: 2989-97 (1996); Tershkikh A et al., Proc Natl Acad Sci USA 94: 1663-8 (1997); Müller K et al., FEBS Lett 422: 259-64 (1998); Cloutier S et al., Mol Immunol 37: 1067-77 (2000); Li S et al., Cancer Immunol Immunother 49: 243-52 (2000); Schmiedl A et al., Protein Eng 13: 725-34 (2000); Schoonjans R et al., J Immunol 165: 7050-7 (2000); Borsi L et al., Int J Cancer 102: 75-85 (2002); Deyev S et al., Nat Biotechnol 21: 1486-92 (2003); Wong W, Scott J, Nat Rev Mol Cell Biol 5: 959-70 (2004); Zhang J et al., J Mol Biol 335: 49-56 (2004); Baillie G et al., FEBS Letters 579: 3264-70 (2005); Rossi E et al., Proc Natl Acad Sci USA 103: 6841-6 (2006); Simmons D et al., J Immunol Methods 315: 171-84 (2006); Braren I et al., Biotechnol Appl Biochem 47: 205-14 (2007); Chang C et al., Clin Cancer Res 13: 5586-91s (2007); Liu M et al., Biochem J 406: 237-46 (2007); Zhang J et al., Protein Expr Purif 65: 77-82 (2009); Bell A et al., Cancer Lett 289: 81-90 (2010); Iqbal U et al., Br J Pharmacol 160: 1016-28 (2010); Asano R et al., FEBS J 280: 4816-26 (2013); Gil D, Schrum A, Adv Biosci Biotechnol 4: 73-84 (2013)).

The skilled worker can engineer multimeric immunoconjugates of the present invention using various scFv-based polypeptide interactions known in the art, such as, e.g., scFv-based dimeric, trimeric, tetrameric complexes, etc. For example, the length of the linker in the scFv can affect the spontaneous assembly of non-covalent based, multimeric, multivalent structures. Generally, linkers of twelve amino acids or less, including the absence of any linker, promote the multimerization of polypeptides or proteins comprising scFvs into higher molecular weight species via favoring intermolecular domain swapping over intra-chain domain pairing (see e.g., Dolezal O et al., Protein Eng 16: 47-56 (2003)). However, scFvs with no linker at all or a linker with an exemplary length of 15 amino acid residues may multimerize (Whitlow M et al., Protein Eng 6: 989-95 (1993); Desplancq D et al., Protein Eng 7: 1027-33 (1994); Whitlow M et al., Protein Eng 7, 1017-26 (1994); Alfthan K et al., Protein Eng 8: 725-31 (1995)). The skilled worker can identify the multimeric structure(s) created and/or purified using techniques known in the art and/or described herein.

In some embodiments, amino acid sequence variants of the immunoconjugates described herein are contemplated. For example, it may be desirable to improve the binding affinity, stability, and/or other biological properties of the immunoconjugate of the present invention (e.g., alter the half-life or therapeutic window, reduce immunogenicity, or increase ease of manufacturing). Amino acid sequence variants of an immunoconjugate may be prepared by introducing appropriate modifications into the nucleotide sequence encoding the immunoconjugate, or by synthesis of the desired immunoconjugate or polypeptide. Such modifications include, for example, fusion of immunoglobulin domains or polypeptide sequences; substitution of hinge, linker(s), and/or chelator components; substitution of radioisotope. Such modifications include, for example, deletions from, and/or insertions into and/or substitutions of residues within the amino acid sequences of the immunoconjugate. Any combination of fusion, deletion, insertion, and substitution can be made to arrive at the final construct, provided that the final construct possesses the desired characteristics, e.g., a certain binding affinity level of antigen binding, a certain level of K_{D}, and/or a certain level of Koff.

Antigen binding antibody fragments and sets of CDRs are provided herein. Such fragments may be truncated at the N-terminus or C-terminus, or may lack internal residues, for example, when compared with a full-length native antibody (e.g., a full-length camelid VHH IgG2 or IgG3). Certain fragments may lack amino acid residues or domain that are not essential for a desired biological activity of the antibody or to reduce the total size of the immunoconjugate of the invention.

In some embodiments, a variant of an immunoconjugate of the present invention is made to be larger by the incorporation of additional structure. In some embodiments, an immunoconjugate is linked to a heterologous moiety or readily detectable moiety. In some further embodiments, the linkage comprises a proteinaceous fusion. In some further embodiments, the heterologous moiety is a cytotoxic agent. In some embodiments, a carboxy-terminal lysine residue is added to provide a site-specific attachment site. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an immunoconjugate with an N-terminal methionyl residue. Other insertional variants of the immunoconjugate molecule include the fusion to the N- or C-terminus of the immunoconjugate to an enzyme (e.g., for ADEPT) or a polypeptide which increases the serum half-life of the immunoconjugate.

Nucleic acids that encode the immunoconjugate of the invention may be modified to produce chimeric or fusion immunoconjugate polypeptides, for example, by substituting human heavy chain and light chain constant domain (CH and CO sequences for the homologous murine sequences (U.S. Pat. No. 4,816,567; and Morrison, et al., Proc Natl Acad Sci USA 81: 6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an immunoconjugate, or they are substituted for the variable domains of one antigen-combining site of an immunoconjugate to create a chimeric bivalent immunoconjugate comprising one antigen-combining site having specificity for an antigen and another antigen-combining site having specificity for a different antigen.

Variations in the antibody constructs used as antigen binding domains in the inventions described herein, can be made, for example, using any of the techniques and guidelines for conservative and non-conservative mutations set forth, for instance, in U.S. Pat. No. 5,364,934. Variations may be a substitution, deletion or insertion of one or more codons encoding the immunoconjugate or polypeptide that results in a change in the amino acid sequence as compared with the native sequence antibody or polypeptide. Optionally the variation is by substitution of at least one amino acid with any other amino acid in one or more of the domains of the immunoconjugate. Guidance in determining which amino acid residue may be inserted, substituted or deleted without adversely affecting the desired activity may be found by comparing the sequence of the immunoconjugate with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

In particular embodiments, conservative substitutions of interest are shown in Tables B and C, including under the heading of preferred substitutions. If such substitutions result in a change in biological activity, then more substantial changes, denominated exemplary substitutions in Table C, or as further described below in reference to amino acid classes, are introduced and the products screened.

TABLE C

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in function or immunological identity of an immunoconjugate of the invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art, such as, e.g., oligonucleotide-mediated (site-directed) mutagenesis, alanine scanning, and PCR mutagenesis. Site-directed mutagenesis (Carter et al., Nucl. Acids Res., 13: 4331 (1986); Zoller et al., Nucl. Acids Res., 10: 6487 (1987)), cassette mutagenesis (Wells et al., Gene, 34: 315 (1985)), restriction selection mutagenesis (Wells et al., Philos. Trans. R. Soc. London SerA, 317: 415 (1986)) or other known techniques can be performed on the cloned DNA to produce DNA molecules encoding an immunoconjugate variant of the invention.

In some embodiments, immunoconjugate variants having one or more amino acid substitutions are provided. Sites of interest for substitutional mutagenesis include the HVRs and FRs of immunoglobulin variable domains as well as within the immunoglobulin constant domains. Amino acid substitutions may be introduced into an immunoconjugate of interest and the products screened for a desired activity, e.g., improved/retained antigen binding, decreased/retained immunogenicity, improved/retained antibody-dependent cellular cytotoxicity (ADCC), improved/retained complement dependent cytotoxicity (CDC), improved/retained target inhibition, and/or improved/retained antibody-dependent cell-mediated phagocytosis (ADCP). Similarly, amino acid substitutions may be introduced into an immunoconjugate of interest and the products screened for the reduction or elimination of an activity, e.g., ADCC, CDC, target inhibition, and/or ADCP.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An illustrative substitutional variant is an affinity-matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve immunoconjugate affinity. Such alterations may be made in HVR “hotspots,” i.e., residues encoded by codons that undergo mutation at high frequency during the somatic maturation process (see e.g., Chowdhury, Methods Mol. Biol. 207: 179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the immunoconjugate to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions.

Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

In some embodiments, the immunoconjugate of the present invention comprises an antibody construct (used as an antigen binding region herein) comprising a humanized immunoglobulin domain(s).

Humanized forms of non-human (e.g., camelid, murine, or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as a camelid, mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin (Jones et al., Nature, 321: 522-5 (1986); Riechmann et al., Nature, 332: 323-9 (1988); and Presta, Curr. Op. Struct. Biol., 2: 593-6 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

According to another method, antigen binding may be restored during humanization of antibodies through the selection of repaired hypervariable regions (see, e.g., U.S. application Ser. No. 11/061,841, filed Feb. 18, 2005). The method includes incorporating non-human hypervariable regions onto an acceptor framework and further introducing one or more amino acid substitutions in one or more hypervariable regions without modifying the acceptor framework sequence. Alternatively, the introduction of one or more amino acid substitutions may be accompanied by modifications in the acceptor framework sequence.

Any cysteine residue not involved in maintaining the proper conformation of the immunoconjugate of the invention also may be substituted, generally with serine, to improve the oxidative stability of the molecule and prevent aberrant crosslinking. Conversely, cysteine bond(s) may be added to the immunoconjugate of the invention to improve its stability (particularly where the antibody is an antibody fragment such as an Fv fragment or VHH fragment).

In some embodiments, it may be desirable to create cysteine engineered immunoconjugates in which one or more residues of an immunoconjugate are substituted with cysteine residues. In some embodiments, the substituted residues occur at accessible sites of the immunoconjugate. By substituting those residues with cysteine, reactive thiol groups are thereby positioned at accessible sites of the immunoconjugate and may be used to conjugate the immunoconjugate to other moieties, such as drug moieties or linker-drug moieties. In some embodiments, any one or more of the following residues may be substituted with cysteine: V205 (Kabat numbering) of the light chain; Al18 (EU numbering) of the heavy chain; and S400 (EU numbering) of the heavy chain Fc region. Cysteine engineered antibodies may be generated as described, e.g., in U.S. Pat. No. 7,521,541.

The skilled worker will appreciate that amino acid changes may alter post-translational processes of the immunoconjugate, such as changing the number or position of glycosylation sites or altering the membrane anchoring characteristics.

In some embodiments, an immunoconjugate provided herein is altered to increase or decrease the extent to which the immunoconjugate is glycosylated and/or to change the glycosylation pattern. “Altering the native glycosylation pattern” is intended for purposes herein to mean deleting one or more carbohydrate moieties found in a parental immunoconjugate of the invention (either by removing the underlying glycosylation site or by deleting the glycosylation by chemical and/or enzymatic means), and/or adding one or more glycosylation sites that are not present in the native sequence immunoconjugate of the invention. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition or deletion of glycosylation sites to an immunoconjugate may be conveniently accomplished by altering the amino acid sequence such that one or more glycosylation sites is created or removed. Addition of glycosylation sites to the immunoconjugate of the invention is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original immunoconjugate of the invention (for O-linked glycosylation sites). The immunoconjugate of the invention amino acid sequence may optionally be altered through changes at the DNA level, particularly by mutating the DNA encoding the immunoconjugate of the invention at preselected bases such that codons are generated that will translate into the desired amino acids.

Where the immunoconjugate comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region (see e.g., Wright et al. TIBTECH 15:26-32 (1997)). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an immunoconjugate of the invention may be made in order to create immunoconjugate variants with certain improved properties.

Another means of increasing the number of carbohydrate moieties on the immunoconjugate of the invention is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the immunoconjugate of the invention may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

In some embodiments, immunoconjugate variants are provided having a carbohydrate structure that lacks fucose attached (directly or indirectly) to an Fc region. For example, the amount of fucose in such immunoconjugate may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function (see e.g., US 2003/0157108; US 2004/0093621). Examples of publications related to “defucosylated” or “fucose-deficient” antibody variants include: US 2003/0157108; WO 2000/61739; WO 2001/29246; US 2003/01 15614; US 2002/0164328; US 2004/0093621; US 2004/0132140; US 2004/01 10704; US 2004/01 10282; US 2004/0109865; WO 2003/0851 19; WO 2003/084570; WO 2005/035586; WO 2005/035778; WO2005/053742; WO2002/031 140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lecl3 CHO cells deficient in protein fucosylation (Ripka et al. Arch. Biochem. Biophys. 249:533-545 (1986); US 2003/0157108; WO 2004/056312, Adams et al., especially at Example 11), and knockout cell lines, such as alpha-1,6-fucosyltransferase gene, FUT8, knockout CHO cells (see e.g., Yamane-Ohnuki et al. Biotech. Bioeng. 87: 614 (2004); Kanda, Y. et al., Biotechnol. Bioeng., 94(4):680-688 (2006); WO2003/085107)).

Immunoconjugate variants are further provided with bisected oligosaccharides, e.g., in which a biantennary oligosaccharide attached to the Fc region of the antibody is bisected by GlcNAc. Such immunoconjugate variants may have reduced fucosylation and/or improved ADCC function. Examples of such antibody variants are described, e.g., in WO 2003/011878; U.S. Pat. No. 6,602,684; US 2005/0123546. Immunoconjugate variants with at least one galactose residue in the oligosaccharide attached to the Fc region are also provided. Such immunoconjugate variants may have improved CDC function. Such antibody variants are described, e.g., in WO 1997/030087; WO 1998/058964; and WO 1999/022764.

### Immunoconjugate Derivatives and Other Modifications

Covalent modifications of the immunoconjugates of the invention are included within the scope of this invention. One type of covalent modification includes reacting targeted amino acid residues of an immunoconjugate of the invention with an organic derivatizing agent that is capable of reacting with selected side chains or the N- or C-terminal residues of the immunoconjugate. Derivatization with bifunctional agents is useful, for instance, for crosslinking an immunoconjugate of the invention to a water-insoluble support matrix or surface for use in the method for purifying the immunoconjugates of the invention, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

In some embodiments, an immunoconjugate provided herein may be further modified to contain additional nonproteinaceous moieties that are known in the art and readily available. The moieties suitable for derivatization of the immunoconjugate include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1, 3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the immunoconjugate may vary, and if more than one polymer are attached, they can be the same or different molecules. In general, the number and/or type of polymers used for derivatization can be determined based on considerations including, but not limited to, the particular properties or functions of the immunoconjugate to be improved, whether the immunoconjugate derivative will be used in a therapy under defined conditions, etc.

PEG derivatized immunoconjugates of the invention may comprise linkers comprising one or more —CH₂CH₂O— and can be used to alter biodistribution and pharmacokinetics of the immunoconjugate. PEGs can be prepared in a polymeric form or as discrete oligomers. Bifunctionalized versions of these polymers can link immunoconjugates with a chelating agent and/or provide additional size and/or solubility to the overall molecule. In some embodiments, the PEG derivatized immunoconjugates exhibit reduced immunogenicity compared to their un-derivatized parental molecules.

### Methods of Producing the Immunoconjugates of the Present Invention

The present invention provides a composition comprising one or more of the immunoconjugates according to any of the above embodiments or described herein. In another aspect, the invention provides an isolated nucleic acid encoding a radioisotope delivering platform as described herein. Also provided herein are nucleic acids encoding the protein components of the immunoconjugates of the present invention, expression vectors comprising the aforementioned nucleic acid, and host cells comprising the aforementioned expression vectors.

In another aspect, the invention provides a host cell comprising a nucleic acid and/or vector as provided herein. In some embodiments, the host cell of the present invention is isolated or purified. In some embodiments, the host cell of the present invention is in a cell culture medium. The nucleic acids, expression vectors, and host cells of the invention may be used to produce a composition comprising one or more of the immunoconjugates of the invention. In some embodiments, the host cell is eukaryotic. In some embodiments, the host cell is mammalian. In some embodiments, the host cell is a Chinese Hamster Ovary (CHO) cell. In some embodiments, the host cell is prokaryotic. In some embodiments, the host cell is E. coli.

A description follows as to illustrative techniques for the production of the immunoconjugates and radioimmunoconjugates of the present invention for use in accordance with the methods of the present invention. In some embodiments, the invention provides a process for making an immunoconjugate of the present invention, the method comprising culturing a host cell as provided herein under conditions suitable for the expression vector encoding the radioisotope delivery platform and recovering or purifying the radioisotope delivery platform. In some embodiments, the method further comprises radiolabeling the radioisotope delivery platform with an appropriate isotope, such as, e.g., an alpha or beta particle emitter.

### Generation and Identification of Antigen Binding Domains, Immunoconjugates and Nucleic Acids

Antigen binding domains useful as antigen binding regions herein may be identified in antibodies that are either monoclonal antibodies and/or polyclonal antibodies. DNA encoding a monoclonal antibody is readily isolated and sequenced using conventional procedures. Once isolated, the DNA may be placed into expression vectors, which are then transfected into host cells such as E. coli cells, simian COS cells, Chinese hamster ovary (CHO) cells, or myeloma cells that do not otherwise produce antibody protein, to obtain the synthesis of monoclonal antibodies in the recombinant host cells (see e.g., Skerra et al., Curr. Opinion in Immunol., 5:256-262 (1993) and Plückthun, Immunol Revs. 130:151-188 (1992)).

In some embodiments, the antigen binding domains of an immunoconjugate of the present invention, or fragments thereof, are isolated by screening phage libraries containing phage that display various fragments of antibody variable region (Fv, scFv, or VHH) fused to phage coat protein. Such phage libraries are screened for binding to the desired target antigen or epitope. Clones expressing Fv fragments, scFv's, or VHH's capable of binding to the desired antigen are adsorbed to the antigen and thus separated from the non-binding clones in the library. The binding clones are then eluted from the antigen, and can be further enriched by additional cycles of antigen adsorption/elution.

In some embodiments, the antibody or antibody fragments thereof are isolated from antibody phage libraries generated using the techniques described in McCafferty et al., Nature, 348:552-554 (1990). Clackson et al., Nature, 352:624-628 (1991) and Marks et al., J Mol Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc Acids Res. 21:2265-2266 (1993)). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12: 433-455 (1994).

Repertoires of VH and VL genes can be separately cloned by polymerase chain reaction (PCR) and recombined randomly in phage libraries, which can then be searched for antigen binding clones as described in Winter et al., Ann. Rev. Immunol., 12: 433-455 (1994). Naïve libraries for screening can be constructed from non-immunized sources to provide high-affinity antibodies to antigens (see e.g., Griffiths et al., EMBO J, 12: 725-734 (1993)). Another example is naive libraries constructed synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227: 381-388 (1992).

Screening of the libraries can be accomplished by various techniques known in the art. For example, target antigen can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (and strong binding affinities) can be promoted by use of long washes and monovalent phage display as described in Bass et al., Proteins, 8: 309-314 (1990) and in WO 1992/09690, and a low coating density of antigen as described in Marks et al., Biotechnol., 10: 779-783 (1992).

Techniques for screening a cDNA library are well known in the art. Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding immunoconjugate of the invention is to use PCR methodology (Sambrook et al., supra; Dieffenbach et al., PCR Primer: A Laboratory Manual (Cold Spring Harbor Laboratory Press, 1995)).

DNA encoding an immunoconjugate of the invention may be obtained from a cDNA library prepared from tissue believed to possess the immunoconjugate of the invention mRNA and to express it at a detectable level. Accordingly, human immunoconjugate of the invention DNA can be conveniently obtained from a cDNA library prepared from human tissue. The immunoconjugate of the invention-encoding gene may also be obtained from a genomic library or by known synthetic procedures (e.g., automated nucleic acid synthesis). For some embodiments, desired polynucleotide sequences encoding antibodies may be isolated and sequenced from antibody producing cells such as hybridoma cells.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein. Any of the antibody CDRs or heavy chain variable fragments of the present invention can be obtained by designing a suitable antigen screening procedure to select for the phage clone of interest followed by construction of an antibody clone using the variable domain and/or CDRs sequences from a phage clone of interest and suitable constant region (Fc) sequences described in Kabat et al., 1991, supra.

### Immunoconjugate Production; Host Cells and Expression Vectors of the Invention

The description below relates primarily to production of the antibody constructs of the invention by culturing cells transformed or transfected with a vector-containing immunoconjugate of the invention-encoding nucleic acid. It is, of course, contemplated that alternative methods, which are well known in the art, may be employed to prepare the antibody constructs of the invention. For instance, the appropriate amino acid sequence, or portions thereof, may be produced by direct peptide synthesis using solid-phase techniques (e.g., Stewart et al., Solid-Phase Peptide Synthesis, W.H. Freeman Co., San Francisco, CA (1969); Merrifield, J, Am. Chem. Soc., 85: 2149-54 (1963)). In vitro protein synthesis may be performed using manual techniques or by automation. Automated synthesis may be accomplished, for instance, using an Applied Biosystems Peptide Synthesizer (Foster City, CA) using manufacturer's instructions. Various portions of the immunoconjugate of the invention may be chemically synthesized separately and combined using chemical or enzymatic methods to produce the desired immunoconjugate of the invention.

Antibody constructs may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VH of the antibody and/or comprising the VL amino acid sequence (e.g., the light and/or heavy chains of the antibody). In a further embodiment, one or more vectors (e.g., expression vectors) comprising such nucleic acid are provided. In a further embodiment, a host cell comprising such nucleic acid is provided. In some embodiments, a host cell comprises (e.g., has been transformed with): (1) a vector comprising a nucleic acid that encodes an amino acid sequence comprising the VH of the antibody. In some other embodiments, a host cell comprises: (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., YO, NSO, Sp20 cell). In one embodiment, a method of making an immunoconjugate of the invention is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an immunoconjugate of the present invention, nucleic acid encoding an antibody construct, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and/or light chains of the antibody). Nucleic acid molecules encoding amino acid sequence of the immunoconjugate of the present invention (including sequence variants) may be prepared by a variety of methods known to the skilled worker. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody construct.

### Manipulation of Host Cells for Immunoconjugate Production

Host cells are transfected or transformed with expression or cloning vectors described herein for immunoconjugate of the invention production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Suitable host cells for cloning or expression of immunoconjugate-encoding nucleic acids and vectors include prokaryotic or eukaryotic cells described herein. For example, antibodies may be produced in bacteria, in particular when glycosylation and Fc effector function are not needed. For expression of antibody fragments and polypeptides in bacteria, see e.g., U.S. Pat. Nos. 5,648,237; 5,789,199; 5,840,523; and Charlton, Methods in Molecular Biology, Vol. 248 (B.K. C. Lo, ed., Humana Press, Totowa, NJ, 2003), pp. 245-254, describing expression of antibody fragments in E. coli). After expression, the immunoconjugate may be isolated from the bacterial cell paste in a soluble fraction and can be further purified.

In addition to prokaryotes, eukaryotic microbes such as filamentous fungi or yeast are suitable cloning or expression hosts for immunoconjugate-encoding vectors, including fungi and yeast strains whose glycosylation pathways have been “humanized,” resulting in the production of an antibody with a partially or fully human glycosylation pattern (see e.g., Gerngross, Nat. Biotech. 22:1409-1414 (2004); Li et al., Nat. Biotech. 24:210-215 (2006)).

Suitable host cells for the expression of glycosylated immunoconjugate are also derived from multicellular organisms (e.g., invertebrates and vertebrates). Examples of invertebrate cells include plant and insect cells. Numerous baculoviral strains have been identified which suitable for use in conjunction with insect cells, particularly for transfection of Spodoptera frugiperda cells. Plant cell cultures can also be utilized as hosts (see e.g., U.S. Pat. Nos. 5,959,177; 6,040,498; 6,420,548; 7,125,978; and 6,417,429.

Vertebrate cells may also be used as hosts. For example, mammalian cell lines that are adapted to grow in suspension may be useful. Other examples of useful mammalian host cell lines are monkey kidney CV 1 line transformed by SV40 (COS-7); human embryonic kidney line (293 or 293 cells as described, e.g., in Graham et al., J Gen Viral. 36:59 (1977); baby hamster kidney cells (BHK); mouse Sertoli cells (TM4 cells as described, e.g., in Mather, Biol. Reprod. 23:243-251 (1980); monkey kidney cells (CV 1); African green monkey kidney cells (VERO-76); human cervical carcinoma cells (HELA); canine kidney cells (MOCK; buffalo rat liver cells (BRL 3A); human lung cells (W138); human liver cells (Hep 02); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFK CHO cells (Urlaub et al., Proc Natl Acad Sci USA 77:4216 (1980)); and myeloma cell lines such as YO, NSO and Sp2/0. For a review of certain mammalian host cell lines suitable for immunoconjugate production, see e.g., Yazaki and Wu, Methods in Molecular Biology, Vol. 248 (B.K. C. Lo, ed., Humana Press, Totowa, NJ), pp. 255-268 (2003).

Methods of eukaryotic cell transfection and prokaryotic cell transformation, which means introduction of DNA into the host so that the DNA is replicable, either as an extrachromosomal or by chromosomal integrant, are known to the skilled worker, for example, CaCl₂, CaPO₄, liposome-mediated, polyethylene-gycol/DMSO and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc Natl Acad Sci USA 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

### Prokaryotic Host Cells

Suitable prokaryotes include but are not limited to archaebacteria and eubacteria, such as Gram-negative or Gram-positive organisms, for example, Enterobacteriaceae such as E. coli. Various E. coli strains are publicly available, such as K12 strain MM294 (ATCC 31,446); X1776 (ATCC 31,537); W3110 (ATCC 27,325) and KS 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. lichenformis (e.g., B. licheniformis 41P disclosed in DD 266,710 published 12 Apr. 1989), Pseudomonas such as P. aeruginosa, Rhizobia, Vitreoscilla, Paracoccus and Streptomyces. These examples are illustrative rather than limiting. E. coli strain W3110 is one advantageous host or parent host because it is a common host strain for recombinant DNA product fermentations. Preferably, the host cell secretes minimal amounts of proteolytic enzymes. For example, strain W3110 (Bachmann, Cellular and Molecular Biology, vol. 2 (Washington, D.C.: American Society for Microbiology, 1987), pp. 1190-1219; ATCC Deposit No. 27,325) may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kanr; E. coli W3110 strain 37D6, which has the complete genotype tonA ptr3 phoA E15 (argF-lac)169 degP ompT rbs7 ilvG kanr; E. coli W3110 strain 40B4, which is strain 37D6 with a non-kanamycin resistant degP deletion mutation; E. coli W3110 strain 33D3 having genotype W3110 ΔfhuA (ΔtonA) ptr3 lac Iq lacL8 ΔompTΔ(nmpc-fepE) degP41 kanR (U.S. Pat. No. 5,639,635) and an E. coli strain having mutant periplasmic protease disclosed in U.S. Pat. No. 4,946,783 issued 7 Aug. 1990. Other strains and derivatives thereof, such as E. coli 294 (ATCC 31,446), E. coli B, E. coli X 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture. Alternatively, in vitro methods of cloning, e.g., PCR or other nucleic acid polymerase reactions, are suitable.

Full length antibody, antibody fragments, and antibody fusion proteins can be produced in bacteria, in particular when glycosylation and Fc effector function are not needed. Full length antibodies have greater half-life in circulation. Production in E. coli is faster and more cost efficient. For expression of antibody fragments and polypeptides in bacteria, see, e.g., U.S. Pat. Nos. 5,648,237; 5,789,199 and 5,840,523, which describe translation initiation region (TIR) and signal sequences for optimizing expression and secretion. After expression, the immunoconjugate is isolated from the E. coli cell paste in a soluble fraction and can be purified through, e.g., a protein A or G column depending on the isotype. Final purification can be carried out similar to the process for purifying antibody expressed e.g., in CHO cells.

### Eukaryotic Host Cells

In addition to prokaryotes, eukaryotic microbes such as filamentous fungi or yeast are suitable cloning or expression hosts for immunoconjugate of the invention-encoding vectors. Saccharomyces cerevisiae is a commonly used lower eukaryotic host microorganism. Others include Schizosaccharomyces pombe (Beach and Nurse, Nature, 290: 140 (1981); EP 139,383 published 2 May 1985); Kluyveromyces hosts (U.S. Pat. No. 4,943,529; Fleer et al., Bio/Technology, 9: 968-75 (1991)) such as, e.g., K. lactis (MW98-8C, CBS683, CBS4574; Louvencourt et al., J. Bacteriol., 154(2):737-742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 (1988)); Candida; Trichoderma reesia (EP 244,234); Neurospora crassa (Case et al., Proc Natl Acad Sci USA 76:5259-5263 (1979)); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 (1983); Tilburn et al., Gene, 26:205-221 (1983); Yelton et al., Proc Natl Acad Sci USA 81: 1470-1474 (1984)) and A. niger (Kelly and Hynes, EMBO J., 4:475-479 (1985)). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated immunoconjugate of the invention are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc Natl Acad Sci USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for immunoconjugate of the invention production and cultured in conventional nutrient media modified as appropriate for inducing promoters, selecting transformants, or amplifying the genes encoding the desired sequences.

### Selection and Use of a Replicable Vector

For recombinant production of a radioisotope delivery platform of the invention, the nucleic acid (e.g., cDNA or genomic DNA) encoding it is isolated and inserted into a replicable vector for further cloning (amplification of the DNA) or for expression. DNA encoding the immunoconjugate is readily isolated and sequenced using conventional procedures (e.g., by using oligonucleotide probes that are capable of binding specifically to genes encoding the heavy and light chains of an antibody). Many vectors are available. The choice of vector depends in part on the host cell to be used. Generally, suitable host cells are of either prokaryotic or eukaryotic (generally mammalian) origin.

The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The immunoconjugate of the invention may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the immunoconjugate of the invention-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

### Culturing Host Cells Producing Radioisotope Delivery Platforms

The host cells used to produce the immunoconjugate of the invention of this invention may be cultured in a variety of media and culture conditions.

### Prokaryotic Host Cell Cultures

Prokaryotic cells used to produce the polypeptides of the invention are grown in media known in the art and suitable for culture of the selected host cells. Examples of suitable media include Luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. In some embodiments, the phosphate-limiting medium is the C.R.A.P medium (see, e.g., Simmons et al., J. Immunol. Methods (2002), 263: 133-47). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, immunoconjugate production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (a preferred carbon/energy source). Small-scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted immunoconjugate polypeptides, additional vectors overexpressing chaperone proteins, such as Dsb proteins (DsbA, DsbB, DsbC, DsbD and or DsbG) or FkpA (a peptidylprolyl cis, trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274: 19601-5; U.S. Pat. Nos. 6,083,715; 6,027,888; Bothmann and Pluckthun (2000) J. Biol. Chem. 275:17100-5; Ramm and Pluckthun (2000) J. Biol. Chem. 275:17106-13; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; U.S. Pat. Nos. 5,264,365; 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

### Eukaryotic Host Cell Cultures

Commercially available media such as Ham's F10 (Sigma), Minimal Essential Medium ((MEM), (Sigma), RPMI-1640 (Sigma), and Dulbecco's Modified Eagle Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO 90/03430; WO 87/00195; or U.S. Patent Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Purification of an Immunoglobulin-Derived Structure of the Invention

Forms of immunoconjugate of the invention may be recovered from culture medium or from host cell lysates. If membrane-bound, it can be released from the membrane using a suitable detergent solution (e.g., Triton-X 100) or by enzymatic cleavage. Cells employed in expression of immunoconjugate of the invention can be disrupted by various physical or chemical means, such as freeze-thaw cycling, sonication, mechanical disruption, or cell lysing agents.

It may be desired to purify immunoconjugate of the invention from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the immunoconjugate of the invention. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular immunoconjugate of the invention produced.

When using recombinant techniques, the immunoconjugate can be produced intracellularly, in the periplasmic space, or directly secreted into the medium. If the immunoconjugate is produced intracellularly, as a first step, the particulate debris, either host cells or lysed fragments, are removed, for example, by centrifugation or ultrafiltration. Carter et al., Bio/Technology 10: 163-7 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the immunoconjugate is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The immunoconjugate composition prepared from the cells can be purified using, for example, hydroxylapatite chromatography, gel electrophoresis, dialysis, and affinity chromatography, with affinity chromatography being a preferred purification technique. The suitability of protein A as an affinity ligand depends on the species and isotype of any immunoglobulin Fc domain that is present in the immunoconjugate. Protein A can be used to purify antibodies that are based on human γ1, γ2 or γ4 heavy chains (Lindmark et al., J. Immunol. Meth. 62: 1-13 (1983)). Protein G is recommended for all mouse isotypes and for human 73 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the immunoconjugate comprises a CH3 domain, the Bakerbond ABX™ resin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the immunoconjugate to be recovered.

Following any preliminary purification step(s), the mixture comprising the immunoconjugate of interest and contaminants may be subjected to low pH hydrophobic interaction chromatography using an elution buffer at a pH between about 2.5-4.5, and generally at low salt concentrations (e.g., from about 0-0.25M salt).

### Immunoconjugates (Including Antibody Drug Conjugates (ADCs))

In a further aspect of the invention, an immunoconjugate of the invention according to any of the above embodiments or described herein is conjugated to a heterologous moiety or agent, such as, e.g., as described below and including any additional exogenous material as described herein.

In one embodiment, the invention provides immunoconjugates comprising an antibody construct of the present invention conjugated to one or more therapeutic agents or radioactive isotopes.

In some embodiments, an immunoconjugate comprises an antibody construct as described herein conjugated to a radioactive atom to form a radioconjugate. As described herein, a variety of radioactive isotopes are available for the production of radioconjugates of the invention.

Conjugates of an immunoconjugate or antibody construct may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate H), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an illustrative chelating agent for conjugation of radionucleotide to the antibody (see e.g., WO 1994/11026). The linker may be a “cleavable linker” facilitating release of a cytotoxic drug in the cell. For example, an acid-labile linker, peptidase-sensitive linker, photolabile linker, dimethyl linker or disulfide-containing linker may be used (see e.g., Chari et al., Cancer Res. 52:127-131 (1992); U.S. Pat. No. 5,208,020).

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., obtainable from Pierce Biotechnology, Inc., Rockford, IL., U.S.).

As recognized by the person of ordinary skill in the art, certain methods above are also useful to the preparation of radioimmunoconjugates and targeted imaging complexes (notwithstanding the textual reference to only immunoconjugates or antibody constructs), and such preparative methods are also embraced by the invention.

Immunoconjugation Using Chelators and/or Linkers

Methods for affixing a radioisotope to an immunoconjugate or antibody construct (i.e., “labeling” an antibody with a radioisotope) are well known to the skilled worker. Certain of these methods are described, for example, in WO 2017/155937.

Bifunctional chelators, such as, e.g., DOTA, DTPA, and related analogs are suitable for coordinating metal ions like α and β-emitting radionuclides. For example, these chelating molecules can be linked to the targeting molecule by forming a new amide bond between an amine on the antibody construct (e.g., a functional group of a lysine residue) and a carboxylate on the DOTA/DTPA. In the case of peptide synthesis, characterization and purification of the linker addition can be part of the overall synthesis of an antibody platform or immunoconjugate for radioisotope conjugation.

For some embodiment, the method of producing an immunoconjugate involves a click chemistry step described by Poty, S et al., Chem Commun. (Camb) 54: 2599 (2018).

For some embodiments, a peptide may be biosynthesized or may be synthesized by chemical amino acid synthesis using suitable amino acid precursors involving, for example, fluorine-19 in place of hydrogen. In some embodiments, radiolabels may be incorporated into peptide. In some embodiments, radiolabels may be linked to peptide. The IODOGEN method (Fraker et al. (1978) Biochem Biophys Res Commun. 80: 49-57 can be used to incorporate iodine-123. “Monoclonal Antibodies in Immunoscintigraphy” (Chatal, CRC Press 1989) describes other methods in detail.

### Characterization of Immunoconjugates of the Present Invention

Immunoconjugates of the present invention may be identified, screened for, or characterized for their physical/chemical properties and/or biological activities by various assays known in the art. The immunoconjugates and antibody constructs of the invention may be characterized for their physical/chemical properties and/or biological activities by various assays known in the art. Immunoconjugates of the invention can be characterized by a series of assays including, but not limited to, polypeptide sequence determination, amino acid analysis, non-denaturing size exclusion high pressure liquid chromatography (HPLC), mass spectrometry, ion exchange chromatography, and papain digestion.

### Antigen Binding

An immunoconjugate of the present invention may be tested for its antigen binding activity by methods known in the art, e.g., ELISA, Western blot, etc. The binding affinity of an antibody can, for example, be determined by the Scatchard analysis described in Munson et al., Anal Biochem. 107: 220 (1980). Further, the antigen binding ability of an immunoconjugate of the invention may be quantitated using methods known in the art, e.g., a quantitative ELISA, quantitative Western blot, surface plasmon resonance assay, and/or a Scatchard analysis.

In one embodiment, the K_{D} of an immunoconjugate is measured using a radiolabeled antigen ELISA performed with the immunoconjugate. According to another embodiment, the K_{D} is measured by using surface-plasmon resonance assays using a BIACORE®-2000 or a BIACORE®-3000 instrument (BIAcore, Inc., Piscataway, N.J.), e.g., using immobilized antigen CM5 chips at 25° C. and 10 response units.

In another aspect, binding competition assays may be used to identify immunoconjugates that compete for binding to the same antigen, or epitope thereof. In some embodiments, such a competing antibody binds to the same epitope (e.g., a linear or a conformational epitope) of an immunoconjugate of the invention (see e.g., Harlow and Lane (1988) Antibodies: A Laboratory Manual, Ch.14 (Cold Spring Harbor Laboratory, Cold Spring Harbor, NY)).

The epitope and/or contact residues within an antigen bound by an immunoconjugate of the invention can be identified or mapped using methods known to the skilled worker. Detailed exemplary methods for mapping an epitope to which an antibody binds are provided in Morris (1996) “Epitope Mapping Protocols,” in Methods in Molecular Biology (3^{rd} ed., Humana Press, Totowa, NJ).

### Pharmaceutical Compositions and Formulations of the Present Invention

As will be recognized by the person of ordinary skill in the art, certain teachings herein below apply to immunoconjugates and radioimmunoconjugates of the invention, notwithstanding the specific textual reference to one type of invention, and such applications are embraced in entirety by the invention.

In another aspect, the invention provides a composition comprising an immunoconjugate or radioimmunoconjugate of the present invention. The invention further provides pharmaceutical compositions and formulations comprising at least one immunoconjugate of the present invention and at least one pharmaceutically acceptable excipient or carrier. In some embodiments, a pharmaceutical formulation comprises (1) an immunoconjugate or radioimmunoconjugate of the invention, and (2) a pharmaceutically acceptable carrier.

An immunoconjugate or radioimmunoconjugate is formulated in any suitable form for delivery to a target cell/tissue. Pharmaceutical formulations of an immunoconjugate of the present invention are prepared by mixing such immunoconjugate having the desired degree of purity with one or more optional pharmaceutically acceptable carriers, diluents, and/or excipients (Remington's Pharmaceutical Sciences 16th edition, Osol, A. Ed. (1980)), in the form of lyophilized formulations or aqueous solutions. Pharmaceutically acceptable carriers, diluents, and excipients are generally nontoxic to recipients at the dosages and concentrations employed, and include, but are not limited to: sterile water, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Pharmaceutical formulations to be used for in vivo administration are generally sterile. This is readily accomplished by filtration through sterile filtration membranes.

Examples of lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

Pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). In one aspect, a sHASEGP is combined with one or more additional glycosaminoglycanases such as chondroitinases.

The formulation herein may also contain more than one active ingredient as necessary for the particular indication being treated, preferably those with complementary activities that do not adversely affect each other. Such active ingredients are suitably present in combination in amounts that are effective for the purpose intended.

The active ingredients may also be entrapped in microcapsules prepared, for example, by coacervation techniques orby interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

In some embodiments, immunoconjugates may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the immunoconjugate are prepared by methods known in the art, such as described in Epstein et al., Proc Natl Acad Sci USA 82: 3688 (1985); Hwang et al., Proc Natl Acad Sci USA 77: 4030 (1980); U.S. Pat. Nos. 4,485,045 and 4,544,545; and WO1997/38731 published Oct. 23, 1997. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent is optionally contained within the liposome (see Gabizon et al., J. National Cancer Inst. 81: 1484 (1989)). Liposomes with enhanced circulation time are disclosed in U.S. Pat. No. 5,013,556.

Sustained-release preparations may be prepared. Suitable examples of sustained-release preparations include semipermeable matrices of solid hydrophobic polymers containing the antibody, which matrices are in the form of shaped articles, e.g., films, or microcapsules.

### Methods of Using Immunoconjugates and Radioimmunoconjugates and Compositions Thereof

In one aspect, the invention provides a method of treating a disease, disorder, or condition in a patient in need thereof, the method comprising administering to a subject in need thereof a pharmaceutically effective amount of an immunoconjugate or radioimmunoconjugate or composition of the present invention. For some further embodiments, the method is for inhibiting the growth and/or the killing of a cancer cell or tumor. In another aspect, the invention provides for the use of an immunoconjugate described herein for the preparation and/or manufacture of a medicament for treating a disease, disorder, or condition in a subject, such as, e.g., cancer.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, an immunoconjugate or radioimmunoconjugate or composition of the invention can be used in a method for binding target antigen in an individual suffering from a disorder associated with increased target antigen expression and/or activity, the method comprising administering to the individual the immunoconjugate or radioimmunoconjugate or composition such that target antigen in the individual is bound. In one embodiment, the target antigen is human target antigen, and the individual is a human individual. An immunoconjugate or radioimmunoconjugate or composition of the invention can be administered to a human for therapeutic purposes. Moreover, an immunoconjugate or radioimmunoconjugate or composition of the invention can be administered to a non-human mammal expressing target antigen with which the immunoconjugate or radioimmunoconjugate cross-reacts (e.g., a primate, pig, rat, or mouse) for veterinary purposes or as an animal model of human disease. Regarding the latter, such animal models may be useful for evaluating the therapeutic efficacy of an immunoconjugate or radioimmunoconjugate or composition of the invention (e.g., testing of dosages and time courses of administration).

An immunoconjugate or radioimmunoconjugate or composition of the invention (and any additional therapeutic agent or adjuvant) can be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Dosing can be by any suitable route, e.g., by injections, such as intravenous or subcutaneous injections, depending in part on whether the administration is brief or chronic.

Immunoconjugate or radioimmunoconjugate or compositions of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunoconjugates of the invention are administered to a human patient, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. For some embodiments, intravenous or subcutaneous administration of the immunoconjugate or radioimmunoconjugate or composition of the invention is preferred.

For the prevention or treatment of disease, the dosage and mode of administration will be chosen by the physician according to known criteria. The appropriate dosage of immunoconjugate or radioimmunoconjugate or composition of the invention will depend on the type of disease to be treated, as defined above, the severity and course of the disease, whether the immunoconjugate or radioimmunoconjugate or composition of the invention is administered for preventive or therapeutic purposes, previous therapy, the patient's clinical history and response to the immunoconjugate or radioimmunoconjugate or composition, and the discretion of the attending physician. The immunoconjugate or radioimmunoconjugate or composition of the invention is suitably administered to the patient at one time or over a series of treatments. Preferably, the immunoconjugate or radioimmunoconjugate or composition is administered by intravenous infusion or by subcutaneous injections. Depending on the type and severity of the disease, about 1 μg/kg to about 50 mg/kg body weight (e.g., about 0.1-15 mg/kg/dose) of immunoconjugate or radioimmunoconjugate or composition can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the immunoconjugate or radioimmunoconjugate or composition of the invention. However, other dosage regimens may be useful. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

The dose and administration schedule may be selected and adjusted based on the level of disease, or tolerability in the subject, which may be monitored during the course of treatment. The conjugates of the present invention may administered once per day, once per week, multiple times per week, but less than once per day, multiple times per month but less than once per day, multiple times per month but less than once per week, once per month, once per five weeks, once per six weeks, once per seven weeks, once per eight weeks, once per nine weeks, once per ten weeks, or intermittently to relieve or alleviate symptoms of the disease. Administration may continue at any of the disclosed intervals until remission of the tumor or symptoms of the cancer being treated. Administration may continue after remission or relief of symptoms is achieved where such remission or relief is prolonged by such continued administration.

For some embodiments, the effective amount of the immunoconjugate or radioimmunoconjugate or composition may be provided as a single dose.

The Immunoconjugates and radioimmunoconjugates of the present invention maybe used in combination with conventional and/or novel methods of treatment or therapy or separately as a monotherapy. In some embodiments, the immunoconjugates and radioimmunoconjugates of the present invention maybe used with one or more radiation sensitizer agents. Such agents include any agent that can increase the sensitivity of cancer cells to radiation therapy. In other embodiments, immunoconjugates and radioimmunoconjugates of the present invention may be used in combination with novel and/or conventional agents that can augment the biological effects of radiotherapy. Irradiation of a tumor can cause a variety of biological consequences which can be exploited by combining immunoconjugates and radioimmunoconjugates of the present invention with agents that target relevant pathways. In some embodiments, such agents may reduce tumor angiogenesis, or inhibit local invasion and metastasis, or prevent repopulation, or augment the immune response, or deregulate cellular energetics, or reduce population, or alter tumor metabolism, or increase tumor damage, or reduce DNA repair. In certain embodiments, agents for use in combination with immunoconjugates and radioimmunoconjugates of the present invention may comprise DDR inhibitors, e.g., PARP, ATR, Chkl, or DNA-PK; or survival signaling inhibitors, e.g., mTOR, PI3k, NF-kB; or antihypoxia agents, e.g., HIF-1-alpha, CAP, or UPR; or metabolic inhibitors, e.g., MCT1, MCT4 inhibitors; or immunotherapeutics, e.g., anti-CTLA4, anti-PD-1; or inhibitors of growth factor signal transduction, e.g., EGFR or MAPK inhibitors; or anti-invasives, e.g., kinase inhibitors, chemokine inhibitors, or integrin inhibitors; or anti-angiogenic agents, e.g., VEGF-inhibitors.

Immunoconjugates and radioimmunoconjugates of the present invention may (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind. In this context, “inhibiting cell growth or proliferation” means decreasing a cell's growth or proliferation by at least 10%, 20%, 30%, 40%, 50%, 60%, 70%, 80%, 90%, 95%, or 100%, and includes inducing cell death.

By way of example, an immunoconjugate that inhibits the growth of a tumor cell is one that results in measurable growth inhibition of a tumor cell (e.g., a cancer cell). In one embodiment, an immunoconjugate or radioimmunoconjugate of the invention is capable of inhibiting the growth of cancer cells displaying the antigen bound by the immunoconjugate or radioimmunoconjugate. Preferred growth inhibitory immunoconjugates or radioimmunoconjugates inhibit growth of antigen-expressing tumor cells by greater than 20%, preferably from about 20% to about 50%, and even more preferably, by greater than 50% (e.g., from about 50% to about 100%) as compared to the appropriate control, the control typically being tumor cells not treated with the immunoconjugate or radioimmunoconjugate being tested.

For some embodiments, a majority of the immunoconjugate or radioimmunoconjugate or composition administered to a subject typically consists of non-labeled immunoconjugate, with the minority being labeled radioimmunoconjugate. The ratio of labeled radioimmunoconjugate to non-labeled immunoconjugate can be adjusted using known methods. Thus, accordingly to certain aspects of the present invention, the immunoconjugate/radioimmunoconjugate may be provided in a total protein amount of up to 100 mg, such as less than 60 mg, or from 5 mg to 45 mg, or a total protein amount of between 0.1 μg/kg to 1 mg/kg patient weight, such as 1 μg/kg to 1 mg/kg patient weight, or 10 μg/kg to 1 mg/kg patient weight, or 100 μg/kg to 1 mg/kg patient weight, or 0.1 μg/kg to 100 μg/kg patient weight, or 0.1 μg/kg to 50 μg/kg patient weight, or 0.1 μg/kg to 10 μg/kg patient weight, or 0.1 μg/kg to 40 μg/kg patient weight, or 1 μg/kg to 40 μg/kg patient weight, or 0.1 mg/kg to 1.0 mg/kg patient weight, such as from 0.2 mg/kg patient weight to 0.6 mg/kg patient weight.

In certain embodiments, the immunoconjugate/radioimmunoconjugate may be administered from about 0.5 mg/kg to about 30 mg/kg. In certain embodiments, the immunoconjugate/radioimmunoconjugate may be administered from about 0.5 mg/kg to about 1 mg/kg, about 0.5 mg/kg to about 2 mg/kg, about 0.5 mg/kg to about 5 mg/kg, about 0.5 mg/kg to about 10 mg/kg, about 0.5 mg/kg to about 3 mg/kg, about 0.5 mg/kg to about 4 mg/kg, about 0.5 mg/kg to about 5 mg/kg, about 0.5 mg/kg to about 10 mg/kg, about 0.5 mg/kg to about 20 mg/kg, about 0.5 mg/kg to about 30 mg/kg, about 1 mg/kg to about 2 mg/kg, about 1 mg/kg to about 5 mg/kg, about 1 mg/kg to about 10 mg/kg, about 1 mg/kg to about 3 mg/kg, about 1 mg/kg to about 4 mg/kg, about 1 mg/kg to about 5 mg/kg, about 1 mg/kg to about 10 mg/kg, about 1 mg/kg to about 20 mg/kg, about 1 mg/kg to about 30 mg/kg, about 2 mg/kg to about 5 mg/kg, about 2 mg/kg to about 10 mg/kg, about 2 mg/kg to about 3 mg/kg, about 2 mg/kg to about 4 mg/kg, about 2 mg/kg to about 5 mg/kg, about 2 mg/kg to about 10 mg/kg, about 2 mg/kg to about 20 mg/kg, about 2 mg/kg to about 30 mg/kg, about 5 mg/kg to about 10 mg/kg, about 5 mg/kg to about 3 mg/kg, about 5 mg/kg to about 4 mg/kg, about 5 mg/kg to about 5 mg/kg, about 5 mg/kg to about 10 mg/kg, about 5 mg/kg to about 20 mg/kg, about 5 mg/kg to about 30 mg/kg, about 10 mg/kg to about 3 mg/kg, about 10 mg/kg to about 4 mg/kg, about 10 mg/kg to about 5 mg/kg, about 10 mg/kg to about 10 mg/kg, about 10 mg/kg to about 20 mg/kg, about 10 mg/kg to about 30 mg/kg, about 3 mg/kg to about 4 mg/kg, about 3 mg/kg to about 5 mg/kg, about 3 mg/kg to about 10 mg/kg, about 3 mg/kg to about 20 mg/kg, about 3 mg/kg to about 30 mg/kg, about 4 mg/kg to about 5 mg/kg, about 4 mg/kg to about 10 mg/kg, about 4 mg/kg to about 20 mg/kg, about 4 mg/kg to about 30 mg/kg, about 5 mg/kg to about 10 mg/kg, about 5 mg/kg to about 20 mg/kg, about 5 mg/kg to about 30 mg/kg, about 10 mg/kg to about 20 mg/kg, about 10 mg/kg to about 30 mg/kg, or about 20 mg/kg to about 30 mg/kg. In certain embodiments, the immunoconjugate/radioimmunoconjugate may be administered at about 0.5 mg/kg, about 1 mg/kg, about 2 mg/kg, about 5 mg/kg, about 10 mg/kg, about 3 mg/kg, about 4 mg/kg, about 5 mg/kg, about 10 mg/kg, about 20 mg/kg, or about 30 mg/kg. In certain embodiments, the immunoconjugate/radioimmunoconjugate may be administered at least about 0.5 mg/kg, about 1 mg/kg, about 2 mg/kg, about 5 mg/kg, about 10 mg/kg, about 3 mg/kg, about 4 mg/kg, about 5 mg/kg, about 10 mg/kg, or about 20 mg/kg. In certain embodiments, the immunoconjugate/radioimmunoconjugate may be administered at most about 1 mg/kg, about 2 mg/kg, about 5 mg/kg, about 10 mg/kg, about 3 mg/kg, about 4 mg/kg, about 5 mg/kg, about 10 mg/kg, about 20 mg/kg, or about 30 mg/kg.

In some embodiments, the method comprises administering the effective amount of a radioimmunoconjugate comprising ²²⁵Ac that is from 0.01 to 0.1 mCi, or 0.1 mCi to 1.0 mCi, or from 1.0 mCi to 2.0 mCi, or from 2.0 mCi to 4.0 mCi.

In some embodiments, the method comprises administering the effective amount of a radioimmunoconjugate comprising ²²⁵Ac that is from 0.1 μCi/kg to 2.0 μCi/kg subject weight, or from 0.1 μCi/kg to 1.0 μCi/kg subject weight, or from 1.0 μCi/kg to 3.0 μCi/kg subject weight, or from 3.0 μCi/kg to 10.0 μCi/kg subject weight, or from 10.0 μCi/kg to 20.0 μCi/kg subject weight, or from 10.0 μCi/kg to 30.0 μCi/kg subject weight.

In certain embodiments, the effective amount of ²²⁵Ac is about 0.1 microcurie to about 20 microcurie. In certain embodiments, the effective amount of ²²⁵Ac is about 0.1 microcurie to about 0.2 microcurie, about 0.1 microcurie to about 0.5 microcurie, about 0.1 microcurie to about 1 microcurie, about 0.1 microcurie to about 2 microcurie, about 0.1 microcurie to about 3 microcurie, about 0.1 microcurie to about 4 microcurie, about 0.1 microcurie to about 5 microcurie, about 0.1 microcurie to about 10 microcurie, about 0.1 microcurie to about 20 microcurie, about 0.2 microcurie to about 0.5 microcurie, about 0.2 microcurie to about 1 microcurie, about 0.2 microcurie to about 2 microcurie, about 0.2 microcurie to about 3 microcurie, about 0.2 microcurie to about 4 microcurie, about 0.2 microcurie to about 5 microcurie, about 0.2 microcurie to about 10 microcurie, about 0.2 microcurie to about 20 microcurie, about 0.5 microcurie to about 1 microcurie, about 0.5 microcurie to about 2 microcurie, about 0.5 microcurie to about 3 microcurie, about 0.5 microcurie to about 4 microcurie, about 0.5 microcurie to about 5 microcurie, about 0.5 microcurie to about 10 microcurie, about 0.5 microcurie to about 20 microcurie, about 1 microcurie to about 2 microcurie, about 1 microcurie to about 3 microcurie, about 1 microcurie to about 4 microcurie, about 1 microcurie to about 5 microcurie, about 1 microcurie to about 10 microcurie, about 1 microcurie to about 20 microcurie, about 2 microcurie to about 3 microcurie, about 2 microcurie to about 4 microcurie, about 2 microcurie to about 5 microcurie, about 2 microcurie to about 10 microcurie, about 2 microcurie to about 20 microcurie, about 3 microcurie to about 4 microcurie, about 3 microcurie to about 5 microcurie, about 3 microcurie to about 10 microcurie, about 3 microcurie to about 20 microcurie, about 4 microcurie to about 5 microcurie, about 4 microcurie to about 10 microcurie, about 4 microcurie to about 20 microcurie, about 5 microcurie to about 10 microcurie, about 5 microcurie to about 20 microcurie, or about 10 microcurie to about 20 microcurie. In certain embodiments, the effective amount of ²²⁵Ac is about 0.1 microcurie, about 0.2 microcurie, about 0.5 microcurie, about 1 microcurie, about 2 microcurie, about 3 microcurie, about 4 microcurie, about 5 microcurie, about 10 microcurie, or about 20 microcurie. In certain embodiments, the effective amount of ²²⁵Ac is at least about 0.1 microcurie, about 0.2 microcurie, about 0.5 microcurie, about 1 microcurie, about 2 microcurie, about 3 microcurie, about 4 microcurie, about 5 microcurie, or about 10 microcurie. In certain embodiments, the effective amount of ²²⁵Ac is at most about 0.2 microcurie, about 0.5 microcurie, about 1 microcurie, about 2 microcurie, about 3 microcurie, about 4 microcurie, about 5 microcurie, about 10 microcurie, or about 20 microcurie. According to aspects where the radioisotope of the radioimmunoconjugate is ¹¹¹In, the effective amount is below, for example, 15.0 mCi (i.e., where the amount of ¹¹¹In administered to the subject delivers a total body radiation dose of below 15.0 mCi).

According to aspects where the radioisotope of the radioimmunoconjugate is ¹¹¹In, the effective amount is below 15.0 mCi, below 14.0 mCi, below 13.0 mCi, below 12.0 mCi, below 11.0 mCi, below 10.0 mCi., below 9.0 mCi, below 8.0 mCi, below 7.0 mCi, below 6.0 mCi, below 5.0 mCi, below 4.0 mCi, below 3.5 mCi, below 3.0 mCi, below 2.5 mCi, below 2.0 mCi, below 1.5 mCi, below 1.0 mCi, below 0.5 mCi, below 0.4 mCi, below 0.3 mCi, below 0.2 mCi, or below 0.1 mCi.

According to aspects where the radioisotope of the radioimmunoconjugate is ¹¹¹In, the effective amount is from 0.1 mCi to 1.0 mCi, from 0.1 mCi to 2.0 mCi, from 1.0 mCi to 2.0 mCi, from 1.0 mCi to 3.0 mCi, from 1.0 mCi to 4.0 mCi, from 1.0 mCi to 5.0 mCi, from 1.0 mCi to 10.0 mCi, from 1.0 mCi to 15.0 mCi, from 1.0 mCi to 20.0 mCi, from 2.0 mCi to 3.0 mCi, from 3.0 mCi to 4.0 mCi, from 4.0 mCi to 5.0 mCi, from 5.0 mCi to 10.0 mCi, from 5.0 mCi to 15.0 mCi, from 5.0 mCi to 20.0 mCi, from 6.0 mCi to 14.0 mCi, from 7.0 mCi to 13.0 mCi, from 8.0 mCi to 12.0 mCi, from 9.0 mCi to 11.0 mCi, or from 10.0 mCi to 15.0 mCi.

According to aspects where the radioisotope of the radioimmunoconjugate is ²²⁵In, the effective amount is 15.0 mCi, 14.0 mCi, 13.0 mCi, 12.0 mCi, 11.0 mCi, 10.0 mCi, 9.0 mCi, 8.0 mCi, 7.0 mCi, 6.0 mCi, 5.0 mCi, 4.0 mCi, 3.5 mCi, 3.0 mCi, 2.5 mCi, 2.0 mCi, 1.5 mCi, 1.0 mCi, 0.5 mCi, 0.4 mCi, 0.3 mCi, 0.2 mCi, or 0.1 mCi.

According to aspects where the radioisotope of the radioimmunoconjugate is ²²⁵Ac, the effective amount is below, for example, 30.0 μCi/kg (i.e., where the amount of ²²⁵Ac administered to the subject delivers a radiation dose of below 30.0 μCi per kilogram of subject's body weight).

According to aspects where the radioisotope of the radioimmunoconjugate is ²²⁵Ac, the effective amount is below 30 μCi/kg, 25 μCi/kg, 20 μCi/kg, 17.5 μCi/kg, 15.0 μCi/kg, 12.5 μCi/kg, 10.0 μCi/kg, 9 μCi/kg, 8 μCi/kg, 7 μCi/kg, 6 μCi/kg, 5 μCi/kg, 4.5 μCi/kg, 4.0 μCi/kg, 3.5 μCi/kg, 3.0 μCi/kg, 2.5 μCi/kg, 2.0 μCi/kg, 1.5 μCi/kg, 1.0 μCi/kg, 0.9 μCi/kg, 0.8 μCi/kg, 0.7 μCi/kg, 0.6 μCi/kg, 0.5 μCi/kg, 0.4 μCi/kg, 0.3 μCi/kg, 0.2 μCi/kg, 0.1 μCi/kg, or 0.05 μCi/kg.

According to aspects where the radioisotope of the radioimmunoconjugate is ²²⁵Ac, the effective amount is from 0.05 μCi/kg to 0.1 μCi/kg, from 0.1 μCi/kg to 0.2 μCi/kg, from 0.2 μCi/kg to 0.3 μCi/kg, from 0.3 μCi/kg to 0.4 μCi/kg, from 0.4 μCi/kg to 0.5 μCi/kg, from 0.5 μCi/kg to 0.6 μCi/kg, from 0.6 μCi/kg to 0.7 μCi/kg, from 0.7 μCi/kg to 0.8 μCi/kg, from 0.8 μCi/kg to 0.9 μCi/kg, from 0.9 μCi/kg to 1.0 μCi/kg, from 1.0 μCi/kg to 1.5 μCi/kg, from 1.5 μCi/kg to 2.0 μCi/kg, from 2.0 μCi/kg to 2.5 μCi/kg, from 2.5 μCi/kg to 3.0 μCi/kg, from 3.0 μCi/kg to 3.5 μCi/kg, from 3.5 μCi/kg to 4.0 μCi/kg, from 4.0 μCi/kg to 4.5 μCi/kg, or from 4.5 μCi/kg to 5.0 μCi/kg.

According to aspects where the radioisotope of the radioimmunoconjugate is ²²⁵Ac, the effective amount is 0.05 μCi/kg, 0.1 μCi/kg, 0.2 μCi/kg, 0.3 μCi/kg, 0.4 μCi/kg, 0.5 μCi/kg, 0.6 μCi/kg, 0.7 μCi/kg, 0.8 μCi/kg, 0.9 μCi/kg, 1.0 μCi/kg, 1.5 μCi/kg, 2.0 μCi/kg, 2.5 μCi/kg, 3.0 μCi/kg, 3.5 μCi/kg, 4.0 μCi/kg or 4.5 μCi/kg, 5.0 μCi/kg, 6.0 μCi/kg, 7.0 μCi/kg, 8.0 μCi/kg, 9.0 μCi/kg, 10.0 μCi/kg, 12.5 μCi/kg, 15.0 μCi/kg, 17.5 μCi/kg, 20.0 μCi/kg, 25 μCi/kg, or 30 μCi/kg.

In certain embodiments where the radioisotope of the radioimmunoconjugate is ¹⁷⁷Lu the effective amount is from 0.1 μCi to 100 mCi per meter squared of body surface area.

In certain embodiments where the radioisotope of the radioimmunoconjugate is ¹⁷⁷Lu the effective amount is from 1 mCi to 100 mCi per meter squared of body surface area. In certain embodiments, the effective amount is about 1 per meter squared to about 100 per meter squared. In certain embodiments, the effective amount is about 1 per meter squared to about 5 per meter squared, about 1 per meter squared to about 10 per meter squared, about 1 per meter squared to about 15 per meter squared, about 1 per meter squared to about 20 per meter squared, about 1 per meter squared to about 25 per meter squared, about 1 per meter squared to about 75 per meter squared, about 1 per meter squared to about 100 per meter squared, about 5 per meter squared to about 10 per meter squared, about 5 per meter squared to about 15 per meter squared, about 5 per meter squared to about 20 per meter squared, about 5 per meter squared to about 25 per meter squared, about 5 per meter squared to about 75 per meter squared, about 5 per meter squared to about 100 per meter squared, about 10 per meter squared to about 15 per meter squared, about 10 per meter squared to about 20 per meter squared, about 10 per meter squared to about 25 per meter squared, about 10 per meter squared to about 75 per meter squared, about 10 per meter squared to about 100 per meter squared, about 15 per meter squared to about 20 per meter squared, about 15 per meter squared to about 25 per meter squared, about 15 per meter squared to about 75 per meter squared, about 15 per meter squared to about 100 per meter squared, about 20 per meter squared to about 25 per meter squared, about 20 per meter squared to about 75 per meter squared, about 20 per meter squared to about 100 per meter squared, about 25 per meter squared to about 75 per meter squared, about 25 per meter squared to about 100 per meter squared, or about 75 per meter squared to about 100 per meter squared. In certain embodiments, the effective amount is about 1 per meter squared, about 5 per meter squared, about 10 per meter squared, about 15 per meter squared, about 20 per meter squared, about 25 per meter squared, about 75 per meter squared, or about 100 per meter squared. In certain embodiments, the effective amount is at least about 1 per meter squared, about 5 per meter squared, about 10 per meter squared, about 15 per meter squared, about 20 per meter squared, about 25 per meter squared, or about 75 per meter squared. In certain embodiments, the effective amount is at most about 5 per meter squared, about 10 per meter squared, about 15 per meter squared, about 20 per meter squared, about 25 per meter squared, about 75 per meter squared, or about 100 per meter squared.

According to certain aspects of the present invention, a preparation of radioimmunoconjugate of the invention, or a composition thereof (e.g., a pharmaceutical composition), may comprise a radiolabeled fraction (radioimmunoconjugate) and an unlabeled fraction (immunoconjugate), wherein the ratio of labeled:unlabeled may be from about 1:1000 to 1:1.

Moreover, the pharmaceutical compositions may be provided as a single dose composition tailored to a specific patient, i.e., as a patient specific therapeutic composition, wherein the amount of labeled and unlabeled immunoconjugate (labeled immunoconjugate, for clarity, being the same as radioimmunoconjugate herein) in the composition may depend on at least a patient weight, height, body surface area, age, gender, and/or disease state or health status. As such, a total volume of the patient specific therapeutic composition may be provided in a vial that is configured to be wholly administered to the patient in one treatment session, such that little to no composition remains in the vial after administration.

Currently, depending on the stage of the cancer, cancer treatment involves one or a combination of the following therapies: surgery to remove the cancerous tissue, radiation therapy, and chemotherapy. Therapy using radioimmunoconjugate of the invention (interchangeably, “radiolabeled immunoconjugate”) may be especially desirable in elderly patients who do not tolerate the toxicity and side effects of chemotherapy well and in metastatic disease where radiation therapy has limited usefulness. For some embodiments, therapy using radiolabeled immunoconjugate of the invention are useful to alleviate target antigen-expressing cancers upon initial diagnosis of the disease or during relapse.

In some embodiments, determining whether a cancer is amenable to treatment by methods disclosed herein involves detecting the presence of the target antigen in a subject or in a sample from a subject. To determine target antigen expression in a cancer, various detection assays are available. In one embodiment, target antigen overexpression is analyzed by immunohistochemistry (IHC). Parrafin embedded tissue sections from a tumor biopsy are subjected to the IHC assay and accorded a target antigen staining intensity criteria. Alternatively, or additionally, FISH assays such as the INFORM® (sold by Ventana, AZ, U.S.A.) or PATHVISION® (Vysis, IL, U.S.A.) may be carried out on formalin-fixed, paraffin-embedded tumor tissue to determine the extent (if any) of target antigen overexpression in the tumor.

Target antigen overexpression or amplification may be evaluated using an in vivo detection assay, e.g., by administering a molecule (such as an antibody construct or immunoconjugate of the invention) which binds the molecule to be detected and is tagged with a detectable label (e.g., a radioactive isotope or a fluorescent label) and externally scanning the patient for localization of the label.

### Using Immunoconjugates and Radioimmunoconjugates of the Invention for Killing a Cell(s)

An immunoconjugate or radioimmunoconjugate of the invention may be used in, for example, in vitro, ex vivo, and in vivo methods. In one aspect, the invention provides methods for inhibiting cell growth or proliferation, either in vivo or in vitro, the method comprising exposing a cell to an immunoconjugate or radioimmunoconjugate of the invention under conditions permissive for binding of the immunoconjugate or radioimmunoconjugate to a target antigen. The immunoconjugate or radioimmunoconjugate of the invention may also (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind.

In one aspect, the invention provides a method of killing an antigen expressing cell, the method comprising contacting the cell with an immunoconjugate or radioimmunoconjugate of the present invention (or a composition thereof). This method can be used, e.g., to kill, deplete, or eliminate target antigen-expressing cells from a population of mixed cells. This method can be used, e.g., to kill, deplete, or eliminate target antigen-expressing cells from a population of mixed cells as a step in the purification of other cells. This method can be performed in vitro or in vivo, including ex vivo on primary patient cell or tissue compositions to prepare such compositions for transplantation.

In one aspect, an immunoconjugate or radioimmunoconjugate of the invention is used to treat or prevent a cell proliferative disorder. In certain embodiments, the cell proliferative disorder comprises a solid tumor cancer. A solid tumor cancer is a cancer comprising an abnormal mass of tissue, e.g., carcinomas and sarcomas. In certain other embodiments, the cell proliferative disorder comprises a liquid tumor cancer or hematological cancer, used interchangeably, such cancers present in the body fluid, e.g., leukemias and lymphomas. In certain embodiments, the cell proliferative disorder is associated with increased expression and/or activity of a target antigen. For example, in certain embodiments, the cell proliferative disorder is associated with increased expression of target antigen on the surface of a cell. In certain embodiments, the cell proliferative disorder is a tumor or a cancer. In certain embodiments, the cell proliferative disorder comprises a solid tumor cancer. A solid tumor cancer is a cancer comprising an abnormal mass of tissue, e.g., carcinomas and sarcomas. In certain other embodiments, the cell proliferative disorder comprises a liquid tumor cancer or hematological cancer. Used interchangeably, such cancers present in the body fluid, e.g., leukemias and lymphomas.

In one aspect, the invention provides methods for treating a cell proliferative disorder comprising administering to an individual an effective amount of an immunoconjugate or radioimmunoconjugate of the invention.

In addition to direct cell killing of target cells expressing cell-surface antigen specifically bound by the immunoconjugate or radioimmunoconjugate of the invention, the immunoconjugate or radioimmunoconjugate of the present invention optionally may be used for delivery of additional cargos to the vicinity of or the interiors of target cells. The delivery of additional exogenous materials may be used, e.g., for cytotoxic, cytostatic, information gathering, and/or diagnostic functions. Non-cytotoxic variants of the immunoconjugate or radioimmunoconjugate of the invention, or optionally toxic variants, may be used to deliver cargos to and/or label the interiors of cells expressing the target antigen. Non-limiting examples of cargos include cytotoxic agents, detection-promoting agents, and small molecule chemotherapeutic agents.

### Using Antibody Constructs, Immunoconjugates Radioimmunoconjugates and Targeted Imaging Complexes of the Invention for Antigen Detection, In Vivo Imaging, Diagnosis, and Prognostication

As described herein, in some embodiments, the antibody constructs, immunoconjugates, radioimmunoconjugates and targeted imaging complexes of the present invention have various non-therapeutic applications. In some embodiments, the compositions of the invention may be used to identify patient populations predicted to benefit from a specific therapeutic approach or modality, such as, e.g., treatment with an immunoconjugates or radioimmunoconjugates of the invention. In some embodiments, the compositions of the invention can be useful for staging of target antigen expressing cancers (e.g., by radioimaging) or as prognostic indicators of disease progression. In some embodiments, the compositions are also useful for detection and quantitation of a target epitope in vitro, e.g., in an ELISA or a Western blot, as well as purification or immunoprecipitation of a target antigen from cells or a tissue sample.

For some embodiments, the immunoconjugate or radioimmunoconjugate of the invention is used in a method to detect the presence of or level of an antigen, such as, e.g., in vitro in a biological sample or in vivo using an imagine technique. Immunoconjugate and radioimmunoconjugate detection can be achieved via different techniques known to the skilled worker and as described herein, e.g., IHC and PET imaging. When an immunoconjugate or radiolabeled immunoconjugate of the invention is used for detection, it may comprise a radioactive atom for scintigraphic studies, for example ^{99m}Tc or ¹¹¹In.

Labelled immunoconjugates of the invention are useful as imaging biomarkers and probes by the various methods and techniques of biomedical and molecular imaging such as: (i) MRI (magnetic resonance imaging); (ii) MicroCT (computerized tomography); (iii) SPECT (single photon emission computed tomography); (iv) PET (positron emission tomography) Chen et al Bioconjugate Chem. 15: 41-9 (2004); (v) bioluminescence; (vi) fluorescence; and (vii) ultrasound. Immunoscintigraphy is an imaging procedure in which antibodies labeled with radioactive substances are administered to an animal or human patient and a picture is taken of sites in the body where the antibody localizes (U.S. Pat. No. 6,528,624). Imaging biomarkers may be objectively measured and evaluated as an indicator of normal biological processes, pathogenic processes, or pharmacological responses to a therapeutic intervention.

Another aspect of the present invention is a method of determining the presence of a target antigen in a sample suspected of containing the target antigen, wherein the method comprises exposing the sample to an immunoconjugate that binds to the target antigen and determining binding of the immunoconjugate to the target antigen in the sample, wherein the presence of such binding is indicative of the presence of the target antigen in the sample. Optionally, the sample may contain cells (which may be cancer cells) suspected of expressing the target antigen. The immunoconjugate employed in the method may optionally be detectably labeled, attached to a solid support, or the like.

Another embodiment of the present invention is directed to a method of diagnosing the presence of a tumor in a subject, wherein the method comprises (a) contacting a test sample comprising tissue cells obtained from the mammal with an immunoconjugate that binds to a target antigen and (b) detecting the formation of a complex between the immunoconjugate and the target antigen in the test sample, wherein the formation of a complex is indicative of the presence of a tumor in the mammal. Optionally, the immunoconjugate is detectably labeled, attached to a solid support, or the like, and/or the test sample of tissue cells is obtained from an individual suspected of having a cancerous tumor.

In some embodiments, the immunoconjugates of the present invention, including compositions comprising the aforementioned and/or provided herein are useful for detecting the presence of a target antigen, e.g., in vivo or in a biological sample. The immunoconjugates of the invention can be used in a variety of different assays, including but not limited to ELISA, bead-based immunoassays, and mass spectrometry.

In some embodiments, the immunoconjugates of the present invention are useful to quantitate target antigen amounts in a sample. In some embodiments, a biological sample is a biological fluid, such as whole blood or whole blood components including red blood cells, white blood cells, platelets, serum and plasma, ascites, vitreous fluid, lymph fluid, synovial fluid, follicular fluid, seminal fluid, amniotic fluid, milk, saliva, sputum, tears, perspiration, mucus, cerebrospinal fluid, urine and other constituents of the body that may contain the target antigen of interest. In various embodiments, the sample is a body sample from any animal. In some embodiments, the sample is from a mammal. In some embodiments, the sample is from a human subject. In some embodiments, the biological sample is serum from a clinical patient. In some embodiments, the biological sample is biopsy material. In some embodiments, the biological sample is biopsy material from a clinical patient. In some embodiments, the biological sample is serum from a clinical patient. In some embodiments, the biological sample is primary cell culture material. In some embodiments, the biological sample is primary cell culture material from a clinical patient. In some embodiments, the biological sample is from clinical patients or patients treated with a therapeutic antibody or antibodies that binds the same target antigen.

In some embodiments, the sample is from a mammal. In some embodiments, the sample is from a human subject, e.g., when measuring antigen expression in a clinical sample. In some embodiments, the biological sample is from clinical patients or a patient treated with a therapy/therapeutic (e.g., an antibody therapy targeting the same target antigen). In some embodiments, the biological sample is serum or plasma. In some embodiments, the biological sample is serum from a clinical patient. In some embodiments, the biological sample is biopsy material. In some embodiments, the biological sample is biopsy material from a clinical patient. In some embodiments, the biological sample is serum from a clinical patient. In some embodiments, the biological sample is primary cell culture material. In some embodiments, the biological sample is primary cell culture material from a clinical patient.

In some embodiments, compositions comprising ‘labeled’ immunoconjugates are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, fluorophores such as rare earth chelates or fluorescein and its derivatives, rhodamine and its derivatives, dansyl, umbelliferone, luciferases, e.g., firefly luciferase and bacterial luciferase, luciferin, 2,3-dihydrophthalazinediones, horseradish peroxidase (HRP), alkaline phosphatase, J3-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Conventional methods are available to bind these labels covalently to proteins or polypeptides. For instance, coupling agents such as dialdehydes, carbodiimides, dimaleimides, bis-imidates, bis-diazotized benzidine, and the like may be used to tag the immunoconjugates or antibody constructs of the invention with the above-described fluorescent, chemiluminescent, and enzyme labels (see e.g., U.S. Pat. No. 3,645,090 (enzymes); U.S. Pat. No. 3,940,475 (fluorimetry), Hunter et al., Nature, 144:945 (1962); David et al., Biochemistry, 13:1014-1021 (1974); Pain et al., J. Immunol. Methods, 40:219-230 (1981); Nygren, J. Histochem and Cytochem, 30:407-412 (1982).

The conjugation of such label, including the enzymes, to the immunoconjugate or antibody construct is a standard manipulative procedure for one of ordinary skill in immunoassay techniques (see e.g., O'Sullivan et al. “Methods for the Preparation of Enzyme-antibody Conjugates for Use in Enzyme Immunoassay,” in Methods in Enzymology, ed. J. Langone and H. Van Vunakis, Vol. 73 (Academic Press, New York, New York, 1981), pp. 147-166). Suitable commercially available labeled antibodies may also be used.

Following the addition of last labeled immunoconjugate, the amount of bound immunoconjugate is determined by removing excess unbound labeled immunoconjugate through washing and then measuring the amount of the attached label using a detection method appropriate to the label, and correlating the measured amount with the amount of the immunoconjugate of interest in the biological sample. For example, in the case of enzymes, the amount of color developed and measured will be a direct measurement of the amount of the immunoconjugate of interest present. Specifically, if HRP is the label, the color may be detected using the substrate TMD, using a 450 nm read wavelength and a 620 or 630 nm reference wavelength.

In one example, after an enzyme-labeled second antibody directed against the unlabeled immunoconjugate is washed from the immobilized phase, color or chemiluminescence is developed and measured by incubating the immobilized capture reagent with a substrate of the enzyme. Then the concentration of the antibody of interest is calculated by comparing with the color or chemiluminescence generated by the immunoconjugate of interest run in parallel.

In some embodiments, the method involves a bead-based immunoassay, an ELISA assay, or a mass spectrometric technique. The mass analyzers of such mass spectrometers include, but are not limited to, quadrupole (Q), time of flight (TOF), ion trap, magnetic sector or Fourier transform ion cyclotron resonance (FT-ICR) or combinations thereof. The ion source of the mass spectrometer should yield mainly sample molecular ions, or pseudo-molecular ions, and certain characterizable fragment ions. Examples of such ion sources include atmospheric pressure ionization sources, e.g., electrospray ionization (ESI) and atmospheric pressure chemical ionization (APCI) and Matrix Assisted Laser Desorption Ionization (MALDI). ESI and MALDI are the two most commonly employed methods to ionize proteins for mass spectrometric analysis of small molecules, such as, e.g., by liquid chromatography mass spectrometry (LC/MS) (Lee, M., LCMS Applications in Drug Development (2002) J. Wiley & Sons, New York). Another example is surface enhanced laser desorption ionization (SELDI). SELDI is a surface-based ionization technique that allows for high-throughput mass spectrometry. Typically, SELDI is used to analyze complex mixtures of proteins and other biomolecules. SELDI employs a chemically reactive surface such as a “protein chip” to interact with analytes, e.g., proteins, in solution. Such surfaces selectively interact with analytes and immobilize them thereon. Thus, the analytes of the invention can be partially purified on the chip and then quickly analyzed in the mass spectrometer. By providing multiple reactive moieties at different sites on a substrate surface, throughput may be increased.

In another aspect, the invention provides a method for detecting in a biological sample an antigen, the method comprising: (a) contacting the biological sample with an immunoconjugate described herein to allow forming an immunocomplex; (b) detecting or measuring the level of the immunoconjugate bound to the sample. In some embodiments, the immunoconjugate is immobilized to a solid support. In some embodiments, the immobilized immunoconjugate is conjugated to biotin and bound to a streptavidin coated microtiter plate.

### Kits and Articles of Manufacture of the Present Invention

Another aspect of the present invention is an article of manufacture containing materials useful for the treatment, prevention and/or diagnosis of diseases and disorders characterized by target antigen-expressing cells (e.g., a cancer cell). The article of manufacture of the invention comprises a container and a label or package insert on or associated with the container. Suitable containers include, for example, bottles, vials, syringes, etc. The containers may be formed from a variety of materials such as glass or plastic. The container holds a composition which is effective for treating, preventing and/or diagnosing the cancer condition and may have a sterile access port (for example the container may be an intravenous solution bag or a vial having a stopper pierceable by a hypodermic injection needle). At least one active agent in the composition is an immunoconjugate of the invention. The label or package insert indicates that the composition is used for treating cancer. The label or package insert will further comprise instructions for administering the immunoconjugate composition to the cancer patient. Additionally, the article of manufacture may further comprise a second container comprising a pharmaceutically-acceptable buffer, such as bacteriostatic water for injection (BWFI), phosphate-buffered saline, Ringer's solution and dextrose solution. The article of manufacture may further include other materials desirable from a commercial and user standpoint, including other buffers, diluents, filters, needles, and syringes.

In another aspect, the invention provides a kit comprising any of the immunoconjugates described herein and an additional reagent or pharmaceutical device. In some further embodiments, the kit comprises a composition as provided herein (e.g., a pharmaceutical or diagnostic composition). Another aspect of the present invention is a kit useful for various purposes, e.g., target antigen-expressing cell killing; for target antigen-expressing cell detection; quantification, purification, or immunoprecipitation of target antigen from cells.

In some embodiments, the kit of the invention is an immunoassay kit for specifically detecting an antigen in a biological sample, comprising: (a) an immunoconjugate as described herein and/or a composition thereof; and (b) instructions for detecting said immunoconjugate. A target antigen detection assays of the present invention can be provided in the form of a kit. In some embodiments, such a kit comprises an immunoconjugate of the present invention, or a composition comprising the aforementioned, such as one described herein. The kit may further comprise a solid support for the capture reagents, which may be provided as a separate element or to which the capture reagents are already immobilized. For isolation and purification of a target antigen, the kit may contain an immunoconjugate of the invention coupled to beads (e.g., sepharose beads). The invention provides kits that contain an antibody for the detection and/or quantitation of target antigen in vitro, e.g., in an ELISA or a Western blot. In some embodiments, the capture reagents (e.g., the immunoconjugate of the invention) are coated on or attached to a solid material (e.g., to beads, a microtiter plate, or a comb). The detectable antibodies may be labeled antibodies detected directly or unlabeled antibodies that are detected by labeled antibodies directed against the unlabeled antibodies, such as, e.g., antibodies raised in a different species. Where the label is an enzyme, the kit will ordinarily include substrates and cofactors required by the enzyme; where the label is a fluorophore, a dye precursor that provides the detectable chromophore; and where the label is biotin, an avidin such as avidin, streptavidin, or streptavidin conjugated to HRP or β-galactosidase with MUG.

As with the article of manufacture of the invention, the kit of the invention comprises a container and a label or package insert on or associated with the container. The container holds a composition comprising at least one immunoconjugate of the invention. Additional containers may be included that contain, e.g., diluents and buffers, control immunoconjugates or antibodies. The label or package insert may provide a description of the composition as well as instructions for the intended in vitro or detection use. The kit also typically contains additives such as stabilizers, washing and incubation buffers, and the like for performing the assay method(s). The components of the kit will be provided in predetermined ratios, with the relative amounts of the various reagents suitably varied to provide for concentrations in solution of the reagents that substantially maximize the sensitivity of the assay(s). Particularly, the reagents may be provided as dry powders, usually lyophilized, including excipients, which on dissolution will provide for a reagent solution having the appropriate concentration for combining with the sample to be tested.

The present invention is further illustrated by the following non-limiting examples of immunoconjugates comprising the aforementioned structures and functions, in particular platforms having VHH polypeptides, a molecular weight between 60 and 110 kDa, a serum half-life of less than 96 hours, which in some embodiments exhibit enhanced stability during the temperatures required for certain radiolabeling processes relative to other antibody fragment platforms, and which in some embodiments exhibit decreased loss of targeting capacity due to radiolysis as compared to other possible delivery platforms.

### Certain Definitions

In this description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The skilled worker will recognize many methods and materials similar or equivalent to those described herein, which could be used in the practice of the present invention. Indeed, the present invention is in no way limited to the methods and materials described. For purposes of the present invention, some terms are defined below.

As used in the specification and the appended claims, the terms “a,” “an” and “the” include both singular and the plural referents unless the context clearly dictates otherwise.

Throughout this specification, the term “including” is used to mean “including but not limited to.” “Including” and “including but not limited to” are used interchangeably.

The term “about” as used herein refers to the usual error range for the respective value readily known to the skilled person in this technical field. Reference to “about” a value or parameter herein includes (and describes) embodiments that are directed to that value or parameter per se. The term “about” when used before a numerical designation, e.g., a numerical temperature, time, amount, or concentration, including a range, indicates approximations which may vary by ±10%, ±5%, or ±1%.

The term “amino acid residue” or “amino acid” includes reference to an amino acid that is incorporated into a protein, polypeptide, and/or peptide. The term “polypeptide” includes any polymer of amino acids or amino acid residues. The term “polypeptide sequence” refers to a series of amino acids or amino acid residues which physically comprise a polypeptide. A “protein” is a macromolecule comprising one or more polypeptides or polypeptide “chains.” A “peptide” is a small polypeptide of a size of 2 to 20 amino acid residues. The term “amino acid sequence” refers to a series of amino acids or amino acid residues which physically comprise a peptide or polypeptide depending on the length. Unless otherwise indicated, polypeptide and protein sequences disclosed herein are written from left to right representing their order from an amino terminus to a carboxy terminus.

The terms “amino acid,” “amino acid residue,” “amino acid sequence,” or polypeptide sequence include naturally occurring amino acids (including L and D isosteriomers) and, unless otherwise limited, also include known analogs of natural amino acids that can function in a similar manner as the common natural amino acids, such as selenocysteine, pyrrolysine, N-formylmethionine, gamma-carboxyglutamate, hydroxyprolinehypusine, pyroglutamic acid, and selenomethionine (see, e.g., Ho J et al., ACS Synth Biol 5: 163-71 (2016); Wang Y, Tsao M, Chembiochem 17: 2234-9 (2016)). The amino acids referred to herein are described by shorthand designations as follows in Table A:

As used herein, the term “radioisotope” includes, but is not limited to, an alpha emitting isotope (interchangeably, α-emitting isotope), beta-emitting isotope (interchangeably, β-emitting isotope), and/or gamma-emitting isotope (interchangeably, γ-emitting isotope), such as, e.g., any one of ⁸⁶Y, ⁹⁰Y, ¹⁷⁷Lu, ¹⁸⁶Re, ¹⁸⁸Re, ⁸⁹Sr, ⁵³Sm, ²²⁵Ac, ²³Bi, ²¹³Po, ²¹²Bi, ²²³Ra, ²²⁴Ra, ²²⁷Th, ¹⁴⁹Tb, ⁶⁸Ga, ⁶⁴Cu, ⁶⁷Cu, 89-Zr, 137-Cs, ²¹²Pb, and ¹⁰³Pd.

As used herein, the term “radioimmunoconjugate” refers to a molecular complex comprising (1) an immunoconjugate according to the present invention and (2) a radioisotope. In a preferred embodiment, the radioisotope is an α-emitting radioisotope. In another embodiment, the radioisotope is a β-emitting radioisotope. In another embodiment, the radioisotope is a γ-emitting isotope. In another embodiment, the invention provides radioimmunoconjugates comprising α-emitting and β-emitting radioisotopes. The term “radioconjugate” is used interchangeably with the term “radioimmunoconjugate” herein. In one embodiment, the radioisotope is associated with a chelating agent of the radioimmunoconjugate. In one embodiment, the radioisotope is directly linked to the immunoconjugate.

As used herein, the term “immunoconjugate” refers to a molecular complex comprising an at least one antigen binding region derived from an antibody (e.g., variable regions or complementarity determining regions) further coupled to at least one non-antibody derived molecule, such as a chelator or cytotoxic agent. Non-antibody derived molecules may for example be conjugated to one or more lysine or cysteine resides of the antigen binding region or to a constant region coupled (by peptide linkage or otherwise) to the antigen binding region. In some embodiments, the immunoconjugate further comprises a chelating agent (interchangeably, “chelator”). In one embodiment, an immunoconjugate comprises an antibody construct of the invention linked directly or indirectly to a cytotoxic agent or radioisotope.

The immunoconjugates and radioimmunoconjugates described herein comprise antigen binding regions. These antigen binding regions can be derived from an “antibody.” The term “antibody” herein is used in the broadest sense and includes monoclonal antibodies, and includes intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

The antigen binding regions of the immunoconjugates described herein may be humanized. “Humanized” in reference to an immunoconjugate refers to an antigen binding region in which all or substantially all CDR amino acid residues are derived from non-human CDRs and all or substantially all FR amino acid residues are derived from human FRs. A humanized immunoconjugate optionally may include at least a portion of an antibody constant region derived from a human antibody.

Among the provided immunoconjugates are human immunoconjugates. A “human immunoconjugates” is an immunoconjugates possessing an antigen binding region with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

The phrase “antigen binding arm”, as used herein, refers to a single polypeptide chain, comprising an “antigen binding region”, a hinge region, and a variant constant region. Other elements (e.g., a chelating agent; an imaging metal) may be attached to the antigen binding arm directly or through one or more linkers in compositions of the invention. Immunoconjugates of the invention comprise two antigen binding arms that are covalently linked together. In one embodiment, the antigen binding arms are linked through the hinge region. In one embodiment, the antigen binding arms are linked through an immunoglobulin heavy chain constant region. In one embodiment, the antigen binding arms are linked through the variant constant region. In one embodiment, the antigen binding arms are linked via a disulfide linkage (e.g., via a cysteine residue in a hinge region).

The phrase “antigen binding region”, as used herein, refers to the region of an immunoconjugate responsible for specific binding to an antigen, such region one or more antigen binding domains comprising complementarity determining regions, variable regions and framework regions, which may be derived from, modeled on, or may mimic, antibodies or fragments thereof, as are known by the person of ordinary skill in the art. In one embodiment, the “antigen binding region’ of an antigen binding arm contains one or two antigen binding domains. In a preferred embodiment, the “antigen binding region” of an antigen binding arm consists of a single antigen binding domain, which antigen binding domain is preferably a VHH polypeptide. In a preferred embodiment, the antigen binding regions of both antigen binding arms of an immunoconjugate independently consist of a single antigen binding domain, which antigen binding domain is preferably a VHH polypeptide, which VHH polypeptides are the same or different.

The term “VHH polypeptide” as used herein encompasses natural and synthetic compositions and refers to a polypeptide constituting a VHH fragment as it is known in the art, i.e., a polypeptide that constitutes a single domain heavy chain only variable domain fragment, or a polypeptide that structurally and functionally resembles a VHH fragment, as such structure is further described below and has the ability to specifically bind antigen is described below, and as both are well known in the art. In preferred embodiments, the VHH polypeptides comprise a heavy chain variable region comprising three heavy chain CDR's; in one embodiment the VHH polypeptide is derived from a camelid; in another embodiment the VHH polypeptide is derived from a library; VHH polypeptides bind to antigens with specificity and high affinity. In a preferred embodiment, the VHH polypeptide is a single heavy chain variable domain comprising the arrangement: FR1, CDR1, FR2, CDR2, FR3, CDR3, and FR4. VHH polypeptides may be obtained, for example, as the antigen binding fragments of heavy chain only antibodies generated in vivo (e.g., in camelids). VHH polypeptides may also be obtained from synthetic libraries, e.g., phage display libraries. For example, see McMahon et al., Nature Structural & Molecular Biology|VOL 25|March 2018|289-296 Yeast surface display platform for rapid discovery of conformationally selective nanobodies; Moutel et al., eLife 2016; 5:e16228 NaLi—H1: A universal synthetic library of humanized nanobodies providing highly functional antibodies and intrabodies. De Genst E, Saerens D, Muyldermans S, Conrath K. Antibody repertoire development in camelids. Dev Comp Immunol. 2006; 30(1-2):187-98. doi: 10.1016/j.dci.2005.06.010. PMID: 16051357. Vincke C, Gutierrez C, Wernery U, Devoogdt N, Hassanzadeh-Ghassabeh G, Muyldermans S. Generation of single domain antibody fragments derived from camelids and generation of manifold constructs. Methods Mol Biol. 2012; 907:145-76. doi: 10.1007/978-1-61779-974-7_8. PMID: 22907350. Arbabi Ghahroudi M, Desmyter A, Wyns L, Hamers R, Muyldermans S. Selection and identification of single domain antibody fragments from camel heavy-chain antibodies. FEBS Lett. 1997 Sep. 15; 414(3):521-6. doi: 10.1016/s0014-5793(97)01062-4. PMID: 9323027.

For VHH humanization, see, for example, Vincke C, Loris R, Saerens D, Martinez-Rodriguez S, Muyldermans S, Conrath K. General strategy to humanize a camelid single-domain antibody and identification of a universal humanized nanobody scaffold. J Biol Chem. 2009 Jan. 30; 284(5):3273-84. doi: 10.1074/jbc.M806889200. Epub 2008 Nov. 14. PMID: 19010777.

For VHH stability, see, for example, Kunz P, Flock T, Soler N, Zaiss M, Vincke C, Sterckx Y, Kastelic D, Muyldermans S, Hoheisel J D. Exploiting sequence and stability information for directing nanobody stability engineering. Biochim Biophys Acta Gen Subj. 2017 September; 1861(9):2196-2205. doi: 10.1016/j.bbagen.2017.06.014. Epub 2017 Jun. 20. PMID: 28642127; PMCID: PMC5548252; Kunz P, Zinner K, Müicke N, Bartoschik T, Muyldermans S, Hoheisel J D. The structural basis of nanobody unfolding reversibility and thermoresistance. Sci Rep. 2018 May 21; 8(1):7934. doi: 10.1038/s41598-018-26338-z. PMID: 29784954; PMCID: PMC5962586.

A “linker” herein is also referred to as “linker sequence” “spacer” “tethering sequence” or grammatical equivalents thereof. A “linker” as referred herein connects two distinct molecules that by themselves possess target binding, catalytic activity, or are naturally expressed and assembled as separate polypeptides or comprise separate domains of the same polypeptide. For example, two distinct binding moieties or a heavy-chain/light-chain pair or an antigen binding region and an immunoglobulin heavy chain constant region. A number of strategies may be used to covalently link molecules together. Linkers described herein may be utilized to join a light chain variable region and a heavy chain variable region in an scFv molecule; or may be used to tether an scFv or other antigen binding fragment on the N- or C-terminus of an antibody heavy chain. These include but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis.

An antibody that “binds” an antigen or epitope of interest is one that binds the antigen or epitope with sufficient affinity that is measurably different from a non-specific interaction. Specific binding can be measured, for example, by determining binding of a molecule compared to binding of a control molecule, which generally is a molecule of similar structure that does not have binding activity.

“Specific binding” refers to an antibody or immunoconjugate that is capable of binding antigen with sufficient affinity such that the antibody is useful as a diagnostic and/or therapeutic agent in targeting that antigen. In one embodiment, the extent of binding of an antibody to an unrelated protein is less than about 10% of the binding of the antibody to its antigen as measured, e.g., by a radioimmunoassay. An “antigen specific” antibody or immunoconjugate, as used herein, is one that specifically binds to the antigen with sufficient specificity and affinity to be useful in targeting a therapeutic, targeting diagnostic, or method of detecting the antigen in a biological sample from a subject. In some embodiments, an immunoconjugate or antibody construct or target imaging complex or radioimmunoconjugate that binds to its target antigen has a dissociation constant (K_{D}) of ≤1 μM, <100 nM, <10 nM, <1 nM, <0.1 nM, <0.01 nM, or <0.001 nM (e.g., 10⁻⁸ M or less, e.g., from 10⁻⁸ M to 10⁻³ M, e.g., from 10⁻⁹ M to 10⁻¹³ M). In some embodiments, an immunoconjugate or antibody construct or target imaging complex or radioimmunoconjugate of the present invention binds to multiple antigens, such as, e.g., an epitope conserved among homologs from different species, such as wherein the amino acid identity of the epitope is non-identical in different species.

As used herein, the term “variant constant region” refers to a polypeptide comprising of a portion of an immunoglobulin heavy chain constant region that has been modified from native immunoglobulin amino acid sequence, preferably at from one to several amino acid positions. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD (1991). Modifications to Fc regions for various purposes are well known in the art. For example, see Kevin O. Saunders, Frontiers in Immunology, June 2019|Volume 10|Article 1296, titled “Conceptual Approaches to Modulating Antibody Effector Functions and Circulation Half-Life”.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways using available computer software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “cytotoxic agent” as used herein refers to a substance that inhibits or prevents a cellular function and/or causes cell death or destruction. Cytotoxic agents include, but are not limited to, radioactive isotopes; chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various cytotoxic agents described herein.

The term “affinity” refers to the strength of the sum total of noncovalent interactions between a single binding site of a molecule (e.g., an antibody) and its binding partner (e.g., an antigen or epitope). Unless indicated otherwise, as used herein, “binding affinity” refers to intrinsic binding affinity which reflects a 1:1 interaction between members of a binding pair (e.g., antibody and antigen or epitope). The affinity of a molecule X for its partner Y can generally be represented by the dissociation constant (K_{D}). Affinity can be measured by common methods known in the art, including those described herein. Specific illustrative embodiments for measuring binding affinity are described herein.

The term “antagonist” is used in the broadest sense, and includes any molecule that partially or fully blocks, inhibits, or neutralizes a biological activity of antigen. Suitable antagonist molecules specifically include antagonist antibodies or antibody fragments, or derivatives thereof.

A “blocking” antibody or an “antagonist” antibody is an antibody that inhibits or reduces biological activity of the antigen it binds or a protein complex comprising the antigen. Preferred blocking antibodies or antagonist antibodies substantially or completely inhibit the biological activity of the antigen or protein complex comprising the antigen.

The term “tumor” as used herein refers to all neoplastic cell growth and proliferation, whether malignant or benign, and all pre-cancerous and cancerous cells and tissues.

The terms “cancer” and “cancerous” as used herein refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), skin cancer, melanoma, lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer (e.g., pancreatic ductal adenocarcinoma), glioblastoma, cervical cancer, ovarian cancer (e.g., high grade serous ovarian carcinoma), liver cancer (e.g., hepatocellular carcinoma (HCC)), bladder cancer (e.g., urothelial bladder cancer), testicular (germ cell tumor) cancer, hepatoma, breast cancer, brain cancer (e.g., astrocytoma), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, hepatic carcinoma, anal carcinoma, penile carcinoma, as well as head and neck cancer. Additional examples of cancer include, without limitation, retinoblastoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, urinary tract carcinomas, anaplastic astrocytoma, basal cell carcinoma (basal cell epithelioma), bile duct cancer, small cell bladder cancer, metastatic breast cancer, metastatic colorectal cancer, epithelial ovarian cancer, fallopian tube cancer, gastric adenocarcinoma, glioblastoma multiforme (GBM), recurrent glioblastoma multiforme (GBM), gliomas, gliosarcoma, head and neck squamous cell carcinoma (HNSCC), recurrent head and neck cancer squamous cell carcinoma, malignant pleural mesothelioma head and neck cancer, Hodgkin lymphoma, metastatic renal cell carcinoma, metastatic renal clear cell carcinoma, squamous non-small cell lung cancer, squamous carcinoma of the lung, relapsed or refractory small-cell lung cancer, treatment-resistant melanoma, metastatic melanoma, Merkel cell carcinoma, neuroendocrine cancer, large cell neuroendocrine cancer, neuroendocrine tumors (NETS), ovarian carcinoma, papillary carcinoma, peritoneal cancer, neuroendocrine prostate cancer, hormone-refractory prostate cancer, castration-resistant prostate cancer, soft tissue sarcoma, and squamous cell carcinoma.

The term “metastatic cancer” means the state of cancer where the cancer cells of a tissue of origin are transmitted from the original site to one or more sites elsewhere in the body, by the blood vessels or lymphatics, to form one or more secondary tumors in one or more organs besides the tissue of origin. A prominent example is a metastatic breast cancer.

The terms “cell proliferative disorder” and “proliferative disorder” refer to disorders that are associated with some degree of abnormal cell proliferation. In one embodiment, the cell proliferative disorder is cancer.

The terms “associated,” “associating,” “linked,” or “linking” with regard to the claimed invention refers to the state of two or more components of a molecule being joined, attached, connected, or otherwise coupled to form a single molecule (or single molecular complex) or the act of making two molecules associated with each other to form a single molecule (or single molecular complex) by creating an association, linkage, attachment, and/or any other connection between the two molecules. For example, the term “linked” may refer to two or more components associated by one or more atomic interactions such that a single molecule is formed and wherein the individual atomic interactions may be covalent or non-covalent. Non-limiting examples of covalent associations between two components include peptide bonds and cysteine-cysteine disulfide bonds. Non-limiting examples of non-covalent associations between two molecular components include ionic bonds.

For purposes of the present invention, the term “fused” refers to two or more proteinaceous components associated by at least one covalent bond which is a peptide bond, regardless of whether the peptide bond involves the participation of a carbon atom of a carboxyl acid group or involves another carbon atom, such as, e.g., the α-carbon, β-carbon, γ-carbon, α-carbon, etc. Non-limiting examples of two proteinaceous components fused together include, e.g., an amino acid, peptide, or polypeptide fused to a polypeptide via a peptide bond such that the resulting molecule is a single, continuous polypeptide. For purposes of the present invention, the term “fusing” refers to the act of creating a fused molecule as described above, such as, e.g., a fusion protein generated from the recombinant fusion of genetic regions which when translated produces a single proteinaceous molecule.

A “bispecific” antibody refers to an antibody that has binding specificities for at least two different epitopes, regardless of whether the plurality of epitopes are in the same molecule and/or partially overlapping. In some embodiments, the bispecific immunoconjugate of the present invention binds to two different epitopes of a single antigen described herein.

As used herein, the terms “expressed,” “expressing,” or “expresses,” and grammatical variants thereof, refer to translation of a polynucleotide or nucleic acid into a protein. The expressed protein may remain intracellular, become a component of the cell surface membrane or be secreted into an extracellular space.

For purposes of the present invention, the phrase “derived from” when referring to a polypeptide or polypeptide region means that the polypeptide or polypeptide region comprises highly similar amino acid sequences originally found in a “parental” protein and which may now comprise certain amino acid residue additions, deletions, truncations, rearrangements, or other alterations relative to the original polypeptide or polypeptide region as long as a certain function(s) (e.g., antigen binding affinity) and a structure(s) of the “parental” molecule are substantially conserved. The skilled worker will be able to identify a parental molecule (e.g., an antibody sequence) from which a polypeptide or polypeptide region (e.g., a VHH polypeptide, CDR, HVR, V_{H}, and/or V_{L}) was derived using techniques known in the art, e.g., protein sequence alignment software.

As used herein, cells which express an extracellular target biomolecule or antigen on at least one cellular surface are “target positive cells” or “target+ cells” and are cells physically coupled to the specified, extracellular target biomolecule. Additional target biomolecule description is provided below. “Target biomolecule”, “target antigen molecule”, “target antigen”, “antigen of interest”, and grammatical variants and equivalents are used interchangeably herein as will be recognized by the person of ordinary skill in the art viewing the context of usage, and include the molecular determinants of antibody binding. Such antigens can be bound by the immunoconjugates described herein though the antigen binding region or antigen binding arm of the immunoconjugate.

The term “selective cytotoxicity” with regard to the cytotoxic activity of a molecule refers to the relative level of cytotoxicity between a biomolecule target positive cell population (e.g., a targeted cell-type) and a non-targeted bystander cell population (e.g., a biomolecule target negative cell-type), which can be expressed as a ratio of the half-maximal cytotoxic concentration (CD₅₀) for a targeted cell-type over the CD₅₀ for an untargeted cell-type to provide a metric of cytotoxic selectivity or indication of the preferentiality of killing of a targeted cell versus an untargeted cell.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

An “isolated” antibody or immunoconjugate or radio immunoconjugate is one which has been separated from a component of its natural environment or artificial production. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). Routine methods for assessment of antibody purity in a composition are known to the skilled worker, see e.g., Flatman et al., J. Chromatogr. B 848:79-87 (2007). In particular, unwanted components (contaminants) to be purified away from are such components that would interfere with desired uses for the antibody, such as, e.g., a therapeutic use, and may include, inter alia, bacterial factors, enzymes, hormones, and other proteinaceous or non-proteinaceous solutes.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present at extrachromosomal location or at a chromosomal location that is different from its natural chromosomal location.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, the term “administer”, with respect to an immunoconjugate or composition thereof (e.g., a radioimmunoconjugate, a pharmaceutical composition, or a diagnostic composition), means to deliver the immunoconjugate, or composition thereof, to a subject's body via any known method suitable for delivery of immunoconjugate or composition thereof. Specific modes of administration include, without limitation, intravenous, transdermal, subcutaneous, intraperitoneal and intrathecal administration.

An “effective amount” of an agent, e.g., a pharmaceutical formulation, refers to an amount effective, at dosages and for periods of time necessary, to achieve the desired therapeutic or prophylactic result.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, radioimmunoconjugates of the invention are used to delay development of a disease or to slow the progression of a disease.

A “therapeutically effective amount” is at least the minimum concentration required to effect a measurable improvement or prevention of a particular disorder. A therapeutically effective amount herein may vary according to factors such as the disease state, age, sex, and weight of the patient, and the ability of a composition of the invention to elicit a desired response in the individual. A therapeutically effective amount is also one in which any toxic or detrimental effects of the composition of the invention are outweighed by the therapeutically beneficial effects.

The terms “predictive” and “prognostic” as used herein are interchangeable. In one sense, the methods for prediction or prognostication are to allow the person practicing a predictive/prognostic method of the invention to select patients that are deemed (usually in advance of treatment, but not necessarily) more likely to respond to treatment with an immunoconjugate of the present invention or a composition of the aforementioned (e.g., a pharmaceutical composition).

The term “detecting” is used in the broadest sense to include both qualitative and quantitative measurements of a target antigen molecule. In one aspect, the detecting method as described herein is used to identify the mere presence of the antigen of interest in a biological sample. In another aspect, the method is used to test whether the antigen of interest in a sample is present at a detectable level. In yet another aspect, the method can be used to quantify the amount of the antigen of interest in a sample and further to compare the antigen levels from different samples. In another aspect, the method can be used in vivo to determine the location of a target cell, for example, using a targeted imaging complex of the invention.

The term “biological sample” refers to any biological substance that might contain an antigen of interest. A sample can be biological fluid, such as whole blood or whole blood components including red blood cells, white blood cells, platelets, serum and plasma, ascites, itreous fluid, lymph fluid, synovial fluid, follicular fluid, seminal fluid, amniotic fluid, milk, saliva, sputum, tears, perspiration, mucus, cerebrospinal fluid, and other constituents of the body that might contain the antigen of interest. In various embodiments, the sample is a biological sample from any animal. In some embodiments, the sample is from a mammal. In some embodiments, the sample is from a human subject. In some embodiments, the biological sample is serum from a clinical patient. In some embodiments, the biological sample is biopsy material. In some embodiments, the biological sample is biopsy material from a clinical patient. In some embodiments, the biological sample is serum from a clinical patient. In some embodiments, the biological sample is primary cell culture material. In some embodiments, the biological sample is primary cell culture material from a clinical patient. In some embodiments, the biological sample is from clinical patients or patients treated with a composition of the invention e.g., a radioimmunoconjugate, or treated with a different therapeutic agent, such as an antibody-drug conjugate targeting the antigen of interest or β-irradiation or a small molecule therapeutics.

The term “package insert” is used to refer to instructions customarily included in commercial packages of therapeutic products, that contain information about the indications, usage, dosage, administration, combination therapy, contraindications and/or warnings concerning the use of such therapeutic products.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkelene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

An “hydroxyalkyl” refers to an alkyl in which one hydrogen atom is replaced by a hydroxyl. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

An “aminoalkyl” refers to an alkyl in which one hydrogen atom is replaced by an amino. In some embodiments, aminoalkyl is a C₁-C₄aminoalkyl. Typical aminoalkyl groups include, but are not limited to, —CH₂NH₂, —CH₂CH₂NH₂, —CH₂CH₂CH₂NH₂, —CH₂CH₂CH₂CH₂NH₂, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahyodronaphthyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclcic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heterocycloalkyl also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —C₀₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

### EXAMPLES

Reagents used in these procedures are available from Macrocyclics, Millipore Sigma, CombiBlocks, Chem-Impex, LabNetwork, ArkPharm and Broadpharm. In some sections, advanced intermediates which are known to the art were prepared according to the literature. All solvents were obtained from VWR or LabNetwork and used as is unless otherwise indicated. Reaction that were carried out under nitrogen atmosphere used dry solvents. Mass spectra were taken with an Agilent HPLC-MS or Waters HPLC-MS with C18 reverse phase column and an acetonitrile/water (+0.1% formic acid) gradient. Flash chromatography was performed using a Biotage IsoleraOne instrument with an appropriately sized normal phase silica gel cartridge with fraction collection at 254 nm. Final compounds were purified by an Agilent prep-HPLC (Agilent, Hanover, CT) using an acetonitrile/water (+0.1% TFA or 0.1% FA) gradient. NMR spectra were taken with a Bruker 400 MHz NMR instrument and processed with MestReNova v.14. Where available, detailed NMR Data was compiled with the multiplet analysis function used in manual mode.

Abbreviations used in this experimental include, but are not limited to:
DCM—methylene chloride/dichloromethane
TFA—Trifluoroacetic acid
HATU—1-[Bis(dimethylamino)methylene]-1H-1,2,3-triazolo[4,5-b]pyridinium 3-oxide hexafluorophosphate
HBTU—3-[Bis(dimethylamino)methyliumyl]-3H-benzotriazol-1-oxide hexafluorophosphate
HOAt—3H-[1,2,3]Triazolo[4,5-b]pyridin-3-ol
EDC/EDCI—3-{[(Ethylimino)methylidene]amino}-N,N-dimethylpropan-1-amine
DCC—N,N′-Dicyclohexylcarbodiimide
ACN/MeCN—acetonitrile
EtOAc—ethyl acetate
Et2O—(Diethyl) ether
DMF—N,N-dimethylformamide
DIPEA—N,N-diisopropylethylamine
MeOH—Methanol

### Example 1. Compound 3-1

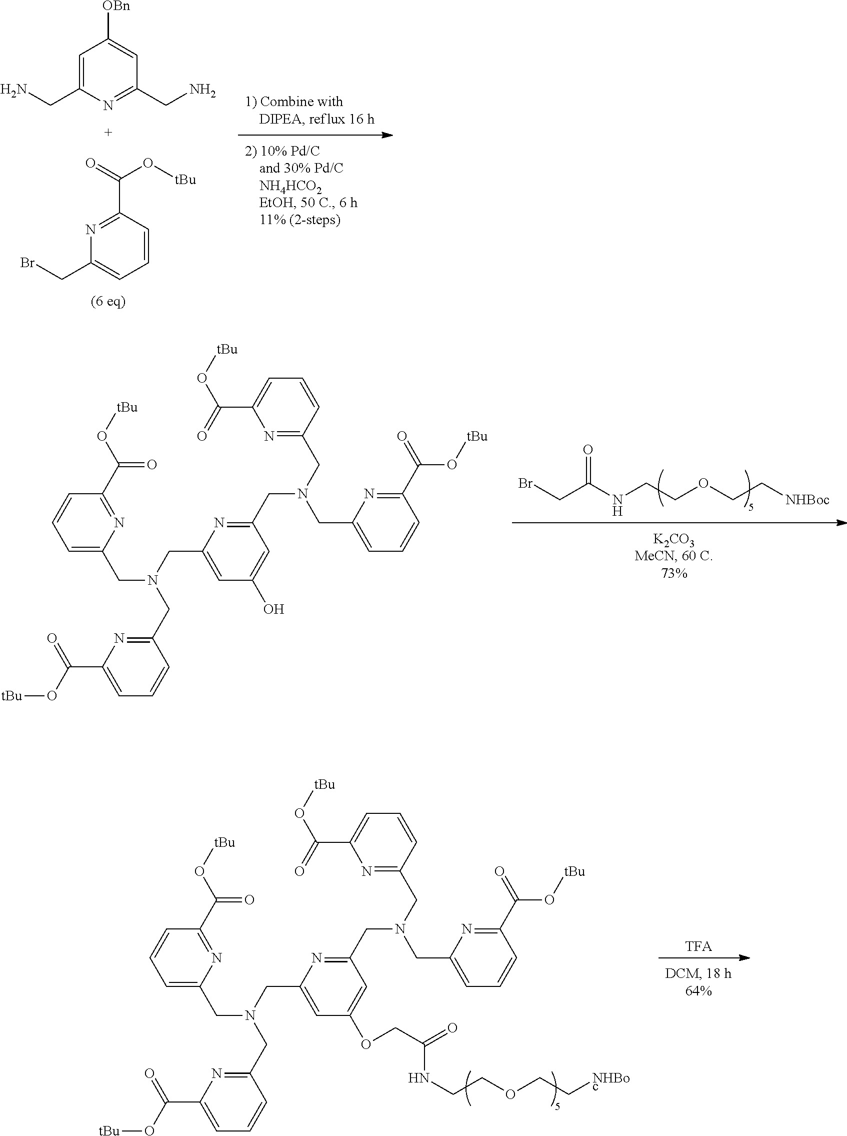

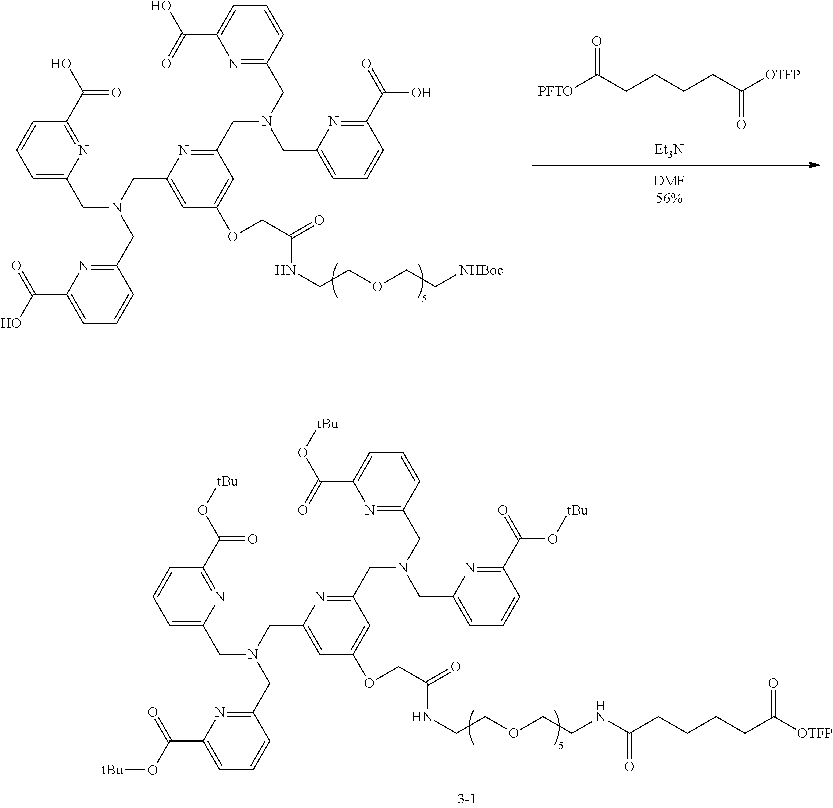

### Synthesis of tetra-tert-butyl 6,6′,6″,6′″((((4-hydroxypyridine-2,6-diyl)bis(inethylene))bis(azanetriyl))tetrakis(methylene))tetrapicolinate

To a stirred solution of 1-[6-(aminomethyl)-4-(benzyloxy)pyridin-2-yl]methanamine (0.65 g, 2.67 mmol) (available from N. Delsuc, et al. Angew Chem. Int. Ed. 2007, 46, 214-217) in acetonitrile (50 mL) was added DIPEA (1.40 mL, 1.04 mg, 8.01 mmol) and tert-butyl 6-(bromomethyl)pyridine-2-carboxylate (4.36 g, 16.0 mmol) (available from P. Coomba, et al. Inorg. Chem. 2016, 55, 12531-12543) and the solution was heated to reflux. After 16 h, the reaction was allowed to cool and the solvent removed in vacuo. The crude was taken up in 200 mL DCM and washed 2×75 mL NaHCO₃₍ₛₐₜ₎ and 2×75 mL saturated brine. The DCM layer was then dried over sodium sulfate, filtered, and concentrated in vacuo to yield a brown crude oil (950 mg) that could be used in the following step without further purification. The intermediate from above was dissolved in EtOH, ammonium formate (297 mg, 4.71 mmol) was added, and the flask was purged with N₂. 10% Pd/C (250 mg, 0.23 mmol) was added followed by another purge with N₂, then 30% Pd/C (50 mg, 0.14 mmol) was added. Following another purge with N₂, the reaction was heated to 50° C. and stirred for 6 h where the reaction was complete by LCMS. The reaction mixture was filtered through celite, washed 3×50 mL MeOH, then concentrated in vacuo to a pale-yellow oil. The crude was purified by flash chromatography using a Biotage Sfar amino D cartridge and a gradient of 40-100% EtOAc/Hexanes followed by 0-20% MeOH/DCM to yield the title compound as a yellow solid (278 mg, 11%). ¹H NMR (400 MHz, Methanol-d₄) δ 7.88 (dd, J=7.7, 1.3 Hz, 4H), 7.82 (t, J=7.7 Hz, 4H), 7.73 (dd, J=7.7, 1.2 Hz, 4H), 6.41 (s, 2H), 4.00 (s, 8H), 3.94 (s, 4H), 1.61 (s, 36H). m/z found=918.4 (M+H).

### Synthesis of tert-butyl (1-bromo-2-oxo-6,9,12,15,18-pentaoxa-3-azaicosan-20-yl)carbamate

A solution of tert-butyl N-(17-amino-3,6,9,12,15-pentaoxaheptadecan-1-yl)carbamate (200 mg, 0.53 mmol) and DIPEA (0.146 mL, 109 mg, 0.84 mmol) in 5 mL DCM was cooled to 0° C. A solution of 2-bromoacetyl bromide (0.069 mL, 159 mg, 0.79 mmol) in 5 mL DCM cooled to 0° C. was added dropwise over 2 min. The reaction was allowed to warm to rt, after 90 min HPLC showed full conversion to product. The reaction was concentrated, partitioned between Et₂O and water, NaHCO₃ (sat) was added, then the mixture was extracted 3×25 mL with Et₂O. The combined organics were washed with brine, dried over sodium sulfate, filtered and concentrated in vacuo. The crude residue was co-evaporated once with acetonitrile to remove water. The title compound was recovered as a brownish oil (261 mg, 99%). ¹H NMR (400 MHz, Chloroform-d) δ 3.90 (s, 2H), 3.75-3.64 (m, 18H), 3.61 (d, J=4.5 Hz, 2H), 3.56 (t, J=5.1 Hz, 2H), 3.52 (t, J=5.2 Hz, 2H), 3.37-3.30 (m, 2H), 1.46 (s, 9H). m/z found=523.2 (M+Na).

### Synthesis of tetra-tert-butyl 6,6,6′,6′″-((((4-((2,2-dimethyl-4,24-dioxo-3,8,11,14,17,20-hexaoxa-5,23-diazapentacosan-25-yl)oxy)pyridine-2,6-diyl)bis(methylene))bis (azanetriyl))tetrakis(methylene))tetrapicolinate

Compound tetra-tert-butyl 6,6′,6″,6′″-((((4-hydroxypyridine-2,6-diyl)bis (methylene)) bis(azanetriyl))tetrakis(methylene))tetrapicolinate (100 mg, 0.11 mmol) and tert-butyl (19-oxo-3,6,9,12,15-pentaoxa-18-azahenicosyl)carbamate (81.9 mg, 0.163 mmol) were taken up in acetonitirile (5 mL), then potassium carbonate (30.1 mg, 0.218 mmol) was added and the reaction was stirred at 60° C. After 24 h, no starting material remained by HPLC. The reaction was concentrated and purified by flash chromatography (Biotage amino D cartridge, gradient 0.2-15% MeOH/DCM) to yield the title compound as a yellow film (106 mg, 73%). ¹H NMR (400 MHz, Methanol-d₄) δ 7.89 (d, J=7.8 Hz, 4H), 7.83 (t, J=7.7 Hz, 4H), 7.66 (d, J=7.6 Hz, 4H), 6.95 (s, 2H), 4.66 (s, 2H), 4.04 (s, 8H), 3.92 (s, 4H), 3.75-3.55 (m, 20H), 3.53-3.43 (m, 2H), 3.30-3.13 (m, 2H), 1.52 (s, 36H), 1.43 (s, 9H). m/z found=670.0 (M+2H/2).

### Synthesis of 6,6′,6″,6′″-((((4-((20-amino-2-oxo-6,9,12,15,18-pentaoxa-3-azaicosyl)oxy) pyridine-2,6-diyl)bis(methylene))bis(azanetriyl))tetrakis(methylene))tetrapicolinic acid

Compound tetra-tert-butyl 6,6′,6″,6′″-((((4-((2,2-dimethyl-4,24-dioxo-3,8,11,14, 17,20-hexaoxa-5,23-diazapentacosan-25-yl)oxy)pyridine-2,6-diyl)bis(methylene))bis (azanetriyl))tetrakis(methylene))tetrapicolinate (125 mg, 0.093 mmol) was taken up in DCM (5 mL) and TFA (5 mL) was added. After 18 h, HPLC showed no starting material or t-butyl intermediates remaining. The reaction was concentrated in vacuo and co-evaporated once with DCM. The crude oil was triturated 2× with Et₂O with sonication and collected by filtration to yield 100 mg (64%, as a 5×TFA salt) of the title compound as a brownish solid. ¹H NMR (400 MHz, Methanol-d₄) δ 8.04 (d, J=7.7 Hz, 4H), 7.96 (t, J=7.8 Hz, 4H), 7.66 (t, J=8.4 Hz, 4H), 7.45 (s, 2H), 4.84 (s, 2H), 4.74-4.49 (m, 12H), 3.74 (t, J=5.0 Hz, 2H), 3.71-3.63 (m, 14H), 3.60

### Synthesis of 6,6′,6″,6′″-((((4-((2,22,27-trioxo-27-(2,3,5,6-tetrafluorophenoxy)-6,9,12,15,18-pentaoxa-3,21-diazaheptacosyl)oxy)pyridine-2,6-diyl)bis(methylene))bis(azanetriyl))tetrakis(methylene))tetrapicolinic acid

To a solution of compound 6,6′,6″,6′″-((((4-((20-amino-2-oxo-6,9,12,15,18-pentaoxa-3-azaicosyl)oxy)pyridine-2,6-diyl)bis(methylene))bis(azanetriyl))tetrakis(methylene))tetrapicolinic acid (80 mg, 0.079 mmol) in DMF (2.5 mL) was added bis(2,3,5,6-tetrafluorophenyl) hexanedioate (140 mg, 0.32 mmol) and triethylamine (0.027 mL, 20 mg, 0.197 mmol). Full conversion to product was confirmed by HPLC. After stirring for 4 h, the reaction was diluted with DMSO (1.5 mL) and purified by direct injection onto prep-HPLC with a gradient of 25-60% MeCN/water+0.1% TFA to yield the title compound (57.5 mg, 56%) as a white powder (3×TFA salt). ¹H NMR (400 MHz, Deuterium Oxide) δ 7.85 (t, J=7.8 Hz, 4H), 7.78 (dd, J=7.8, 1.2 Hz, 4H), 7.50 (dd, J=7.8, 1.2 Hz, 4H), 7.11 (tt, J=10.4, 7.2 Hz, 1H), 6.99 (s, 2H), 4.59 (s, 2H), 4.49 (s, 8H), 4.45 (s, 4H), 3.60-3.45 (m, 18H), 3.46 (t, J=5.3 Hz, 2H), 3.36 (t, J=5.3 Hz, 2H), 3.22 (t, J=5.3 Hz, 2H), 2.59 (t, J=6.7 Hz, 2H), 2.14 (t, J=6.7 Hz, 2H), 1.61-1.46 (m, 4H). m/z found=1290.3 (M+H).

### Example 2. Compound 3-2

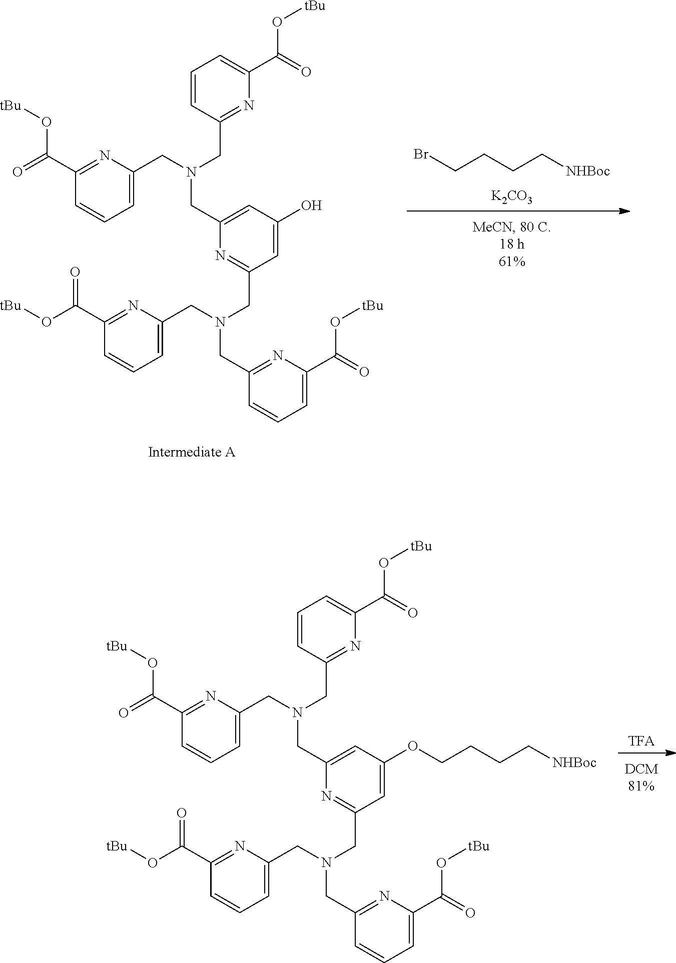

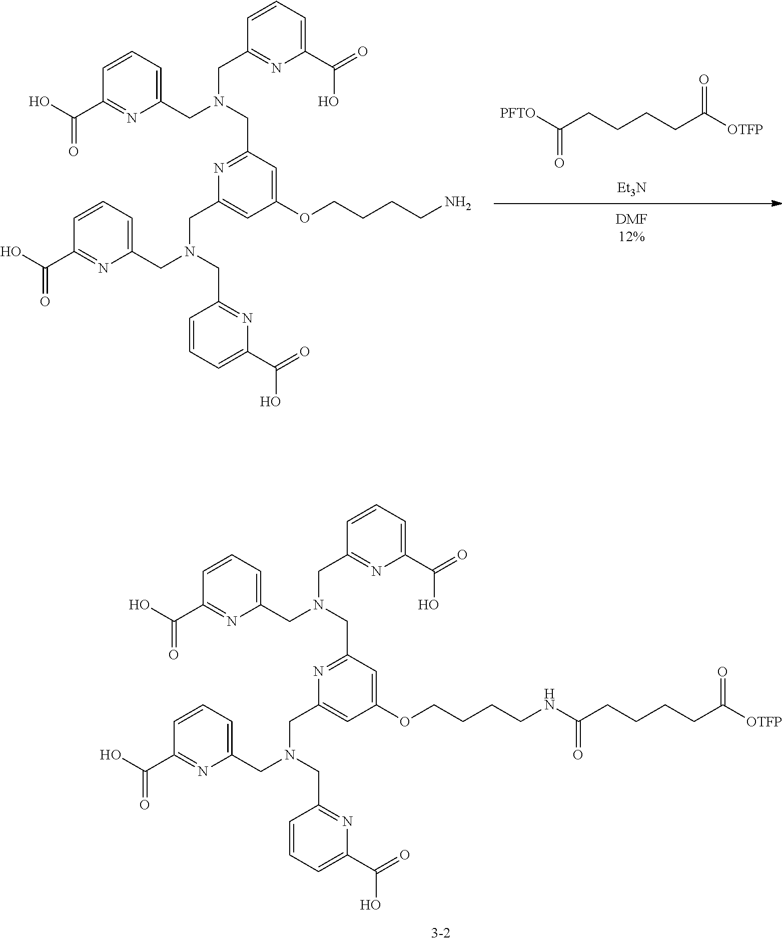

### Synthesis of tetra-tert-butyl 6,6′,6″,6′″-((((4-(4-((tert-butoxycarbonyl)amino)butoxy)pyridine-2,6-diyl)bis(mnethylene))bis(azanetriyl))tetrakis(methylene))tetrapicolinate

Intermediate A (43 mg, 0.047 mmol) and tert-butyl N-(4-bromobutyl)carbamate (23.6 mg, 0.094 mmol) were taken up in acetonitrile (5 mL), then potassium carbonate (12.9 mg, 0.094 mmol) was added and the reaction was stirred at 80° C. After 24 h, no starting material remained by HPLC. The reaction was filtered through cotton, washing with MeCN, concentrated and purified by flash chromatography (20-100% EtOAc/Hex, then 0-15% MeOH/DCM) to yield the title compound as a clear film (31.1 mg, 61%). ¹H NMR (400 MHz, Methanol-d₄) δ 7.87 (s, 12H), 7.05 (s, 2H), 4.12-4.04 (m, 2H), 3.97 (s, 8H), 3.83 (s, 4H), 3.20-3.04 (m, 2H), 1.86-1.74 (m, 2H), 1.70-1.58 (m, 38H), 1.44 (s, 9H). m/z found=545.4 (M+2H/2).

### Synthesis of 6,6′,6″,6′″-((((4-(4-aminobutoxy)pyridine-2,6-diyl)bis(methylene))bis(azanetriyl)) tetrakis(methylene))tetrapicolinic acid

Compound tetra-tert-butyl 6,6′,6″,6′″-((((4-(4-((tert-butoxycarbonyl)amino)butoxy) pyridine-2,6-diyl)bis(methylene))bis(azanetriyl))tetrakis(methylene))tetrapicolinate (31.1 mg, 0.029 mmol) was taken up in DCM (5 mL) and TFA (5 mL) was added. After 24 h, the reaction was concentrated in vacuo and co-evaporated once with DCM. The crude oil was triturated with Et₂O with sonication and collected by filtration to yield the title compound (36.7 mg, 81%, 77% w/w with Et₂O) as an off-white solid (assumed to be a 5×TFA salt). ¹H NMR (400 MHz, Methanol-d₄) δ 8.04 (d, J=7.7 Hz, 4H), 7.96 (t, J=7.5 Hz, 4H), 7.65 (d, J=7.5 Hz, 4H), 7.31 (s, 2H), 4.72 (s, 4H), 4.70 (s, 8H), 4.25 (s, 2H), 3.14-2.96 (m, 2H), 1.95 (s, 2H), 1.92-1.86 (m, 2H). m/z found=383.3 (M+2H/2).

### Synthesis of 6,6′,6″,6′″-((((4-(4-(6-oxo-6-(2,3,5,6-tetrafluorophenoxy)hexanamido)butoxy) pyridine-2,6-diyl)bis(methylene))bis(azanetriyl))tetrakis(methylene))tetrapicolinic acid

To a solution of 6,6′,6″,6′″-((((4-(4-aminobutoxy)pyridine-2,6-diyl)bis(methylene))bis (azanetriyl))tetrakis(methylene))tetrapicolinic acid (12 mg, 0.0099 mmol) in DMF (1.0 mL) was added bis(2,3,5,6-tetrafluorophenyl) hexanedioate (21.7 mg, 0.049 mmol) and triethylamine (0.0041 mL, 8.0 mg, 0.080 mmol). Full conversion to product was confirmed by HPLC after 22 h. At that point, the reaction was quenched by addition of 2% TFA by volume, diluted with DMSO and purified by direct injection onto prep-HPLC with a gradient of 35-65% MeCN/water+0.1% TFA to yield the title compound (1.6 mg, 12%) as a white powder (2×TFA salt). ¹H NMR (400 MHz, Methanol-d₄) δ 8.01 (d, J=7.7 Hz, 4H), 7.91 (td, J=7.7, 1.8 Hz, 4H), 7.69 (d, J=7.7 Hz, 4H), 7.48-7.36 (m, 1H), 7.31 (s, 2H), 4.45-4.35 (m, 4H), 4.28 (dd, J=16.5, 7.2 Hz, 12H), 2.77 (t, J=6.8 Hz, 2H), 2.29 (t, J=6.6 Hz, 2H), 1.97-1.84 (m, 1H), 1.84-1.62 (m, 7H). m/z found=1041.2 (M+H).

### Example 3. Compound 3-3

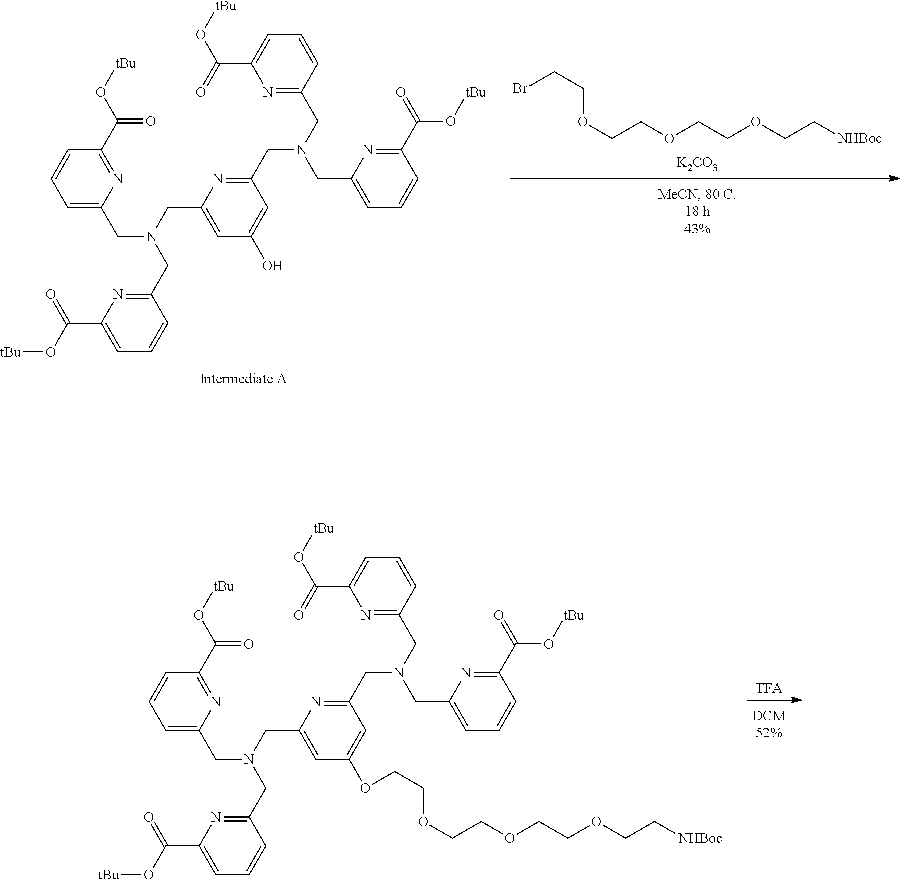

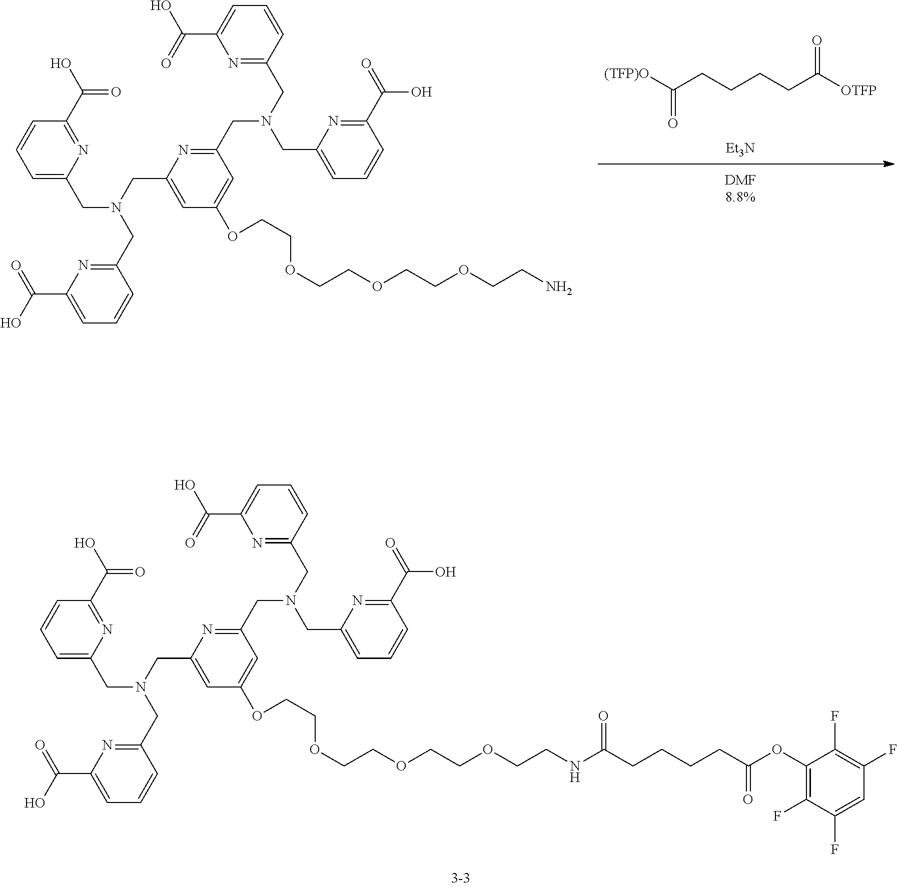

### Synthesis of tetra-tert-butyl 6,6′,6′,6′″-((((4-((2,2-dimethyl-4-oxo-3,8,11,14-tetraoxa-5-azahexadecan-16-yl)oxy)pyridine-2,6-diyl)bis(methylene))bis(azanetriyl))tetrakis(methylene)) tetrapicolinate

Intermediate A (40 mg, 0.044 mmol) and tert-butyl N-(2-{2-[2-(2-bromoethoxy)ethoxy]ethoxy}ethyl)carbamate (31 mg, 0.087 mmol) were taken up in acetonitrile (1.5 mL), then potassium carbonate (12.0 mg, 0.087 mmol) was added and the reaction was stirred at 80° C. After 24 h, no starting material remained by HPLC. The reaction was filtered through cotton, washing with MeCN, concentrated and purified by flash chromatography (using Biotage Amino D cartridge, 20100% EtOAc/Hex, then 0-15% MeOH/DCM) to yield the title compound as a clear film (22.1 mg, 43). ¹H NMR (400 MHz, Chloroform-d) δ 8.04-7.86 (d, 8H), 7.86-7.74 (m, 6H), 5.03 (s, 1H), 3.98 (s, 14H), 3.79-3.72 (m, 2H), 3.69 (dt, J=6.2, 2.7 Hz, 2H), 3.66-3.59 (9, 6H), 3.54 (t, J=5.3 Hz, 2H), 3.32 (q, J=5.6 Hz, 2H), 1.64 (s, 36H), 1.44 (s, 9H). m/z found=597.5 (M+2H1-2).

### Synthesis of 6,6′,6″,6′″-((((4-(2-(2-(2-(2-aminoethoxy)ethoxy)ethoxy)ethoxy)pyridine-2,6-diyl)bis(inethylene))bis(azanetriyl))tetrakis(methylene))tetrapicolinic acid

Compound tetra-tert-butyl 6,6′,6″,6′″-((((4-((2,2-dimethyl-4-oxo-3,8,11,14-tetraoxa-5-azahexadecan-16-yl)oxy)pyridine-2,6-diyl)bis(methylene))bis(azanetriyl))tetrakis(methylene))tetrapicolinate (22.1 mg, 0.019 mmol) was taken up in DCM (3 mL) and TFA (3 mL) was added. After 24 h, the reaction was concentrated in vacuo and co-evaporated once with DCM. The crude oil was triturated with Et₂O with sonication and collected by filtration to yield the title compound (22.1 mg, 52% o as a white solid (assumed to be a 5×TFA salt). ¹H NMR (400 MHz, Methanol-d₄) δ 8.03 (d, J=7.7 Hz, 4H), 7.94 (t, J=7.7 Hz, 4H), 7.67 (d, J=7.7 Hz, 4H), 7.37 (s, 2H), 4.61-4.56 (m, 4H), 4.54-4.47 (m, 8H), 4.39 (t, J=4.9 Hz, 2H), 3.92 (t, J=4.4 Hz, 2H), 3.78-3.65 (m, 10H), 3.15 (t, J=4.9 Hz, 2H). m/z found=869.3 (M+H).

### Synthesis of 6,6′,6″,6′″-((((4-((13,18-dioxo-18-(2,3,5,6-tetrafluorophenoxy)-3,6,9-trioxa-12-azaoctadecyl)oxy)pyridine-2,6-diyl)bis(methylene))bis(azanetriyl))tetrakis(methylene)) tetrapicolinic acid

To a solution of compound 6,6′,6″,6′″-((((4-(2-(2-(2-(2-aminoethoxy)ethoxy)ethoxy) ethoxy)pyridine-2,6-diyl)bis(methylene))bis(azanetriyl))tetrakis(methylene))tetrapicolinic acid (11 mg, 0.0076 mmol) in DMF (1.0 mL) was added bis(2,3,5,6-tetrafluorophenyl) hexanedioate (16.9 mg, 0.038 mmol) and triethylamine (0.0053 mL, 3.9 mg, 0.038 mmol). Full conversion to product was confirmed by HPLC after 16 h. At that point, the reaction was diluted with DMSO and purified by direct injection onto prep-HPLC with a gradient of 25-60% MeCN/water+0.1% TFA to yield the title compound (1.0 mg, 8.8%) as a white powder (2×TFA salt). ¹H NMR (400 MHz, Deuterium Oxide) δ 7.94-7.86 (m, 4H), 7.81 (d, J=7.5 Hz, 4H), 7.55 (d, J=7.8 Hz, 4H), 7.18-7.02 (m, 1H), 6.95 (s, 2H), 4.40 (s, 8H), 4.34 (s, 4H), 4.21-4.01 (m, 2H), 3.83-3.74 (m, 2H), 3.69-3.62 (m, 2H), 3.62-3.57 (m, 2H), 3.57-3.49 (m, 4H), 3.46 (t, J=5.4 Hz, 2H), 3.22 (t, J=5.3 Hz, 2H), 2.54 (t, J=6.9 Hz, 2H), 2.11 (t, J=6.5 Hz, 2H), 1.54-1.44 (m, 4H). m/z found=1145.3 (M+H).

### Example 4. Compound 3-4

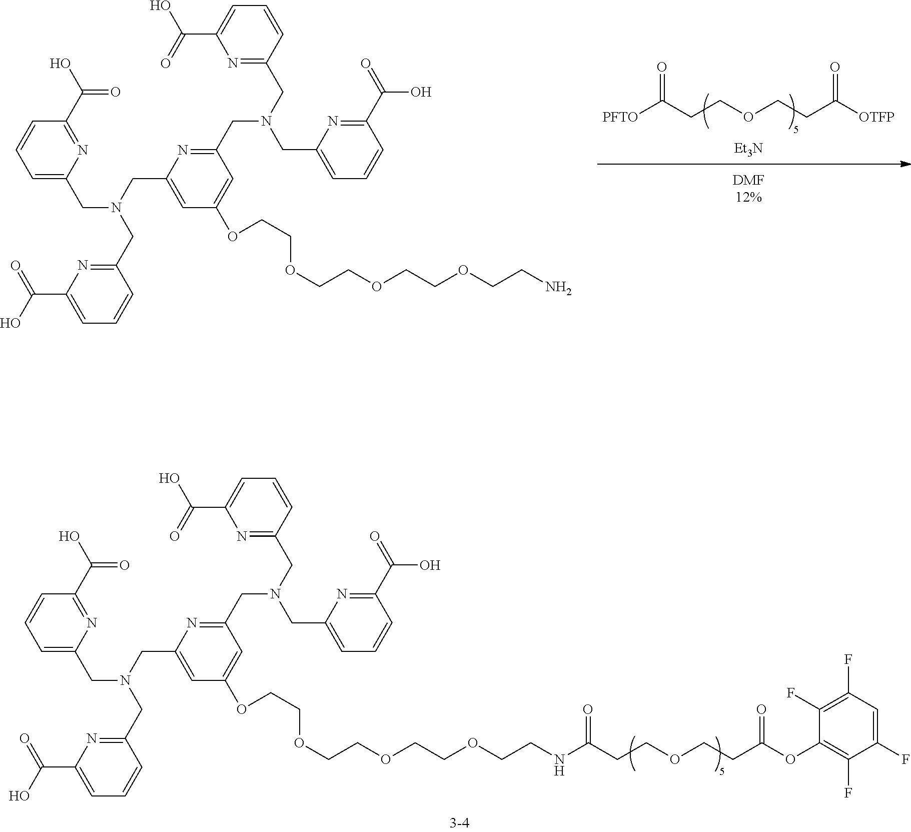

### 6,6′,6″,6′″-((((4-((13,31-dioxo-31-(2,3,5,6-tetrafluorophenoxy)-3,6,9,16,19,22,25,28-octaoxa-12-azahentriacontyl)oxy)pyridine-2,6-diyl)bis(methylene))bis(azanetriyl))tetrakis(methylene)) tetrapicolinic acid

Compound 6,6′,6″,6′″-((((4-(2-(2-(2-(2-aminoethoxy)ethoxy)ethoxy)ethoxy)pyridine-2,6-diyl)bis(methylene))bis(azanetriyl))tetrakis(methylene))tetrapicolinic acid (11 mg, 0.0076 mmol) in DMF (1.0 mL) was added bis(2,3,5,6-tetrafluorophenyl) 4,7,10,13,16-pentaoxanonadecanedioate (14.5 mg, 0.023 mmol) and triethylamine (0.0053 mL, 3.9 mg, 0.038 mmol). Full conversion to product was confirmed by HPLC after overnight incubation. After stirring for 16 h, the reaction was diluted with DMSO and purified by direct injection onto prep-HPLC with a gradient of 25-60% MeCN/water+0.1% TFA to yield the title compound (1.5 mg, 12%) as a white powder (3×TFA salt). ¹H NMR (400 MHz, Deuterium Oxide) δ 7.89 (dt, J=7.8, 4.0 Hz, 4H), 7.82 (d, J=9.2 Hz, 4H), 7.55 (d, J=7.0 Hz, 4H), 7.29-7.08 (m, 1H), 6.96 (d, J=2.9 Hz, 2H), 4.45 (s, 8H), 4.41 (s, 4H), 4.21-4.09 (m, 2H), 3.89-3.72 (m, 4H), 3.65 (s, 2H), 3.63-3.39 (m, 26H), 3.21 (s, 2H), 2.99-2.82 (m, 2H), 2.43-2.26 (m, 2H). m/z found=1337.4 (M+H).

### Example 5. Compound 3-5

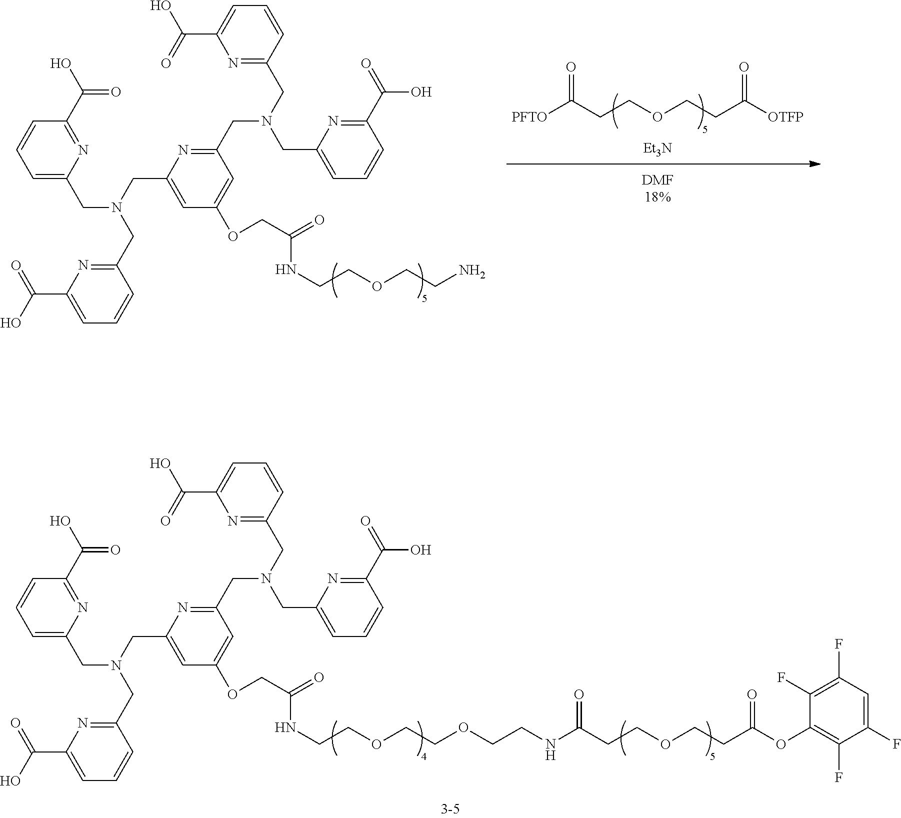

### 6,6′,6″,6′″-((((4-((2,22,40-trioxo-40-(2,3,5,6-tetrafluorophenoxy)-6,9,12,15,18,25,28,31,34,37-decaoxa-3,21-diazatetracontyl)oxy)pyridine-2,6-diyl)bis(methylene))bis(azanetriyl))tetrakis (methylene))tetrapicolinic acid

Compound 6,6′,6″,6′″-((((4-(2-(2-(2-(2-aminoethoxy)ethoxy)ethoxy)ethoxy)pyridine-2,6-diyl)bis(methylene))bis(azanetriyl))tetrakis(methylene))tetrapicolinic acid (11.2 mg, 0.0076 mmol) in DMF (0.5 mL) was added bis(2,3,5,6-tetrafluorophenyl) 4,7,10,13,16-pentaoxanonadecanedioate (14.5 mg, 0.023 mmol) and triethylamine (0.0053 mL, 3.9 mg, 0.038 mmol). Full conversion to product was confirmed by HPLC after overnight incubation. After stirring for 24 h, the reaction was diluted with DMSO and purified by direct injection onto prep-HPLC with a gradient of 25-60% MeCN/water+0.1% TFA to yield the title compound (2.0 mg, 18%) as a white powder (2×TFA salt). ¹H NMR (400 MHz, Deuterium Oxide) δ 7.90 (td, J=7.9, 2.4 Hz, 4H), 7.82 (d, J=7.8 Hz, 4H), 7.54 (d, J=7.1 Hz, 2H), 7.19 (d, J=9.5 Hz, 1H), 7.00 (d, J=2.4 Hz, 2H), 4.50 (s, 8H), 4.45 (s, 4H), 3.82 (s, 2H), 3.73-3.43 (m, 42H), 3.43-3.34 (m, 2H), 3.27 (t, J=6.7 Hz, 2H), 2.93 (t, J=7.2 Hz, 2H), 2.40 (t, J=5.3 Hz, 2H). m/z found=742.0 (M+2H/2).

### Example 6. Compound 3-6

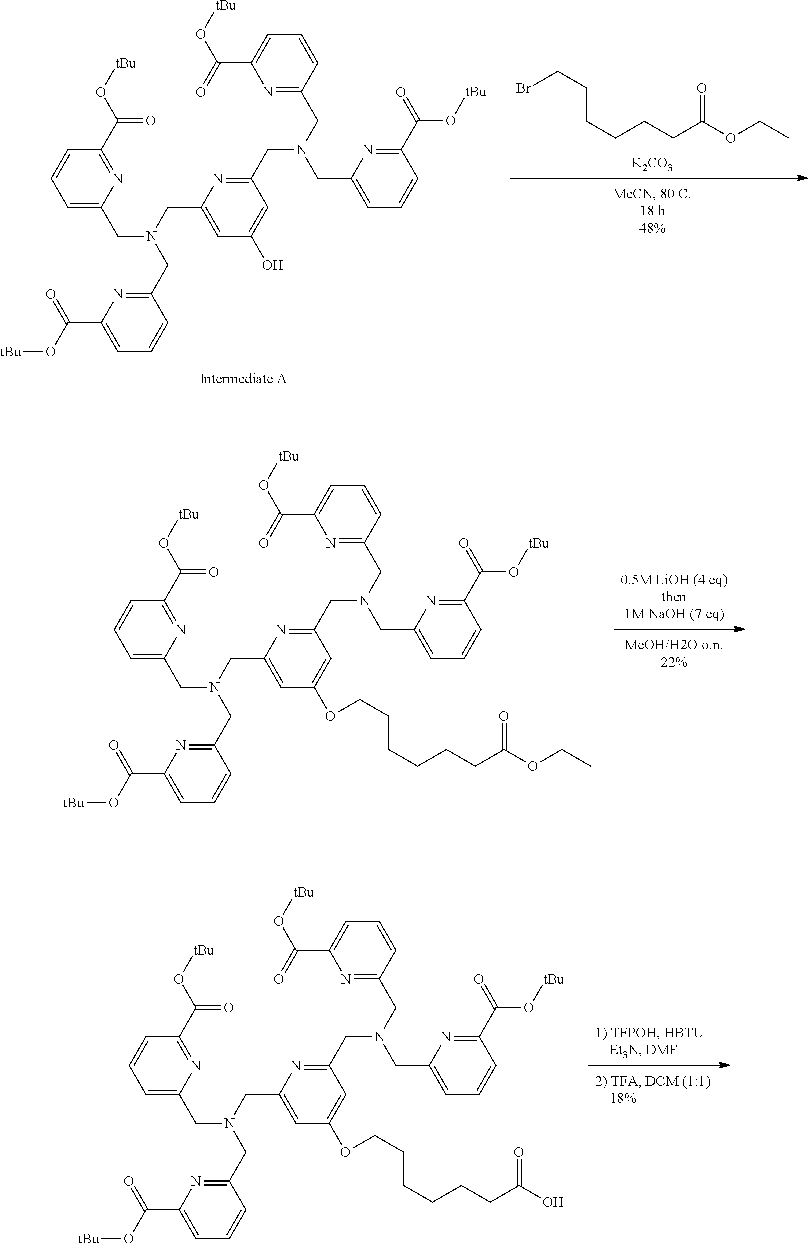

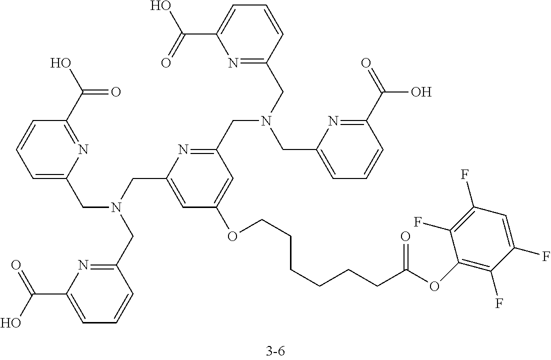 [SMILES: O=C(CCCCCCOc1cc(CN(Cc2cccc(C(=O)O)n2)Cc2cccc(C(=O)O)n2)nc(CN(Cc2cccc(C(=O)O)n2)Cc2cccc(C(=O)O)n2)c1)Oc1c(F)c(F)cc(F)c1F]

### Synthesis of tetra-tert-butyl 6,6′,6″,6′″-((((4-((7-ethoxy-7-oxoheptyl)oxy)pyridine-2,6-diyl)bis(methylene))bis(azanetriyl))tetrakis(methylene))tetrapicolinate

Intermediate A (100 mg, 0.109 mmol) and ethyl 7-bromoheptanoate (0.032 mL, 38.7 mg, 0.163 mmol) were taken up in acetonitirile (2 mL), then potassium carbonate (30.1 mg, 0.218 mmol) was added and the reaction was stirred at 80° C. After 1 h, HPLC indicated significant conversion, so the heat was reduced to 60° C. and allowed to stir overnight. After 16 h, no starting material remained by HPLC. The reaction was filtered through cotton, washing with MeCN, concentrated and purified by flash chromatography (using Biotage Amino D cartridge, 10-100% EtOAc/Hex) to yield the title compound as a clear film (55.7 mg, 48%). ¹H NMR (400 MHz, Methanol-d₄) δ 7.88 (s, 12H), 7.06 (s, 2H), 4.11 (q, J=7.2 Hz, 2H), 4.07-4.02 (m, 2H), 3.96 (s, 8H), 3.82 (s, 4H), 2.32 (t, J=7.2 Hz, 2H), 1.78 (t, J=7.5 Hz, 2H), 1.62 (s, 38H), 1.54-1.44 (m, 2H), 1.39 (dd, J=9.2, 6.1 Hz, 2H), 1.24 (t, J=7.2 Hz, 3H). m/z found=537.9 (M+2H/2).

### Synthesis of 7-((2,6-bis((bis((6-(tert-butoxycarbonyl)pyridin-2-yl)methyl)amino)methyl) pyridin-4-yl)-oxy)heptanoic acid

Compound tetra-tert-butyl 6,6′,6″,6′″-((((4-((7-ethoxy-7-oxoheptyl)oxy)pyridine-2,6-diyl)bis(methylene))bis(azanetriyl))tetrakis(methylene))tetrapicolinate (55.7 mg, 0.052 mmol) was taken up in MeCN/water (3.1, 2.7 mL) with stirring. To this, a solution of LiOH (0.5 M, 0.104 mmol) was added. After 2 h, additional LiOH (0.5 M, 0.104 mmol) was added. At 20 h, with limited conversion, NaOH (1 M, 0.35 mmol) was added. After an additional 2 h, HCl (1 M, 0.45 mmol) was added to quench the base, then the reaction was concentrated in vacuo until most of the MeCN evaporated. The remaining aqueous mixture was purified by dilution 50% with DMSO and loading directly onto C₁₈ flash chromatography (10-100% MeCN/water+0.1% TFA) to yield the title compound (15.5 mg, 22%) after lyophilization. ¹H NMR (400 MHz, Methanol-d₄) δ 7.98-7.80 (m, 8H), 7.75-7.59 (m, 4H), 7.49-7.34 (m, 2H), 4.56 (s, 4H), 4.46 (s, 8H), 4.35-4.24 (m, 2H), 2.32 (t, J=7.4 Hz, 2H), 1.88 (dt, J=11.9, 7.0 Hz, 2H), 1.60 (s, 40H), 1.45 (d, J=7.7 Hz, 2H). m/z found=523.9 (M+2H/2).

### Synthesis of 6,6′,6″,6′″-((((4-((7-oxo-7-(2,3,5,6-tetrafluorophenoxy)heptyl)oxy)pyridine-2,6-diyl)bis(methylene))bis(azanetriyl))tetrakis(methylene))tetrapicolinic acid

Compound 7-((2,6-bis((bis((6-(tert-butoxycarbonyl)pyridin-2-yl)methyl)amino) methyl)pyridin-4-yl)oxy)heptanoic acid (15.5 mg, 0.011 mmol) and HBTU (8.5 mg, 0.022 mmol) were taken up in DCM (2 mL). Next, triethylamine (0.0062 mL, 4.5 mg, 0.045 mmol) then 2,3,5,6-tetrafluorophenol (3.7 mg, 0.022 mmol) were added to the stirring solution. After 16 h, HPLC indicated complete conversion to the TFP ester. The reaction was placed in the −30° C. freezer for 24 h, then thawed and diluted in DMSO and purified by C18 flash chromatography (30-80% MeCN/water+0.1% TFA), collecting at 254 nm.

After lyophilization, the tBu-protected intermediate was taken to the next step directly by dissolution in DCM (3 mL) and adding TFA (3 mL). After 24 h, the reaction was concentrated in vacuo, co-evaporated with DCM, then purified by prep-HPLC (20-60% MeCN/water+0.1% TFA) to yield the title compound (2.7 mg, 18%) as a white solid. ¹H NMR (400 MHz, Methanol-d₄) δ 8.01 (d, J=7.7 Hz, 4H), 7.90 (t, J=7.7 Hz, 4H), 7.69 (d, J=7.7 Hz, 4H), 7.44 (d, J=7.3 Hz, 1H), 7.34 (s, 2H), 4.38 (s, 4H), 4.34-4.18 (m, 10H), 2.78 (t, J=7.2 Hz, 2H), 1.95-1.78 (m, 4H), 1.57 (s, 4H). m/z found=970.2 (M+H).

### Example 7. Compound 3-7

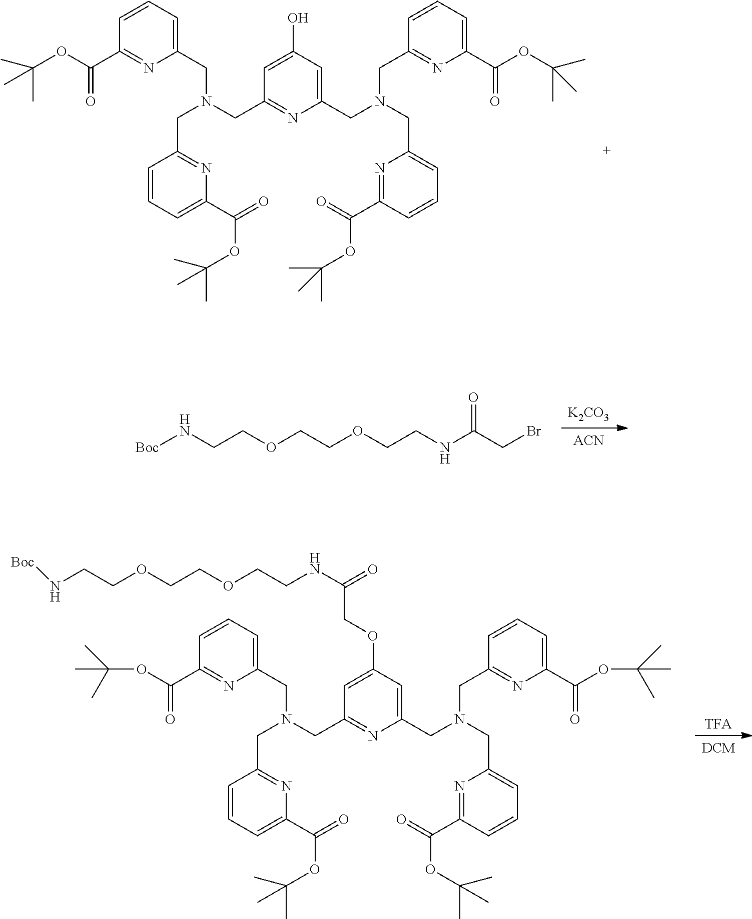

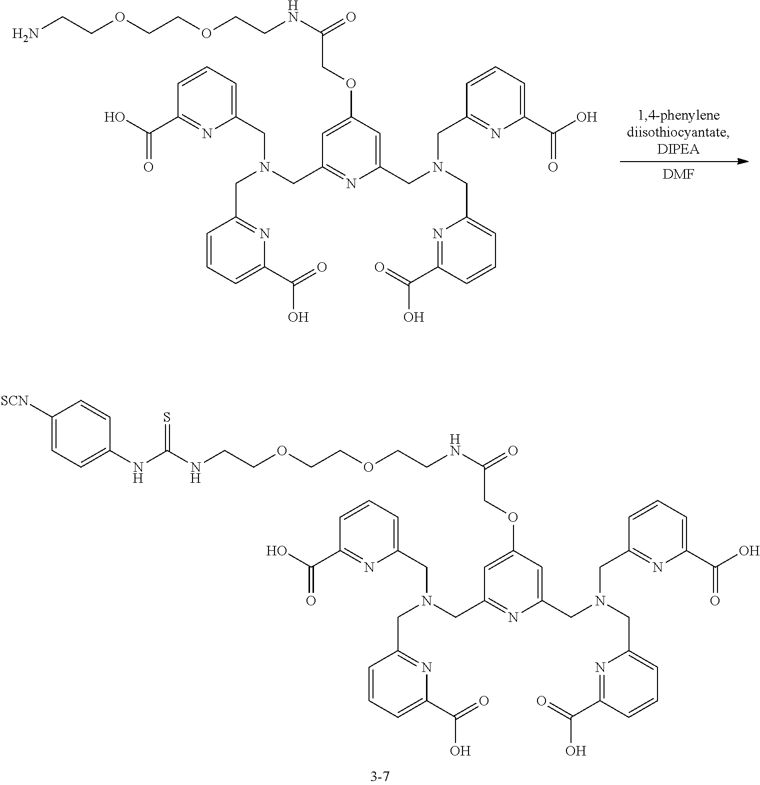

### Synthesis of tetra-tert-butyl 6,6′,6″,6′″-((((4-((2,2-dimethyl-4,15-dioxo-3,8,11-trioxa-5,14-diazahexadecan-16 yl)oxy)pyridine-2,6-diyl)bis(methylene))bis(azanetriyl))tetrakis (methylene))tetrapicolinate

To a stirred solution of Intermediate A (650 mg, 0.708 mmol) in dry ACN (10 mL) were added anhydrous K₂CO₃ (294 mg, 0.850 mmol) and tert-butyl N-(2-{2-[2-(2-bromoacetamido)ethoxy]ethoxy}ethyl)carbamate (313 mg, 0.850 mmol). The mixture was stirred at RT for 72 h. After the reaction was completed, the solvent was evaporated, and the product mixture was purified through a silica column (Biostage, 25 g silica column, 1-20% DCM/Methanol) to yield a yellow oil (750 mg, 88%). ¹H NMR (400 MHz, DMSO-d₆) δ 8.00-7.87 (m, 4H), 7.85 (t, J=8.0 Hz, 4H), 7.70 (dd, J=10.7, 7.4 Hz, 2H), 6.83-6.71 (m, 2H), 4.61 (s, 2H), 3.87 (d, J=5.7 Hz, 3H), 3.45-3.41 (m, 5H), 3.35 (s, 12H), 3.26 (s, 4H), 3.10-2.99 (m, 4H), 1.56 (s, 9H), 1.53 (s, 36H). m/z found=1206.0 (M+H).

### Synthesis of 6,6′,6″,6′″-((((4-(2-((2-(2-(2-aminoethoxy)ethoxy)ethyl)amino)-2-oxoethoxy)pyridine-2,6-diyl)bis(methylene))bis(azanetriyl))tetrakis(methylene))tetrapicolinic acid

Compound tetra-tert-butyl 6,6′,6″,6′″-((((4-((2,2-dimethyl-4,15-dioxo-3,8,11-trioxa-5,14-diazahexadecan-16-yl)oxy)pyridine-2,6-diyl)bis(methylene))bis(azanetriyl))tetrakis (methylene))tetrapicolinate (200 mg, 0.166 mmol) was taken up in DCM (1 mL) and TFA (2 mL) was added. After 18 h, HPLC showed no starting material or t-butyl intermediates remaining. The reaction was concentrated in vacuo and co-evaporated once with DCM. The crude oil was triturated 2× with Et₂O with sonication and collected by filtration to yield a crude residue (120 mg, 82%). m/z found=881.8 (M+H).

### Synthesis of 6,6′,6″,6′″-((((4-((1-((4-isothiocyanatophenyl)amino)-12-oxo-1-thioxo-5,8-dioxa-2,11-diazatridecan-13-yl)oxy)pyridine-2,6-diyl)bis(methylene))bis(azanetriyl))tetrakis (methylene))tetrapicolinic acid

Compound 6,6′,6″,6′″-((((4-(2-((2-(2-(2-aminoethoxy)ethoxy)ethyl)amino)-2-oxoethoxy)pyridine-2,6-diyl)bis(methylene))bis(azanetriyl))tetrakis(methylene))tetrapicolinic acid (120 mg, 0.136 mmol) and 1,4-phenylene diisothiocyanate (104 mg, 0.544 mmol) were dissolved in 2.5 mL of DMF. To this mixture, DIPEA (71 μL, 0.408 mmol) in DMF (0.5 mL) was added dropwise over 5 min. The reaction mixture was stirred for 3 h. HPLC showed the reaction was complete. The reaction mixture was diluted with 50% MeCN/water (2.0 mL) with 0.1% TFA. Purified by prep-HPLC 20-60% MeCN/water (0.1% TFA) to yield the title compound (12 mg, 8%) as a white powder (3×TFA salt). ¹H NMR (400 MHz, DMSO-d₆) δ 7.96-7.84 (m, 4H), 7.76 (dd, J=5.8, 3.0 Hz, 5H), 7.56 (d, J=8.7 Hz, 4H), 7.46-7.27 (m, 5H), 4.78 (s, 2H), 4.08-4.03 (m, 12H), 3.74-3.34 (m, 15H). m/z found=1074.20 [M+H]; Calcd=1074.32.

### Example 8. Compound 3-8

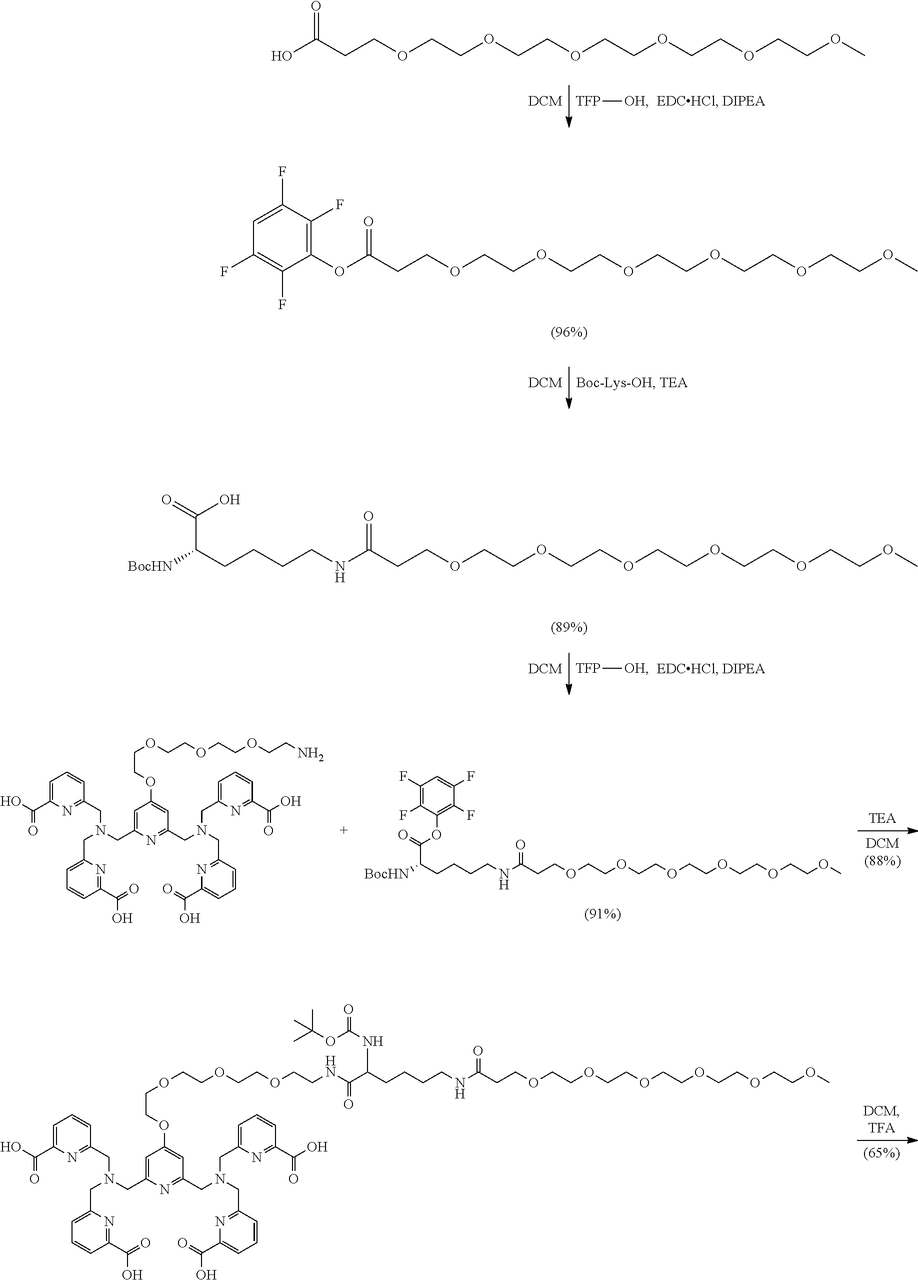

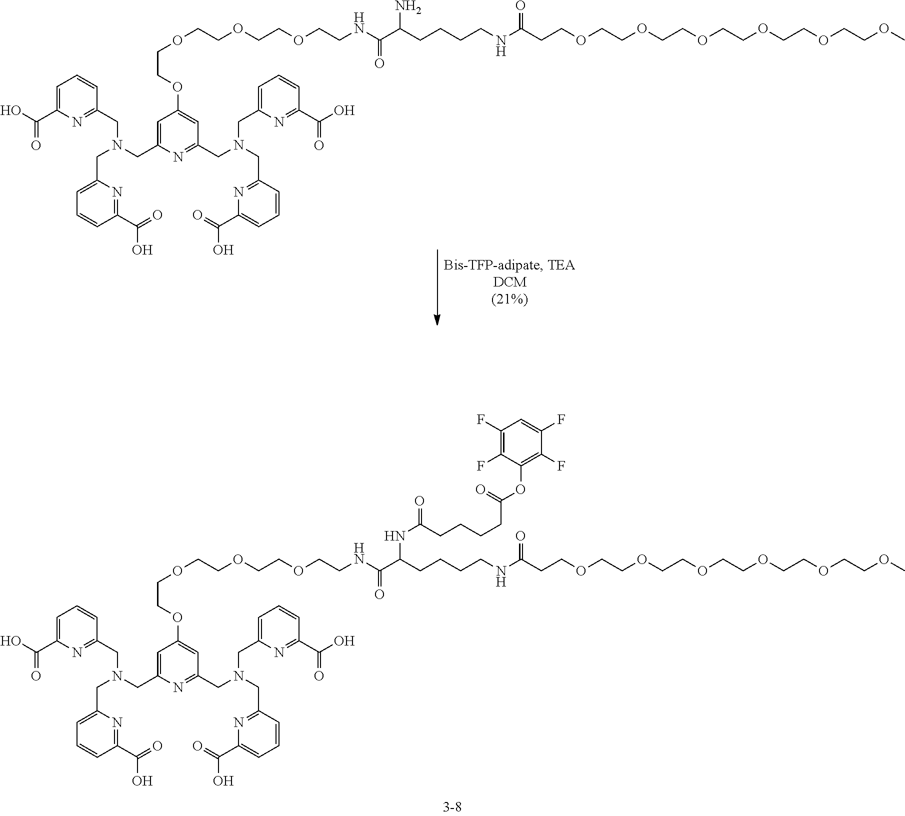 [SMILES: COCCOCCOCCOCCOCCOCCC(=O)NCCCCC(N)C(=O)NCCOCCOCCOCCOc1cc(CN(Cc2cccc(C(=O)O)n2)Cc2cccc(C(=O)O)n2)nc(CN(Cc2cccc(C(=O)O)n2)Cc2cccc(C(=O)O)n2)c1 ; COCCOCCOCCOCCOCCOCCC(=O)NCCCCC(NC(=O)CCCCC(=O)Oc1c(F)c(F)cc(F)c1F)C(=O)NCCOCCOCCOCCOc1cc(CN(Cc2cccc(C(=O)O)n2)Cc2cccc(C(=O)O)n2)nc(CN(Cc2cccc(C(=O)O)n2)Cc2cccc(C(=O)O)n2)c1]

### Synthesis of 2,3,5,6-tetrafluorophenyl 2,5,8,11,14,17-hexaoxaicosan-20-oate

DIPEA (752 μL, 4.32 mmol) was added to a solution of EDC·HCl (827 mg, 4.32 mmol) in DCM (2.5 mL). This solution was added to a solution of m-PEG6-COOH (394 mg, 1.21 mmol) in DCM (2.5 mL). 2,3,5,6-tetrafluorophenol (827 mg, 4.32 mmol) was dissolved in DCM (1.0 mL). The first mixture was added dropwise into it. The reaction was stirred at room temperature for 18 h. The desired product formation was confirmed by HPLC. The crude reaction was evaporated under a high vacuum, and the residue was taken in DCM and washed with sat. NaHCO₃ (2×) and brine (2×) solution. The DCM layer was dried over sodium sulfate, filtered, and concentrated in vacuo to obtain a crude product. Performed Biotage (25 g Silica column, 2-40% DCM/Methanol). Yield (1.4 g, 96%), ¹H NMR (400 MHz, CDCl₃) δ 6.22 (tt, J=10.5, 7.1 Hz, 1H), 3.91 (t, J=6.3 Hz, 2H), 3.74-3.60 (m, 20H), 3.40 (s, 3H), 2.98 (t, J=6.3 Hz, 2H). m/z found=490.2 (M+18).

### Synthesis of (S)-26-((tert-butoxycarbonyl)amino)-20-oxo-2,5,8,11,14,17-hexaoxa-21-azaheptacosan-27-oic acid

To a solution of 2,3,5,6-tetrafluorophenyl 2,5,8,11,14,17-hexaoxaicosan-20-oate (500 mg, 0.847 mmol) in DCM (2.0 mL) were added Boc-Lys-OH (250 mg, 0.847 mmol) and TEA (139 μL, 0.847 mmol). After stirring for 24 h, the desired product formation was confirmed by HPLC. The reaction mixture was washed with brine solution (2×), dried over sodium sulfate, filtered, and concentrated in vacuo to the obtained residue, purified by Biotage (10 g Silica column, 5-40% DCM/Methanol). Yield (440 mg, 95%), m/z found=553.3 (M+H).

### Synthesis of 2,3,5,6-tetrafluorophenyl (S)-26-((tert-butoxycarbonyl)amino)-20-oxo-2,5,8,11,14,17-hexaoxa-21-azaheptacosan-27-oate

DIPEA (307 μL, 1.76 mmol) was added to a solution of EDC·HCl (271 mg, 1.41 mmol) in DCM (2.5 mL). This solution was added to a solution of (S)-26-((tert-butoxycarbonyl)amino)-20-oxo-2,5,8,11,14,17-hexaoxa-21-azaheptacosan-27-oic acid (650 mg, 1.18 mmol) in DCM (2.5 mL). 2,3,5,6-tetrafluorophenol (273 mg, 1.65 mmol) was dissolved in DCM (1.0 mL). The first mixture was added dropwise into it. The reaction was stirred at RT for 18 h. The desired product formation was confirmed by HPLC. The crude reaction was evaporated under a high vacuum, and the residue was taken in DCM and washed with sat. NaHCO₃ (2×) and brine (2×) solution. The DCM layer was dried over sodium sulfate, filtered, and concentrated in vacuo to obtain a crude product. Performed Biotage (10 g Silica column, 2-40% DCM/Methanol). Yield (600 mg, 73%), ¹H NMR (400 MHz, CDCl₃) δ 6.57 (tt, J=10.2, 7.0 Hz, 1H), 4.66-4.50 (m, 1H), 3.74 (td, J=5.7, 3.0 Hz, 2H), 3.69-3.62 (m, 20H), 3.39 (s, 3H), 3.35-3.20 (m, 4H), 3.28 (s, OH), 2.02-1.78 (m, 4H), 1.68-1.54 (m, 2H), 1.48 (d, J=2.1 Hz, 9H), 1.43-1.31 (m, 2H). m/z found=701.3 (M+H).

### Synthesis of 6,6′,6″,6′″-((((4-((26-((tert-butoxycarbonyl)amino)-20,27-dioxo-2,5,8,11,14,17, 31,34,37-nonaoxa-21,28-diazanonatriacontan-39-yl)oxy)pyridine-2,6-diyl)bis(methylene)) bis(azanetriyl))tetrakis(methylene))tetrapicolinic acid

To a solution of 2,3,5,6-tetrafluorophenyl (S)-26-((tert-butoxycarbonyl)amino)-20-oxo-2,5,8,11,14,17-hexaoxa-21-azaheptacosan-27-oate (35 mg, 0.040 mmol) in DCM (1.0 mL), Intermediate A (40 mg, 0.056 mmol) and DIPEA (10 μL, 0.056 mmol) were added. After stirring for 18 h, the desired product formation was confirmed by HPLC. The crude product was triturated 2× with Et₂O with sonication and collected by filtration to yield a crude residue (50 mg, 88%). m/z found=702.4 (M+2H/2).

### Synthesis of 6,6′,6″,6′″-((((4-((26-amino-20,27-dioxo-2,5,8,11,14,17,31,34,37-nonaoxa-21,28-diazanonatriacontan-39-yl)oxy)pyridine-2,6-diyl)bis(methylene))bis(azanetriyl))tetrakis (methylene))tetrapicolinic acid

Compound 6,6′,6″,6′″-((((4-((26-((tert-butoxycarbonyl)amino)-20,27-dioxo-2,5,8,11,14,17,31,34,37-nonaoxa-21,28-diazanonatriacontan-39-yl)oxy)pyridine-2,6-diyl)bis(methylene))bis(azanetriyl))tetrakis(methylene))tetrapicolinic acid (50 mg, 0.036 mmol) was taken up in DCM (0.5 mL) and TFA (1 mL) was added. After 18 h, HPLC showed no starting material or t-butyl intermediates remaining. The reaction was concentrated in vacuo and co-evaporated once with DCM. The crude product was triturated 2× with Et₂O with sonication and collected by filtration to yield a crude residue (35 mg, 65%). m/z found=652.4 (M+2H/2).

### Synthesis of 6,6′,6″,6′″-((((4-((20,27-dioxo-26-(6-oxo-6-(2,3,5,6-tetrafluorophenoxy) hexanamido)-2,5,8,11,14,17,31,34,37-nonaoxa-21,28-diazanonatriacontan-39-yl)oxy)pyridine-2,6-diyl)bis(methylene))bis(azanetriyl))tetrakis(methylene))tetrapicolinic acid

To a solution of 6,6′,6″,6′″-((((4-((26-amino-20,27-dioxo-2,5,8,11,14,17,31,34,37-nonaoxa-21,28-diazanonatriacontan-39-yl)oxy)pyridine-2,6-diyl)bis(methylene))bis(azanetriyl)) tetrakis(methylene))tetrapicolinic acid (60.0 mg, 0.046 mmol) in DMF (1.0 mL), bis(2,3,5,6-tetrafluorophenyl) hexanedioate (81 mg, 0.184 mmol) and TEA (16 μL, 0.115 mmol). were added. After stirring for 18 h, the desired product formation was confirmed by HPLC. The reaction mixture was evaporated under a high-vacuum and obtained residue diluted with 50% MeCN/water (2.0 mL) with 0.1% TFA. Purified by prep-HPLC 20-60% MeCN/water (0.1% TFA) to yield the title compound (9 mg, 21%) as a white powder (2×TFA salt). ¹H NMR (400 MHz, D₂O) δ 7.91 (t, J=7.8 Hz, 4H), 7.79 (d, J=7.3 Hz, 4H), 7.55 (d, J=7.8 Hz, 4H), 7.21-7.09 (m, 1H), 6.91 (s, 2H), 4.36 (s, 8H), 4.29 (s, 4H), 4.16-4.09 (m, 2H), 4.07 (dd, J=9.0, 5.5 Hz, 1H), 3.87-3.73 (m, 2H), 3.68-3.58 (m, 4H), 3.58-3.50 (m, 26H), 3.50-3.42 (m, 4H), 3.29-3.20 (m, 5H), 3.10-3.02 (m, 1H), 2.99 (t, J=6.9 Hz, 2H), 2.60 (t, J=6.2 Hz, 2H), 2.40-2.30 (m, 3H), 2.27-2.15 (m, 2H), 1.93 (s, 2H), 1.66-1.47 (m, 4H). m/z found=1580.57 [M+H]; Calcd=1579.65.

### Example 9. Compound 3-9

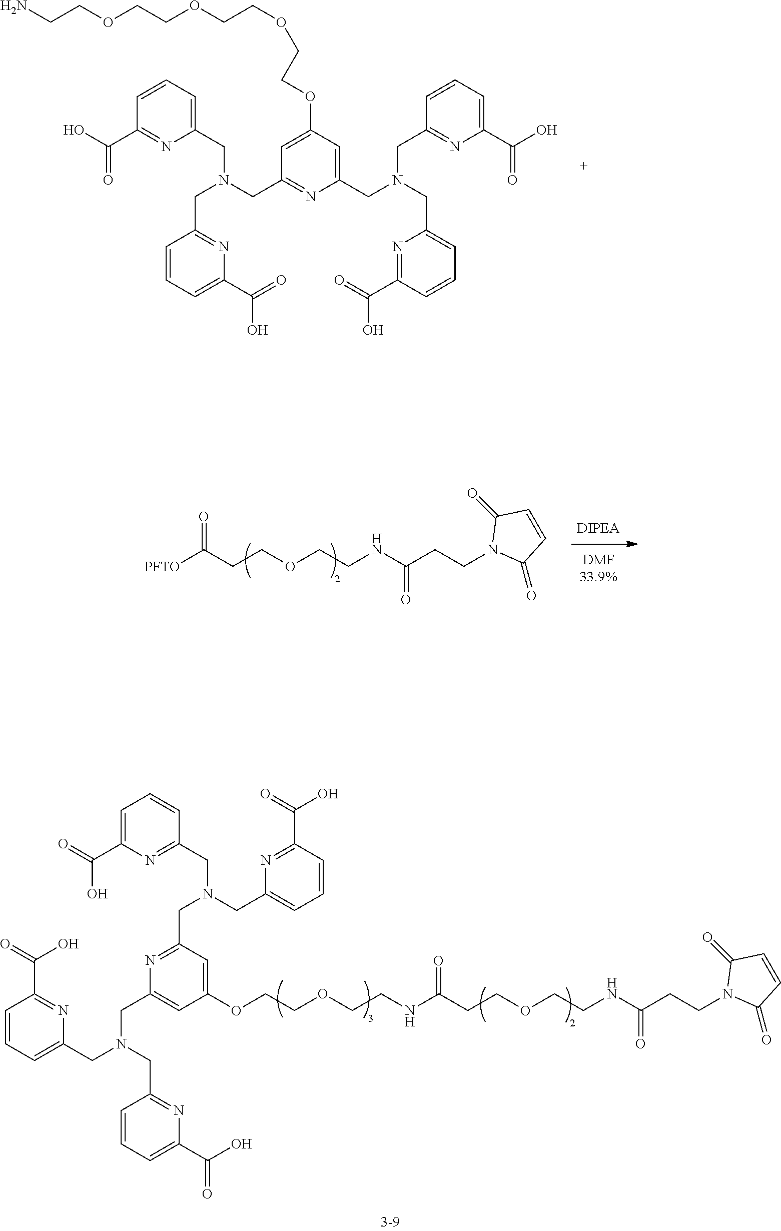

### Synthesis of compound (448126) 6,6′,6″,6′″-((((4-((25-(2,5-dioxo-2,5-dihydro-1H-pyrrol-1-yl)-13,23-dioxo-3,6,9,16,19-pentaoxa-12,22-diazapentacosyl)oxy)pyridine-2,6-diyl)bis(methylene))bis(azanetriyl))tetrakis(methylene))tetrapicolinic acid

To a stirred solution of 6,6′,6″,6′″-((((4-(2-(2-(2-(2-aminoethoxy)ethoxy)ethoxy)ethoxy)pyridine-2,6-diyl)bis(methylene))bis(azanetriyl))tetrakis(methylene))tetrapicolinic acid (50 mg, 0.058 mmol) in DMF (1 mL), MaI-PEG2-TFP (33 mg, 0.069 mmol) and DIPEA (20 μL, 0.115 mmol) were added. The mixture was stirred at RT for 4 h. After the reaction was completed, the solvent was evaporated and obtained residue diluted with 50% MeCN/water (2.0 mL) with 0.1% TFA. Purified by prep-HPLC 20-60% MeCN/water (0.1% TFA) to yield the title compound (23 mg, 34%) as a white powder (2.5×TFA salt). ¹H NMR (400 MHz, D₂O) δ 7.91 (t, J=7.8 Hz, 4H), 7.80 (d, J=7.8 Hz, 4H), 7.56 (d, J=7.6 Hz, 4H), 6.94 (s, 2H), 6.69 (s, 2H), 4.39 (s, 8H), 4.33 (s, 4H), 4.16-4.12 (m, 2H), 3.82-3.78 (m, 2H), 3.64 (d, J=5.5 Hz, 2H), 3.62-3.56 (m, 8H), 3.56-3.52 (m, 2H), 3.45 (t, J=5.7 Hz, 6H), 3.37 (t, J=5.4 Hz, 2H), 3.21 (t, J=5.4 Hz, 2H), 3.13 (t, J=5.4 Hz, 2H), 2.39-2.28 (m, 4H). m/z found=1179.30 [M+H]; Calcd=1179.46.

### Example 10. Compound 4-1 (p-SCN-Bn-DOTA)

In some examples, the chelator-linker is compound 4-1 ((R)-2,2′,2″,2′″-(2-(4-isothiocyanatobenzyl)-1,4,7,10-tetraazacyclododecane-1,4,7,10-tetrayl)tetraacetic acid), which was sourced from Macrocyclics (Plano, TX; Cat #: B-205)

### Example 11. Synthesis of Representative Comparative Examples

### Compound 4-2 (TFP-adipate-PEG5-DOTAGA)

In some examples, the chelator-linker is compound 4-2, which was prepared according to the examples in PCT/IB2022/000077.

### Compound 4-3 (TFP-adipate-PEG5-Py4Pa)

In some examples, the chelator-linker is compound 4-3, which was prepared according to the examples in PCT/IB2022/000077.

Compound 4-4 (p-SCN-Bn-Et-Py4Pa)

In some examples, the chelator-linker is compound 4-4, which was prepared by following the conditions in L Li et al. Bioconjugate Chem. 2021, 32, 1348-1363.

### BIOLOGICAL EXAMPLES

The Examples below describe radioisotope-delivering platforms having sizes between 60 and 110 kDa and which have shorter half-lives (e.g., 4 days or less) compared to traditional IgGs but longer half-lives than smaller monomeric antibody fragment formats (e.g., greater than 10 hours). Furthermore, certain radioisotope-delivering platforms provided herein exhibit high stability in vitro or in vivo, low immunogenicity, and suitable therapeutic windows. These radioisotope-delivering platforms are preferred for targeting radioisotopes in vivo in order to treat disease. These radioisotope-delivering platforms are particularly useful for targeted delivery of alpha emitters safely and effectively in a subject by exhibiting reduced adverse effects as compared to antibodies having half-lives over 4 days and/or molecular weights under 60 kDa.

Below, in certain phrases, “Fc portion” is used in reference to variant constant domain and “hinge” is used in reference to “hinge region” as will be understood by the person of ordinary skill in the art.

### Example B-1. Antibody Production

VHH-Fc plasmids were generated by cloning the VHH sequence, with a hinge and Fc portion (human IgG1 CH2-CH3) into a mammalian expression vector. In some instances, mutations were introduced into the Fc portion. To produce recombinant VHH-Fc and variants thereof, plasmid was transfected into HEK293. SUS cells (ATUM, or similar). After 3-5 days of secretion, the antibody-containing supernatant was cleared of cells by centrifugation and sterile filtration. Antibodies were purified using Mab Select SuRe PCC column (GE, Cat #: 11003495) and buffer exchange into PBS, pH 7.0. Proteins were quantified using A280 or BCA. The purity of the antibodies were tested by SDS-PAGE, capillary electrophoresis, HPLC-SEC and LC-MS using standard protocols. Regarding VHH polypeptides, see, for example, McMahon et al., Nature Structural & Molecular Biology|VOL 25|March 2018|289-296 Yeast surface display platform for rapid discovery of conformationally selective nanobodies; Moutel et al., eLife 2016; 5:e16228 NaLi—H1: A universal synthetic library of humanized nanobodies providing highly functional antibodies and intrabodies. De Genst E, Saerens D, Muyldermans S, Conrath K. Antibody repertoire development in camelids. Dev Comp Immunol. 2006; 30(1-2):187-98. doi: 10.1016/j.dci.2005.06.010. PMID: 16051357. Vincke C, Gutierrez C, Wernery U, Devoogdt N, Hassanzadeh-Ghassabeh G, Muyldermans S. Generation of single domain antibody fragments derived from camelids and generation of manifold constructs. Methods Mol Biol. 2012; 907:145-76. doi: 10.1007/978-1-61779-974-7_8. PMID: 22907350. Arbabi Ghahroudi M, Desmyter A, Wyns L, Hamers R, Muyldermans S. Selection and identification of single domain antibody fragments from camel heavy-chain antibodies. FEBS Lett. 1997 Sep. 15; 414(3):521-6. doi: 10.1016/s0014-5793(97)01062-4. PMID: 9323027.

For VHH humanization, see, for example, Vincke C, Loris R, Saerens D, Martinez-Rodriguez S, Muyldermans S, Conrath K. General strategy to humanize a camelid single-domain antibody and identification of a universal humanized nanobody scaffold. J Biol Chem. 2009 Jan. 30; 284(5):3273-84. doi: 10.1074/jbc.M806889200. Epub 2008 Nov. 14. PMID: 19010777.

For VHH stability, see, for example, Kunz P, Flock T, Soler N, Zaiss M, Vincke C, Sterckx Y, Kastelic D, Muyldermans S, Hoheisel J D. Exploiting sequence and stability information for directing nanobody stability engineering. Biochim Biophys Acta Gen Subj. 2017 September; 1861(9):2196-2205. doi: 10.1016/j.bbagen.2017.06.014. Epub 2017 Jun. 20. PMID: 28642127; PMCID: PMC5548252; Kunz P, Zinner K, Mücke N, Bartoschik T, Muyldermans S, Hoheisel J D. The structural basis of nanobody unfolding reversibility and thermoresistance. Sci Rep. 2018 May 21; 8(1):7934. doi: 10.1038/s41598-018-26338-z. PMID: 29784954; PMCID: PMC5962586.

A number of VHH-Fc prototypes and variants were engineered using VHH sequences such as the anti-HER2 clone 2RS15d VHH (See. e.g., WO2016/016021) (SEQ ID NO: 20), and the anti-DLL3 clone hz10D9v7.251 VHH sequences (See e.g., WO2020/07967) (SEQ ID NO: 30), unless otherwise stated herein the data collected and shown was obtained using VHH antigen binding regions of these clones.

TABLE 1
Constructs

| VHH Fc | FcRn | Fc Effector | |
|---|---|---|---|
| name | Mutant | Mutant | Target |
| H101 | wt | wt | HER2 |
| D102 | wt | wt | DLL3 |
| H105 | I253A | wt | HER2 |
| H106 | S254A | wt | HER2 |
| H107 | H310A | wt | HER2 |
| H108 | H435Q | wt | HER2 |
| H109 | Y463A | wt | HER2 |
| D111 | I253A | wt | DLL3 |
| D112 | S254A | wt | DLL3 |
| D113 | H310A | wt | DLL3 |
| D114 | H435Q | wt | DLL3 |
| D115 | Y463A | wt | DLL3 |
| H133 | wt | AEASS | HER2 |
| D134 | wt | AEASS | DLL3 |
| H135 | H310A | AEASS | HER2 |
| D136 | H310A | AEASS | DLL3 |
| H137 | H435Q | AEASS | HER2 |
| D138 | H435Q | AEASS | DLL3 |

Variants per EU numbering; AEASS = L234A, L235E, G237A, A330S, and P331S

### Example B-2. Antibody Binding Properties: Assays for Target Protein and Target Cells

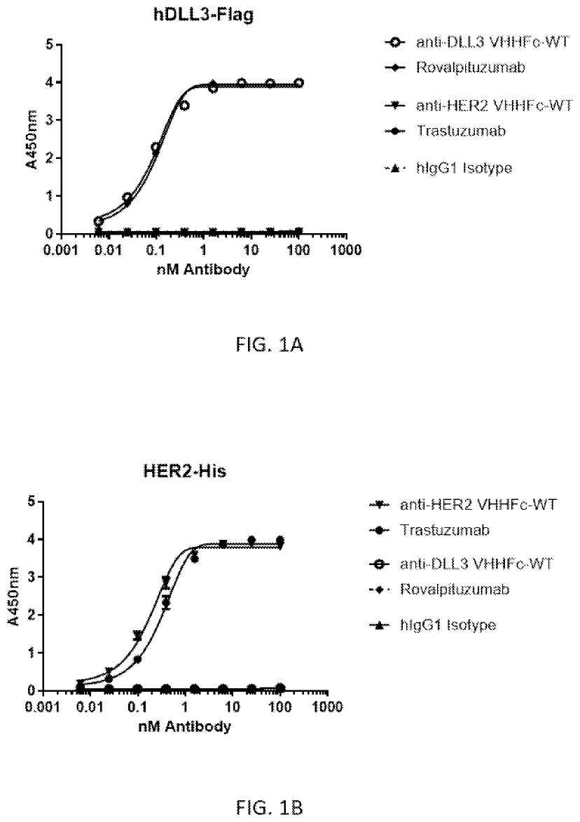

The VHH-Fcs were assessed by ELISA for binding to Target soluble protein-human, murine and cynomolgous orthologs as appropriate, according to standard protocols. Antigens were sourced commercially or produced by cloning known antigen sequences (Uniprot) into mammalian expression vectors with a HIS, FLAG or equivalent tag for purification and detection purposes. A commercially available control anti-target IgG was included. Plates (96-well maxisorp, Corning 3368) were coated with 50 to 100 μL of each Target protein of interest at a concentration optimized for coating. Purified VHH-Fc and hIgG1 isotype control (Sigma, Cat #15154) were prepared at starting concentrations of 200 to 400 nM and titrated 1:4 down. Following primary antibody incubation for 1 hour at room temperature (RT), and washing, 0.2 ug/ml of secondary HRP-labelled antibody was added and incubated for 1 h at RT (goat anti human-IgG-Fc-HRP Jackson, Cat #109-035-098). Reaction was detected using 50 μL/well of TMB (Neogen, Cat #308177). The color development was stopped with 1 M HCl (50 μl). Optical density (OD) was measured at 450 nm using Spectromax plate reader and data were processed using SoftMaxPro. Data shows anti-Target VHH-Fcs bind to human, murine and cynomolgous target protein. Recombinant DLL3 protein used was human DLL3.FLAG (Adipogen #AG-40B-0151, amino acid 27-466), or human DLL3.HIS (abcam #ab255797, amino acid 27-492), or murine DLL3.HIS (IPA custom, amino acid 25-477) or cynomolgous DLL3.HIS (Acrobiosystems #, amino acid 27-490). Control antibodies for DLL3 binding was Rovalpituzumab (Creative Biolabs #TAB-216CL) Recombinant HER2 protein used was human Her2.HIS (Sinobiologics, #10004-H08H) and murine HER2.HIS (Sinobiologics #50714-M08H). Control antibody for HER2 binding was Trastuzumab (DIN: 02240692, ROCHE).). FIGS. 1A and 1B show Anti-Her2 and anti-DLL3 VHH-Fcs binding specifically to soluble target antigen in an ELISA, additional VHH-Fcs comprising mutations in the Fc region to decrease effector function and/or FcRn binding were tested but did not significantly affect binding to target antigen.

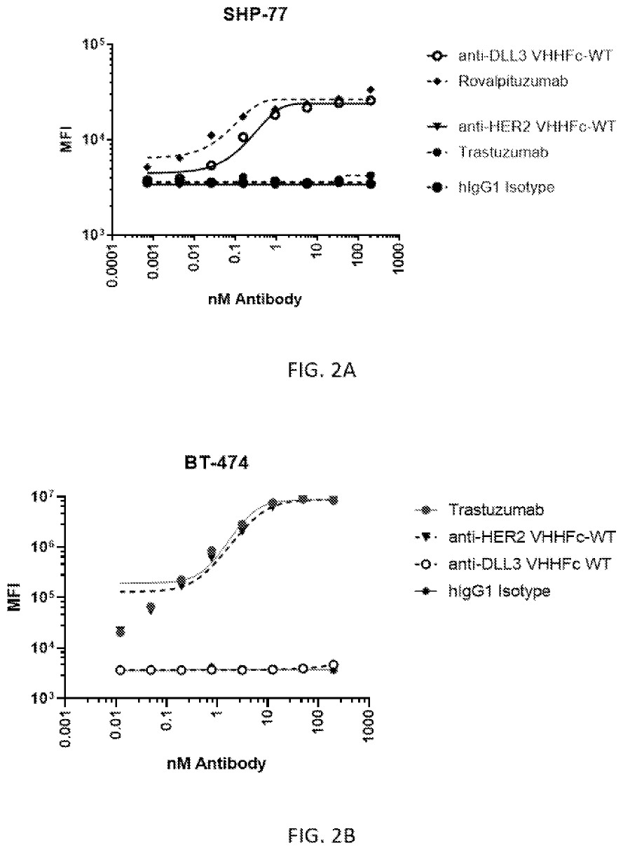

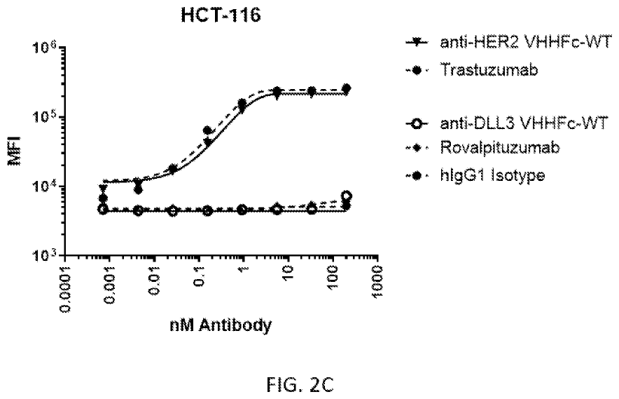

VHH-Fcs were screened for binding to a range of target-positive cancer cell lines by flow cytometry. All cell lines were sourced from ATCC unless otherwise noted, and cultured according to manufacturer's instructions and recommended media. HER2-positive cell lines used were SKBR3(ATCC #HTB-30) and BT474(ATCC #HTB-20) and HEK293-6E(NRC) cells. DLL3-positive cell lines tested include SHP-77(ATCC CRl-2195), NCI-H82(ATCC HTB-175), NCI-H69(ATCC HTB-119), HEK-DLL3 (Creative Biogene #CSC-R00531). HER2-negative cell lines tested included SHP-77. DLL3-negative cell lines tested included HCT-116 (CCL-247), BT-474 and SKBR3. Primary antibodies diluted in same manner as for ELISA were added to cells and incubated for 1 hour on ice. Cells were washed twice with 1% FBS in PBS, centrifuged at 450G for 4 minutes and incubated with 2 μg/mL AlexaFluor 647 conjugated anti-human IgG (Jackson, Cat #109-605-098) or AlexaFluor 647 conjugated anti-mouse IgG (Jackson, Cat #115-605-164) with 1:1000 DAPI (Biolegend, Cat #422801) for 30 minutes on ice. Following two further washes, cells were resuspended, and analyzed by flow cytometry on the iQue screener platform (Intellicyt), and data was processed with Forecyt, according to standard protocols. FIGS. 2A, 2B and 2C show binding to target-positive cell lines and shows that binding was specific to Target-positive cells (i.e., through binding comparison to negative controls cells). Further experiments indicated that Fc mutations to reduce effector function and/or FcRn binding did not impact binding to cancer cells as compared to wildtype Fcs.

### Example B-3. Internalization Assays

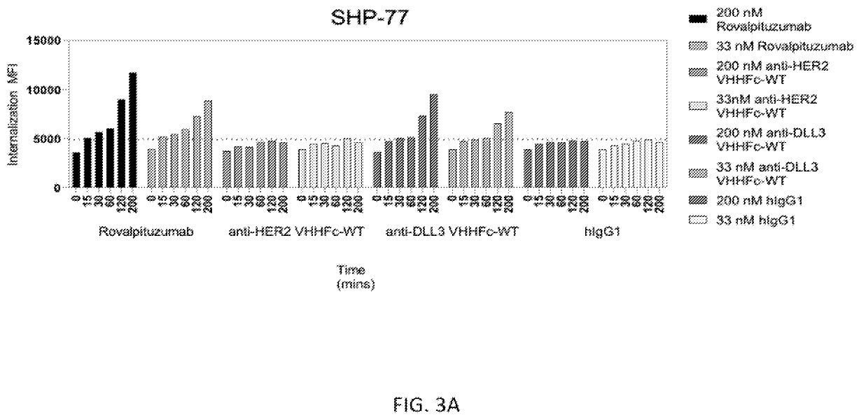

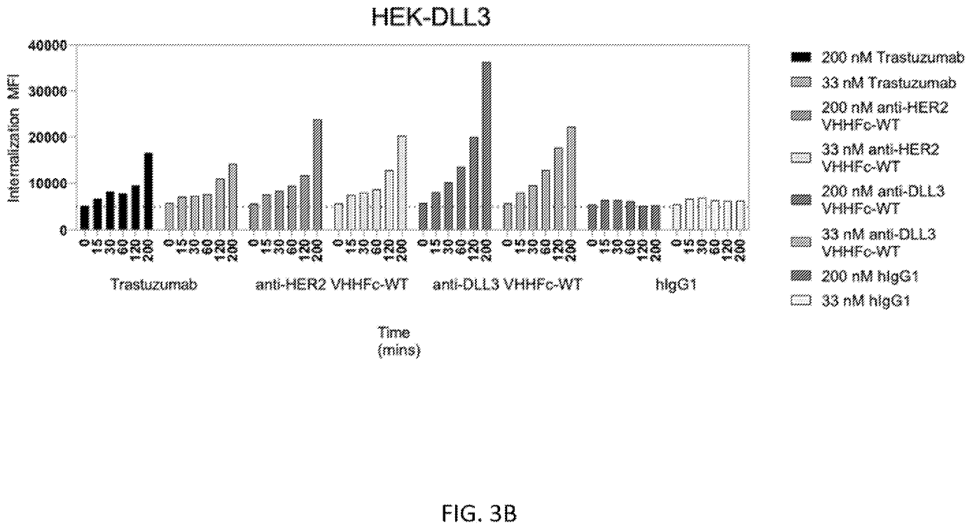

VHH-Fcs were tested for internalization by target-expressing cells using a secondary antibody conjugated to a pH sensitive dye. Goat anti-hu IgG-Fc secondary antibody was amine-conjugated to a pH sensitive pHAb dye (Promega Cat #G9845) according to the manufacturer's instructions. The pHAb dye has low or no fluorescence at pH>7 but fluoresces in acidic environment upon antibody internalization. Target-positive cells and target-negative cells were plated at 1.0×106/mL in a 96-well V bottom plate. V_{H}H-Fcs and hIgG1 isotype control were diluted in media to 75 nM. Cells were spun to remove supernatant, resuspended with the prepared primary antibodies and incubated on ice for 1 hour. Excess primary antibody was washed off from cells and then incubated with pHAb labelled secondary antibody on ice for 30 minutes. Excess secondary was then washed off and cells were resuspended in media. One set of samples was placed in an incubator at 37° C. to allow internalization, and another set was left on ice (0° C.) as a binding only control. Cells were sampled at different time points ranging from 0 to 24 hours. Cells were stained with DAPI and read by flow cytometry on 572/28 channel with iQue screener platform. The VHH-Fcs show higher fluorescence than the negative controls (isotype, buffer) on target-positive cells. FIGS. 3A and 3B show that H101 and were D102 internalized by SHP-77 and HEK-DLL3 cells.

### Example B-4. Antibody Thermal Stability Determination

Denaturing temperatures (Tm) of VHH-Fes were determined from differential scanning fluorimetry (DSF) using Protein Thermo Shift Dye Kit™ (ThermoFisher, Cat #: 4461146). Briefly, A total of 1 μg of antibody was used in each reaction. Melting curves of the antibodies were generated using an Applied Biosystems QuantStudio 7 Flex Real-Time PCR System with the recommended settings stated in the kit manual. The Tm's of the antibodies in Table 1 were then determined by using the ThermoFisher Protein Thermal Shift software (v.1.3). Tm1 of the VHH-Fcs was determined by DSF. Both H101 and D102 showed good thermostability of 67.5±0.1 Celsius. Additional, VHH-Fcs comprising mutations in the Fc region to decrease effector function and/or FcRn binding were tested for thermostability and resulted in slightly lower thermostability (1 to 2 degrees Celsius), but were still within acceptable ranges.

### Example B-5. Receptor Density Determination

In order to test efficacy of the immunoconjugate binding with respect to target density receptor density was measured on target positive cell lines. Target density was measured using the ABC (Antibody Binding Capacity) assay. Cancer cells expressing the target of interest, as well as a negative control cell line, were harvested with cell dissociation buffer, seeded at about 5×104 cells per well into 96-well V bottom plate (Sarstedt 82.1583.001). Cells were tested for receptor expression using QuantiBRITE PE beads (BD Cat #340495) and a PE-conjugated anti-hu IgG (Biolegend clone HP6017) following the manufacturers' instructions. In brief, VHH-Fc and isotype control antibodies were prepared at suitable saturating concentrations based on previous experiments. Antibody sample dilutions were incubated with the panel of cell lines on ice for 1 hour. Cells were washed twice with 1% FBS in 1×PBS (FACS buffer), centrifuged at 400 G for 4 min. Cells were then incubated with 4 μg/mL mouse PE-conjugated anti-hu and DAPI (1:1000) for 30 minutes on ice. Cells were washed twice with FACS buffer, centrifuged at 400 G for 4 minutes and resuspended in FACS buffer. Fluorescence intensity on the PE channel was measured on the iQue Screener platform, and data were processed with ForeCyt software. The amount of PE signal generated from the different primary antibody was then fit to a standard curve based off of known PE molecules/Quantibrite bead samples to determine the number of antibody-binding sites per cell. Relative antibody binding sites correlate to the number of antigens or receptors on cell surface. Table 2 shows receptor density numbers for anti-DLL3 and anti-HER2 VHH-Fcs binding to a panel of cancer cell lines and were similar ranges to those reported in literature.

TABLE 2
Estimated number of epitopes/cell for each binder and cell line

| | | SHP- | HEK- | | | HEK293- | HCT- |
|---|---|---|---|---|---|---|---|
| | | 77 | DLL3 | BT474 | H82 | 6E | 116 |
| Anti- | Rova | 969 | 1679 | — | 936 | — | — |
| DLL3 | D102 | 807 | 1734 | — | 794 | — | — |
| Anti- | Tmab | 625 | 1575 | 356690 | — | 1969 | 2790 |
| HER2 | H101 | 572 | 1490 | 401604 | — | 1935 | 2604 |

### Example B-6. Affinity of Antibodies to Target Protein

Antibody affinity was assessed using Octet Red96e (ForteBio). The association rate constant (ka), dissociation rate constant (kd) and affinity constant (KD) were measured by biolayer interferometry with anti-hIgG Fc (AHC) capture biosensors (Fortebio cat #18-5063). Each cycle was performed with orbital shake speed of 1,000 rpm. Antigen was titrated 1:2 from a suitable starting concentration in kinetics buffer (Fortebio, Cat #18-1105). A set of AHC biosensors was dipped in kinetics buffer for baseline step of 60s. Anti-Target VHH-Fc (5 μg/mL, in kinetics buffer) was loaded onto the biosensors for 240 s followed by a second baseline step of 30 s. The IgG captured sensors were dipped into buffer for single reference subtraction to compensate natural dissociation of capture IgG. Each biosensor was then dipped into corresponding concentration of target protein (human, murine or cynomolgus monomeric protein) for 600 s, followed by 1800 s of dissociation time in kinetics buffer, or conditions as optimized. A new set of AHC biosensors was used for every VHH-Fc. The data was analyzed by global fit 1:1 model for the association and dissociation step, (Octet software version v11.0). Table 3 shows binding affinity data.

TABLE 3
Affinity of H101 and D102 to target proteins

| VHH-Fc | Analyte | KD (nM) |
|---|---|---|
| D102 | Human DLL3-Flag | 0.472 |
| D102 | Mouse DLL3-His | 8.75 |
| H101 | Human HER2-His | 3.79 |

### Example B-7. FcRn and Fc Effector Mutation Affinity Determination

FcRn affinity of VHH-Fc can generally be used to predict the half-life of antibody serum clearance. (See, e.g., Datta-Mannan A et al. “FcRn affinity-pharmacokinetic relationship of five human IgG4 antibodies engineered for improved in vitro FcRn binding properties in cynomolgus monkeys.” Drug Metab Dispos. 2012 August; 40(8):1545-55). Briefly, 10 nM of biotinylated hFcRn (Sino Biological, Cat #: CT071-H27H-B) was captured with the SA biosensor using Octet RED96e (Fortebio). The hFcRN coated biosensor was dipped into the sample solutions in sodium phosphate buffer (100 mM Na₂HPO₄, 150 mM NaCl w/ 0.05% Tween-20, pH 6.0) with serial concentrations of tested antibodies and the association measured. The dissociation was measured by dipping the biosensors into sodium phosphate buffer without antibody. The K_{D} values were determined using Octet Data Analysis HT 11.0 software. 2:1 (Heterogeneous Ligand) binding model was used in analysis. Table 4 shows FCRN affinity for wildtype VHH-Fcs, and the impact of specific mutations in the Fc on affinity for the mutants. Changes in FcRn affinity were consistent across targets. Constructs with Fc Effector mutation only have no impact on FcRn affinity. Addition of Fc Effector mutations to FcRn mutation constructs does not affect FcRn affinity. Table 4 shows affinities of VHH-Fcs and Fc variants to FcRn.

TABLE 4a
Affinity of FcRn VHH-Fcs and Fc variants to FcRn

| VHH.Fc | FcRn Mutant | KD (nM) |
|---|---|---|
| H101 | wt | 3.7 |
| D102 | wt | 3.8 |
| H105 | I253A | Weak |
| H106 | S254A | 13 |
| H107 | H310A | No binding |
| H108 | H435Q | Weak |
| H109 | Y463A | 13 |
| H110 | H310A/H435Q | No binding |
| D111 | I253A | Weak |
| D112 | S254A | 19 |
| D113 | H310A | No binding |
| D114 | H435Q | Weak |
| D115 | Y463A | 20 |
| D116 | H310A/H435Q | No binding |
| H133 | wt | 2.1 |
| D134 | wt | 1.9 |
| H135 | H310A | No binding |
| D136 | H310A | No binding |
| H137 | H435Q | Weak |
| D138 | H435Q | Weak |

VHH-Fcs were also tested for affinity to FcγRs by biolayer interferometry using the Octet Red96e platform. Each cycle is performed with orbital shake speed of 1,000 rpm. Streptavidin (SA) biosensors (Sartorius 18-5019) were rehydrated for 10 mins using kinetics buffer (PBS+0.1% BSA+0.02% Tween-20). Biotinylated-FcγRs (Acro Biosystems) were then loaded for 40-100 s onto SA biosensors at concentrations ranging between 1-5 μg/mL diluted in PBS. VHH-Fcs were serially diluted 1:2 in sample buffer (PBS+0.02% Tween-20) with starting concentrations ranging between 5000 nM to 37.5 nM. Loaded biosensors were then associated with VHH-Fcs for 60-120 s. VHH-Fc dissociation was measured for 30-900 s in sample buffer. Bound VHH-Fcs were then removed using 3 cycles of 5 s regeneration buffer (150 mM NaCl, 300 mM Sodium Citrate) and 5 s sample buffer. The data was analyzed either using a globally-fitted 1:1 Langmuir binding model (FcγRI) or steady state analysis (Octet software version HT v11.1).

Analysis shows reduction in binding (represented by a higher K_{D}) to FcγRs for constructs with those mutations incorporated as shown in Table 4b.

TABLE 4b
Affinity of FcRn VHH-Fcs and Fc variants to Fc receptors

| | | | FcγRIIa | FcγRIIa | | FcγRIIIa | FcγRIIIa |
|---|---|---|---|---|---|---|---|
| | Fc | FcγRI | (H167) | (R167) | FcγRIIb/c | (F176) | (V176) |
| | mutation | nM KD | nM KD | nM KD | nM KD | nM KD | nM KD |
| Trastuzumab | wt | 0.92 | 270 | 520 | 3700 | 630 | 110 |
| H101 | wt | 1.01 | 340 | 160 | 450 | 1600 | 480 |
| H133 | AEASS | 1 | — | 2300 | weak | — | — |
| H135 | AEASS + | — | — | 1200 | weak | — | — |
| | H310A | | | | | | |
| H137 | AEASS + | — | — | 1200 | weak | — | — |
| | H435Q | | | | | | |
| D102 | wt | 1.27 | 390 | 530 | 430 | 1200 | 730 |
| D134 | AEASS | — | weak | 460 | 1100 | — | — |
| D136 | AEASS + | — | weak | 570 | 2200 | — | — |
| | H310A | | | | | | |
| D138 | AEASS + | | weak | 520 | 770 | — | — |
| | H435Q | | | | | | |

(—) indicates no binding detected

### Example B-8. Self-Association Studies Using AC-SINS

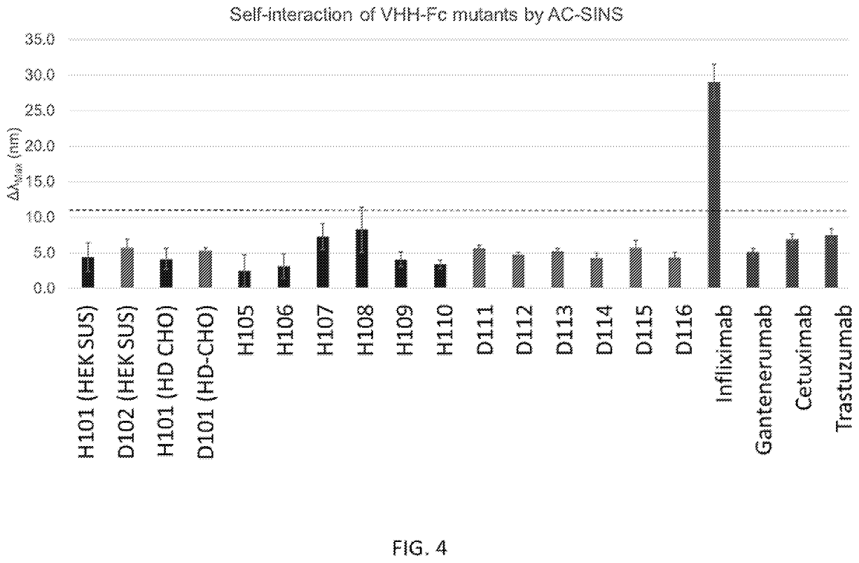

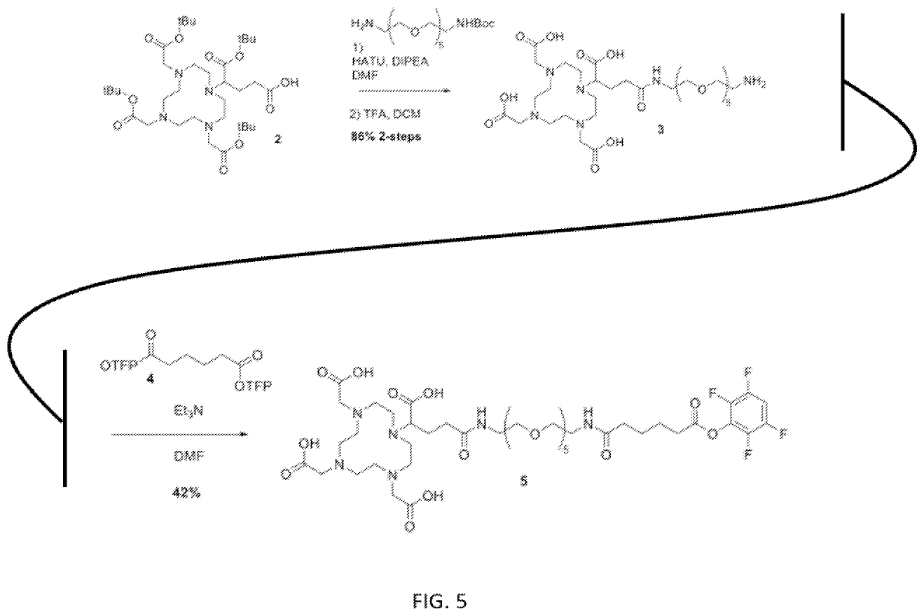

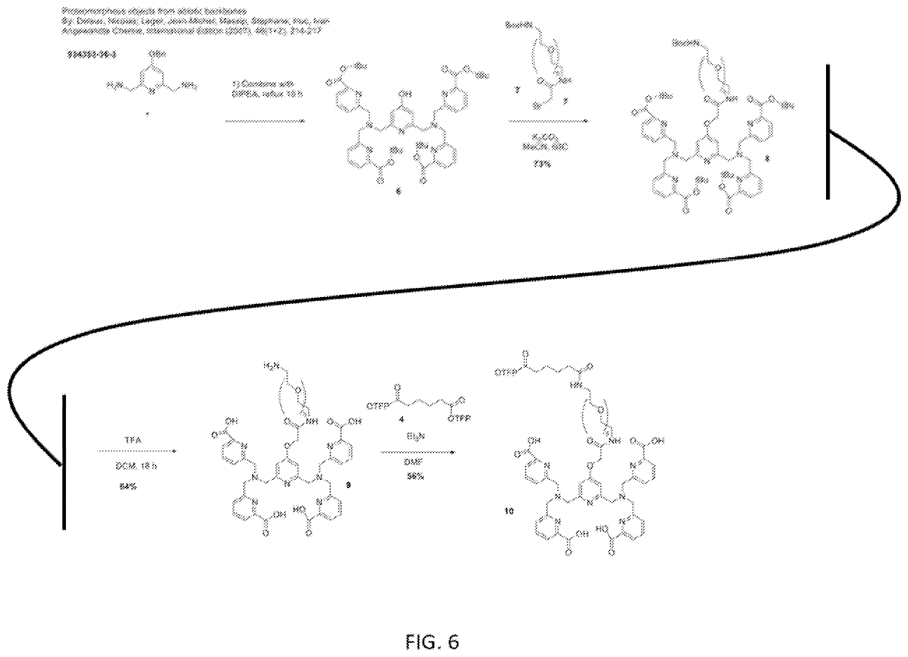

Propensities of self-association of VHH-Fcs was determined from affinity-Capture self-interaction nanoparticle spectroscopy (AC-SINS) using gold nanoparticles (Au—NP) (Ted Pella, Cat #: 15705). (PMID: 24492294, 30395473) Briefly, goat IgG and goat anti-human Fc IgG (1:4 mole ratio) were used to coat the Au—NP. Conjugated Au—NP was mixed with 5 ag of each VHH-Fc, in quadruplicates, in a 96-well plate. The wavelength scan was measured with Synergy Neo2 plate reader. The difference of maximum absorbance (Δλmax) was calculated by subtracting λmax of each reaction with that of PBS buffer. The data was analyzed with Linest function in Excel using second-order polynomial fitting. Control antibodies with known high ACSINS score (above the literature established cut-off of 11 for IgGs) were included in the assay. FIG. 4 shows ACSINS scores for test articles and controls.

### Example B-9. Polyreactivity Studies

Polyreactivity of VHH-Fcs against negatively charged biomolecules was determined by ELISA (As in Avery et al., “Establishing in vitro in vivo correlations to screen monoclonal antibodies for physicochemical properties related to favorable human pharmacokinetics.” MAbs. 2018 February/March; 10(2):244-255). Briefly, ELISA plate was coated with 5 μg/mL of human insulin (SigmaAlrich, Cat #: I9278) and 10 μg/mL of double stranded DNA (SigmaAlrich, Cat #: D1626-250MG) overnight. The plate was blocked with ELISA buffer (PBS, 1 mM EDTA, 0.05% Tween-20, pH 7.4). 10 μg/mL of test VHH-Fcs was loaded onto the plates in quadruplicates and incubated for 2 hours. Goat anti-human Fc(0.01 ug/ml) conjugated with HRP was then added and the plate incubated for 1 hour. The signal was developed with TMB and A450 absorbance was measured with Synergy Neo2 plate reader. The signal was normalized with the signal of non-coated well for each antibody tested. Table 5 shows the polyreactivity score, in comparison to control antibodies.

TABLE 5
Polyreactivity Assay Scores

| VHH.Fc | Insulin | dsDNA |
|---|---|---|
| H101 | 1.176 | 1.406 |
| D102 | 2.311 | 2.248 |
| H105 | 1.207 | |
| H106 | 1.321 | 1.446 |
| H107 | 1.306 | 1.678 |
| H108 | 1.420 | 1.663 |
| H109 | 1.244 | 1.579 |
| H110 | 1.181 | 1.317 |
| D111 | 2.202 | |
| D112 | 3.461 | 2.970 |
| D113 | 2.829 | 2.594 |
| D114 | 3.161 | 3.015 |
| D115 | 2.503 | 2.252 |
| D116 | 2.446 | 2.302 |
| Gantenerumab | >10 | >10 |

### Example B-10. Fc Variants Effectively Reduce VHH-Fc Half-Life

In certain instances, reducing the drug half-life of alpha emitters is important for safety and to avoid unwanted toxicity associated with treatment. However, antibodies generally have a half-life upwards of 14 days or greater. Therefore, the half-life of the VHH-Fc variants was tested in order to observe and measure any reductions in half-life.

Twenty eight (28) 8 week old male B6. Cg-Fcgrt^{tm1Der} Tg(FCGRT)32Dcr/DcrJ (Tg32 hom, JAX stock #014565) mice were distributed into 7 groups with 4 mice per group as outlined in the table. Tg32 mice comprise a humanized FcRn and are generally viewed as a surrogate for human pharmacokinetics of antibodies when compared to non-human primates. (See, e.g., Avery L B et al. “Utility of a human FcRn transgenic mouse model in drug discovery for early assessment and prediction of human pharmacokinetics of monoclonal antibodies.” MAbs. 2016 August-September; 8(6):1064-78). On Day 0, body weights were measured and test articles were IV administered to all mice at 3 mg/kg and 5 mL/kg. 25 μL blood samples were collected from each mouse at time intervals. The blood samples were collected into 1 μL K₃EDTA, processed to plasma, diluted 1/10 in 50% glycerol in PBS, transferred into specialized 96 well storage plates, and stored at −20° C. All plasma samples were assessed via a hIgG ELISA chosen for its high sensitivity for all seven test articles.

TABLE 6
Pharmacokinetic parameter summary for HER2 VHH-Fc

| | Terminal | | | AUC | Volume of |
|---|---|---|---|---|---|
| | Half-Life | Clearance | Cmax | μg- | Distribution |
| | days | mL/days | μg/mL | days/mL | mL |
| H105 | 1.12 | 152.1 | 63.9 | 841 | 137 |
| sem | 0.03 | 3.4 | 3.6 | 29 | 1 |
| H106 | 7.10 | 19.8 | 53.5 | 2193 | 177 |
| sem | 0.31 | 0.8 | 0.4 | 29 | 3 |
| H107 | 0.41 | 304.4 | 62.4 | 516 | 82 |
| sem | 0.01 | 15.0 | 4.3 | 22 | 3 |
| H108 | 1.57 | 117.5 | 46.6 | 903 | 174 |
| sem | 0.10 | 6.6 | 0.7 | 40 | 6 |
| H109 | 6.92 | 18.2 | 52.2 | 2519 | 152 |
| sem | 0.34 | 0.6 | 0.8 | 28 | 4 |
| H101 | 6.91 | 28.7 | 57.0 | 1946 | 218 |
| sem | 0.77 | 5.2 | 1.6 | 231 | 35 |
| trastuzumab | 14.54 | 5.9 | 59.0 | 4108 | 108 |
| sem | 1.12 | 0.5 | 2.3 | 109 | 2 |

As observed in Table 6, the introduction of mutations within the FcRn was generally able to reduce the half-life of the anti-HER2 VHH-Fc. Interestingly, contrary to published results in the field, not all Fc variants when included in the immunoconjugates tested showed a reduction in half-life consistent with previously published results found in the literature. (See, e.g., Burvenich I J et al., “Cross-species analysis of Fc engineered anti-Lewis-Y human IgG1 variants in human neonatal receptor transgenic mice reveal importance of S254 and Y436 in binding human neonatal Fc receptor.” MAbs. 2016 May-June; 8(4) 775-86).

TABLE 7
Pharmacokinetic summary for DLL3 VHH-Fc

| | Terminal | |
|---|---|---|
| | Half-Life | Clearance |
| | days | mL/days |
| D111 | 10.2 | 10.8 |
| SEM | 4.4 | 10.5 |
| D112 | 14.2 | 7.8 |
| SEM | 3.2 | 1.0 |
| D113 | 1.1 | 254.8 |
| SEM | 0.2 | 27.0 |
| D114 | 2.5 | 46.9 |
| SEM | 0.1 | 3.6 |
| D115 | 11.0 | 6.9 |
| SEM | 13.2 | 1.1 |
| D102 | 13.3 | 10.3 |
| SEM | 3.3 | 3.8 |
| trastuzumab | 18.4 | 3.7 |
| SEM | 5.9 | 0.9 |

As observed in Table 7, the introduction of mutations within the FcRn was generally able to reduce the half-life of the anti-DLL3 VHH-Fc. Similarly to HER2 binding immunoconjugates and contrary to published results, not all Fc variants showed a reduction in half-life consistent with previously published results found in the literature.

### Example B-11. VHH-Fc Intact Mass Analysis

VHH-Fc antibodies were deglycosylated prior to analysis with in-house Endo-S enzyme (final concentration of 10 μg/mL) or commercial Endo-S(New England BioLabs Inc., Cat #: P0741L) at 37° C. for 1 hour.

For analysis of the intact mass, 8 μL samples were injected on a Waters Acquity UPLC-Q-TOF with a UPLC BEH200 SEC 1.7 μM 4.6×150 mm column. These samples were eluted with a mobile phase of water/ACN (70/30, v/v) with 0.1% TFA and 0.1% FA (formic acid) for 11 min with a flow rate of 0.25 mL/min.

### Example B-12. Sourcing Bifunctional Chelators

Conjugations can be carried out using many of the methods available for preparation of IgG radioconjugates and IgG antibody-drug conjugates. For information on the range of applicable methodologies, see PW Howard Antibody-Drug Conjugates (ADCs), Protein Therapeutics, First Edition, chapter 9, pp. 278-279 (2017).

For a typical lysine-based conjugation, an antibody was buffer-exchanged into 0.1 M NaHCO₃, pH 9.0 by either Microsep Advance Centrifugal Device (Pall, 10K MWCO, Omega modified PES membrane, Cat #: MCP010C41), Amicon Ultra Centrifugal Filter Unit (Millipore, 40 Kda MWCO, Regenerated Cellulose membrane, Cat #: UFC803096), or Vivaspin Turbo 4 Ultrafiltration Unit (Sartorius, 30 kDa MWCO, High flux PES membrane, Cat #: VS04T21) followed by sterilization with a Costar Spin-X Centrifuge Tube, 0.22 μm (Corning, Cat #: 8160). The buffer-exchanged antibody was quantified by either Pierce BCA assay (Thermo Scientific, Cat #: 23225) or A280 (Unchained Labs, Lunatic). In some cases, the antibody may have been concentrated such that it could be adjusted to a concentration of 5 mg/mL after buffer exchange into the 0.1 M NaHCO₃, pH 9.0; in most procedures the antibody was diluted to 2 mg/mL in 0.1 M NaHCO₃, pH 9.0 by adding additional 0.1 M NaHCO₃ after buffer exchange.

Next, an appropriate molar excess (5-30 eq) of chelator-linker (20-50 mM in DMSO) was added to the mAb solution (2 or 5 mg/mL final concentration). In some cases, DMSO was added to make the solution a consistent 2-5% DMSO. The reaction was incubated at 25° C. overnight in an Eppendorf thermomixer C (with constant shaking at 300 rpm). After the reaction was complete, the sample was passed through a Zeba spin desalting column (40 KDa MWCO, ThermoFisher, Cat #: 87770) according to the manufacturer's protocol to remove unused chelator-linker and buffer-exchange into PBS (pH 7.4) (LifeTechnologies, Cat #: 10010-023). Each antibody-chelator conjugate (ACC) was stored at 4° C. until analysis and purification.

Representative results of conjugation experiments are presented in Table 8A.

TABLE 8A

| | | | | | | | | Chelator- | |
|---|---|---|---|---|---|---|---|---|---|
| | VHH | Chelator/ | Reaction | MS- | Monomer | Conc. | Amount | linker | Protein |
| Cmpd | Type | Ab Ratio | time | CAR | (%) | (mg/mL) | (mg) | Efficiency | Recovery |
| 3-2 | DLL3 | 2 | 2 hr | 0.4 | 96% | 1.7 | 0.16 | 27% | 78% |
| | DLL3 | 7 | 2 hr | 2.4 | 96% | 0.8 | 0.07 | 34% | 63% |

### Example B-13. Purification with SEC

To remove high molecular weight species (HMWS) and low molecular weight species (LMWS), VHH-Fcs were purified by SEC using an AKTA Pure FPLC system with a Cytiva HiLoad 16/600 Superdex 200 μg column. TBS buffer (50 mM Tris, 150 mM NaCl, OmniTrace Ultra water [VWR, Cat #: CAWX0003-2]), pH 7.6 was used for the SEC buffer. The fractions containing intact VHH-Fcs were pooled together and concentrated using Microsep Advance Centrifugal Device (Pall 10k MWCO, Cat #: MCP010C41). The concentrated sample was transferred to an Ultrafree-MC GV Centrifugal Filter, 0.22 μm 0.5 mL (Millipore, Cat #: UFC30GV0S) and spun at 3,000×g for 3 minutes.

### Example B-14. Protein Quantification

For some samples, VHH-Fc protein or VHH-Fc conjugate content was quantified with a Pierce BCA Protein Assay Kit (Thermo, Cat #: 23225) standardized by Cetuximab (LIST/E: 094822, DIN 02271249, 2 mg/mL).

For other samples, VHH-Fc protein or VHH-Fc conjugate content was quantified by measuring the absorbance of the solution at 280 nm and the protein's calculated extinction coefficient at 280 nm using a 2 μL sample with a Lunatic UV/Vis spectrophotometer (Unchained Labs).

### Example B-15. Chelator to VHH-Fc Ratio (CAR) Analysis

The chelator loading, herein described as CAR (chelator-antibody ratio), can be analyzed through methods applicable to practitioners of the art of antibody conjugates. For a review of these methods in the context of ADCs, see A Wakankar et al., mAbs 3:161 (2011).

VHH-Fc antibodies were de-glycosylated prior to analysis with in-house Endo-S enzyme (final concentration of 10 μg/mL) of commercial Endo-S(New England BioLabs Inc., Cat #: P0741L) at 37° C. for 1 hour. For analysis of the intact mass, 8 μL samples were injected on to a Waters Acquity UPLC-Q-TOF with a BEH200 SEC 1.7 μM 4.6×150 mm column. These samples were eluted with a mobile phase of water/ACN (70/30, v/v) with 0.1% TFA and 0.1% FA (formic acid) for 11 min with a flow rate of 0.25 mL/min). Deconvoluted spectra showed a distribution of masses representing species with a certain number of chelator-linkers attached. The intensities of the peaks were used to calculate the average CAR of the conjugate preparation by taking the sum of each peak intensity multiplied by the number of modification represented by that mass, then dividing by the total intensity of all antibody masses. (could also put in the equation). In this case, a distribution of masses is obtained after spectrum deconvolution that allows calculation of the average CAR of the preparation.

Alternatively, some conjugates were analyzed by UPLC-Q-TOF-MS using an Agilent AdvanceBio 6545XT LC/QTOF mass spectrometer with a mobile phase gradient of starting at 80% mobile phase A (Milli-Q water with 0.1% Formic Acid) and 20% mobile phase B (Acetonitrile with 0.1% Formic Acid) for 4 min. then linearly changing to 90% mobile phase B over the next 7 min. at a constant flow rate of 0.6 mL/min on an Agilent PLRP-S column (ID. 2.1 mm/Length: 50 mm/Particle size: 5 μm/Pore size: 1000 Å, Part #: PL1912-1502PK). De-convolution of raw data and CAR calculations were done with Agilent's BioConfirm software (Version 12.0) on Agilent's MassHunter workstation.

### Example B-16. Binding of VHH-Fc Conjugates to Cells Expressing Target Protein

In some instances, conjugation can negatively impact binding of the VHH-Fc to the target protein. Binding of VHH-Fc conjugates was therefore tested, similar to as described above.

TABLE 8
Cell binding data of VHH-Fc chelator conjugates
EC50 (nM)

| | | SHP- | HCT- | HEK- | HEK- |
|---|---|---|---|---|---|
| | Antibody | 77 | 116 | DLL3 | 293 |
| Controls | Rovalpituzumab | 0.11 | — | 0.06 | — |
| | Trastuzumab | — | 1.16 | 1.06 | 0.69 |
| | hIgG1 | — | — | — | — |
| Compound | H101 (CAR 0) | — | 2.21 | 1.62 | 1.14 |
| 4-1 | H101 (CAR 0.6) | — | 1.96 | 1.73 | 1.16 |
| | H101 (CAR 1.1) | — | 2.46 | 1.39 | 1.42 |
| | H101 (CAR 2.3) | — | 3.34 | 2.05 | 1.68 |
| | H101 (CAR 2.7) | — | 2.96 | 1.88 | 1.58 |
| | H101 (CAR 4.6) | — | 5.63 | 2.99 | 2.27 |
| | H101 (CAR 8.3) | — | 5.32 | 4.43 | 3.52 |
| | D102 (CAR 0) | 0.53 | >100 | 1.42 | >10 |
| | D102 (CAR 0.9) | 0.41 | — | 0.48 | — |
| | D102 (CAR 4.7) | 0.38 | — | 0.56 | — |
| Compound | H101 (CAR 0) | — | 2.21 | 1.62 | 1.14 |
| 4-2 | H101 (CAR 2.0) | — | 4.01 | 3.99 | 3.11 |
| | H101 (CAR 8.9) | — | 40.11 | 28.37 | 28.89 |
| | D102 (CAR 0) | 0.53 | >100 | 1.42 | >10 |
| | D102 (CAR 2.7) | 0.50 | — | 0.58 | — |
| | D102 (CAR 9.3) | 0.60 | — | 0.91 | — |

H101 = Her2 antigen binding;
D102 = DLL3 antigen binding;
CAR = Chelator to VHH ratio

As observed in Table 8, binding was observed for both conjugates. As also shown in Table 8, binding was also observed across increasing chelator VHH-Fc ratios (CAR).

### Example B-17. Percent Intact Analysis

The percent intact immunoconjugate was established by HPLC-SEC. 12 μL of conjugate was added to a glass vial insert in a standard HPLC vial. 10 μL of sample was injected onto an Agilent HPLC-SEC with a Wyatt Technology WTC-050S5 SN:0429 BN WBD129 column and eluted with 1×PBS (100% o) for 40 min at a flow rate of 0.5 mL/min.

Alternatively, the same analysis is performed using a Tosoh Bioscience TSKgel G3000SW XL (ID. 7.8 mm/Length: 300 mm/Particle size: 5 μm/Pore size: 250 Å, Part #: 08541) column combined with a guard column (TSKgel SWXL Guard Column ID. 6.0 mm/Length: 40 mm/Particle size: 7 μm, Part #: 08543) using an isocratic elution with a mobile phase consisting of 78 mM KH₂PO₄, 122 mM K₂HPO₄, 250 mM KCl, 15% 2-Propanol, pH 7.0, 0.2 μm filtered, over 45 min at a constant flow rate of 0.5 mL/min.

### Example B-18. Endotoxin Level Determination

Endotoxin test was performed using Wako's Limulus Amebocyte Lysate Pyrostar™ ES-F Single Test (Cat #: WPESK-0015) according to manufactural protocol. The QC cutoff was set based on the maximum injection dose projected for each animal in the study while following appropriate animal care and FDA guidelines.

### Example B-19. Radiolabeling with In-111

40 μg of each of the 4 test articles was diluted to 100 μL with 0.1 M ammonium acetate buffer in a 500 μL lo-bind Eppendorf tube and 18-25 μL (20-22 MBq) of [¹¹¹In]InCl₃ was added and mixed with a pipette. The reaction mixtures were incubated at 37° C. in an incubator for 1 hour. The tubes were then transferred to a 4° C. fridge.

Incorporation of radionuclides was determined by spotting 0.5 μL of sample at the origin of a 1.5×10 cm iTLC strip. The strip was then placed in a 50 mL Falcon tube containing 2 mL of mobile phase (25 mM EDTA in pH 5 0.1 M sodium acetate buffer) until the solvent had reached the top of the strip. The strip was removed and exposed to a phosphor imaging plate which was then scanned in a Cyclone phosphor imager. Regions of interest were drawn over spots corresponding to migration of protein-bound and unbound ¹¹¹In and proportion in each calculated.

Radioconjugates were also analyzed by SEC-HPLC: A volume corresponding to 0.1-0.2 MBq of the sample was pipetted into a 500 μL lo-bind Eppendorf tube and the radioactivity measured in an ionization chamber. The sample was drawn up into a syringe and injected onto the HPLC system. Samples were eluted with PBS. The eluate from the system was collected and the radioactivity measured in order to determine the recovery from the column (corrected for activity remaining in the sample tube and the injection syringe).

TABLE 9
Indium-111 Radiolabeling Efficiency

| | |
|---|---|
| Chelator- | Labelling efficiency post-synthesis |

| Linker | Antibody | Attempt 1 | Attempt 2 |
|---|---|---|---|
| 4-1 | H101 | 95.9% | 96.5% |
| 4-2 | | 97.5% | 97.7% |
| 4-3 | D102 | 97.5% | 97.0% |
| 4-4 | | 98.1% | 97.2% |

### Example B-20. Radiolabeling with ²²⁵Ac

800 μg of each of the 4 test articles was diluted to 200 μL with 0.2 M ammonium acetate buffer pH 6.5 in a 500 μL lo-bind Eppendorf tube and 2 μL (400 kBq) of 225-Actinium chloride was added and mixed with a pipette. The reaction mixtures were incubated at 37° C. in an incubator for 1 hour in the case of the Py4Pa conjugates and 2 hours for the DOTA conjugates. The tubes were then transferred to a 4° C. fridge.

Incorporation was measured by spotting 0.5 μL of sample at the origin of a 1.5×10 cm iTLC strip and allowing it to dry for a few minutes. The strip was then placed in a 50 mL Falcon tube containing 2 mL of mobile phase (25 mM EDTA in pH 5 0.1 M sodium acetate buffer) until the solvent had reached the top of the strip. The strip was removed and allowed to equilibrate for at least 2 hours, after which it was exposed to a phosphor imaging plate which was then scanned in a Cyclone phosphor imager. Regions of interest were drawn over spots corresponding to the migration of protein-bound and un-bound ²¹⁵Ac- and the proportion in each calculated.

Alternately, samples could be assayed by HPLC-SEC: HPLC of DOTA conjugates used a BioSEP SEC 5 μm s3000 3007.88 mm column with 20% acetonitrile in PBS elution. HPLC of Py4Pa conjugates used a Wyatt 050S5 5 μm 500 Å 7.8×300 mm column with 20% acetonitrile in PBS elution).

50 μL of each sample was drawn up into a Hamilton syringe and injected onto the HPLC system. From 10-30 minutes post injection, 30 second fractions of the eluate (0.25 mL) were collected by hand into counting tubes. The fractions were allowed to reach secular equilibrium for 24 hours and then measured in a gamma counter. A 5 μL sample of each preparation was also counted to enable the recovery from the HPLC system to be calculated. Radiochemical purity was determined by determining the area under the peak for 18.5-22.5 mins and 19.5-23.5 mins for DOTA and Py4Pa conjugates, respectively, as a percentage of total counts. As shown in Table 10 all chelator-linker combinations showed-good labeling efficiency.

TABLE 10
Ac-225 Radiolabeling Efficiency

| Chelator- | | iTLC Labelling efficiency |
|---|---|---|
| Linker | Antibody | immediately after preparation |
| 4-1 | H101 | 92.0% |
| 4-2 | | 96.3% |
| 4-3 | | 93.1% |
| 4-4 | | 96.0% |
| 4-1 | D102 | 98.5% |
| 4-2 | | 99.5% |
| 4-3 | | 98.0% |
| 4-4 | | 100% |

### Example B-21. Stability of Radioconjugates

The stability of the radiolabeled immunoconjugates was tested, both for ²²⁵Ac and ¹¹¹In. VHH-Fc chelator-conjugates were radiolabeled (either ¹¹¹In or ²²⁵Ac) as described above. For stability in PBS, 50 μL of each labelled test article was then added to either 200 μL of PBS (with ¹¹¹In) or 200 μL PBS/ascorbate (with ²²⁵Ac) and stored at 4° C. For stability in serum, 50 μL of each labelled test article was added to 200 μL of mouse serum and incubated at 37° C. Aliquots of were taken at different time points and analyzed for radiochemical purity using iTLC and/or HPLC-SEC as described above. The results of these stability experiments are shown in Table 11 and Table 12 below and indicated that the radio conjugates were stable in both PBS and serum.

TABLE 11
Stability of Her2 and DLL3 conjugates labeled with ¹¹¹In

| Radiochemical | | | | |
|---|---|---|---|---|
| purity by | DLL3 (D102) | | HER2 (H101) | |
| HPLC (iTLC) | 4-1 | 4-2 | 4-1 | 4-2 |
| PBS 1 h | 97.5% | 98.1% | 97.5% | 98.4% |
| PBS 24 h | 89.1% | 95.2% | 96.5% | 98.4% |
| Serum 24 h | 94% (94%) | 98% (94%) | 97% | 94% |
| Serum 72 h | 92% (92%) | 96% (94%) | 100% (87%) | 100% (84%) |
| Serum 168 h | 92% (94%) | 95% | 95% (91%) | 92% |

TLC radiochemical incorporation values presented in parentheses. iTLC incorporation >95% except where shown

TABLE 12
Stability of Her2 and DLL3 conjugates labeled with ²²⁵Ac

| Radio- | | | | | | | | |
|---|---|---|---|---|---|---|---|---|
| chemical | | | | | | | | |
| purity by | | | | | | | | |
| HPLC | DLL3 (D102) | | | | HER2 (H101) | | | |
| (iTLC) | 4-1 | 4-2 | 4-3 | 4-4 | 4-1 | 4-2 | 4-3 | 4-4 |
| PBS 1h | 91% | 92% | 83% | 82% | 93% | 93% | 84% | N/D |
| PBS 24 h | 92% | 92% | 83% | 83% | 93% | 91% | 82% | 82% |
| Serum | 88% | 91% | 78% | 69% | 91% | 90% | 75% | 68% |
| 24 h | (94%) | | | | | | | |
| Serum | 89% | 90% | 73% | 65% | 89% | 85% | 74% | 61% |
| 72 h | (90%) | (94%) | | | (87%) | (94%) | | |
| Serum | 81% | 86% | 71% | 59% | 85% | 80% | 70% | 56% |
| 168 h | (91%) | | | | (89%) | | | |

TLC radiochemical incorporation values presented in parentheses. iTLC incorporation >95% except where shown

### Example B-22. Immunoreactivity of VHH-Fc Radioconjugates

The immunoreactive fraction (IRF) was determined though a method described by SK Sharma et al. in Nucl. Med. Biol. 2019, 71, 32-38. Samples were incubated overnight in PBS at 4° C. for analysis and before in vivo experiments, while some samples were incubated in serum at 37° C. for 3 and 7 days as an alternate measure of stability.

### Example B-23. Bead Coating

Dynabeads and antigen (0.15 nmol per 0.125 μg beads) were incubated in B/W buffer (25 μL/0.125 μg beads) at room temperature on a tube rotator for 30 minutes. The Eppendorfs were spun at 100×g for 15 seconds and placed on a magnetic rack for 3 minutes. The supernatant was removed and the beads washed with PBSF. 1 mg of beads was then resuspended in 200 μL of B/W buffer and 2 mg in 400 μL of B/W buffer. Control beads were prepared the same way, except with no antigen added to the tubes.

### Example B-24. Immunoreactive Fraction (IRF) Assay

The appropriate volume of beads (25 μL/0.125 mg beads) generated above was added to microcentrifuge tubes, prewashed with 1 mL PBSF. Radiolabeled VHH-Fc-conjugate (10 ng), block (10 or 50 μg unconjugated antibody; if required), and PBSF were added to each reaction to achieve a final volume of 350 μL. The samples were incubated at room temperature on a rotor for 30 minutes. After this the tubes were centrifuged at 100×g for 15 seconds and placed on a magnetic rack for 3 minutes. The supernatant was collected in a gamma counter tube. The beads were washed twice with 400 μL PBSF and collected in a separate gamma counter tube. The beads were finally resuspended in 500 μL PBSF and transferred to a gamma counter tube. The reaction tube was washed with 500 μL PBSF and this was added to the gamma counter tube containing the beads.

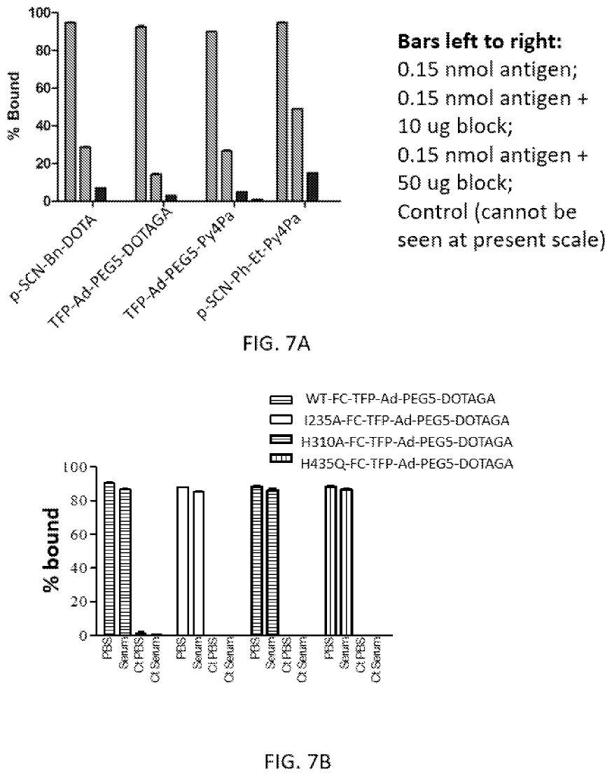

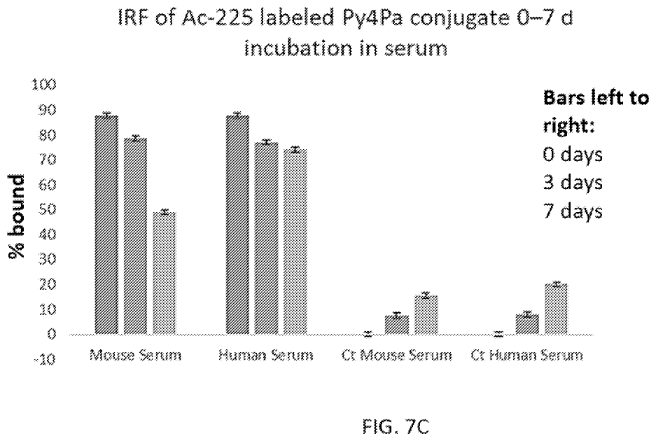

As shown in FIG. 7A for DLL3 all linker chelator combinations showed a similar immunoreactive fraction indicating no bias in labeling based upon the specific linker chelator combination, FIG. 7B shows that there was no effect due to Fc region mutations in immunoreactive fraction after 24 hours in PBS or serum, and FIG. 7C shows the immunoreactive fraction of ²²⁵Ac labeled anti-DLL3 VHH-Fc (D102) and stability in serum and plasma.

### Example B-25. Stability of Conjugates as Determined by IRF

The IRF assay presented above is used to probe the stability of the conjugates formed from the novel linkers. Using immunoreactivity to measure the % of radioactivity that binds to immobilized target antigen allows for an empirical determination of how much radioactivity remains targeted to bind to the antigen. Reduction in on-target binding of radioactivity allows for a comparative analysis of different chelator-linkers, different targeting vectors, and shows the degradation over time of the conjugate solution's ability to effectively target the desired antigen.

In brief, aliquots of the solutions in the stability analysis from Example B-21 (stability study) can also be applied to the IRF assay described in Example B-22-24 (IRF assay), and % bound reactivity is calculated and compared across various timepoints. In general, samples are taken at 0, 1, 3 and/or 7 days for the various assays.

### Example B-26. In Vivo Imaging Experiment with ¹¹¹In in Naïve Mice to Measure Total Body Clearance of Radioactivity Using Different Conjugates

Imaging (e.g., using ¹¹¹In) provides for the ability to collect pharmacokinetic and biodistribution data that can be used to perform dosimetry calculations for treatment planning. (See, e.g., Sgouros G, Hobbs R F. “Dosimetry for radiopharmaceutical therapy.” Semin Nucl Med. 2014 May; 44(3):172-8). Without being bound by theory, a quantitative demonstration of targeting observed with an imaging label is indicative of the ability to target with a radiolabel (e.g., an alpha emitter) capable of causing targeted cell death. A comparative analysis of the radioactivity present in the various tissues allows for a comparison of the relative performance and excretion profiles of conjugates prepared with different chelator-linkers.

In brief, CD-1 mice, female (n=21) are injected via the tail vein with one of 7 selected chelator-linker conjugates (7 groups), radiolabeled as above (Example B-19, scaled for study) with In-111 10 MBq at a specific activity of 0.5 MBq/ug (n=3 per group). The animals are imaged with whole-body SPECT under anesthesia followed by CT. Three time points (24 h, 72 h, and 144/168 h) are chosen. Data are analyzed to show whole body ID % (percent injected dose) and clearance. Images can be reprocessed later by drawing regions of interest (ROIs) to determine activity isolated in individual organs.

### Example B-27. Biodistribution of VHH-Fc Radioimmunoconjugates with Novel Chelator-Linkers

Similar to the experiment described above, purified conjugates from Examples B-12 and B-13 can be tested through a biodistribution experiment with tumor bearing mice. This study would be executed with similar group sizes and timepoints with SHP77 or BT474 tumor xenografts. The readouts from this study would suggest which compounds form conjugates with more favorable tumor to normal tissue uptake of the radioactivity.

In brief, this study will be executed with the DLL3 binding chelator-linker-VHH-Fc conjugates above with an H82 or SHP-77 xenograft model. Female athymic nude mice, 6-8 weeks old (n=30, 6 groups n=5) will be inoculated by injection of 5×10\hat{ }6 H82 cells into the lower flank. When tumors have reached the target volume of 150-250 mm3, the mice will be injected via the tail vein with one of 7 selected chelator-linker conjugates (7 groups), radiolabeled as above (Example B-19, scaled for study) with In-111 10 MBq at a specific activity of 0.5 MBq/ug (n=5 per group). Four animals from each group will be imaged with whole-body SPECT under anesthesia followed by CT. Three time points (24 h, 72 h, and 168 h) will be chosen. After the terminal imaging timepoint, the four animals will be euthanized and the following tissues will be analyzed for their residual activity to provide data on ex vivo biodistribution at 168 h: tumor, blood, liver, kidney, spleen, lymph nodes, femur head (knee), bone marrow, and tail.

### Example B-28. Biodistribution of VHH-Fc Radioimmunoconjugates

### Biodistribution and Tissue Accumulation Over Time in HER2+BT474 Tumors

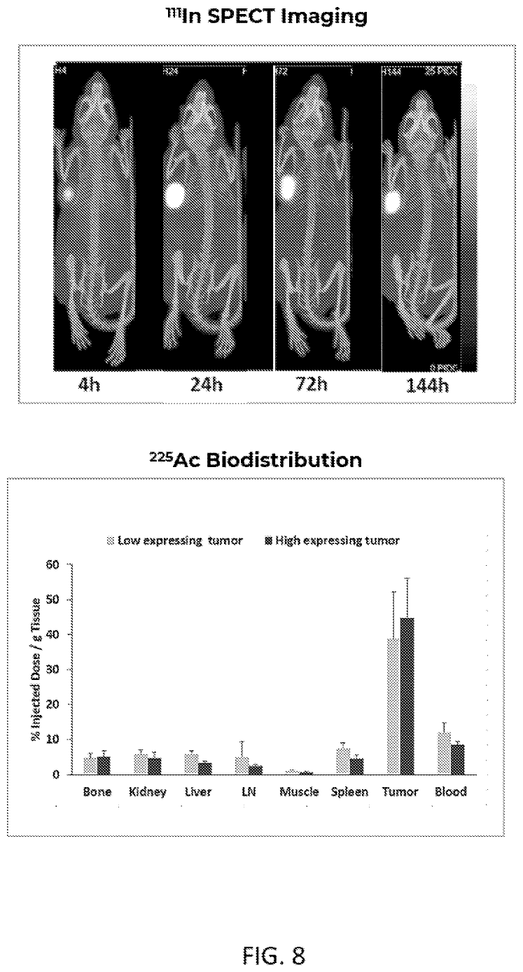

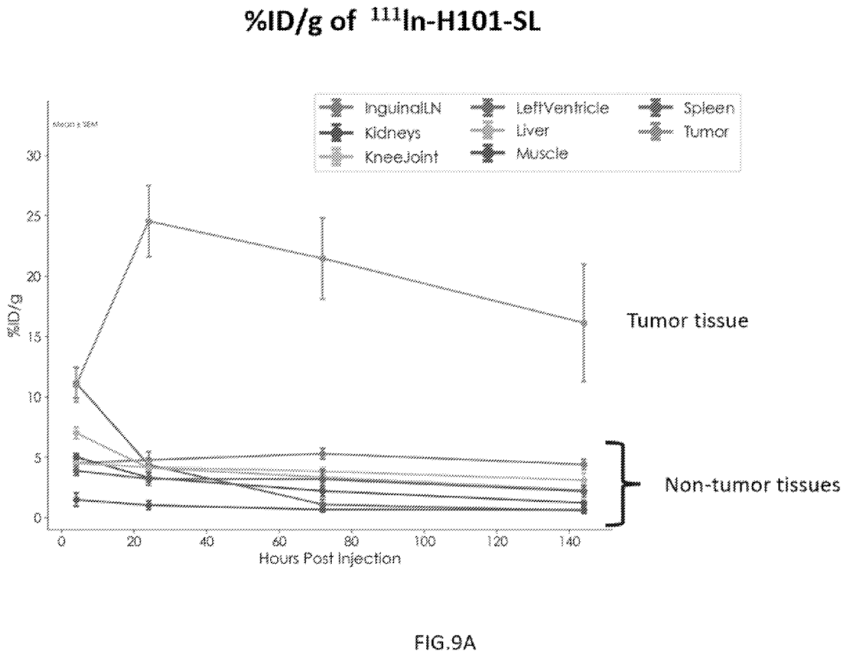

Imaging (e.g., using ¹¹¹In) provides for the ability to collect pharmacokinetic and biodistribution data that can be used to perform dosimetry calculations for treatment planning. (See, e.g., Sgouros G, Hobbs R F. “Dosimetry for radiopharmaceutical therapy.” Semin Nucl Med. 2014 May; 44(3):172-8).). Without being bound by theory, a quantitative demonstration of targeting observed with an imaging label is indicative of the ability to target with a radiolabel (e.g., an alpha emitter) capable of causing targeted cell death. Such phenomena is illustrated by FIG. 8, which illustrates that mice labeled with the imaging isotope ¹¹¹In (top), exhibit accumulation of the therapeutic isotope ²²⁵Ac in tumors that express low amounts of antigen and high amounts of antigen, in this example DLL3 expressing SHP77 tumors and HER2 expressing BT474 tumors respectively.

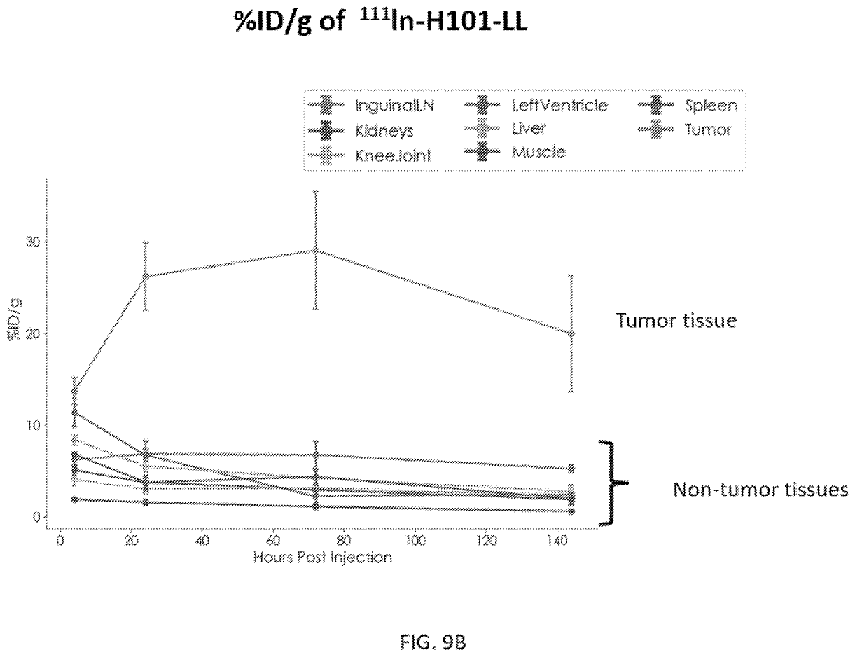

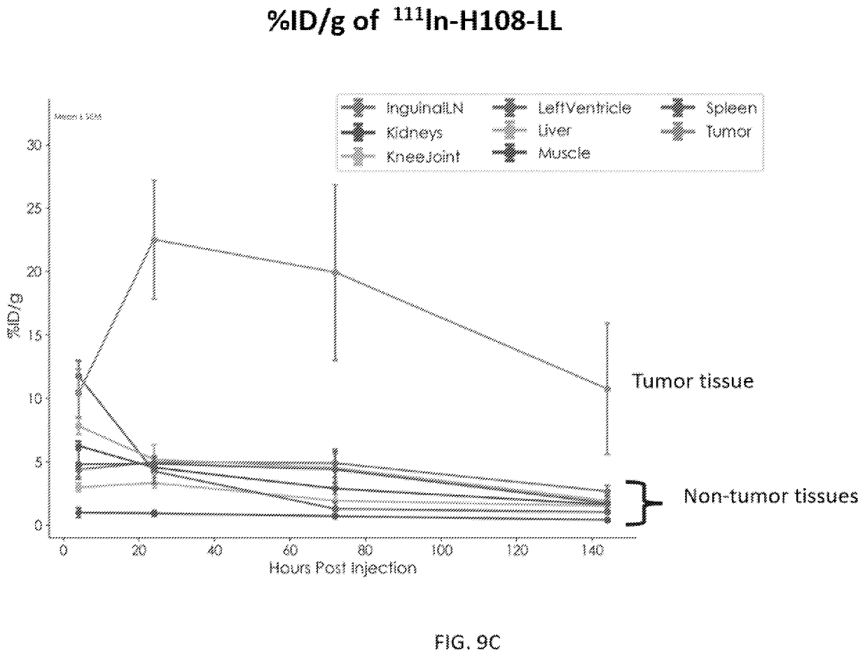

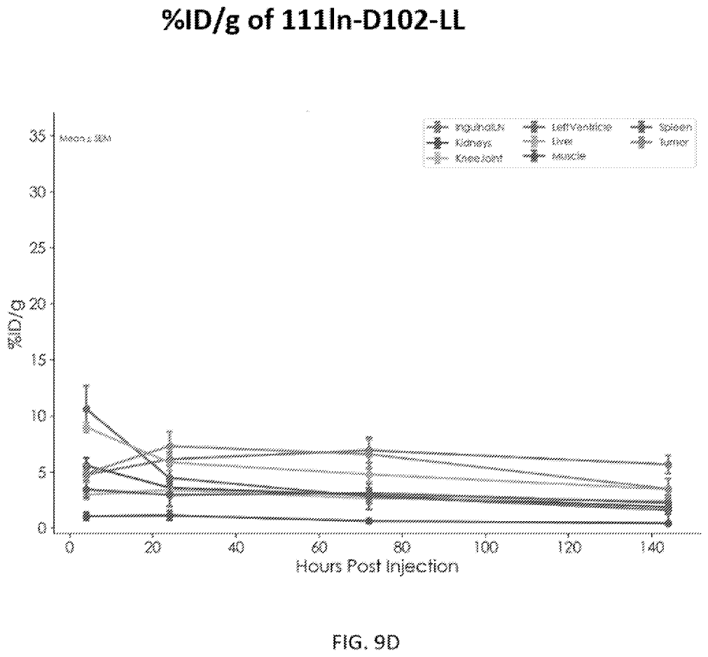

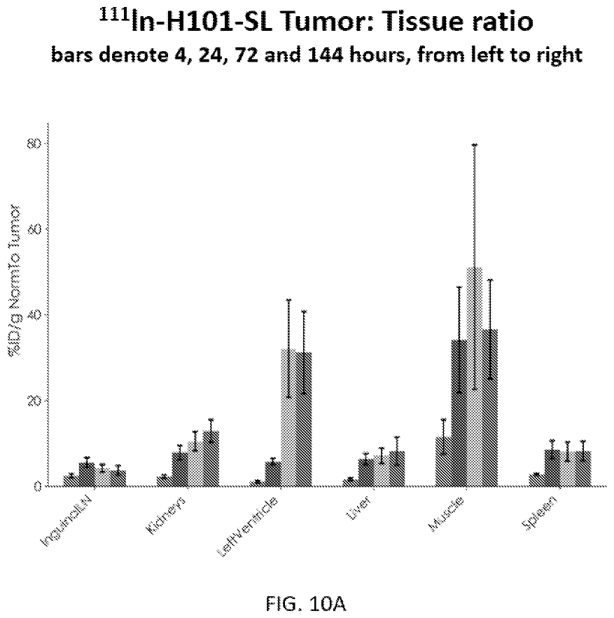

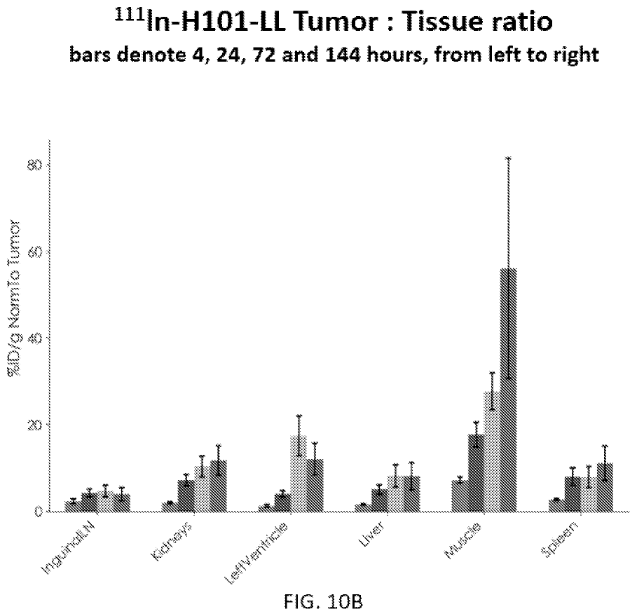

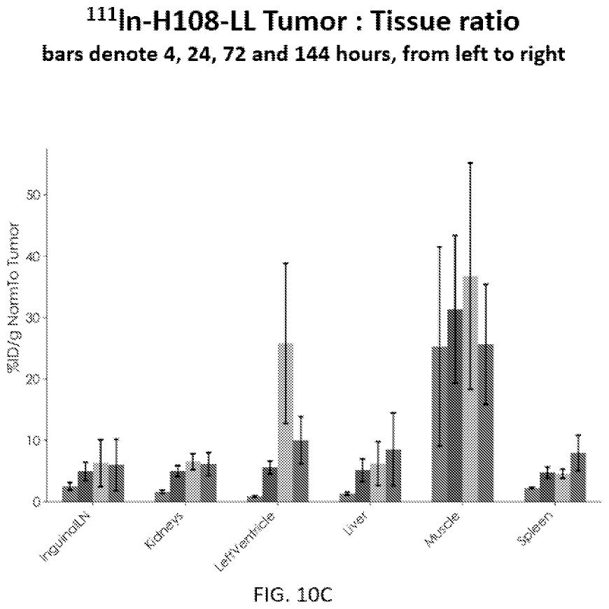

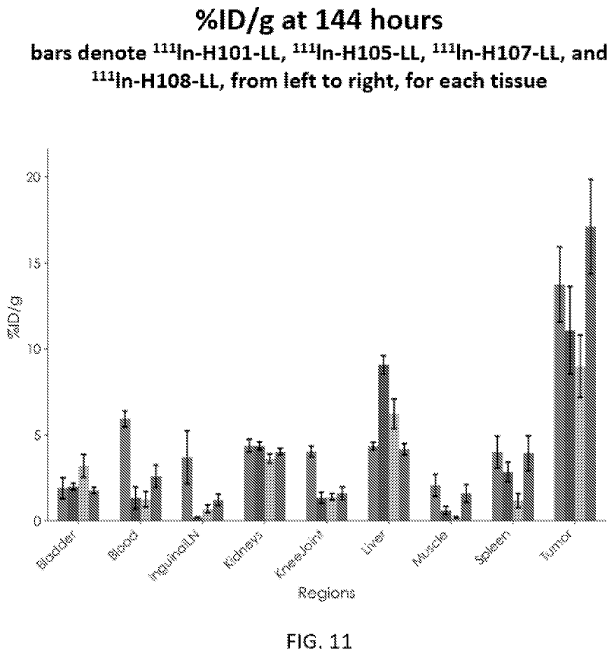

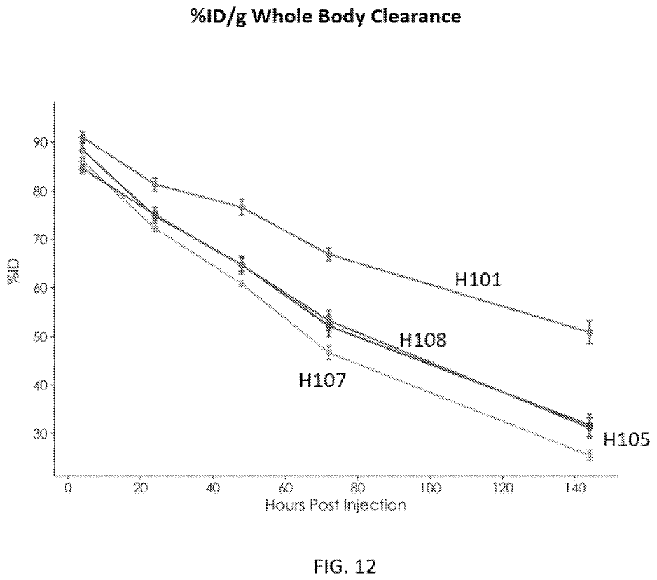

The objective of this study was to observe the biodistribution of ¹¹¹In radiolabeled SPECT/CT imaging across select test articles in BT-474 tumor (breast cancer cells) bearing nude mice. The following articles were tested at a CAR of about 4: ¹¹¹In-H101-4-1, ¹¹¹In-H1014-2, ¹¹¹In-H105-4-2, ¹¹¹In-H107-4-2, and ¹¹¹In-H108-4-2. FIGS. 9A, 9B, and 9C show tissue accumulation over time for ¹¹¹In-H101-SL, ¹¹¹In-H101-4-2, and ¹¹¹In-H108-4-2. FIG. 9D shows minimal tumor accumulation with DLL3 targeting VHH-Fc in HER2+ tumor model, further demonstrating specificity of the HER2 targeting VHH-Fcs. FIGS. 10A, 10B, and 10C show tumor:tissue ratios. In each case, the tumor:tissue ratios were greater than 5, indicating increased tumor accumulation and better profiles used for determining safety (e.g., as compared lower tumor:tissue ratios). FIG. 11 shows % ID/g at 144 hours for ¹¹¹In-H101-4-2, ¹¹In-H105-4-2, ¹¹¹In-H107-4-2, and ¹¹¹In-H108-4-2. In each case, the VHH-Fc variants show advantageous targeting of tumor tissue. FIG. 12 shows whole body clearance of VHH-Fc (H101) and VHH-Fc variants (H105, H107, and H108), wherein the VHH-Fc variants show increased clearance which can further be advantageous when considering safety and preventing unwanted tissue toxicity. In all cases, all test articles avoided significant kidney accumulation, further demonstrating favorable profiles for safety and avoiding unwanted tissue toxicity. Table 13 specifically shows the tumor accumulation for ¹¹¹In-H101-4-2, ¹¹¹In-H105-4-2, ¹¹¹In-H107-4-2, and ¹¹¹In-H108-4-2 over time.

TABLE 13
Tumor accumulation of anti-HER2 VHH-Fc variants
(mean % ID/g; n = 4)

| | | 4 h | 24 h | 48 h | 72 h | 144 h |
|---|---|---|---|---|---|---|
| ¹¹¹In-H108-4-2 | mean | 4.7 | 12.2 | 14.4 | 12.7 | 13.7 |
| | SEM | 0.6 | 1.8 | 2.1 | 0.9 | 2.2 |
| ¹¹¹In-H101-4-2 | mean | 4.9 | 9.3 | 14.2 | 14.1 | 11.1 |
| | SEM | 0.5 | 1.1 | 2.0 | 2.6 | 2.6 |
| ¹¹¹In-H105-4-2 | mean | 4.9 | 7.1 | 9.0 | 9.4 | 9.0 |
| | SEM | 1.1 | 2.0 | 1.9 | 2.2 | 1.8 |
| ¹¹¹Tn-H107-4-2 | mean | 6.2 | 12.6 | 18.6 | 18.0 | 17.1 |
| | SEM | 1.1 | 1.9 | 2.3 | 2.6 | 2.8 |

### Biodistribution and Tissue Accumulation Over Time in DLL3+ SHP-77 Tumors

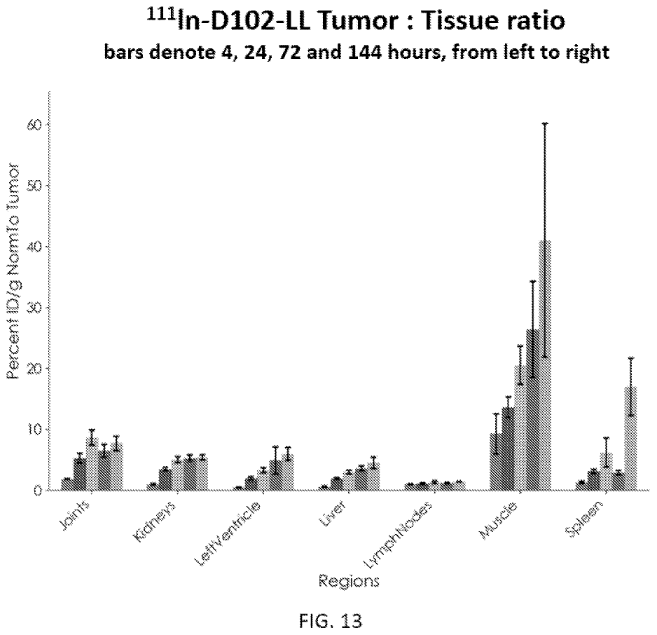

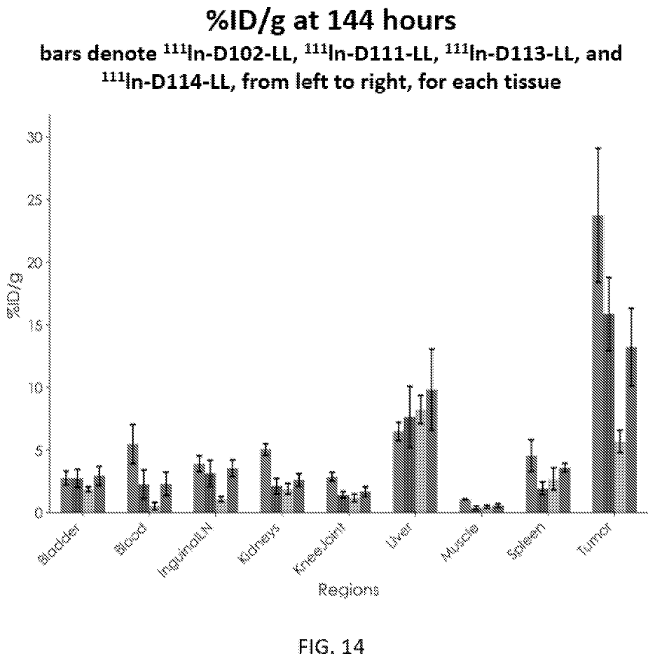

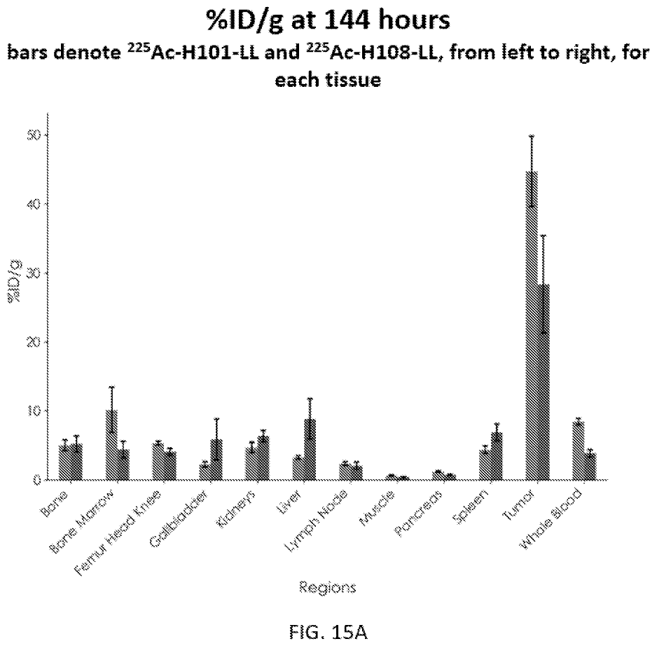

he objective of this study was to observe the biodistribution of ¹¹¹In SPECT/CT across select test articles in SHP-77 tumor bearing nude mice. In contrast to HER2, DLL3 is generally present at lower copy numbers on the cell surface. Accordingly, the DLL3 represents the ability to target low copy number target proteins, whereas HER2 represents the ability to safely and effectively target high copy number target proteins. The following articles were tested: ¹¹¹In-D102-4-2, ¹¹¹In-D11114-2, ¹¹¹In-D1134-2, and ¹¹¹In-D1144-2. Interestingly, similar targeting profiles and observations to the HER2 model were observed for the DLL3 model, demonstrating the ability to target high and low copy number targets. FIG. 13 shows ¹¹¹In-D1024-2 Tumor: Tissue ratios and FIG. 14 shows % ID/g at 144 hours for ¹¹¹In-D102-4-2, ¹¹¹In-D111-4-2, ¹¹¹In-D113-4-2, and ¹¹¹In-D114-4-2. As observed for HER2, anti-DLL3 VHH-Fc variants showed advantageous targeting of tumor tissue. Additionally, liver accumulation is indicative of increased clearance, which can further be advantageous when considering safety and preventing unwanted tissue toxicity. In all cases, all test articles avoided significant kidney accumulation, further demonstrating favorable profiles for safety and avoiding unwanted tissue toxicity. Table 14 specifically shows the tumor accumulation for ¹¹¹In-D102-4-2, ¹¹¹In-D111-4-2, ¹¹¹In-D113-4-2, and ¹¹¹In-D114-4-2 over time.

TABLE 14
Tumor accumulation of anti-DLL3 VHH-Fc variants
(mean % ID/g; n = 4)

| | | 4 h | 24 h | 48 h | 72 h | 144 h |
|---|---|---|---|---|---|---|
| ¹¹¹In-D102-4-2 | mean | 6.0 | 12.8 | 18.0 | 19.0 | 23.7 |
| | SEM | 0.7 | 1.7 | 2.1 | 2.1 | 5.4 |
| ¹¹¹In-D111-4-2 | mean | 5.5 | 12.8 | 16.6 | 16.8 | 15.9 |
| | SEM | 1.4 | 1.1 | 2.0 | 2.3 | 2.9 |
| ¹¹¹In-D113-4-2 | mean | 4.5 | 8.7 | 10.0 | 9.4 | 5.7 |
| | SEM | 0.6 | 1.2 | 1.4 | 1.2 | 0.9 |
| ¹¹¹In-D114-4-2 | mean | 5.1 | 10.9 | 14.6 | 15.8 | 13.2 |
| | SEM | 0.5 | 0.9 | 1.6 | 2.4 | 3.1 |

Taken together, the ¹¹¹In imaging results show that targeting of both high copy number and low copy number targets can be achieved with the radiolabeled VHH-Fcs and VHH-Fc variants. These results further indicate favorable safety and specificity profiles for targeting tumor tissue, avoiding non-tumor tissue, and in certain instances, effectively clearing radiolabeled VHH-Fcs (e.g., VHH-Fcs having mutations that reduced FcRn affinity).

### Biodistribution and Tissue Accumulation of ²²⁵Ac Radiolabeled VHH-Fcs

The objective of this study was to observe biodistribution of (i) ²²⁵Ac radiolabeled HER2 VHH-Fcs in a BT-474 tumor mouse model, as described above, and (ii) ²²⁵Ac radiolabeled DLL3 VHH-Fcs in a SHP-77 tumor mouse model, as described above. Ex vivo radioactive quantitation in tumor and normal tissues was achieved by gamma counting.

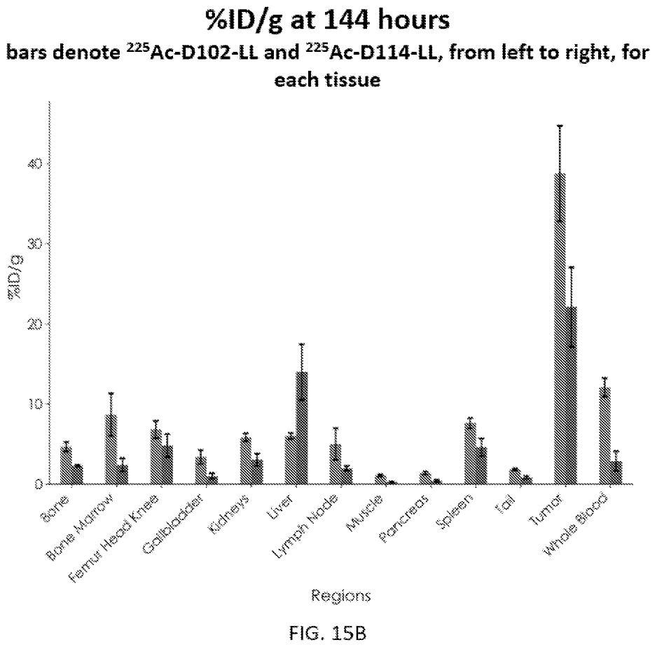

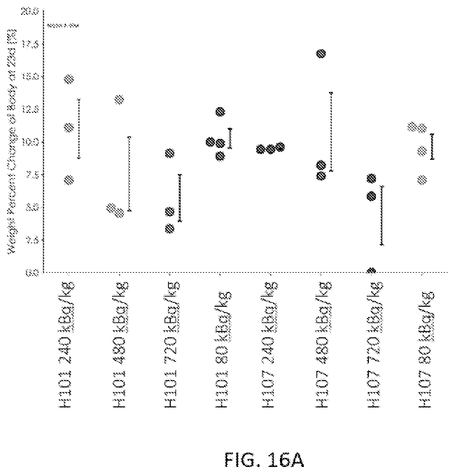

As described herein, the HER2 model represents a target with high receptor density on cancer cells (e.g., ˜300,000 copies/cell). FIG. 15A shows % ID/g at 144 hours for ²²⁵Ac-H101-4-2 and ²²⁵Ac-H1084-2. Both test articles showed advantageous targeting profiles, consistent with the ¹¹¹In imaging data. Notably, specific targeting of tumor tissue was achieved with a favorable tumor:tissue ratio consistent with the imaging data. For the VHH-Fc variant ²²⁵Ac-H108-4-2, lower radioactivity was detected in blood indicating more rapid clearance of the VHH-Fc variant (consistent with results in Example 10). ²²⁵Ac-H108-4-2 also demonstrated lesser kidney accumulation and greater liver accumulation indicating increased clearance through the hepatic route and avoidance of the kidneys which further supports an increase in the safety profile of VHH-Fcs with FcRn mutations. The lower tumor accumulation for ²²⁵Ac-H108-4-2 can be attributed to the decreased serum half-life (i.e., more rapid clearance). Table 15 further shows tumor volume through Day 6 post injection, wherein tumor volumes decreased after administration of ²²⁵Ac-H101-4-2 and ²²⁵Ac-H108-4-2. Table 15 indicates that mice injected with VHH immunoconjugates with wild-type Fc or with FcRn mutations both saw tumor shrinkage by 6 days post injection.

TABLE 15
Tumor volumes before and after anti-HER2 VHH-Fc treatment
(mean mm³; n = 5)

| | Day | −15 | −1 | −8 | −6 | −4 | −1 | 0 (dose) | 3 | 6 |
|---|---|---|---|---|---|---|---|---|---|---|
| 225 Ac- | mean | 57.4 | 66.5 | 51.9 | 54.7 | 65.6 | 73.9 | 74.4 | 31.3 | 47.0 |
| H101-4-2 | SD | 19 | 10 | 10 | 10 | 20 | 36 | 22 | 11 | 12 |
| 225 Ac- | mean | 46.4 | 56.5 | 67.9 | 63.2 | 67.3 | 62.6 | 78.1 | 46.2 | 51.2 |
| H108-4-2 | SD | 9 | 11 | 16 | 12 | 14 | 14 | 27 | 19 | 23 |

As also described herein, DLL3 represents a target with low target density on cancer cells (e.g., ˜3,000 copies/cell). FIG. 15B shows % ID/g at 144 hours for ²²⁵Ac-D1024-2 and ²²⁵Ac-D1144-2. Both test articles showed advantageous targeting profiles, consistent with the ¹¹¹Ln imaging data. Additionally, specific targeting of tumor tissue was achieved with a favorable tumor:tissue ratio consistent with the imaging data. As observed with the anti-HER2 VHH-Fc variants, for the VHH-Fc variant ²²⁵Ac-D1144-2, the VHH-Fc variants show increased clearance and decreased kidney exposure which can further be advantageous when considering safety and preventing unwanted tissue toxicity. The lower tumor accumulation for ²²⁵Ac-D1144-2 can be attributed to the decreased serum half-life (i.e., more rapid clearance).

### Example B-29. Low Toxicity Associated with VHH-Fc Radioimmunoconjugates

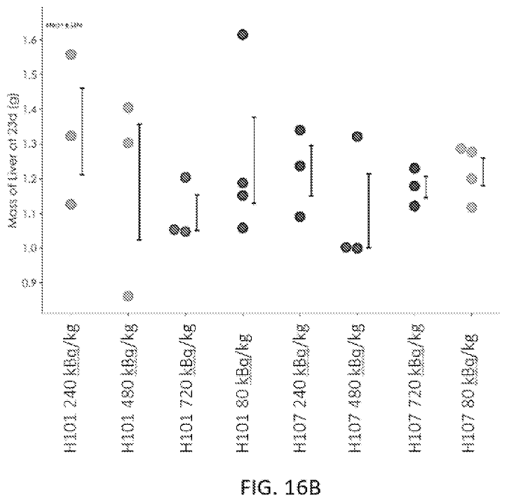

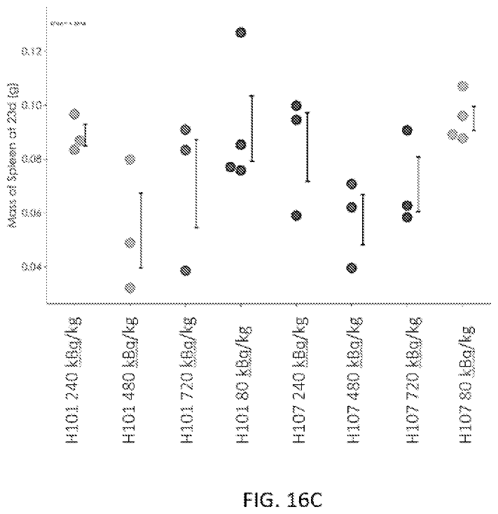

A study was undertaken to determine the tolerability of VHH-Fc loaded with ²²⁵Ac. Naïve female athymic nude mice were injected intravenously (IV) into the tail vein with ²²⁵Ac-H101-4-2 (anti-HER2 with wildtype Fc, chelator-linker 4-2) or ²²⁵Ac-H107-4-2 (anti-HER2 with H310A Fc, chelator-linker 4-2) at four different activity dose levels (18.5 kBq, 12 kBq, 6 kBq, 2 kBq). Activity dose volume was adjusted for body weights measured on the injection day. All animals were monitored for adverse effects daily. Body weights were recorded three (occasionally two or four) times a week for all animals until end of study at 23 days post-injection. 23 Days post-injection all animals were sacrificed. Carcasses underwent necropsy. Whole body, spleen, and liver weights were recorded. FIGS. 16A, 16B, and 16C show that, as measured by percent weight change (16A), liver mass (16B), and spleen mass (16C) All doses of ²²⁵Ac-labeled antibodies of up to 740 kBq/kg were well tolerated and no indications of radiation sickness were observed.

### Example B-30. Efficacy Testing in a SHP77 Xenograft Mice

An efficacy study of anti-DLL3 VHH-Fc (WT and different variants) using the SHP77 lung cancer cell line is conducted. Eighty (80) animals with similar sized tumors will be selected for test article injection. Animals on study will be assigned to the following groups and will be injected with a single bolus intravenous injection (IV) in the tail vein with the labeled test article. Target injection volume 150 μL per mouse, a) Group 1: IV injection of vehicle (PBS), n=8; b); Group 2: IV injection of V002 (no radiolabel), n=8; Group 3: IV injection of ²²⁵Ac-V002-4-2, low dose, n=8; Group 4: IV injection of ²²⁵Ac-V002-4-2, high dose, n=8; Group 5: IV injection of ²²⁵Ac-V014-4-2, low dose, n=8; Group 6: IV injection of ²²⁵Ac-V014-4-2, high dose, n=8; Group 7: IV injection of ¹⁷⁷Lu-V002-4-2, low dose, n=8; Group 8: IV injection of ¹⁷⁷Lu-V002-4-2, high dose, n=8; Group 9: IV injection of ¹⁷⁷Lu-V014-4-2, low dose, n=8; Group 10: IV injection of ¹⁷⁷Lu-V014-4-2, high dose, n=8.

Activity dose levels for both test articles are: a) ²²⁵Ac—: 6 kBq/mouse (low), 18.5 kBq/mouse (high); b) ¹⁷⁷Lu 350 kBq (low), 700 kBq (high).

Mass dose levels for both test articles: based on activity dose and specific activity. a) for ²²⁵Ac groups: 10 μg/mouse (low), 31 μg/mouse (high); b) for ¹⁷⁷Lu groups: 10 μg (low), 20 μg (high).

Animals will be weighed and tumors measured on day of dosing or on the day before (reference data). All animals will be monitored for adverse effects daily. For any animal with adverse effects, scoring will commence for the affected animal on the welfare scoring sheet (Appendix). After dosing, mice will be inspected daily, weighed twice per week, and tumor measurements taken with calipers three times per week for up to 12 weeks (but expecting only ˜4 weeks for control groups 1 and 2). Frequency of weight measurements will be increased when reaching a body weight loss of 10% or more. Actions will be taken such as providing mashed food or gel food. License limit is weight loss of 15%. Animals will be euthanized before planned end of study if tumors exceed the limit (length×width=144 mm²). While the foregoing invention has been described in some detail for purposes of clarity and understanding, it will be clear to the skilled worker from a reading of this disclosure that various changes in form and detail can be made without departing from the true scope of the invention. For example, all the techniques and apparatus described above can be used in various combinations. All publications, patents, patent applications, and/or other documents cited in this application are each incorporated by reference in their entirety for all purposes to the same extent as if each individual publication, patent, patent application, and/or other document were individually indicated to be incorporated by reference for all purposes.

### Example B-31. Radiolabeling with ¹⁷⁷Lu

50 μg of test article (D102) was diluted to 100 μL with 0.1 M ammonium acetate buffer pH 5.5 in a 500 μL lo-bind Eppendorf tube and 51 MBq in 3.2 μL-3.5 μL of ¹⁷⁷Lutetium chloride was added and mixed with a pipette. The reaction mixtures were incubated at 37° C. in an incubator for 3 hours and samples taken at 30 min, and 1, 2, and 3 h for iTLC analysis. Results of the labeling are shown in Table 16 below, and indicate efficient labeling with ¹⁷⁷Lu.

TABLE 16

| | |
|---|---|
| Test article | Incubation time at 37° C. |

| D102 | 30 min | 60 min | 2 hr | 3 hr |
|---|---|---|---|---|
| Compound 4-2 | 99.0% | 99.2% | 99.2% | 99.3% |

After dilution in PBS/ascorbate and storage at 4° C. the purity as assessed by iTLC analysis as in Example 22.

To analyze stability, 50 μL of test article was added to 200 μL of PBS/ascorbate and stored at 4° C. The samples were analyzed by iTLC and SEC-HPLC after 1-4 h and 18-24 h. Results are shown in Table 17 below, and indicate stability of the construct.

TABLE 17

| | |
|---|---|
| Test article | Incubation time |

| D102 | 1 hr | 1 d |
|---|---|---|
| Compound 4-2 | 98.8% | 98.6% |

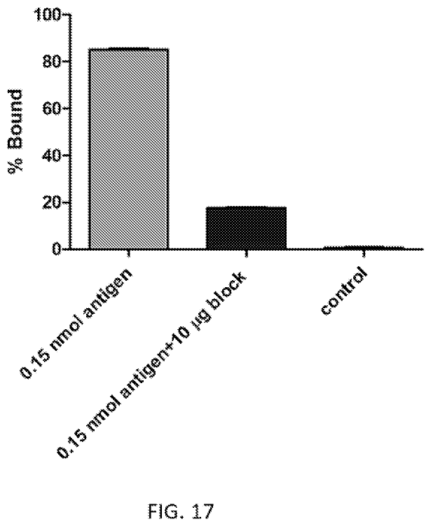

The ¹⁷⁷Lu conjugate was analyzed by the IRF assay described above in Example 23 and the results are shown in FIG. 17. In this example, the control is beads with no antigen loaded.

### Example B-32. Binding Determination on the Py4Pa Compounds

In some instances, conjugation can negatively impact binding of the VHH-Fc to the target protein. Binding of VHH-Fc conjugates was therefore tested,

ACCs conjugates were screened for binding to a range of target-positive cancer cell lines by flow cytometry. All cell lines were sourced from ATCC unless otherwise noted, and cultured according to manufacturer's instructions and recommended media. The HER2-positive cell line (For V001 conjugates) was BT474(ATCC #HTB-20). SHP-77 (ATCC CRl-2195) cells were used as the DLL3-positive cell line (For V002 and V038 conjugates). The HER2-negative cell line was SHP-77. The DLL3-negative cell line was BT-474. Primary antibodies diluted in same manner as for ELISA were added to cells and incubated for 1 hour on ice. Cells were washed twice with 1% FBS in PBS, centrifuged at 450G for 4 minutes and incubated with 2 μg/mL AlexaFluor 647 conjugated anti-human IgG (Jackson, Cat #109-605-098) or AlexaFluor 647 conjugated anti-mouse IgG (Jackson, Cat #115-605-164) with 1:1000 DAPI (Biolegend, Cat #422801) for 30 minutes on ice. Following two further washes, cells were resuspended, and analyzed by flow cytometry on the iQue screener platform (Intellicyt), and data was processed with Forecyt, according to standard protocols.

### Example B-33. In Vitro Plasma Stability Assay

To evaluate the stability of the novel chelator-linkers as they would circulate in the bloodstream, fragments of the test articles can be prepared and subjected to mass guided assays known to the art.

Compounds can be evaluated by a plasma stability assay according to Choi, T. A., et al. J. Pharm. Sci., 2015, 104, 1832-1838. Mouse and human plasma are diluted 1:1 with PBS (pH 7.4, 100 mL). To this solution, compound is added and diluted 30× to a final concentration of 15 μM. Test articles are prepared in duplicate. At the desired timepoints, 100 μL aliquots of test samples are quenched by adding to 2× volume of chilled acetonitrile which may contain an internal standard. Proteins are removed either by centrifugation at 4000 g or filtration using a 0.2 μm membrane centrifugal filter and centrifuging at 3000 g for 10 min. The supernatant is then injected for analysis on an LC-MS/MS to analyze the amount of parent compound remaining. The % remaining is calculated by comparing standard peak areas using a method that may be specifically designed for each compound analyzed. Alternatively, an internal standard may be used for more precise measurements.

### Example B-34. Liver S9 Metabolic Stability Assay

To evaluate any changes in the metabolic properties of chelator-linker fragments which may be liberated in the course of administering a radioconjugate, including increased or decreased liver clearance, metabolic stability assays including the Liver S9 fraction assay can be carried out according to methods known to practitioners of the art.

Liver S9 metabolic stability can be performed according to Richardson, S. J. et al. Drug Metabolism Letters, 2016, 10, 83-90. In brief, human liver S9 fraction (gender pooled) is a commercial product and should be procured. A 200 nM tris solution containing 2 mM MgCl₂ is prepared in dH2O and adjusted with 1 M NaOH to pH 7.4. Stock solutions of the compounds to be analyzed are prepared in DMSO to 50 mM. These are diluted to 0.3 mM immediately before use in dH2O. NADPH, UDPGA and GSH solutions are prepared at 40, 20, and 2 mM respectively in the tris buffer described above. PAPS is prepared at 2 mg/mL in the above tris buffer. The four solutions in tris were mixed in a 1:1:1:1 ratio immediately prior to use.

For the assay, each compound is pre-incubated with the S9 fraction in tris buffer pH 7.4 for 5 min at 37° C. in a well of a 96-well plate. Next, the freshly prepared and mixed co-factor solution above is added. Aliquots are removed at the appropriate timepoints, they are quenched by the addition of two volumes of ice-cold 50:50 MeCN/H₂O. The plate is centrifuged at 4000 g for 10 min to sediment precipitated proteins. The compounds are injected for analysis on an LC-MS/MS to analyze the amount of parent compound remaining. The % remaining is calculated by comparing standard peak areas using a method that may be specifically designed for each compound analyzed.

### Example B-35. CACO-2 Permeability Assay

To evaluate any changes in the metabolic properties of chelator-linker fragments which may be liberated in the course of administering a radioconjugate, including increased or decreased cell permeability or transport through clearance organs, cell permeability assays can be carried out according to methods known to practitioners of the art.

The CACO-2 permeability assay can be performed according to Richardson, S. J. et al. Drug Metabolism Letters, 2016, 10, 83-90. Polarized cultures of Caco-2 cells are available on polycarbonate micro-porous filters in HTS Transwell plates. These have a 21-day cell barrier. Other places of similar types are available for purchase.

Hank's balanced salt solution (HBSS) is the transport medium used for the permeability study. HBSS contains 1.1 mM MgCl₂, 1.3 mM CaCl₂, and 5 mM D-glucose. Prior to each experiment, the monolayer should be washed with warm buffer. A 10 μM solution of Lucifer yellow is used to check the monolayer integrity of all wells, and three control compounds were used in each assay (atenolol, propranolol and vinblastine). Test samples are diluted to a final concentration of 10 μM in the HBSS buffer. The buffer solution is added to either the apical or basolateral side of the monolayer, each test article done in duplicate. The monolayers are incubated in a standard cell-culture incubator (5% CO₂, 37° C.) for 2 h. At the end of incubation, samples are taken from each side of the monolayer and analyzed by LC-MS/MS.

### Example B-36. In Vivo BioD with ²²⁵Ac

Dosing animals and ex vivo quantification of radioactivity in tissues is a way to precisely monitor the disposition of radiation dose that can occur to an organism during treatment with radioactive substances. Using ²²⁵Ac will give a more accurate simulation of how the clinical treatment will work that includes the factors affecting the stability of the chelator-²²⁵Ac complex, and the nature of the radioactive daughter ions.

The study will be executed as in Example B-15, with different timepoints and counting factors that account for the techniques required to measure activity of ²²⁵Ac and its daughters.

### Example B-37. MTD Studies Comparing Maximum Dose Achievable with Different Chelator-Linkers

This experiment compares the MTD that can be achieved with conjugates using different chelator-linkers in order to observe any differences that new excretion profiles can achieve.

### Example B-38. Efficacy Comparison with Xenograft Model to Test Lead Chelator-Linker Candidates with ²²⁵Ac and 177Lu

An efficacy study of anti-DLL3 VHH-Fc (WT and different variants) using the SHP77 lung cancer cell line is conducted. Eighty (80) animals with similar sized tumors are selected for test article injection. Animals on study are assigned to groups and will be injected with a single bolus intravenous injection (IV) in the tail vein with the ²²⁵Ac labeled test article. Target injection volume 150 μL per mouse, two dose levels for each chelator-linker are chosen that are below the MTD established in Example B-17, with a comparison between 4 candidate chelator-linkers conjugated to the same DLL3 targeting antibody.

Activity dose levels for both test articles, if based on previous experience, would be: a) ²²⁵Ac: 6 kBq/mouse (low), 18.5 kBq/mouse (high); b) ¹⁷⁷Lu: 350 kBq (low), 700 kBq (high).

Mass dose levels for both test articles: based on activity dose and specific activity. a) for ²²⁵Ac-225 groups: 10 μg/mouse (low), 31 μg/mouse (high); b) for ¹⁷⁷Lu groups: 10 μg (low), 20 μg (high).

Animals are weighed and tumors measured on day of dosing or on the day before (reference data). All animals are monitored for adverse effects daily. For any animal with adverse effects, scoring commences for the affected animal on an animal welfare scoring sheet. After dosing, mice are inspected daily, weighed twice per week, and tumor measurements are taken with calipers three times per week for up to 12 weeks (but expecting only to ˜4 weeks for control groups 1 and 2). Frequency of weight measurements are increased when reaching a body weight loss of 10% or more. Actions are taken such as providing mashed food or gel food. License limit is weight loss of 15%. Animals are euthanized before planned end of study if tumors exceed the limit (length×width=144 mm²).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Fc1
I253A
(SEQ ID NO: 1)
APELLGGPSVFLFPPKPKDTLMASRTPEVTCVVVDVSHEDPEVKFNWYVDG
VEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG
Fc2
S254A
(SEQ ID NO: 2)
APELLGGPSVFLFPPKPKDTLMIARTPEVTCVVVDVSHEDPEVKFNWYVDGV
EVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
Fc3
H310A
(SEQ ID NO: 3)
APELLGGPSVFLFPPKPKDTLMISRTPEVTCVVVDVSHEDPEVKFNWYVDGV
EVHNAKTKPREEQYNSTYRVVSVLTVLAQDWLNGKEYKCKVSNKALPAPIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
Fc4
H435Q
(SEQ ID NO: 4)
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNQYTQKSLSLSPG
Fc5
Y436A
(SEQ ID NO: 5)
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHATQKSLSLSPG
Fc6
H310A/H435Q
(SEQ ID NO: 6)
APELLGGPSVFLFPPKPKDTLMISRTPEVTCVVVDVSHEDPEVKFNWYVDGV
EVHNAKTKPREEQYNSTYRVVSVLTVLAQDWLNGKEYKCKVSNKALPAPIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNQYTQKSLSLSPG
Fc7
AEASS
(SEQ ID NO: 7)
APEAEGAPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPSSIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
Fc8
AEASS/H310A
(SEQ ID NO: 8)
APEAEGAPSVFLFPPKPKDTLMISRTPEVTCVVVDVSHEDPEVKFNWYVDGV
EVHNAKTKPREEQYNSTYRVVSVLTVLAQDWLNGKEYKCKVSNKALPSSIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
Fc9
AEASS/H435Q
(SEQ ID NO: 9)
APEAEGAPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPSSIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNQYTQKSLSLSPG
Fc wild type
(SEQ ID NO: 10)
APELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
2RS15d
(SEQ ID NO: 20)
QVQLQESGGGSVQAGGSLKLTCAASGYIFNSCGMGWYRQSPGRERELVSRIS
GDGDTWHKESVKGRFTISQDNVKKTLYLQMNSLKPEDTAVYFCAVCYNLETY
WGQGTQVTVSS
2RS15d CDR1
(SEQ ID NO: 21)
GYIFNSCG
2RS15d CDR2
(SEQ ID NO: 22)
ISGDGDT
2RS15d CDR3
(SEQ ID NO: 23)
AVCYNLETY
hz10D9v7.251
(SEQ ID NO: 30)
EVQLVESGGGEVQPGGSLRLSCAASGSIFSINAMGWYRQAPGKQRELVAG
FTGDTNTIYAESVKGRFTISRDNAKNTVYLQMSSLRAEDTAVYYCAADVQLFSRDYEF
YWGQGTLVTVKP
hz10D9v7.251 CDR1
(SEQ ID NO: 31)
GSIFSINA
hz10D9v7.251 CDR2
(SEQ ID NO: 32)
FTGDTNT
hz10D9v7.251 CDR3
(SEQ ID NO: 33)
AADVQLFSRDYEFY

1-71. (canceled)

72. A compound of Formula (I), or a pharmaceutically acceptable salt thereof: 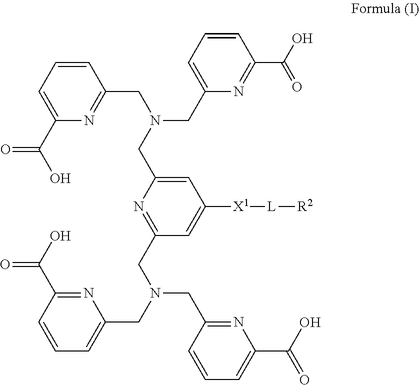
wherein:
X¹ is —O—, —S—, —S(═O)—, —S(═O)₂, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR^{a}C(═S)NR^{a}—, —NR^{a}C(═O)O—, -(unsubstituted or substituted C₁-C₆alkylene)-X²—, —O-(unsubstituted or substituted C₁—(C₆alkylene)-X²—, or —(C₄-C₂₀polyethylene glycol)-X²;
X² is absent, —C(═O)—, —NR^{a}C(═O)—, —C(═O)NR^{a}—, or —C(═O)X⁴—;
each R is independently selected from hydrogen and C₁-C₄alkyl;
X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(O)R⁵, and wherein any free carboxylic acid of any amino acid is optionally replaced with —C(═O)NH—R⁵;
R² is a moiety that is capable of reacting with an amine (—NH₂) of the tumor targeting moiety R³ and comprises a tetrafluorophenyl ester, pentafluorophenyl ester, dinitrophenyl ester, succinimide ester, sulfosuccinimide ester, or isothiocyanate; L is a linker that is -L¹-L²-L³-L⁴-L⁵-;
L¹ is absent, unsubstituted or substituted C₁-C₁₀alkylene, unsubstituted or substituted C₁-C₁₀heteroalkylene, unsubstituted or substituted C₂-C₂₀alkenylene, unsubstituted or substituted C₂-C₂₀alkynylene, C₄-C₂₀polyethylene glycol, —(X³CH₂CH₂)ₜ—, unsubstituted or substituted cycloalkylene, unsubstituted or substituted heterocycloalkylene, unsubstituted or substituted arylene, or unsubstituted or substituted heteroarylene,
each X³ is independently selected from O and NR⁴;
each t is independently 1, 2, 3, 4, 5, 6, 7, 8, 9, 10, 11, or 12;
L² is absent, unsubstituted or substituted C₁-C₁₀alkylene, unsubstituted or substituted C₁-C₁₀heteroalkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₘ—(CH₂)_{P}—, —C(═O)NR⁴—(CH₂CH₂X³)ₘ, —(CH₂)ₚ—, —NR⁴C(═O)—(CH₂CH₂X³)ₘ—(CH₂)ₚ—, or —(CH₂CH₂X³)ₘ—(CH₂)ₚ,
each R⁴ is independently selected from hydrogen and C₁-C₆alkyl;
each m is independently 1, 2, 3, 4, 5, 6, 7, 8, 9, 10, 11, or 12;
each p is independently 0, 1, or 2;
L³ is absent, or one or more independently selected groups selected from, natural or unnatural amino acids, and an optional amino(unsubstituted or substituted benzyl)carbamate, wherein any free amine (—NH₂) of an amino acid is optionally independently substituted with R⁵ or —C(═O)R⁵, wherein any free carboxylic acid of any amino acid is optionally replaced with —C(═O)NH—R⁵, and wherein substituted benzyl is substituted with 1 or 2 groups selected from halogen, —OH, —OR, —CO₂H, —NHR⁵, —C(═O)NHR⁵, —NHC(═O)R⁵ and substituted C₁-C₆alkyl, wherein the substituted C₁-C₆alkyl is substituted with —OH, —CO₂H, —NHR⁵, —C(═O)NHR⁵, and —NHC(═O)R⁵;
L⁴ is absent, unsubstituted or substituted C₁-C₁₀alkylene, —C(═O)-(unsubstituted or substituted C₁-C₁₀alkylene)-, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂X³)ₙ—(CH₂)_{q}—, —C(═O)NR⁴—(CH₂CH₂X³)ₙ—(CH₂)_{q}—, —NR⁴C(═O)—(CH₂CH₂X³)ₙ—(CH2)_{q}—, —(CH₂CH₂X³)ₙ—(CH₂)_{q}—, or —(X³CH₂CH₂)ₙ—;
each n is independently 1, 2, 3, 4, 5, 6, 7, 8, 9, 10, 11, or 12;
each q is independently 0, 1, or 2;
L⁵ is absent, —C(═O)—(CH₂)ₙ—, —C(═O)NR⁴—(CH₂)ₙ—, —NR⁴C(═O)—(CH₂)ₙ—, —C(═O)—(CH₂CH₂X³)ₙ(CH₂)_{q}—, —C(═O)NR⁴—(CH₂CH—X³)ₙ—((CH₂)_{q}—, —NR⁴C(═O)—(CH₂CH₂X³)ₙ—(CH₂)_{q}—, —(CH₂CH₂X³)ₙ—(CH₂)_{q}—, —C(═O)—(X³CH₂CH₂)ₙ—, or —(X³CH₂CH₂)ₙ—;
each n is independently 1, 2, 3, 4, 5, 6, 7, 8, 9, 10, 11, or 12;
each q is independently 0, 1, or 2;
each R^{a} is independently selected from hydrogen and C₁-C₄alkyl;
wherein heteroalkylene is an alkylene where one carbon atom is replaced with —O—, —S—, —S(═O)—, —S(O)₂—, —S(═O)(═NH)—, —S(═O)(═NR⁶)—, —NR⁶—, —P(═O)OH—, —NHC(═O)—, —C(═O)NH—, —OC(═O)NH—, —NHC(═N—CN)NH—, or —NHC(═N—R⁶)NH—;
wherein when any one of -L¹-, -L²-, -L³-, -L⁴-, and -L⁵- is substituted then -L¹-, -L²-, -L³-, -L⁴-, and -L⁵- is substituted with 1, 2, 3, or 4 groups selected from halogen, —OH, —OR⁵, —CO₂H, —NHR⁵, —C(═O)NHR⁵, —NHC(═O)R⁵ and unsubstituted or substituted C₁-C₆alkyl, wherein the substituted C₁-C₆alkyl is substituted with —OH, —CO₂H, —NHR⁵, —C(O)NHR⁵, or —NHC(═O)R⁵;
each R⁵ is independently selected from C₁-C₁₀alkyl, C₄-C₃₀polyethyleneglycol, unsubstituted or substituted arylene, and unsubstituted or substituted heteroarylene;
each R⁶ is independently selected from C₄-C₃₀polyethylene glycol, unsubstituted or substituted arylene, unsubstituted or substituted heteroarylene, and unsubstituted or substituted C₁-C₆alkyl, wherein the substituted C₁-C₆alkyl is substituted with halogen, —OH, —CO₂H, —NHR⁵, —C(═O)NHR⁵, or —NHC(═O)R⁵;
provided that when L³ is absent then at least one R⁵ is present or —NH-L¹- is one or more independently selected natural or unnatural amino acids; or
provided that —X¹-L-R² is not 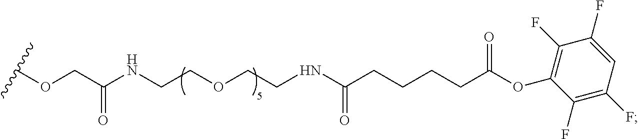
or a radionuclide complex thereof.

73. The compound of claim 72, or a pharmaceutically acceptable salt thereof, wherein: R² is a moiety that is capable of reacting with an amine (—NH₂) of the tumor targeting moiety R³ and comprises: 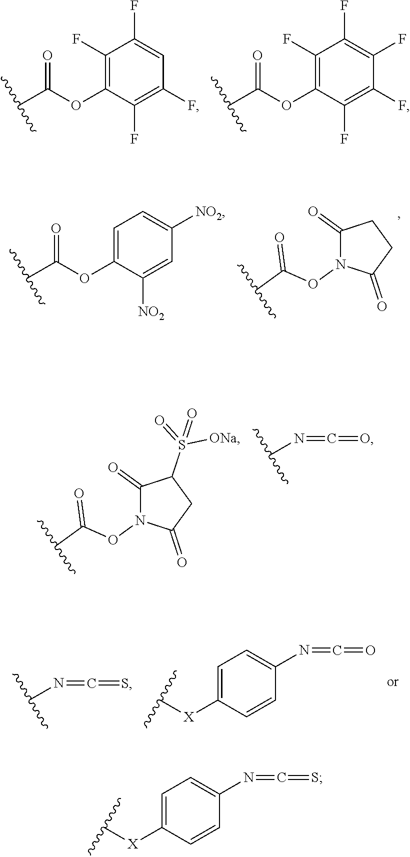
X is absent, —O—, —S—, —S(═O)—, —S(═O)₂—, —NR^{a}—, —C(═O)—, —NR^{a}C(═O)—, —C(═O)NR^{a}—, —C(═O)O—, —OC(═O)—, —OC(═O)NR^{a}—, —NR^{a}C(═O)NR^{a}—, —NR⁴C(═S)NR^{a}—, —NR^{a}C(═O)O—;
each R^{a} is independently selected from hydrogen and C₁-C₄alkyl.

74. The compound of claim 72, or a pharmaceutically acceptable salt thereof, wherein:
X¹ is —O—, —S—, —S(═O)—, —S(═O)₂—, —NR^{a}—, —C(═O)—, —NR^{a}C(═O)—, —C(═O)NR^{a}—, —(C₁-C₆alkylene)-X²—, —O—(C₁-C₆alkylene)-X²—, or —(C₄-C₂₀polyethylene glycol)-X²—;
X² is absent, —C(═O)—, —C(═O)NR^{a}—, or —C(═O)X⁴—;
X⁴ is —NR^{a}—, —NR^{a}S(═O)₂—, —NR^{a}S(═O)₂NR^{a}—, or one or more independently selected natural or unnatural amino acids, wherein any free amine of an amino acid is optionally independently substituted with R⁵ or —C(═O)R⁵, and wherein any free carboxylic acid of any amino acid is optionally replaced with —C(═O)NH—R⁵—.

75. The compound of claim 72, or a pharmaceutically acceptable salt thereof, wherein:
X¹ is —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —OCH₂—X²—, —OCH₂CH₂—X²—, —OCH₂CH₂CH₂—X²—, —OCH₂CH₂CH₂CH₂—X²—, —OCH₂CH₂CH₂CH₂CH₂—X²—, or —OCH₂CH₂CH₂CH₂CH₂CH_{F}—X²—.

76. The compound of claim 72, or a pharmaceutically acceptable salt thereof, wherein:
X¹ is —OCH₂—, —OCH₂—X²—, —OCH₂CH₂— or —OCH₂CH₂—X²—;
X² is —C(═O)X⁴—;
X⁴ is —NH—, —N(CH₃)—, —N(CH₂CH₃)—, —NHS(═O)₂—, —N(CH₃)S(═O)₂, —N(CH₂CH₃)S(═O)₂—, lysine, glutamine, glutamate, glycine, leucine, lysine, methionine, phenylalanine, serine, tyrosine, valine, citrulline, methionine-valine-lysine, glycine-phenylalanine-glycine-glycine, tyrosine-arginine-valine, arginine-valine, or combination thereof; wherein any free amine (—NH₂) of an amino acid is optionally independently substituted with R⁵ or —C(═O)R⁵, and wherein any free carboxylic acid of any amino acid is optionally replaced with —C(═O)NH—R⁵.

77. The compound of claim 72, or a pharmaceutically acceptable salt thereof, wherein:
X¹ is —OCH₂—, —OCH₂—X²—, —OCH₂CH₂— or —OCH₂CH₂—X²—.
X² is —C(═O)X⁴—;
X⁴ is —NH—, —N(CH₃)—, —N(CH₂CH₃)—, —NHS(═O)₂—, —N(CH)S(═O)₂, or —N(CH₂CH₃)S(═O)₂—.

78. The compound of claim 72, or a pharmaceutically acceptable salt thereof, wherein:
L¹ is unsubstituted or substituted C₁-C₆alkylene, unsubstituted or substituted C₁-C₁₀heteroalkylene, C₄-C₂₀polyethylene glycol, unsubstituted or substituted cyclohexylene, or unsubstituted or substituted phenylene;
L² is absent, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂O)ₘ, —(CH₂)_{P}—, —C(═O)NR⁴—(CH₂CH₂O)ₘ—(CH₂)_{P}—, —NR⁴C(═O)—(CH₂CH₂O)ₘ—(CH₂)_{P}—, or (CH₂CH₂O)ₘ—(CH₂)_{P}—;
each m is independently 1, 2, 3, 4, 5, or 6;
each p is independently 1 or 2;
L⁴ is absent, —C(═O)-(unsubstituted or substituted C₁-C₁₀alkylene)-, —C(═O)NR⁴-(unsubstituted or substituted C₁-C₆alkylene)-, —NR⁴C(═O)-(unsubstituted or substituted C₁-C₆alkylene)-, —C(═O)—(CH₂CH₂O)ₙ—(CH₂)_{q}—, —C(═O)NR⁴—(CH₂CH₂O)ₙ—(CH₂)_{q}—, —NR⁴C(═O)—(CH₂CH₂O)ₙ—(CH2)_{q}—, or —(CH₂CH₂O)ₙ—(CH₂)_{q}—,
L⁵ is —NR⁴C(═O)—(CH₂)ₙ—, —C(═O)(CH₂CH₂O)ₙ—(CH₂)_{q}—, —C(═O)NR⁴—(CH₂CH₂O)ₙ—(CH₂)_{q}—, or —NR⁴C(═O)—(CH₂CH₂O)ₙ—(CH₂)_{q}—;
each n is independently 1, 2, 3, 4, 5, or 6;
each q is independently 1 or 2.

79. The compound of claim 72, or a pharmaceutically acceptable salt thereof, wherein:
L¹ is unsubstituted or substituted C₁-C₆alkylene, unsubstituted or substituted C₁-C₁₀heteroalkylene, C₄-C₂₀polyethylene glycol, unsubstituted or substituted cyclohexylene, or unsubstituted or substituted phenylene;
L² is absent, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂O)ₘ—(CH₂)_{P}—, —C(═O)NR⁴—(CH₂CH₂O)ₘ—(CH₂)_{P}—, —NR⁴C(═O)—(CH₂CH₂O)ₘ—(CH₂)_{P}—, or —(CH₂CH₂O)ₘ—(CH₂)_{P}—,
each m is independently 1, 2, 3, 4, 5, or 6,
each p is independently 1 or 2;
L⁴ is absent;
L⁵ is absent, —NR⁴C(═O)—(CH₂)ₙ—, —C(═O)—(CH₂CH₂O)ₙ—(CH₂)_{q}—, —C(═O)NR⁴—(CH₂CH₂O)ₙ—(CH₂)_{q}—, or —NR⁴C(═O)—(CH₂CH₂O)ₙ—(CH₂)_{q};
each n is independently 1, 2, 3, 4, 5, or 6;
each q is independently 1 or 2.

80. The compound of claim 72, or a pharmaceutically acceptable salt thereof, wherein:
L¹ is unsubstituted or substituted C₁-C₆alkylene, unsubstituted or substituted C₁-C₁₀heteroalkylene, C₄-C₂₀polyethylene glycol, unsubstituted or substituted cyclohexylene, or unsubstituted or substituted phenylene;
L² is absent, —C(═O)NR⁴-(unsubstituted or substituted C₁-C₁₀alkylene)-, —NR⁴C(═O)-(unsubstituted or substituted C₁-C₁₀alkylene)-, —C(═O)—(CH₂CH₂O)ₘ—(CH₂)_{P}—, —C(═O)NR⁴—(CH₂CH₂O)ₙ—(CH₂)_{P}—, —NR⁴C(═O)—(CH₂CH₂O)ₙ—(CH₂)_{P}—, or —(CH₂CH₂)ₙ—(CH₂)_{P}—,
each m is independently 1, 2, 3, 4, 5, or 6;
each p is independently 1 or 2,
L³ is one or more independently selected groups selected from: alanine, arginine, asparagine, aspartate, cysteine, glutamine, glutamate, glycine, leucine, lysine, methionine, phenylalanine, proline, serine, tyrosine, valine, and amino(unsubstituted or substituted benzyl)carbamate; wherein any free amine of an amino acid is optionally substituted with R⁵ or —C(═O)(R⁵) and any free carboxylic acid of an amino acid is optionally replaced with —C(═O)NH(R⁵);
each R⁵ is independently an unsubstituted or substituted C₁-C₁₀alkylene, C₄-C₂₀polyethyleneglycol, or an unsubstituted or substituted phenyl, wherein the substituted phenyl is substituted with 1, 2, 3, 4, or 5 groups independently selected from F, Cl, Br, I, —CH₂, and CF₃;
L⁴ is absent;
L⁵ is —NR⁴C(═O)—(CH₂)ₙ—, —C(═O)—(CH₂CH₂O)ₙ—(CH₂)_{q}—, —C(═O)NR⁴—(CH₂CH₂O)ₙ—(CH₂)_{q}—, or —NR⁴C(═O)—(CHCH₂O)ₙ—(CH₂)_{q}—;
each n is independently 1, 2, 3, 4, 5, or 6;
each q is independently 1 or 2.

81. The compound of claim 72, or a pharmaceutically acceptable salt thereof, wherein —X¹-L- is —O—CH₂C(═O)NH-L- and —CH₂C(═O)NH-L- is:

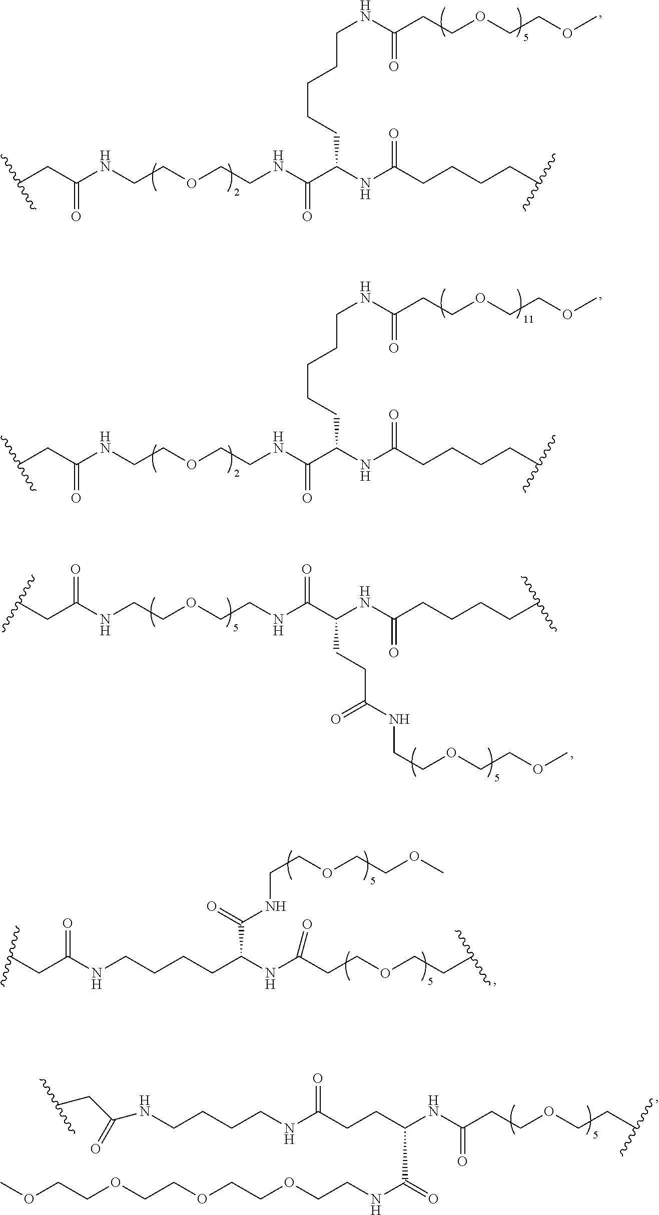

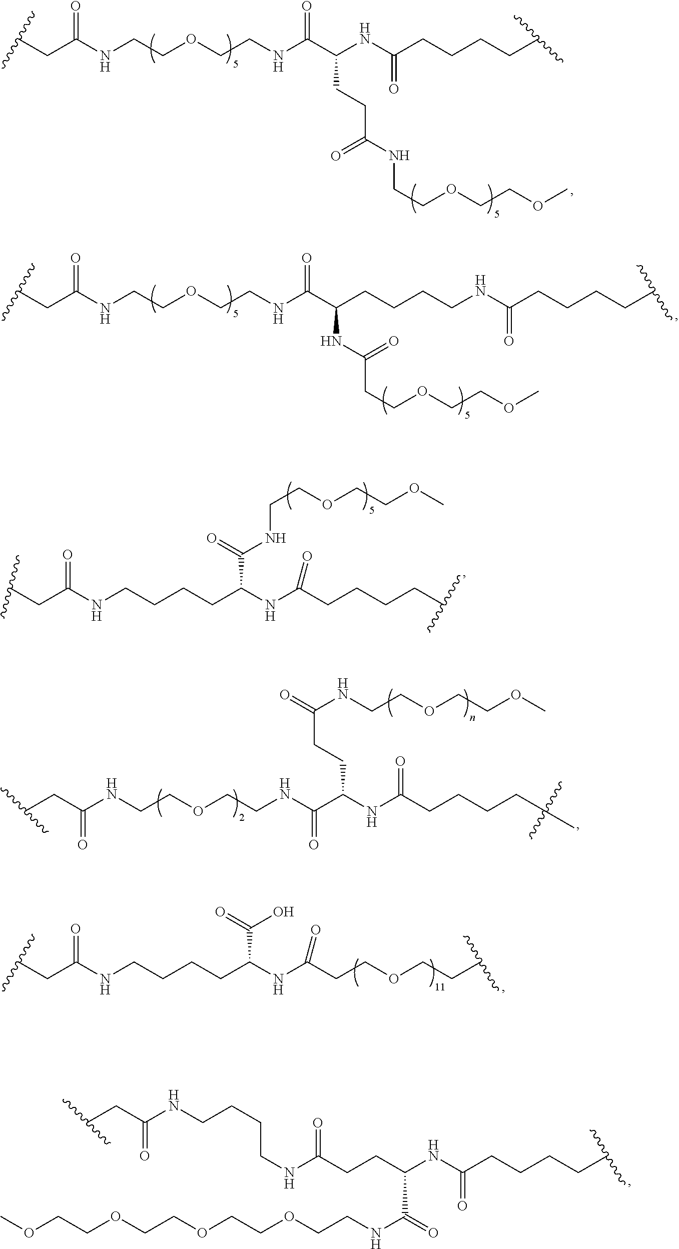

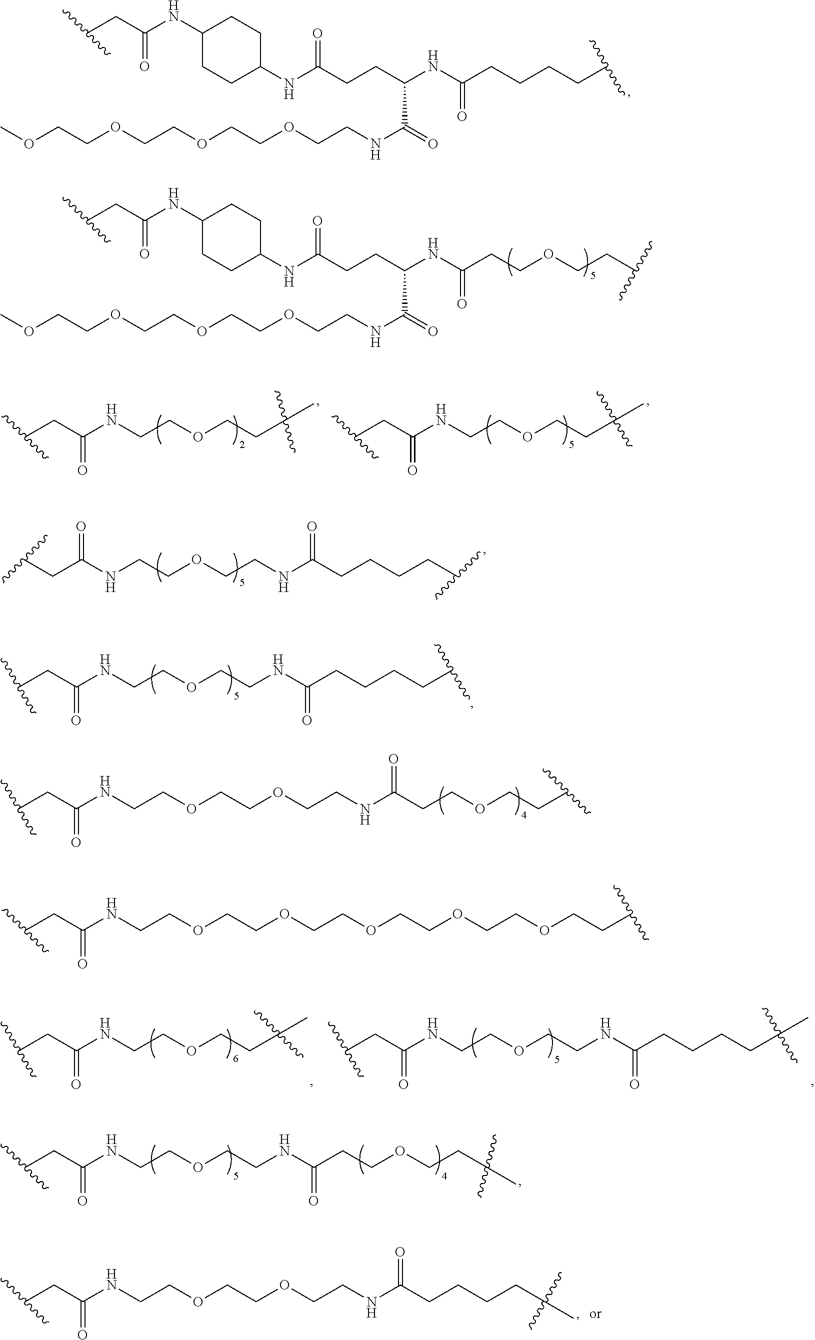

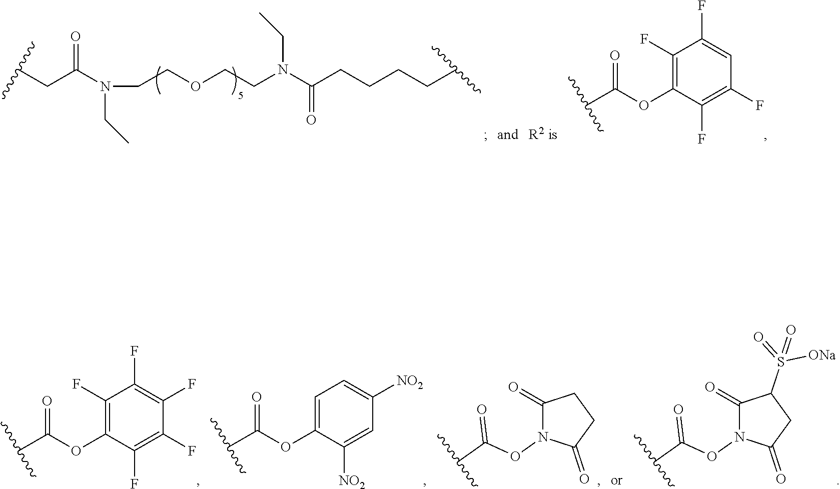

82. The compound of claim 72, or a pharmaceutically acceptable salt thereof, wherein —X¹-L- is —O—CH₂C(═O)NH-L- and —CH₂C(═O)NH-L- is:

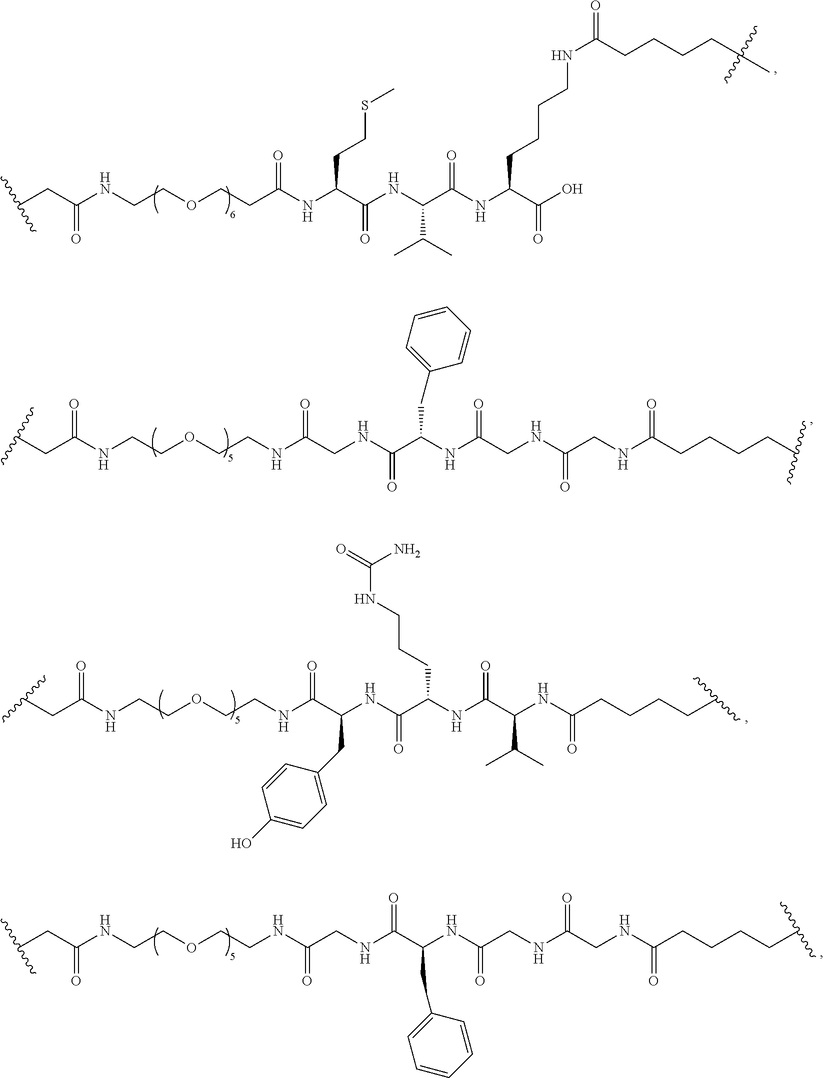

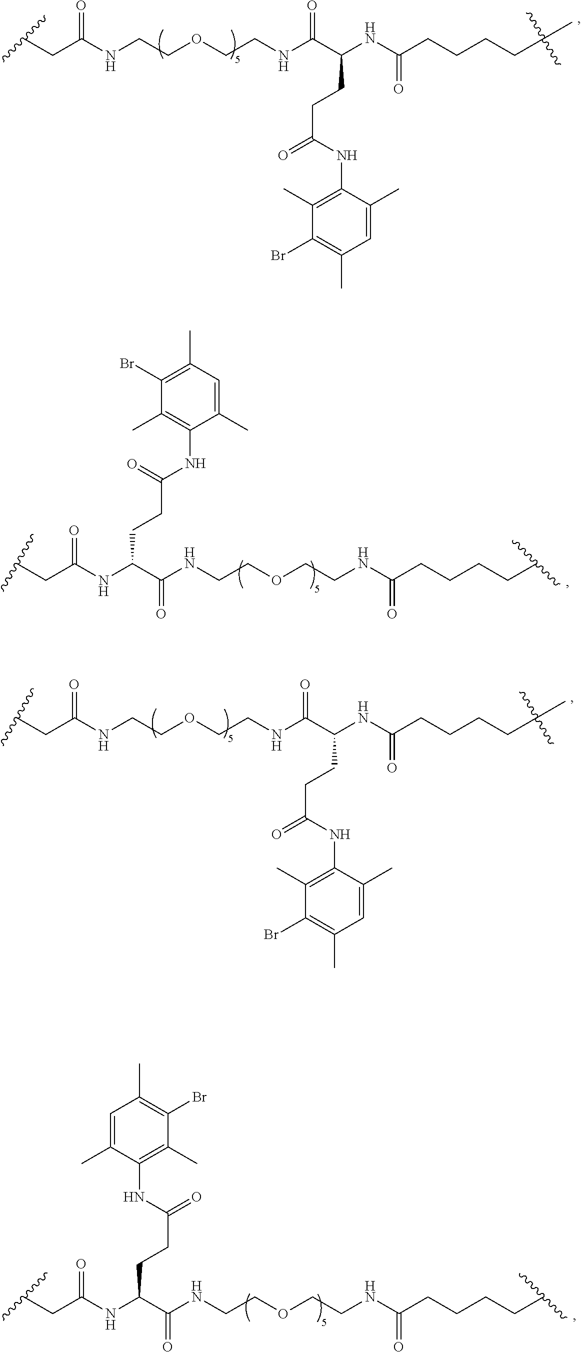

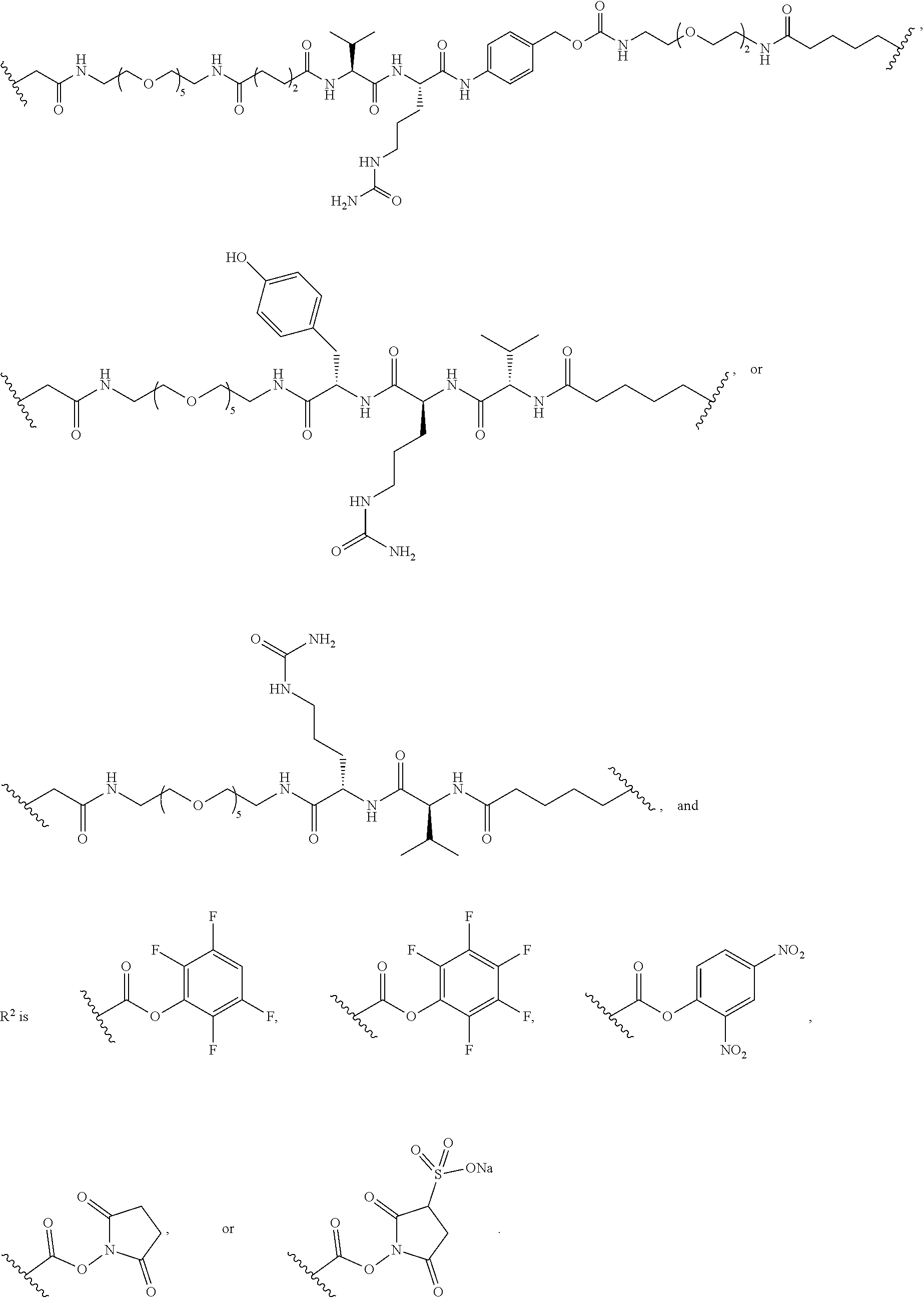

83. The compound of claim 72, or a pharmaceutically acceptable salt thereof, wherein X¹ is —O-L- and —O-L- is:

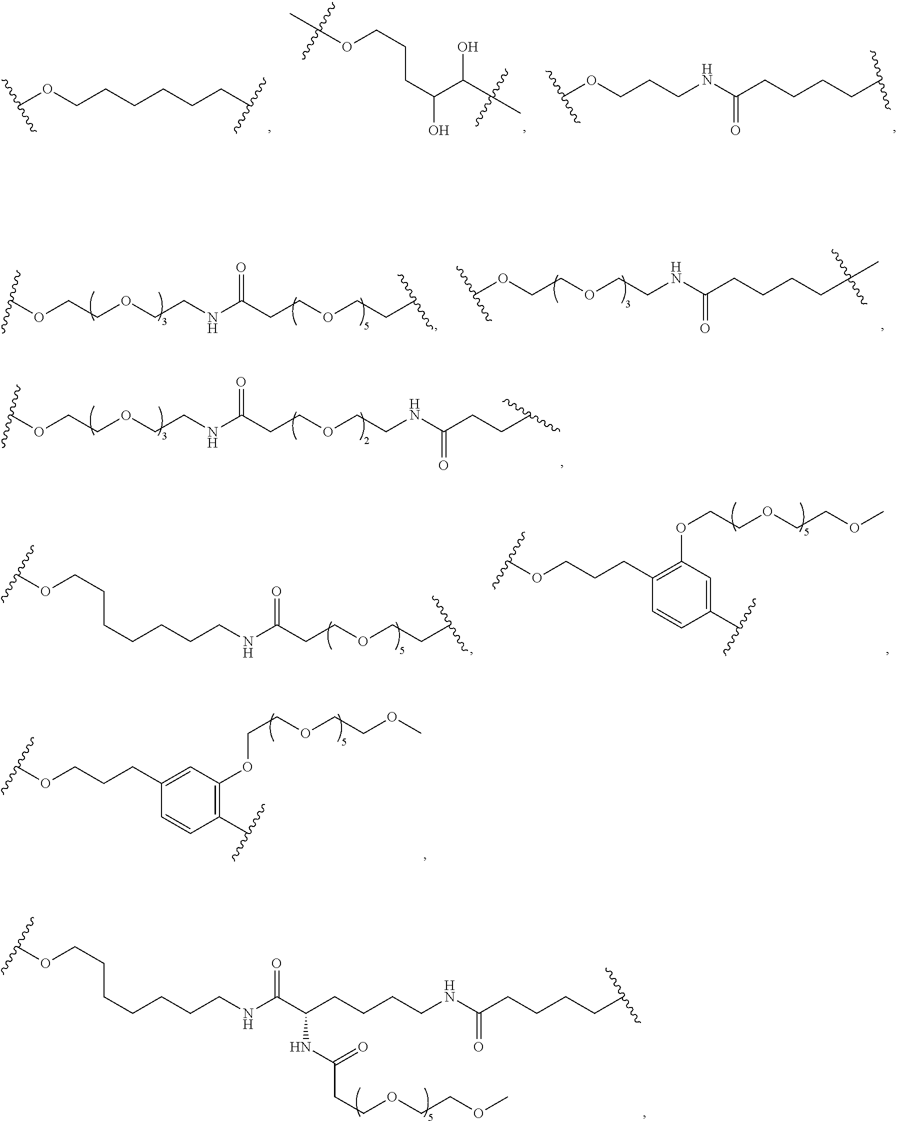

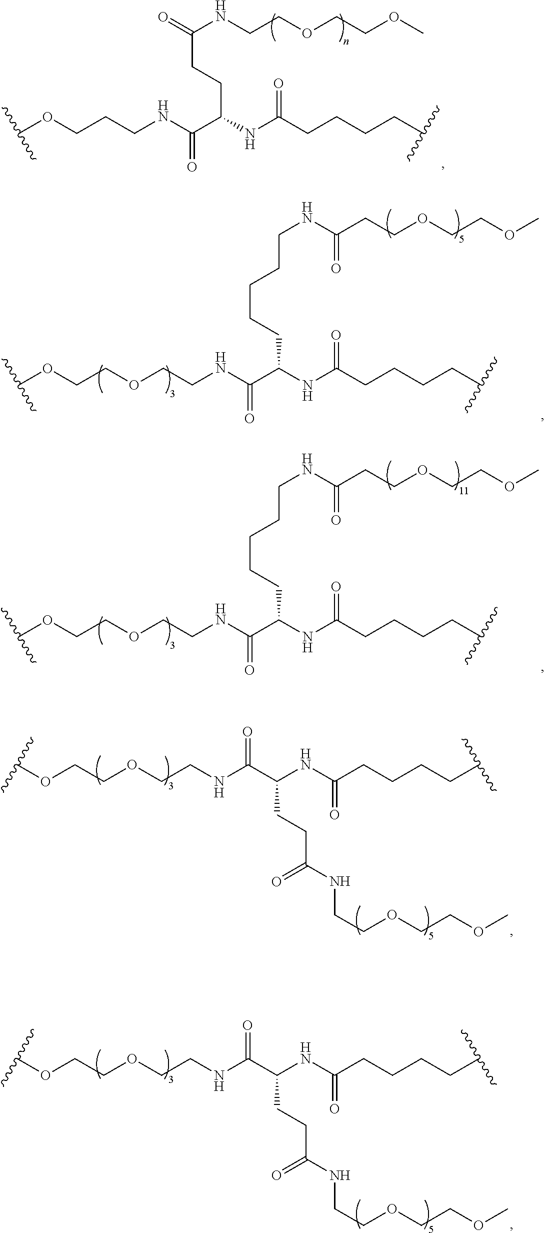

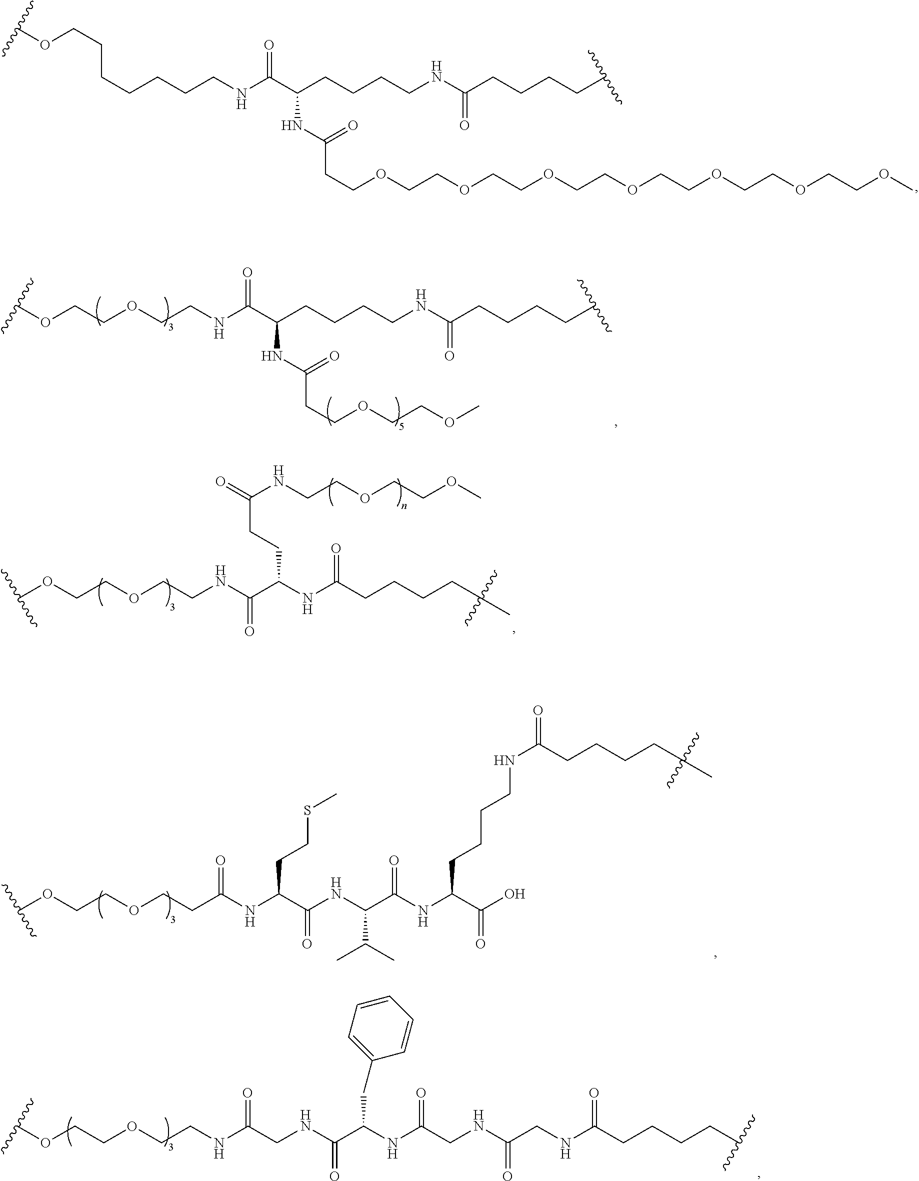

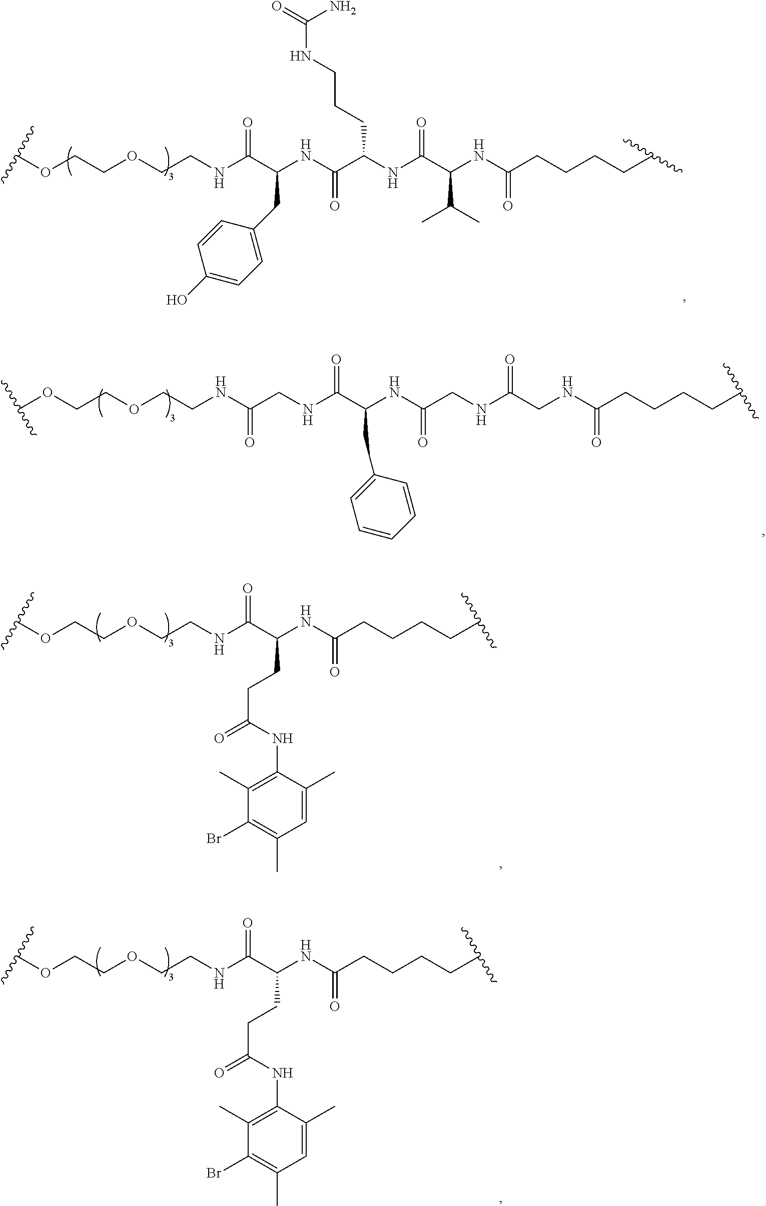

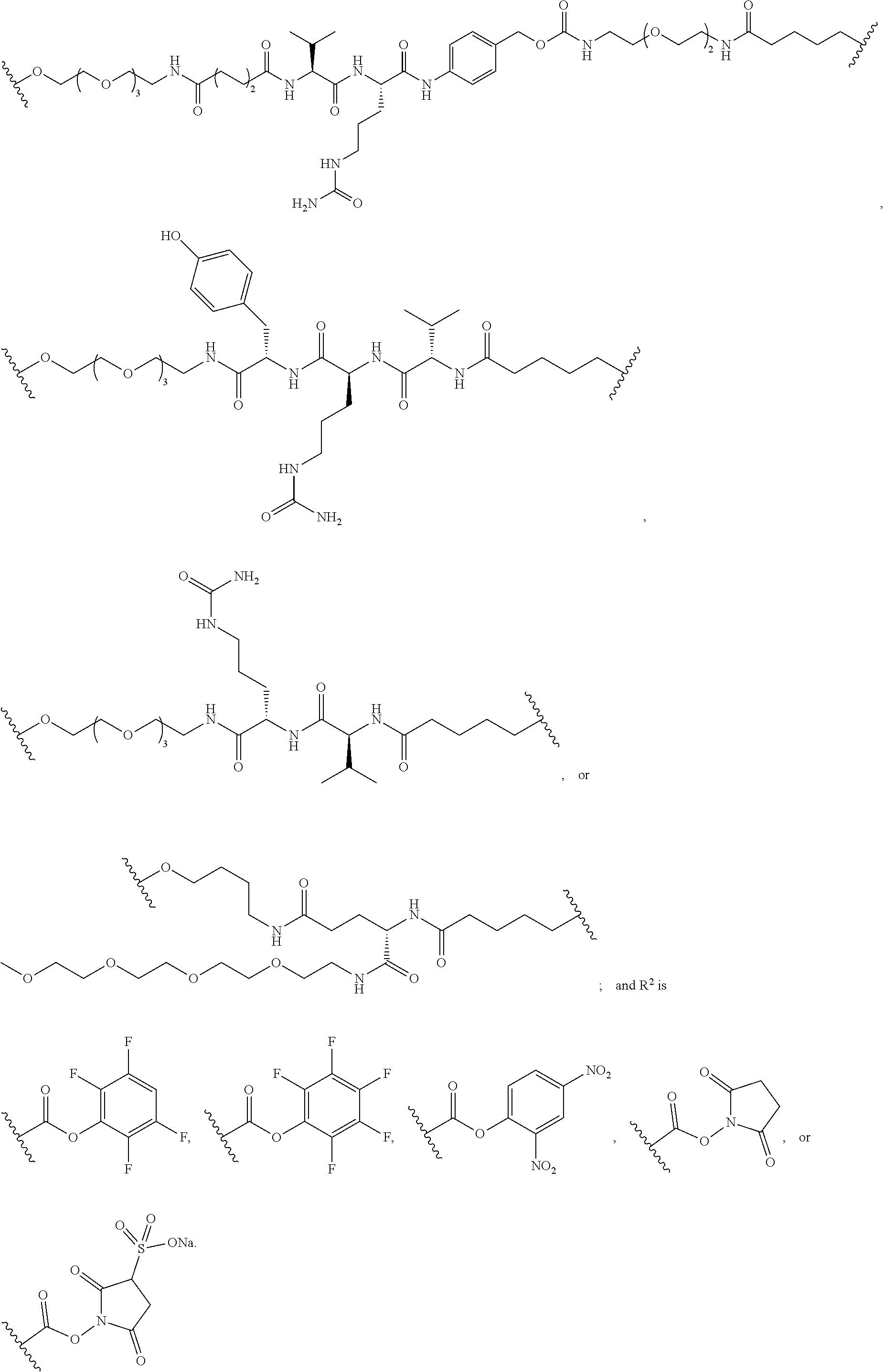

84. The compound of claim 72, or a pharmaceutically acceptable salt thereof, wherein, the radionuclide complex comprises a radionuclide that is an Auger electron-emitting radionuclide, α-emitting radionuclide, β-emitting radionuclide, or γ-emitting radionuclide; wherein
the α-emitting radionuclide is actinium-225 (²²⁵Ac), bismuth-213 (²¹³Bi), radium-223 (²²³Ra), or lead-212 (²¹²Pb); or
the a β-emitting radionuclide is yttrium-90 (⁹⁰Y), lutetium-177 (¹⁷⁷Lu), rhenium-186 (¹¹⁶Re), rhenium-188 (¹⁸⁸Re), copper-64 (⁶⁴Cu), copper-67 (⁶⁷Cu), samarium-153 (¹⁵³Sm), strontium-89 (⁸⁹Sr), gold-198 (¹⁹⁸Au), erbium (¹⁶⁹Er), dysprosium-165 (¹⁶⁵Dy), technetium-99m (^{99m}Tc), zirconium-89 (⁸⁹Zr), or manganese-52 (⁵²Mn); or
the γ-emitting radionuclide is cobalt-60 (⁶⁰Co), palldium-103 (¹⁰³Pd), cesium-137 (¹³⁷Cs), ytterbium-169 (¹⁶⁹Yb), iridium-192 (¹⁹²Ir), or radium-226 (²²⁶Ra).

85. A pharmaceutical composition comprising a compound of claim 72, or a pharmaceutically acceptable salt thereof, and at least one pharmaceutically acceptable excipient.

86. A method of treating a tumor in a mammal comprising administering to the mammal an immunoconjugate comprising a tumor targeting polypeptide linked to the compound of claim 72 with a lysine residue of tumor targeting polypeptide, thereby treating the tumor; wherein compound of claim 72 comprises a therapeutic α-emitting radionuclide or a therapeutic β-emitting radionuclide

87. A method for the in vivo imaging of tissues or organs in a mammal with tumors comprising:
i) administering to the mammal an immunoconjugate comprising a tumor targeting polypeptide linked to the compound of claim 72 with a lysine residue of tumor targeting polypeptide, or a pharmaceutically acceptable salt thereof;
wherein the compound of claim 72 comprises a diagnostic radionuclide that is indium-111 (¹¹¹In), gallium-67 (⁶⁷Ga), gallium-68 (⁶⁸Ga), technetium-99m (^{99m}Tc), or platinum-195m (^{195m}Pt); and
ii) performing positron emission tomography (PET) analysis, single-photon emission computerized tomography (SPECT), or magnetic resonance imaging (MRI);
wherein the tumor cells express an antigen specifically bound by the antigen binding region of the tumor targeting polypeptide R³.

88. The method of claim 87, wherein: step (ii) is initiated after an amount of time following step (i) sufficient for interaction between the compound of claim 72, or a pharmaceutically acceptable salt thereof, the antigen expressed on the tumor cells.

89. The compound of claim 72, or a pharmaceutically acceptable salt thereof, wherein
X¹ is —OCH₂—, —OCH₂—X²—, —OCH₂CH₂— or —OCH₂CH₂—X²;
X² is —C(═O)X⁴—;
X⁴ is —NH—, —N(CH₃)—, or —N(CH₂CH₃);
R² is

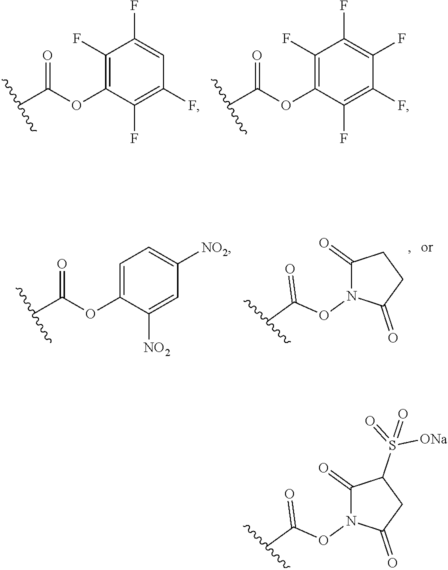

90. The method of claim 18, or a pharmaceutically acceptable salt thereof, wherein
L¹ is unsubstituted or substituted C₁-C₆alkylene, unsubstituted or substituted C₁-C₁₀heteroalkylene, C₄-C₂₀polyethylene glycol, unsubstituted or substituted cyclohexylene, or unsubstituted or substituted phenylene:
L² is absent;
L³ is absent;
L⁴ is absent;
L⁵ is —NR⁴C(═O)—(CH₂)ₙ—, —C(═O)—(CH₂CH₂O)ₙ—(CH₂)_{q}—, —C(═O)NR⁴—(CH₂CH₂O)ₙ—(CH₂)_{q}—, or —NR⁴C(═O)—(CH₂CH₂O)ₙ—(CH₂)_{q}—;
each n is independently 1, 2, 3, 4, 5, or 6;
each q is independently 1 or 2.

91. The compound of claim 72, or a pharmaceutically acceptable salt thereof, wherein —X¹-L- is —O—CH₂C(═O)NH-L- and —CH₂C(═O)NH-L- is: 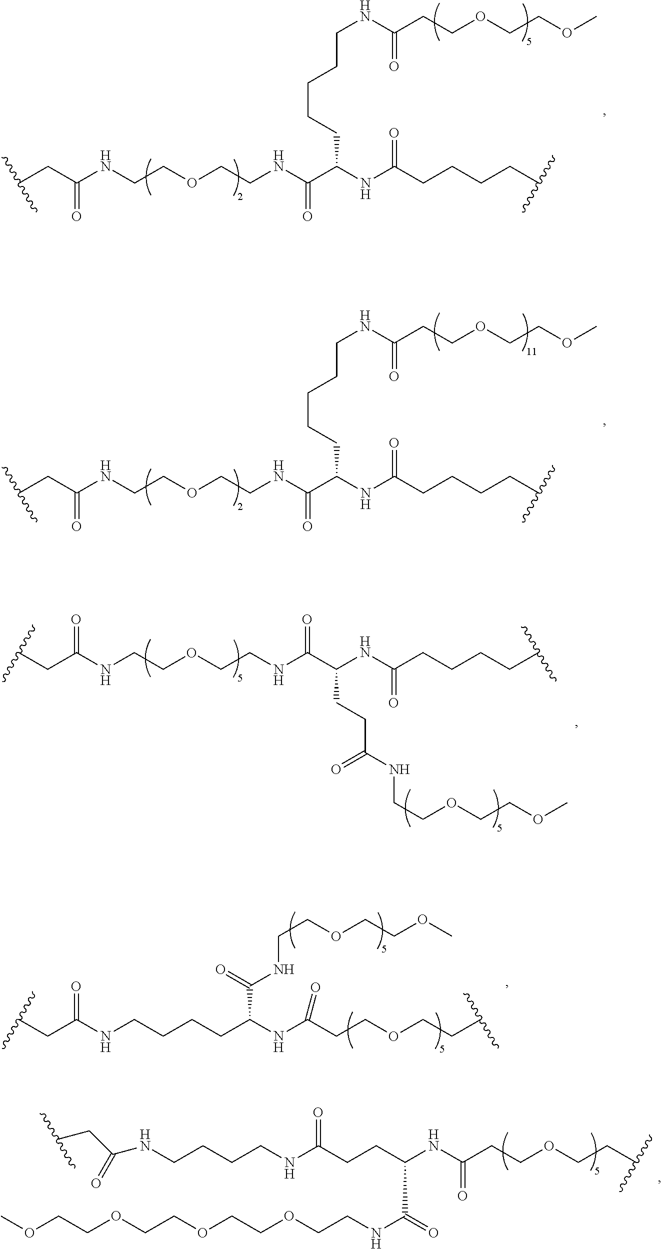 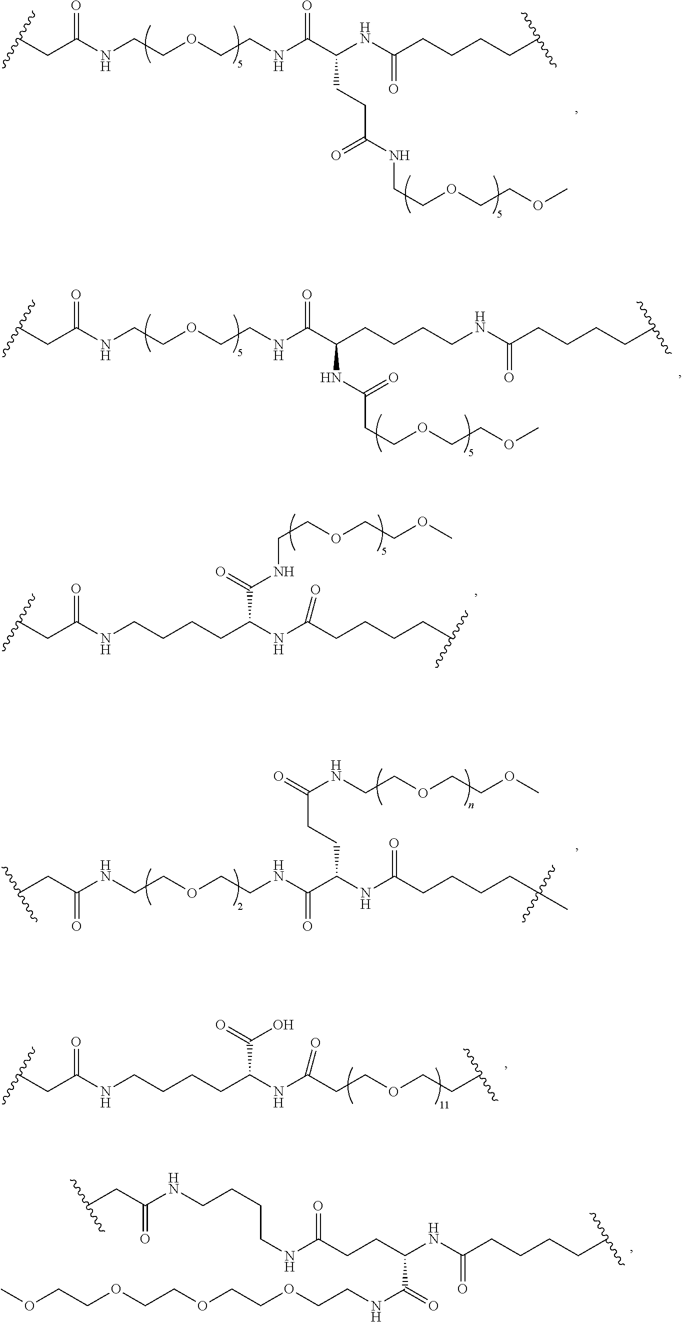 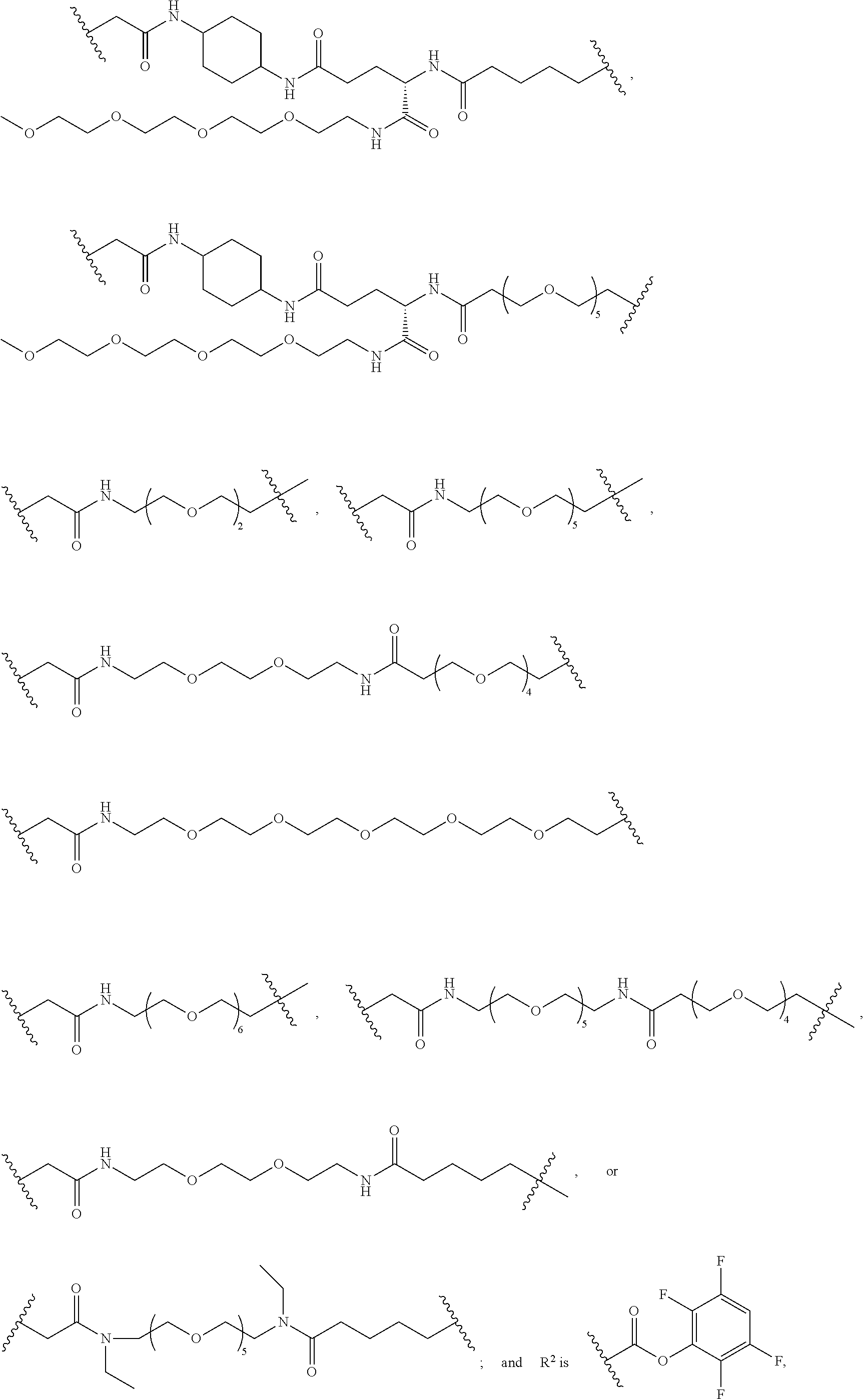 [chemical structure]
